(19) United States
(12) Patent Application Publication
(10) Pub. No.: US 2025/0057202 A1
(43) Pub. Date: Feb. 20, 2025

(54) OLIGOSACCHARIDE COMPOSITIONS FOR USE AS FOOD INGREDIENTS AND METHODS OF PRODUCING THEREOF

(71) Applicant: DSM Nutritional Products, LLC, Parsippany, NJ (US)

(72) Inventors: John M. GEREMIA, Watertown, MA (US); Raffi MARDIROSIAN, Bedford, MA (US); Michael J. GIDDING, Bedford, MA (US)

(21) Appl. No.: 18/933,558

(22) Filed: Oct. 31, 2024

Related U.S. Application Data
(63) Continuation of application No. 17/576,161, filed on Jan. 14, 2022.
Application No. 17/576,161 is a continuation of application No. 16/440,261, filed on Jun. 13, 2019, now abandoned.
Application No. 16/440,261 is a continuation of application No. 15/546,438, filed on Jul. 26, 2017, now abandoned.
(60) Provisional application No. 62/108,036, filed on Jan. 26, 2015.

(51) Int. Cl. A23L 33/125; A21D 2/18; A23L 7/10; A23L 29/30

(52) U.S. Cl. CPC A23L 33/125; A21D 2/181; A23L 7/10; A23L 29/30; A23V 2002/00

(57) ABSTRACT

Described herein are food ingredients made up of oligosaccharide compositions, and methods of producing such food ingredients, as well as methods of using such food ingredients in food products. The present application addresses this need in the art by providing oligosaccharide compositions that have similar physical characteristics to commercially available carbohydrate sources, such as fiber, but lower metabolic energy. Methods of producing such oligosaccharide compositions suitable for use in food products are also provided herein.

### CROSS-REFERENCE TO RELATED APPLICATIONS

This application claims priority to U.S. Provisional Patent Application No. 62/108,036 filed Jan. 26, 2015, the disclosure of which is hereby incorporated by reference in its entirety.

### FIELD

The present disclosure relates generally to food ingredients suitable for human consumption, and more specifically to food ingredients made up of oligosaccharide compositions, as well as methods of using such food ingredients in various food products and methods of producing such oligosaccharide compositions, food ingredients and food products.

### BACKGROUND

Food products often contain a variety of carbohydrates, including various sugars and starches. Several of these carbohydrates are digested by humans in the stomach and small intestine. In contrast, dietary fiber is often not digested in the stomach or small intestine, but can be fermented by microorganisms in the large intestine. Some dietary fibers have health benefits, including for example aiding the passage of food through the digestive tract. Furthermore, some complex carbohydrates, including certain oligosaccharides that are not digestible by humans, contribute little or no caloric value to food products.

A commercial interest exists to replace a portion of the raw sugar ingredients in food products with oligosaccharides to reduce the caloric content of those food products. Oligosaccharides can also be added to food products to impart favorable flavor, mouth feel, and consistency. The functional performance of oligosaccharides, including the effect on food texture, digestibility, and health effects, depend on the particular structure or range of structural properties of the oligosaccharides. Thus, there is a need in the art for compositions suitable for human consumption that have a reduced content of easily digestible carbohydrates.

### BRIEF SUMMARY

The present application addresses this need in the art by providing oligosaccharide compositions that have similar physical characteristics to commercially available carbohydrate sources, such as fiber, but lower metabolic energy. Methods of producing such oligosaccharide compositions suitable for use in food products are also provided herein.

In one aspect, provided is a food ingredient that includes an oligosaccharide composition, wherein:
(a) the oligosaccharide composition has a glycosidic bond type distribution of:
   at least 10 mol % α-(1,3) glycosidic linkages; and
   at least 10 mol % β-(1,3) glycosidic linkages; and
(b) at least 10 dry wt % of the oligosaccharide composition has a degree of polymerization of at least 3; and
(c) a metabolizable energy content, on a dry matter basis, of less than 4 kcal/g.

In some variations, the metabolizable energy content, on a dry matter basis, is less than 2.7 kcal/g, or less than 2 kcal/g, or less than 1.5 kcal/g; or between 1 kcal/g and 2.7 kcal/g, or between 1.1 kcal/g and 2.5 kcal/g, or between 1.1 and 2 kcal/g.

In some embodiments, the oligosaccharide composition has a glycosidic bond type distribution of less than 9 mol % α-(1,4) glycosidic linkages, and less than 19 mol % α-(1,6) glycosidic linkages.

In another aspect, provided is a food ingredient that includes an oligosaccharide composition, wherein:
(a) the oligosaccharide composition has a glycosidic bond type distribution of:
   less than 9 mol % α-(1,4) glycosidic linkages; and
   less than 19 mol % α-(1,6) glycosidic linkages; and
(b) at least 10 dry wt % of the oligosaccharide composition has a degree of polymerization of at least 3; and
(c) a metabolizable energy content, on a dry matter basis, of less than 4 kcal/g.

In some variations, the metabolizable energy content, on a dry matter basis, is less than 2.7 kcal/g, or less than 2 kcal/g, or less than 1.5 kcal/g; or between 1 kcal/g and 2.7 kcal/g, or between 1.1 kcal/g and 2.5 kcal/g, or between 1.1 and 2 kcal/g.

In some variations, the oligosaccharide composition has a glycosidic bond type distribution of at least 15 mol % β-(1,2) glycosidic linkages.

Provided is also a food product that incorporates the food ingredient described herein. Examples of suitable food products include a breakfast cereal, granola, yogurt, ice cream, bread, cookie, candy, cake mix, a nutritional shake, or a nutritional supplement.

In other aspects, provided is a method of producing a polished oligosaccharide composition, by: combining feed sugar with a catalyst to form a reaction mixture; producing an oligosaccharide composition from at least a portion of the reaction mixture; and polishing the oligosaccharide composition to produce a polished oligosaccharide composition. Such polished oligosaccharide composition can be incorporated into a food ingredient or a food product.

In another aspect, provided is a method of producing a food ingredient, by: combining feed sugar with a catalyst to form a reaction mixture; producing an oligosaccharide composition from at least a portion of the reaction mixture; polishing the oligosaccharide composition to produce a polished oligosaccharide composition; and forming a food ingredient from the polished oligosaccharide composition.

In yet another aspect, provided is a method of manufacturing a food product, by: combining a food ingredient produced according to any of the methods described herein with other ingredients to manufacture a food product. In one variation, provided is a method of manufacturing a food product, by: producing a polished oligosaccharide composition according to any of the methods described herein; and combining the polished oligosaccharide composition with other food ingredients to manufacture a food product.

In yet another aspect, provided is an oligosaccharide composition for use as a food ingredient or for use in a food product, wherein the oligosaccharide composition is produced by: combining feed sugar with a catalyst to form a reaction mixture; and producing the oligosaccharide composition from at least a portion of the reaction mixture.

In some embodiments of the foregoing aspects, the catalyst is a polymeric catalyst that includes acidic monomers and ionic monomers connected to form a polymeric backbone; or the catalyst is a solid-supported catalyst that includes a solid support, acidic moieties attached to the solid support, and ionic moieties attached to the solid support.

Provided is a polished oligosaccharide composition produced according to any of the methods described herein. Provided is also a food ingredient or a food product produced according to any of the methods described herein.

### DESCRIPTION OF THE FIGURES

The present application can be understood by reference to the following description taken in conjunction with the accompanying figures.

FIG. 1 depicts an exemplary process to produce an oligosaccharide composition from sugars in the presence of a catalyst.

FIG. 2A illustrates a portion of a catalyst with a polymeric backbone and side chains.

FIG. 2B illustrates a portion of an exemplary catalyst, in which a side chain with the acidic group is connected to the polymeric backbone by a linker and in which a side chain with the cationic group is connected directly to the polymeric backbone.

FIG. 3 depicts a reaction scheme to prepare a dual-functionalized catalyst from an activated carbon support, in which the catalyst has both acidic and ionic moieties.

FIG. 4 illustrates a portion of a polymeric catalyst, in which the monomers are arranged in blocks of monomers, and the block of acidic monomers alternates with the block of ionic monomers.

FIG. 5A illustrates a portion of a polymeric catalyst with cross-linking within a given polymeric chain.

FIG. 5B illustrates a portion of a polymeric catalyst with cross-linking within a given polymeric chain.

FIG. 6A illustrates a portion of a polymeric catalyst with cross-linking between two polymeric chains.

FIG. 6B illustrates a portion of a polymeric catalyst with cross-linking between two polymeric chains.

FIG. 6C illustrates a portion of a polymeric catalyst with cross-linking between two polymeric chains.

FIG. 6D illustrates a portion of a polymeric catalyst with cross-linking between two polymeric chains.

FIG. 7 illustrates a portion of a polymeric catalyst with a polyethylene backbone.

FIG. 8 illustrates a portion of a polymeric catalyst with a polyvinylalcohol backbone.

FIG. 9 illustrates a portion of a polymeric catalyst, in which the monomers are randomly arranged in an alternating sequence.

FIG. 10 illustrates two side chains in a polymeric catalyst, in which there are three carbon atoms between the side chain with the Bronsted-Lowry acid and the side chain with the cationic group.

FIG. 11 illustrates two side chains in a polymeric catalyst, in which there are zero carbons between the side chain with the Bronsted-Lowry acid and the side chain with the cationic group.

FIG. 12 illustrates a portion of a polymeric catalyst with an ionomeric backbone.

FIG. 13 depicts a graph showing the glass transition temperature (Tg) at different moisture contents for various oligosaccharides produced according to the methods described herein, compared to oligosaccharides produced by other methods.

FIG. 14 depicts a graph showing the moisture content at different water activity values for various oligosaccharides produced according to the methods described herein, compared to oligosaccharides produced by other methods.

FIG. 15 is a graph depicting the changes in distribution of degree of polymerization over time of corn syrup during refactoring with a catalyst with both acidic and ionic moieties.

FIG. 16 depicts an exemplary process to produce a functionalized oligosaccharide composition, wherein a portion of an oligosaccharide comprising pendant functional groups and bridging functional groups is shown.

### DETAILED DESCRIPTION

The following description sets forth exemplary methods, parameters and the like. It should be recognized, however, that such description is not intended as a limitation on the scope of the present disclosure but is instead provided as a description of exemplary embodiments.

In some aspects, provided herein are food ingredients made up of oligosaccharide compositions. Such food ingredients have same or similar physical characteristics to commercially available carbohydrate sources, such as fiber, but have lower metabolic energy. Such food ingredients may be incorporated to various food products, and are suitable for use as lower energy substrates having application in food products where lower caloric ingredients are desired.

In other aspects, provided herein are methods of producing oligosaccharide compositions suitable for use as food ingredients. Such methods described herein use catalysts that have acidic and ionic groups. In some variations, the oligosaccharide compositions produced by such methods have a reduced content of easily digestible carbohydrates, and are slowly digestible by the human digestive system. Thus, such oligosaccharide compositions may be used to enhance dietary fiber content and/or reduce the caloric content of food for human consumption.

The food ingredients, including the oligosaccharide compositions, and the method of producing thereof are described in further detail below.

### Food Ingredients

As used herein, “food ingredient” refers to any substance used in the production, processing, treatment, packaging, transportation or storage of food. In certain embodiments, a food ingredient may be a substance incorporated into food to maintain of improve safety and freshness, improve or maintain nutritional value, or to improve the taste, texture, or appearance of the food. The food ingredients provided herein are made up of oligosaccharide compositions. The oligosaccharide compositions may be produced according to the methods described herein, and the properties of such compositions may vary depending on the type of sugars as well as the reaction conditions used. The oligosaccharide compositions may be characterized based on the type of oligosaccharides present, degree of polymerization, digestibility (e.g., by the human digestive system), glass transition temperature, hygroscopicity, fiber content, glycosidic bond type distribution, and metabolizable energy content.

### Oligosaccharide Composition

In some embodiments, the oligosaccharide compositions include an oligosaccharide comprising one type of sugar monomer. For example, in some embodiments, the oligosaccharide compositions may include a gluco-oligosaccharide, a galacto-oligosaccharide, a fructo-oligosaccharide, a manno-oligosaccharide, an arabino-oligosaccharide, or a xylo-oligosaccharide, or any combinations thereof. In some embodiments, the oligosaccharide compositions include an oligosaccharide comprising two different types of sugar monomers. For example, in some embodiments, the oligosaccharide compositions may include a gluco-galacto-oligosaccharide, a gluco-fructo-oligosaccharide, a gluco-manno-oligosaccharide, a gluco-arabino-oligosaccharide, a gluco-xylo-oligosaccharide, a galacto-fructo-oligosaccharide, a galacto-manno-oligosaccharide, a galacto-arabino-oligosaccharide, a galacto-xylo-oligosaccharide, a fructo-manno-oligosaccharide, a fructo-arabino-oligosaccharide, a fructo-xylo-oligosaccharide, a manno-arabino-oligosaccharide, a manno-xylo-oligosaccharide, or an arabino-xylo-oligosaccharide, or any combinations thereof. In some embodiments, the oligosaccharide compositions include an oligosaccharide comprising more than two different types of sugar monomers. In some variations, the oligosaccharide compositions include an oligosaccharide comprising 3, 4, 5, 6, 7, 8, 9, or 10 different types of sugar monomers. For example, in certain variations the oligosaccharide compositions include an oligosaccharide comprising a galacto-arabino-xylo-oligosaccharide, a fructo-galacto-xylo-oligosaccharide, a arabino-fructo-manno-xylo-oligosaccharide, a gluco-fructo-galacto-arabino-oligosaccharide, a fructo-gluco-arabino-manno-xylo oligosaccharide, or a gluco-galacto-fructo-manno-arabino-xylo-oligosaccharide.

In some embodiments, the oligosaccharide compositions include a gluco-oligosaccharide, a manno-oligosaccharide, a gluco-galacto-oligosaccharide, a xylo-oligosaccharide, an arabino-galacto-oligosaccharide, a gluco-galacto-xylo-oligosaccharide, an arabino-xylo-oligosaccharide, a gluco-xylo-oligosaccharide, or a xylo-gluco-galacto-oligosaccharide, or any combinations thereof. In one variation, the oligosaccharide compositions include a gluco-galacto-oligosaccharide. In another variation, the oligosaccharide compositions include a xylo-gluco-galacto-oligosaccharide.

As used herein, “oligosaccharide” refers to a compound containing two or more monosaccharide units linked by glycosidic bonds.

In some embodiments, at least one of the two or more monosaccharide units is a sugar in L-form. In other embodiments, at least one of the two or more monosaccharides is a sugar in D-form. In yet other embodiments, the two or more monosaccharide units are sugars in L- or D-form according to their naturally-abundant form (e.g., D-glucose, D-xylose, L-arabinose).

In some embodiments, the oligosaccharide composition comprises a mixture of L- and D-forms of monosaccharide units, e.g. of a ratio, such as: 1:1, 1:2, 1:3, 1:4, 1:5, 1:6, 1:7, 1:8, 1:9, 1:10, 1:12, 1:14, 1:16, 1:18, 1:20, 1:25, 1:30, 1:35, 1:40, 1:45, 1:50, 1:55, 1:60, 1:65, 1:70, 1:75, 1:80, 1:85, 1:90, 1:100, 1:150 L- to D-forms or D- to L-forms. In some embodiments, the oligosaccharide comprises monosaccharide units with substantially all L- or D-forms of glycan units, optionally comprising 1%, 2%, 3%, 4% 5%, 6%, 7%, 8%, 9%, 10%, 11%, 12%, 13%, 14%, 15%, 16%, 17%, 18%, 19%, or 20% of the respective other form.

As used herein, “gluco-oligosaccharide” refers to a compound containing two or more glucose monosaccharide units linked by glycosidic bonds. Similarly, “galacto-oligosaccharide” refers to a compound containing two or more galactose monosaccharide units linked by glycosidic bonds.

As used herein, “gluco-galacto-oligosaccharide” refers to a compound containing one or more glucose monosaccharide units linked by glycosidic bonds, and one or more galactose monosaccharide units linked by glycosidic bonds. In some embodiments, the ratio of glucose to galactose on a dry mass basis is between 10:1 glucose to galactose to 0.1:1 glucose to galactose, 5:1 glucose to galactose to 0.2:1 glucose to galactose, 2:1 glucose to galactose to 0.5:1 glucose to galactose. In one embodiment, the ratio of glucose to galactose is 1:1.

In one variation, the oligosaccharide composition is a long oligosaccharide composition, while in another variation the oligosaccharide composition is a short oligosaccharide composition. As used herein, the term “long oligosaccharide composition” refers to an oligosaccharide composition with an average degree of polymerization (DP) of about 8, about 9, about 10, about 11, about 12, about 13, about 14, about 15, about 16, about 17, about 18, about 19, or about 20. As used herein, the term “short oligosaccharide composition” refers to oligosaccharide composition with an average DP of about 2, about 3, about 4, about 5, about 6, or about 7.

### Functionalized Oligosaccharide Compositions

In some variations, the oligosaccharide compositions described herein are functionalized oligosaccharide compositions. Functionalized oligosaccharide compositions may be produced by, for example, combining one or more sugars (e.g., feed sugars) with one or more functionalizing compounds in the presence of a catalyst, including, for example, polymeric catalysts and solid-supported catalysts as described in WO 2012/118767 and WO 2014/031956. In certain variations, a functionalized oligosaccharide is a compound comprising two or more monosaccharide units linked by glycosidic bonds in which one or more hydroxyl groups in the monosaccharide units are independently replaced by a functionalizing compound, or comprise a linkage to a functionalizing compound. The functionalizing compound may be a compound that can attach to the oligosaccharide through an ether, ester, oxygen-sulfur, amine, or oxygen-phosphorous bond, and which does not contain a monosaccharide unit.

### Functionalizing Compounds

In certain variations, the functionalizing compound comprises one or more functional groups independently selected from amine, hydroxyl, carboxylic acid, sulfur trioxide, sulfate, and phosphate. In some variations, one or more functionalizing compounds are independently selected from the group consisting of amines, alcohols, carboxylic acids, sulfates, phosphates, or sulfur oxides.

In some variations, the functionalizing compound has one or more hydroxyl groups. In some variations, the functionalizing compound with one or more hydroxyl groups is an alcohol. Such alcohols may include, for example, alkanols and sugar alcohols.

In certain variations, the functionalizing compound is an alkanol with one hydroxyl group. For example, in some variations, the functionalizing compound is selected from ethanol, propanol, butanol, pentanol, and hexanol. In other variations, the functionalizing compound has two or more hydroxyl groups. For example, in some variations, the functionalizing compound is selected from propanediol, butanediol, and pentanediol.

For example, in one variation, one or more sugars (e.g., feed sugars) may be combined with a sugar alcohol in the presence of a polymeric catalyst to produce a functionalized oligosaccharide composition. Suitable sugar alcohols may include, for example, sorbitol (also known as glucitol), xylitol, lacitol, arabinatol (also known as arabitol), glycerol, erythritol, mannitol, galacitol, fucitol, iditol, inositol, or volemitol, or any combinations thereof.

In another variation, wherein the functionalizing compound comprises a hydroxyl group, the functionalizing compound may become attached to the monosaccharide unit through an ether bond. The oxygen of the ether bond may be derived from the monosaccharide unit, or from the functionalizing compound.

In yet other variations, the functionalizing compound comprises one or more carboxylic acid functional groups. For example, in some variations, the functionalizing compound is selected from lactic acid, acetic acid, citric acid, pyruvic acid, succinic acid, glutamic acid, itaconic acid, malic acid, maleic acid, propionic acid, butanoic acid, pentanoic acid, hexanoic acid, adipic acid, isobutyric acid, formic acid, levulinic acid, valeric acid, and isovaleric acid. In other variations, the functionalizing compound is a sugar acid. For example, in one embodiment, the functionalizing compound is gluconic acid. In certain variations, wherein the functionalizing compound comprises a carboxylic acid group, the functionalizing compound may become attached to the monosaccharide unit through an ester bond. The non-carbonyl oxygen of the ester bond may be derived from the monosaccharide unit, or from the functionalizing compound.

In still other variations, the functionalizing compound comprises one or more amine groups. For example, in some variations, the functionalizing compound is an amino acid, while in other variations the functionalizing compound is an amino sugar. In one variation, the functionalizing compound is selected from glutamic acid, aspartic acid, glucosamine and galactosamine. In certain variations, wherein the functionalizing compound comprises an amine group, the functionalizing compound may become attached to the monosaccharide unit through an amine bond.

In yet other variations, the functionalizing compound comprises a sulfur trioxide group or a sulfate group. For example, in one variation, the functionalizing compound is dimethylformamide sulfur trioxide complex. In another variation, the functionalizing compound is sulfate. In one embodiment, the sulfate is produced in situ, from, for example, sulfur trioxide. In certain variations wherein the functionalizing compound comprises a sulfur trioxide or sulfate group, the functionalizing compound may become attached to the monosaccharide unit through an oxygen-sulfur bond.

In still other variations, the functionalizing compound comprises a phosphate group. In certain variations wherein the functionalizing compound comprises a phosphate group, the functionalizing compound may become attached to the monosaccharide unit through an oxygen-phosphorous bond.

It should be understood that the functionalizing compounds described herein may contain a combination of functional groups. For example, the functionalizing compound may comprise one or more hydroxyl groups and one or more amine groups (for example, amino sugars). In other embodiments, the functionalizing compound may comprise one or more hydroxyl groups and one or more carboxylic acid groups (for example, sugar acids). In yet other embodiments, the functionalizing compound may comprise one or more amine groups and one or more carboxylic acid groups (for example, amino acids). In still other embodiments, the functionalizing compound comprises one or more additional functional groups, such as esters, amides, and/or ethers. For example, in certain embodiments, the functionalizing compound is a sialic acid (for example, N-acetylneuraminic acid, 2-keto-3-deoxynonic acid, and other N- or O-substituted derivatives of neuraminic acid).

It should further be understood that a functionalizing compound may belong to one or more of the groups described above. For example, a glutamic acid is both an amine and a carboxylic acid, and a gluconic acid is both a carboxylic acid and an alcohol.

In some variations, the functionalizing compound forms a pendant group on the oligosaccharide. In other variations, the functionalizing compound forms a bridging group between an oligomer backbone and a second oligomer backbone; wherein each oligomer backbone independently comprises two or more monosaccharide units linked by glycosidic bonds; and the functionalizing compound is attached to both backbones. In other variations, the functionalizing compound forms a bridging group between an oligomer backbone and a monosaccharide; wherein the oligomer backbone comprises two or more monosaccharide units linked by glycosidic bonds; and the functionalizing compound is attached to the backbone and the monosaccharide.

### Pendant Functional Groups

In certain variations, combining one or more sugars (e.g., feed sugars) and one or more functionalizing compounds in the presence of a catalyst, including polymeric catalysts and solid-supported catalysts as described in WO 2012/118767 and WO 2014/031956, produces a functionalized oligosaccharide composition. In certain embodiments, a functionalizing compound is attached to a monosaccharide subunit as a pendant functional group.

A pendant functional group may include a functionalization compound attached to one monosaccharide unit, and not attached to any other monosaccharide units. In some variations, the pendant functional group is a single functionalization compound attached to one monosaccharide unit. For example, in one variation, the functionalizing compound is acetic acid, and the pendant functional group is acetate bonded to a monosaccharide through an ester linkage. In another variation, the functionalizing compound in propionic acid, and the pendant functional group is propionate bonded to a monosaccharide through an ester linkage. In yet another variation, the functionalizing compound is butanoic acid, and the pendant functional group is butanoate bonded to a monosaccharide through an ester linkage. In other variations, a pendant functional group is formed from linking multiple functionalization compounds together. For example, in some embodiments, the functionalization compound is glutamic acid, and the pendant functional group is a peptide chain of two, three, four, five, six, seven, or eight glutamic acid residues, wherein the chain is attached to a monosaccharide through an ester linkage. In other embodiments, the peptide chain is attached to the monosaccharide through an amine linkage.

The pendant functional group may comprise a single linkage to the monosaccharide, or multiple linkages to the monosaccharide. For example, in one embodiment, the functionalization compound is ethanediol, and the pendant functional group is ethyl connected to a monosaccharide through two ether linkages.

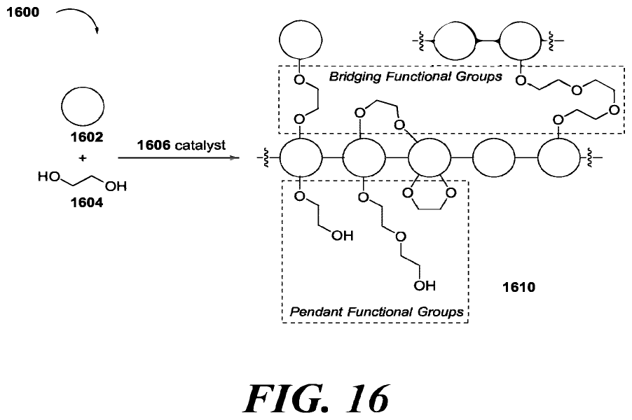

Referring to FIG. 16, process 1600 depicts an exemplary scheme to produce an oligosaccharide containing different pendant functional groups. In process 1600, monosaccharides 1602 (represented symbolically) are combined with the functionalizing compound ethane diol 1604 in the presence of catalyst 1606 to produce an oligosaccharide. Portion 1610 of the oligosaccharide is shown in FIG. 16, wherein the monosaccharides linked through glycosidic bonds are represented symbolically by circles and lines. The oligosaccharide comprises three different pendant functional groups, as indicated by the labeled section. These pendant functional groups include a single functionalization compound attached to a single monosaccharide unit through one linkage; two functionalization compounds linked together to form a pendant functional group, wherein the pendant functional group is linked to a single monosaccharide unit through one linkage; and a single functionalization compound attached to a single monosaccharide unit through two linkages. It should be understood that while the functionalization compound used in process 1600 is ethanediol, any of the functionalization compounds or combinations thereof described herein may be used. It should be further understood that while a plurality of pendant functional groups is present in portion 1610 of the oligosaccharide, the number and type of pendant functional groups may vary in other variations of process 1600.

It should be understood that any functionalization compounds may form a pendant functional group. In some variations, the functionalized oligosaccharide composition contains one or more pendant groups selected from the group consisting of glucosamine, galactosamine, citric acid, succinic acid, glutamic acid, aspartic acid, glucuronic acid, butyric acid, itaconic acid, malic acid, maleic acid, propionic acid, butanoic acid, pentanoic acid, hexanoic acid, adipic acid, isobutyric acid, formic acid, levulinic acid, valeric acid, isovaleric acid, sorbitol, xylitol, arabitol, glycerol, erythritol, mannitol, galacitol, fucitol, iditol, inositol, volemitol, lacitol, ethanol, propanol, butanol, pentanol, hexanol, propanediol, butanediol, pentanediol, sulfate and phosphate.

### Bridging Functional Groups

In certain variations, combining one or more sugars (e.g., feed sugars) and one or more functionalizing compounds in the presence of a catalyst, including polymeric catalysts and solid-supported catalysts as described in WO 2012/118767 and WO 2014/031956, produces a functionalized oligosaccharide comprising a bridging functional group.

Bridging functional groups may include a functionalization compound attached to one monosaccharide unit and attached to at least one additional monosaccharide unit. The monosaccharide units may independently be monosaccharide units of the same oligosaccharide backbone, monosaccharide units of separate oligosaccharide backbones, or monosaccharide sugars that are not bonded to any additional monosaccharides. In some variations, the bridging functional compound is attached to one additional monosaccharide unit. In other variations, the bridging functional compound is attached to two or more additional monosaccharide units. For example, in some embodiments, the bridging functional compound is attached to two, three, four, five, six, seven, or eight additional monosaccharide units. In some variations, the bridging functional group is formed by linking a single functionalization compound to two monosaccharide units. For example, in one embodiment, the functionalization compound is glutamic acid, and the bridging functional group is a glutamate residue attached to one monosaccharide unit through an ester bond, and an additional monosaccharide unit through an amine bond. In other embodiments, the bridging functionalization group is formed by linking multiple functionalization compound molecules to each other. For example, in one embodiment, the functionalization compound is ethanediol, and the bridging functional group is a linear oligomer of four ethanediol molecules attached to each other through ether bonds, the first ethanediol molecule in the oligomer is attached to one monosaccharide unit through an ether bond, and the fourth ethanediol molecule in the oligomer is attached to an additional monosaccharide unit through an ether bond.

Referring again to FIG. 16, portion 1610 of the oligosaccharide produced according to process 1600 comprises three different bridging functional groups, as indicated by the labeled section. These bridging functional groups include a single functionalization compound attached to a monosaccharide unit of an oligosaccharide through one linkage, and attached to a monosaccharide sugar through an additional linkage; a single functionalization compound attached to two different monosaccharide units of the same oligosaccharide backbone; and two functionalization compounds linked together to form a bridging functional group, wherein the bridging functional group is linked to one monosaccharide unit through one linkage and to an additional monosaccharide unit through a second linkage. It should be understood that while the functionalization compound used in process 1600 is ethanediol, any of the functionalization compounds or combinations thereof described herein may be used. It should be further understood that while a plurality of bridging functional groups is present in portion 1610 of the oligosaccharide, the number and type of bridging functional groups may vary in other variations of process 1600.

It should be understood that any functionalization compounds with two or more functional groups able to form bonds with a monosaccharide may form a bridging functional group. For example, bridging functional groups may be selected from polycarboxylic acids (such as succinic acid, itaconic acid, malic acid, maleic acid, and adipic acid), polyols (such as sorbitol, xylitol, arabitol, glycerol, erythritol, mannitol, galacitol, fucitol, iditol, inositol, volemitol, and lacitol), and amino acids (such as glutamic acid). In some variations, the functionalized oligosaccharide composition comprises one or more bridging groups selected from the group consisting of glucosamine, galactosamine, lactic acid, acetic acid, citric acid, pyruvic acid, succinic acid, glutamic acid, aspartic acid, glucuronic acid, itaconic acid, malic acid, maleic acid, adipic acid, sorbitol, xylitol, arabitol, glycerol, erythritol, mannitol, galacitol, fucitol, iditol, inositol, volemitol, lacitol, propanediol, butanediol, pentanediol, sulfate and phosphate.

Functionalized oligosaccharide compositions comprising a mixture of pendant functional groups and bridging functional groups may also be produced using the methods described herein. For example, in certain embodiments, one or more sugars are combined with a polyol in the presence of a catalyst, and a functionalized oligosaccharide composition is produced wherein at least a portion of the composition comprises pendant polyol functional groups attached to oligosaccharides through ether linkages, and at least a portion comprises bridging polyol functional groups wherein each group is attached to a first oligosaccharide through a first ether linkage and a second oligosaccharide through a second ether linkage.

It should further be understood that the one or more functionalization compounds combined with the sugars, oligosaccharide composition, or combination thereof may form bonds with other functionalization compounds, such that the functionalized oligosaccharide composition comprises monosaccharide units bonded to a first functionalization compound, wherein the first functionalization compound is bonded to a second functionalization compound.

### Degree of Polymerization

The oligosaccharide content of reaction products can be determined, e.g., by a combination of high performance liquid chromatography (HPLC) and spectrophotometric methods. For example, the average degree of polymerization (DP) for the oligosaccharides can be determined as the number average of species containing one, two, three, four, five, six, seven, eight, nine, ten to fifteen, and greater than fifteen, anhydrosugar monomer units.

In some embodiments, the oligosaccharide degree of polymerization (DP) distribution for the one or more oligosaccharides after combining the one or more sugars with the catalyst (e.g., at 2, 3, 4, 8, 12, 24, or 48 hours after combining the one or more sugars with the catalyst) is: DP2=0%-40%, such as less than 40%, less than 30%, less than 20%, less than 10%, less than 5%, or less than 2%; or 10%-30% or 15%-25%; DP3=0%-20%, such as less than 15%, less than 10%, less than 5%; or 5%-15%; and DP4+=greater than 15%, greater than 20%, greater than 30%, greater than 40%, greater than 50%; or 15%-75%, 20%-40% or 25%-35%.

In some embodiments, the oligosaccharide degree of polymerization (DP) distribution for the one or more oligosaccharides after combining the one or more sugars with the catalyst (e.g., at 2, 3, 4, 8, 12, 24, or 48 hours after combining the one or more sugars with the catalyst) is any one of entries (1)-(192) of Table 1A.

TABLE 1A

| Entry | DP4+ (%) | DP3 (%) | DP2 (%) |
|---|---|---|---|
| 1 | 20-25 | 0-5 | 0-5 |
| 2 | 20-25 | 0-5 | 5-10 |
| 3 | 20-25 | 0-5 | 10-15 |
| 4 | 20-25 | 0-5 | 15-20 |
| 5 | 20-25 | 0-5 | 20-25 |
| 6 | 20-25 | 0-5 | 25-30 |
| 7 | 20-25 | 5-10 | 0-5 |
| 8 | 20-25 | 5-10 | 5-10 |
| 9 | 20-25 | 5-10 | 10-15 |
| 10 | 20-25 | 5-10 | 15-20 |
| 11 | 20-25 | 5-10 | 20-25 |
| 12 | 20-25 | 5-10 | 25-30 |
| 13 | 20-25 | 10-15 | 0-5 |
| 14 | 20-25 | 10-15 | 5-10 |
| 15 | 20-25 | 10-15 | 10-15 |
| 16 | 20-25 | 10-15 | 15-20 |
| 17 | 20-25 | 10-15 | 20-25 |
| 18 | 20-25 | 10-15 | 25-30 |
| 19 | 20-25 | 15-20 | 0-5 |
| 20 | 20-25 | 15-20 | 5-10 |
| 21 | 20-25 | 15-20 | 10-15 |
| 22 | 20-25 | 15-20 | 15-20 |
| 23 | 20-25 | 15-20 | 20-25 |
| 24 | 20-25 | 15-20 | 25-30 |
| 25 | 20-25 | 20-25 | 0-5 |
| 26 | 20-25 | 20-25 | 5-10 |
| 27 | 20-25 | 20-25 | 10-15 |
| 28 | 20-25 | 20-25 | 15-20 |
| 29 | 20-25 | 20-25 | 20-25 |
| 30 | 20-25 | 20-25 | 25-30 |
| 31 | 25-30 | 0-5 | 0-5 |
| 32 | 25-30 | 0-5 | 5-10 |
| 33 | 25-30 | 0-5 | 10-15 |
| 34 | 25-30 | 0-5 | 15-20 |
| 35 | 25-30 | 0-5 | 20-25 |
| 36 | 25-30 | 0-5 | 25-30 |
| 37 | 25-30 | 5-10 | 0-5 |
| 38 | 25-30 | 5-10 | 5-10 |
| 39 | 25-30 | 5-10 | 10-15 |
| 40 | 25-30 | 5-10 | 15-20 |
| 41 | 25-30 | 5-10 | 20-25 |
| 42 | 25-30 | 5-10 | 25-30 |
| 43 | 25-30 | 10-15 | 0-5 |
| 44 | 25-30 | 10-15 | 5-10 |
| 45 | 25-30 | 10-15 | 10-15 |
| 46 | 25-30 | 10-15 | 15-20 |
| 47 | 25-30 | 10-15 | 20-25 |
| 48 | 25-30 | 10-15 | 25-30 |
| 49 | 25-30 | 15-20 | 0-5 |
| 50 | 25-30 | 15-20 | 5-10 |
| 51 | 25-30 | 15-20 | 10-15 |
| 52 | 25-30 | 15-20 | 15-20 |
| 53 | 25-30 | 15-20 | 20-25 |
| 54 | 25-30 | 15-20 | 25-30 |
| 55 | 25-30 | 20-25 | 0-5 |
| 56 | 25-30 | 20-25 | 5-10 |
| 57 | 25-30 | 20-25 | 10-15 |
| 58 | 25-30 | 20-25 | 15-20 |
| 59 | 25-30 | 20-25 | 20-25 |
| 60 | 25-30 | 20-25 | 25-30 |
| 61 | 30-35 | 0-5 | 0-5 |
| 62 | 30-35 | 0-5 | 5-10 |
| 63 | 30-35 | 0-5 | 10-15 |
| 64 | 30-35 | 0-5 | 15-20 |
| 65 | 30-35 | 0-5 | 20-25 |
| 66 | 30-35 | 0-5 | 25-30 |
| 67 | 30-35 | 5-10 | 0-5 |
| 68 | 30-35 | 5-10 | 5-10 |
| 69 | 30-35 | 5-10 | 10-15 |
| 70 | 30-35 | 5-10 | 15-20 |
| 71 | 30-35 | 5-10 | 20-25 |
| 72 | 30-35 | 5-10 | 25-30 |
| 73 | 30-35 | 10-15 | 0-5 |
| 74 | 30-35 | 10-15 | 5-10 |
| 75 | 30-35 | 10-15 | 10-15 |
| 76 | 30-35 | 10-15 | 15-20 |
| 77 | 30-35 | 10-15 | 20-25 |
| 78 | 30-35 | 10-15 | 25-30 |
| 79 | 30-35 | 15-20 | 0-5 |
| 80 | 30-35 | 15-20 | 5-10 |
| 81 | 30-35 | 15-20 | 10-15 |
| 82 | 30-35 | 15-20 | 15-20 |
| 83 | 30-35 | 15-20 | 20-25 |
| 84 | 30-35 | 15-20 | 25-30 |
| 85 | 30-35 | 20-25 | 0-5 |
| 86 | 30-35 | 20-25 | 5-10 |
| 87 | 30-35 | 20-25 | 10-15 |
| 88 | 30-35 | 20-25 | 15-20 |
| 89 | 30-35 | 20-25 | 20-25 |
| 90 | 30-35 | 20-25 | 25-30 |
| 91 | 35-40 | 0-5 | 0-5 |
| 92 | 35-40 | 0-5 | 5-10 |
| 93 | 35-40 | 0-5 | 10-15 |
| 94 | 35-40 | 0-5 | 15-20 |
| 95 | 35-40 | 0-5 | 20-25 |
| 96 | 35-40 | 0-5 | 25-30 |
| 97 | 35-40 | 5-10 | 0-5 |
| 98 | 35-40 | 5-10 | 5-10 |
| 99 | 35-40 | 5-10 | 10-15 |
| 100 | 35-40 | 5-10 | 15-20 |
| 101 | 35-40 | 5-10 | 20-25 |
| 102 | 35-40 | 5-10 | 25-30 |
| 103 | 35-40 | 10-15 | 0-5 |
| 104 | 35-40 | 10-15 | 5-10 |
| 105 | 35-40 | 10-15 | 10-15 |
| 106 | 35-40 | 10-15 | 15-20 |
| 107 | 35-40 | 10-15 | 20-25 |
| 108 | 35-40 | 10-15 | 25-30 |
| 109 | 35-40 | 15-20 | 0-5 |
| 110 | 35-40 | 15-20 | 5-10 |
| 111 | 35-40 | 15-20 | 10-15 |
| 112 | 35-40 | 15-20 | 15-20 |
| 113 | 35-40 | 15-20 | 20-25 |
| 114 | 35-40 | 15-20 | 25-30 |
| 115 | 35-40 | 20-25 | 0-5 |
| 116 | 35-40 | 20-25 | 5-10 |
| 117 | 35-40 | 20-25 | 10-15 |
| 118 | 35-40 | 20-25 | 15-20 |
| 119 | 35-40 | 20-25 | 20-25 |
| 120 | 35-40 | 20-25 | 25-30 |
| 121 | 40-45 | 0-5 | 0-5 |
| 122 | 40-45 | 0-5 | 5-10 |
| 123 | 40-45 | 0-5 | 10-15 |
| 124 | 40-45 | 0-5 | 15-20 |
| 125 | 40-45 | 0-5 | 20-25 |
| 126 | 40-45 | 0-5 | 25-30 |
| 127 | 40-45 | 5-10 | 0-5 |
| 128 | 40-45 | 5-10 | 5-10 |
| 129 | 40-45 | 5-10 | 10-15 |
| 130 | 40-45 | 5-10 | 15-20 |
| 131 | 40-45 | 5-10 | 20-25 |
| 132 | 40-45 | 5-10 | 25-30 |
| 133 | 40-45 | 10-15 | 0-5 |
| 134 | 40-45 | 10-15 | 5-10 |
| 135 | 40-45 | 10-15 | 10-15 |
| 136 | 40-45 | 10-15 | 15-20 |
| 137 | 40-45 | 10-15 | 20-25 |
| 138 | 40-45 | 10-15 | 25-30 |
| 139 | 40-45 | 15-20 | 0-5 |
| 140 | 40-45 | 15-20 | 5-10 |
| 141 | 40-45 | 15-20 | 10-15 |
| 142 | 40-45 | 15-20 | 15-20 |
| 143 | 40-45 | 15-20 | 20-25 |
| 144 | 40-45 | 15-20 | 25-30 |
| 145 | 40-45 | 20-25 | 0-5 |
| 146 | 40-45 | 20-25 | 5-10 |
| 147 | 40-45 | 20-25 | 10-15 |
| 148 | 40-45 | 20-25 | 15-20 |
| 149 | 40-45 | 20-25 | 20-25 |
| 150 | 40-45 | 20-25 | 25-30 |
| 151 | >50 | 0-5 | 0-5 |
| 152 | >50 | 0-5 | 5-10 |
| 153 | >50 | 0-5 | 10-15 |
| 154 | >50 | 0-5 | 15-20 |
| 155 | >50 | 0-5 | 20-25 |
| 156 | >50 | 0-5 | 25-30 |
| 157 | >50 | 5-10 | 0-5 |
| 158 | >50 | 5-10 | 5-10 |
| 159 | >50 | 5-10 | 10-15 |
| 160 | >50 | 5-10 | 15-20 |
| 161 | >50 | 5-10 | 20-25 |
| 162 | >50 | 5-10 | 25-30 |
| 163 | >50 | 10-15 | 0-5 |
| 164 | >50 | 10-15 | 5-10 |
| 165 | >50 | 10-15 | 10-15 |
| 166 | >50 | 10-15 | 15-20 |
| 167 | >50 | 10-15 | 20-25 |
| 168 | >50 | 10-15 | 25-30 |
| 169 | >50 | 15-20 | 0-5 |
| 170 | >50 | 15-20 | 5-10 |
| 171 | >50 | 15-20 | 10-15 |
| 172 | >50 | 15-20 | 15-20 |
| 173 | >50 | 15-20 | 20-25 |
| 174 | >50 | 15-20 | 25-30 |
| 175 | >50 | 20-25 | 0-5 |
| 176 | >50 | 20-25 | 5-10 |
| 177 | >50 | 20-25 | 10-15 |
| 178 | >50 | 20-25 | 15-20 |
| 179 | >50 | 20-25 | 20-25 |
| 180 | >60 | 10-20 | 10-20 |
| 181 | >60 | 5-10 | 10-20 |
| 182 | >60 | 0-10 | 0-10 |
| 183 | >70 | 10-20 | 10-20 |
| 184 | >70 | 5-10 | 10-20 |
| 185 | >70 | 0-10 | 0-10 |
| 186 | >80 | 10-20 | 10-20 |
| 187 | >80 | 5-10 | 10-20 |
| 188 | >80 | 0-10 | 0-10 |
| 189 | >85 | 10-20 | 10-20 |
| 190 | >85 | 0-10 | 0-10 |
| 191 | >85 | 0-10 | 0-5 |
| 192 | >90 | 0-10 | 0-10 |

The yield of conversion for the one or more sugars to the one or more oligosaccharides in the methods described herein can be determined by any suitable method known in the art, including, for example, high performance liquid chromatography (HPLC). In some embodiments, the yield of conversion to one or more oligosaccharides to with DP>1 after combining the one or more sugars with the catalyst (e.g., at 2, 3, 4, 8, 12, 24, or 48 hours after combining the one or more sugars with the catalyst) is greater than about 50% (or greater than about 55%, 60%, 65%, 70%, 75%, 80%, 85%, 90%, 95%, or 98%). In some embodiments, the yield of conversion to one or more oligosaccharides of >DP2 after combining the one or more sugars with the catalyst (e.g., at 2, 3, 4, 8, 12, 24, or 48 hours after combining the one or more sugars with the catalyst) is greater than 30% (or greater than 35%, 40%, 45%, 50%, 55%, 60%, 65%, 70%, 75%, 80%, 85%, 90%, 95%, or 98%).

In some embodiments, the methods described herein produce an oligosaccharide composition having lower levels of degradation products, resulting in relatively higher selectivity. The molar yield to sugar degradation products and selectivity may be determined by any suitable method known in the art, including, for example, HPLC. In some embodiments, the amount of sugar degradation products after combining the one or more sugars with the catalyst (e.g., at 2, 3, 4, 8, 12, 24, or 48 hours after combining the one or more sugars with the catalyst) is less than about 10% (or less than about 9%, 8%, 7%, 6%, 5%, 4%, 3%, 2%, 1%, 0.75%, 0.5%, 0.25%, or 0.1%), such as less than about 10% of any one or combination of 1,6-anhydroglucose (levoglucosan), 5-hydroxymethylfurfural, 2-furaldehyde, acetic acid, formic acid, levulinic acid and/or humins. In some embodiments, the molar selectivity to oligosaccharide product after combining the one or more sugars with the catalyst (e.g., at 2, 3, 4, 8, 12, 24, or 48 hours after combining the one or more sugars with the catalyst) is greater than about 90% (or greater than about 95%, 97%, 98%, 99%, 99.5%, or 99.9%).

In some variations, at least 10 dry wt % of the oligosaccharide composition produced according to the methods described herein has a degree of polymerization of at least 3. In some embodiments, at least 10 dry wt %, at least 20 dry wt %, at least 30 dry wt %, at least 40 dry wt %, at least 50 dry wt %, at least 60 dry wt %, at least 70 wt %, between 10 to 90 dry wt %, between 20 to 80 dry wt %, between 30 to 80 dry wt %, between 50 to 80 dry wt %, or between 70 to 80 dry wt % of the oligosaccharide composition has a degree of polymerization of at least 3.

In some variations, the oligosaccharide composition produced according to methods described herein has a DP3+ of at least 10% on a dry-weight basis. In certain variations, the oligosaccharide composition produced according to methods described herein has a DP3+ of at least 10% on a dry-weight basis, at least 20% on a dry-weight basis, at least 30% on a dry-weight basis, at least 40% on a dry-weight basis, at least 50% on a dry-weight basis, at least 60% on a dry-weight basis, at least 70% on a dry-weight basis, between 10 to 90% on a dry-weight basis, between 20 to 80% on a dry-weight basis, between 30 to 80% on a dry-weight basis, between 50 to 80% on a dry-weight basis, or between 70 to 80% on a dry-weight basis.

In some variations, the oligosaccharide composition has an average molecular weight of between 100 g/mol and 2000 g/mol, or between 300 g/mol and 1800 g/mol, or between 300 g/mol and 1700 g/mol, or between 500 g/mol and 1500 g/mol; or about 300 g/mol, 350 g/mol, 400 g/mol, 450 g/mol, 500 g/mol, 550 g/mol, 600 g/mol, 650 g/mol, 700 g/mol, 750 g/mol, 800 g/mol, 850 g/mol, 900 g/mol, 950 g/mol, 1000 g/mol, 1100 g/mol, 1200 g/mol, 1300 g/mol, 1400 g/mol, 1500 g/mol, 1600 g/mol, 1700 g/mol, or about 1800 g/mol. In certain variations of the foregoing, the average molecular weight of the oligosaccharide composition is determined as the number average molecular weight. In other variations, the average molecular weight of the oligosaccharide composition is determined as the weight average molecular weight. In yet another variation, the oligosaccharide composition contains only monosaccharide units that have the same molecular weight, in which case the number average molecular weight is identical to the product of the average degree of polymerization and the molecular weight of the monosaccharide unit.

### Digestibility

In some variations, the “digestibility” of a compound refers to the ability of the human digestive system (e.g., mouth, esophagus, stomach and/or small intestine) to absorb either a compound or the digestion products that result from the action of the digestive system (e.g. hydrolysis by digestive acids and/or enzymes) on the compound. Examples of digestible compounds include monosaccharides, certain disaccharides such as sucrose and maltose, certain oligosaccharides, such as malto-dextrins, and certain polysaccharides such as starch. Compounds that are resistant to digestion include, for example, dietary fiber.

The digestibility of the one or more oligosaccharides produced according to the methods described herein can be determined by standard methods known to one skilled in the art, e.g., by the in vitro method AOAC 2009.01 or the in vitro Englyst Assay. The AOAC 2009.01 is an enzyme assays that can determine the amount of a carbohydrate composition that is dietary fiber. See Official Methods of Analysis of AOAC International, AOAC International, Gaithersberg, USA. For example, the Englyst Assay is an enzyme assay that can determine the amount of a carbohydrate composition that is rapidly digestible, slowly digestible, or resistant to digestion. See European Journal of Clinical Nutrition (1992) Volume 46, Suppl. 2, pages S33-S60. In certain embodiments, the digestibility of a carbohydrate can be determined as the mass fraction of the carbohydrate that is hydrolyzed to monosaccharides under the hydrolysis steps of the AOAC 2009.01 method. For example, the digestibility of a monosaccharide is 1 g/g. The digestibility of a disaccharide (DP2) is the mass fraction of the disaccharide that is hydrolyzed to monosaccharides under the hydrolysis steps of the AOAC 2009.01 method. The digestibility of a trisaccharide (DP3) is the mass fraction of the trisaccharide that is hydrolyzed to monosaccharides under the hydrolysis steps of the AOAC 2009.01 method. In certain embodiments, the digestibility of a mixture of carbohydrates is the mass weighted sum of the digestibilities of its components. For example, the digestibility of a carbohydrate composition is the mass fraction of the DP1 component of the carbohydrate composition plus the mass fraction of the DP2 component of the carbohydrate composition times the digestibility of the DP2 component of the carbohydrate composition plus the mass fraction of the DP3 component of the carbohydrate composition times the digestibility of the DP3 component of the carbohydrate composition, up to and including the maximum DP component of the carbohydrate composition.

In some embodiments, greater than 50%, greater than 55%, greater than 60%, greater than 70%, greater than 80%, greater than 90%, or greater than 99% of the one or more oligosaccharides produced by the methods described herein is dietary fiber. In some embodiments, less than 50%, less than 40%, less than 30%, less than 20%, less than 10%, less than 5%, or less than 1% of the oligosaccharide composition with a DP of 3 or greater is hydrolyzed to oligosaccharides with a DP of 2 and/or monosaccharides.

In some variations, the oligosaccharide composition has a digestibility of less than 0.60 g/g, less than 0.55 g/g, less than 0.50 g/g, less than 0.45 g/g, less than 0.40 g/g, less than 0.35 g/g, less than 0.30 g/g, less than 0.25 g/g, less than 0.20 g/g, less than 0.15 g/g, less than 0.10 g/g, or less than 0.05 g/g. In certain variations, the oligosaccharide composition has a digestibility between 0.05 g/g and 0.60 g/g, between 0.05 g/g and 0.30 g/g, or between 0.05 g/g and 0.20 g/g.

### Glass Transition Temperature

In some variations, “glass transition” refers to the reversible transition of some compounds from a hard and relatively brittle state to a softer, flexible state. In some variations, “glass transition temperature” refers to the temperature determined by differential scanning calorimetry.

The glass transition temperature of a material can impart desirable characteristics to that material, and/or can impart desirable characteristics to a composition comprising that material. In some embodiments, the methods described herein are used to produce one or more oligosaccharides with a specific glass transition temperature, or within a glass transition temperature range. In some variations, the glass transition temperature of one or more oligosaccharides produced according to the methods described herein imparts desirable characteristics to the one or more oligosaccharides (e.g., texture, storage, or processing characteristics). In certain variations, the glass transition temperature of the one or more oligosaccharides imparts desirable characteristics to a composition including the one or more oligosaccharides (e.g., texture, storage, or processing characteristics).

For example, in some variations, foods including the one or more oligosaccharides with a lower glass transition temperature have a softer texture than foods including the one or more oligosaccharides with a higher glass transition temperature, or foods that do not include the one or more oligosaccharides. In other variations, foods including the one or more oligosaccharides with a higher glass transition temperature have reduced caking and can be dried at higher temperatures than foods including the one or more oligosaccharides with a lower glass transition temperature, or foods that do not include the one or more oligosaccharides.

In some embodiments, the glass transition temperature of the one or more oligosaccharides when prepared in a dry powder form with a moisture content below 6% is at least −20 degrees Celsius (° C.), at least −10 degrees Celsius, at least 0 degrees Celsius, at least 10 degrees Celsius, at least 20 degrees Celsius, at least 30 degrees Celsius, at least 40 degrees Celsius, at least 50 degrees Celsius, at least 60 degrees Celsius, at least 70 degrees Celsius, at least 80 degrees Celsius, at least 90 degrees Celsius, or at least 100 degrees Celsius. In certain embodiments, the glass transition temperature of the one or more oligosaccharides is between 40 degrees Celsius and 80 degrees Celsius.

In some variations, the oligosaccharide composition has a glass transition temperature of at least −20 degrees Celsius (° C.), at least −10 degrees Celsius, at least 0 degrees Celsius, at least 10 degrees Celsius, at least 20 degrees Celsius, at least 30 degrees Celsius, at least 40 degrees Celsius, at least 50 degrees Celsius, at least 60 degrees Celsius, at least 70 degrees Celsius, at least 80 degrees Celsius, at least 90 degrees Celsius, or at least 100 degrees Celsius, when measured at less than 10 wt % water. In certain embodiments, the oligosaccharide composition has a glass transition temperature of between 40 degrees Celsius and 80 degrees Celsius, when measured at less than 10 wt % water. In one variation, the oligosaccharide composition has a glass transition temperature between −20 and 115 degrees Celsius, when measured at less than 10 wt % water.

### Hygroscopicity

In some variations, “hygroscopicity” refers to the ability of a compound to attract and hold water molecules from the surrounding environment. The hygroscopicity of a material can impart desirable characteristics to that material, and/or can impart desirable characteristics to a composition comprising that material. In some embodiments, the methods described herein are used to produce one or more oligosaccharides with a specific hygroscopicity value or a range of hygroscopicity values. In some variations, the hygroscopicity of one or more oligosaccharides produced according to the methods described herein imparts desirable characteristics to the one or more oligosaccharides (e.g., texture, storage, or processing characteristics). In certain variations, the hygroscopicity of the one or more oligosaccharides imparts desirable characteristics to a composition including the one or more oligosaccharides (e.g., texture, storage, or processing characteristics).

For example, in some variations, foods including the one or more oligosaccharides with a higher hygroscopicity have a softer texture than foods including the one or more oligosaccharides with a lower hygroscopicity, or foods without the one or more oligosaccharides. In certain variations, the one or more oligosaccharides with a higher hygroscopicity are included in food products to reduce water activity, increase shelf life, produce a softer product, produce a moister product, and/or enhance the surface sheen of the product.

In other variations, foods including the one or more oligosaccharides with a lower hygroscopicity have reduced caking and can be dried at a higher temperature than foods including the one or more oligosaccharides with a higher hygroscopicity, or foods without the one or more oligosaccharides. In certain variations, the one or more oligosaccharides with a lower hygroscopicity are included in food products to increase crispness, increase shelf life, reduce clumping, reduce caking, improve, and/or enhance the appearance of the product.

The hygroscopicity of a composition, including the one or more oligosaccharides, can be determined by measuring the mass gain of the composition after equilibration in a fixed water activity atmosphere (e.g., a desiccator held at a fixed relative humidity).

In some embodiments, the hygroscopicity of the one or more oligosaccharides is at least 5% moisture content at a water activity of at least 0.6, at least 10% moisture content at a water activity of at least 0.6, at least 15% moisture content at a water activity of at least 0.6, at least 20% moisture content at a water activity of at least 0.6, or at least 30% moisture content at a water activity of at least 0.6. In certain embodiments, the hygroscopicity of the one or more oligosaccharides is between 5% moisture content and 15% moisture content at a water activity of at least 0.6.

In certain variations, the oligosaccharide composition has a hygroscopicity of at least 5%, at least 10%, at least 15%, at least 20%, or at least 30% moisture content, when measured at a water activity of at least 0.6. In certain embodiments, the oligosaccharide composition has a hygroscopicity of between 5% moisture content and 15% moisture content, when measured at a water activity of at least 0.6.

In one variation, the oligosaccharide composition has a hygroscopicity of at least 0.05 g/g, when measured at a water activity of 0.6.

### Fiber Content

In some variations, “dietary fiber” refers to a carbohydrate (i.e., an oligosaccharide or a polysaccharide) with a degree of polymerization of at least 3 that is not effectively hydrolyzed to its constituent sugars in humans by enzymes in the stomach or small intestine (e.g., α-amylase, amyloglucosidase, and protease). In some embodiments, the dietary fiber is insoluble in water. In other embodiments, the dietary fiber is soluble in water. In certain embodiments, the dietary fiber is soluble in water up to a maximum concentration of at least 10 Brix, of at least 20 Brix, of at least 30 Brix, of at least 40 Brix, of at least 50 Brix, of at least 60 Brix, of at least 70 Brix, of at least 80 Brix, or of at least 80 Brix. In one embodiment, the dietary fiber is soluble with a maximum concentration between 75 and 90 Brix.

The dietary fiber content of a composition, including, for example, the dietary fiber content of the one or more oligosaccharides described herein, can be determined by the in vitro method AOAC 2009.01 (Official Methods of Analysis of AOAC International, AOAC International, Gaithersberg, USA) to quantify the fraction of oligosaccharides in the composition that have a degree of polymerization (DP) of at least three and that are not hydrolyzed by a combination the enzymes: α-amylase, amyloglucosidase, and protease.

In some embodiments, the dietary fiber content of the one or more oligosaccharides is at least 50% on a dry mass basis, at least 60% on a dry mass basis, at least 70% on a dry mass basis, at least 80% on a dry mass basis, or at least 90% on a dry mass basis. In certain embodiments, the dietary fiber content of the one or more oligosaccharides is between 70% and 80% on a dry mass basis.

In one variation, the oligosaccharide composition has a fiber content of at least 80 g/g.

In some embodiments, the mean degree of polymerization (DP), glass transition temperature (Tg), hygroscopicity, and fiber content of the oligosaccharide composition produced by combining the one or more sugars with the catalyst (e.g., at 2, 3, 4, 8, 12, 24, or 48 hours after combining the one or more sugars with the catalyst) is any one of entries (1)-(180) of Table 1B.

TABLE 1B

| | | Tg at <10 | Hygroscopicity | Fiber |
|---|---|---|---|---|
| | | wt % H2O | (wt % H2O @ | Content |
| Number | Mean DP | (° C.) | 0.6 Aw) | (wt %) |
| 1 | 5-10 | >50 | >5% | >50% |
| 2 | 5-10 | >50 | >5% | >60% |
| 3 | 5-10 | >50 | >5% | >70% |
| 4 | 5-10 | >50 | >5% | >80% |
| 5 | 5-10 | >50 | >5% | >90% |
| 6 | 5-10 | >50 | >10% | >50% |
| 7 | 5-10 | >50 | >10% | >60% |
| 8 | 5-10 | >50 | >10% | >70% |
| 9 | 5-10 | >50 | >10% | >80% |
| 10 | 5-10 | >50 | >10% | >90% |
| 11 | 5-10 | >50 | >15% | >50% |
| 12 | 5-10 | >50 | >15% | >60% |
| 13 | 5-10 | >50 | >15% | >70% |
| 14 | 5-10 | >50 | >15% | >80% |
| 15 | 5-10 | >50 | >15% | >90% |
| 16 | 5-10 | >50 | >5% | >50% |
| 17 | 5-10 | >50 | >5% | >60% |
| 18 | 5-10 | >50 | >5% | >70% |
| 19 | 5-10 | >50 | >5% | >80% |
| 20 | 5-10 | >50 | >5% | >90% |
| 21 | 5-10 | >50 | >10% | >50% |
| 22 | 5-10 | >50 | >10% | >60% |
| 23 | 5-10 | >50 | >10% | >70% |
| 24 | 5-10 | >50 | >10% | >80% |
| 25 | 5-10 | >50 | >10% | >90% |
| 26 | 5-10 | >50 | >15% | >50% |
| 27 | 5-10 | >50 | >15% | >60% |
| 28 | 5-10 | >50 | >15% | >70% |
| 29 | 5-10 | >50 | >15% | >80% |
| 30 | 5-10 | >50 | >15% | >90% |
| 31 | 5-10 | >75 | >5% | >50% |
| 32 | 5-10 | >75 | >5% | >60% |
| 33 | 5-10 | >75 | >5% | >70% |
| 34 | 5-10 | >75 | >5% | >80% |
| 35 | 5-10 | >75 | >5% | >90% |
| 36 | 5-10 | >75 | >10% | >50% |
| 37 | 5-10 | >75 | >10% | >60% |
| 38 | 5-10 | >75 | >10% | >70% |
| 39 | 5-10 | >75 | >10% | >80% |
| 40 | 5-10 | >75 | >10% | >90% |
| 41 | 5-10 | >75 | >15% | >50% |
| 42 | 5-10 | >75 | >15% | >60% |
| 43 | 5-10 | >75 | >15% | >70% |
| 44 | 5-10 | >75 | >15% | >80% |
| 45 | 5-10 | >75 | >15% | >90% |
| 46 | 5-10 | >75 | >5% | >50% |
| 47 | 5-10 | >75 | >5% | >60% |
| 48 | 5-10 | >75 | >5% | >70% |
| 49 | 5-10 | >75 | >5% | >80% |
| 50 | 5-10 | >75 | >5% | >90% |
| 51 | 5-10 | >75 | >10% | >50% |
| 52 | 5-10 | >75 | >10% | >60% |
| 53 | 5-10 | >75 | >10% | >70% |
| 54 | 5-10 | >75 | >10% | >80% |
| 55 | 5-10 | >75 | >10% | >90% |
| 56 | 5-10 | >75 | >15% | >50% |
| 57 | 5-10 | >75 | >15% | >60% |
| 58 | 5-10 | >75 | >15% | >70% |
| 59 | 5-10 | >75 | >15% | >80% |
| 60 | 5-10 | >75 | >15% | >90% |
| 61 | 5-10 | >100 | >5% | >50% |
| 62 | 5-10 | >100 | >5% | >60% |
| 63 | 5-10 | >100 | >5% | >70% |
| 64 | 5-10 | >100 | >5% | >80% |
| 65 | 5-10 | >100 | >5% | >90% |
| 66 | 5-10 | >100 | >10% | >50% |
| 67 | 5-10 | >100 | >10% | >60% |
| 68 | 5-10 | >100 | >10% | >70% |
| 69 | 5-10 | >100 | >10% | >80% |
| 70 | 5-10 | >100 | >10% | >90% |
| 71 | 5-10 | >100 | >15% | >50% |
| 72 | 5-10 | >100 | >15% | >60% |
| 73 | 5-10 | >100 | >15% | >70% |
| 74 | 5-10 | >100 | >15% | >80% |
| 75 | 5-10 | >100 | >15% | >90% |
| 76 | 5-10 | >100 | >5% | >50% |
| 77 | 5-10 | >100 | >5% | >60% |
| 78 | 5-10 | >100 | >5% | >70% |
| 79 | 5-10 | >100 | >5% | >80% |
| 80 | 5-10 | >100 | >5% | >90% |
| 81 | 5-10 | >100 | >10% | >50% |
| 82 | 5-10 | >100 | >10% | >60% |
| 83 | 5-10 | >100 | >10% | >70% |
| 84 | 5-10 | >100 | >10% | >80% |
| 85 | 5-10 | >100 | >10% | >90% |
| 86 | 5-10 | >100 | >15% | >50% |
| 87 | 5-10 | >100 | >15% | >60% |
| 88 | 5-10 | >100 | >15% | >70% |
| 89 | 5-10 | >100 | >15% | >80% |
| 90 | 5-10 | >100 | >15% | >90% |
| 91 | 10-15 | >50 | >5% | >50% |
| 92 | 10-15 | >50 | >5% | >60% |
| 93 | 10-15 | >50 | >5% | >70% |
| 94 | 10-15 | >50 | >5% | >80% |
| 95 | 10-15 | >50 | >5% | >90% |
| 96 | 10-15 | >50 | >10% | >50% |
| 97 | 10-15 | >50 | >10% | >60% |
| 98 | 10-15 | >50 | >10% | >70% |
| 99 | 10-15 | >50 | >10% | >80% |
| 100 | 10-15 | >50 | >10% | >90% |
| 101 | 10-15 | >50 | >15% | >50% |
| 102 | 10-15 | >50 | >15% | >60% |
| 103 | 10-15 | >50 | >15% | >70% |
| 104 | 10-15 | >50 | >15% | >80% |
| 105 | 10-15 | >50 | >15% | >90% |
| 106 | 10-15 | >50 | >5% | >50% |
| 107 | 10-15 | >50 | >5% | >60% |
| 108 | 10-15 | >50 | >5% | >70% |
| 109 | 10-15 | >50 | >5% | >80% |
| 110 | 10-15 | >50 | >5% | >90% |
| 111 | 10-15 | >50 | >10% | >50% |
| 112 | 10-15 | >50 | >10% | >60% |
| 113 | 10-15 | >50 | >10% | >70% |
| 114 | 10-15 | >50 | >10% | >80% |
| 115 | 10-15 | >50 | >10% | >90% |
| 116 | 10-15 | >50 | >15% | >50% |
| 117 | 10-15 | >50 | >15% | >60% |
| 118 | 10-15 | >50 | >15% | >70% |
| 119 | 10-15 | >50 | >15% | >80% |
| 120 | 10-15 | >50 | >15% | >90% |
| 121 | 10-15 | >75 | >5% | >50% |
| 122 | 10-15 | >75 | >5% | >60% |
| 123 | 10-15 | >75 | >5% | >70% |
| 124 | 10-15 | >75 | >5% | >80% |
| 125 | 10-15 | >75 | >5% | >90% |
| 126 | 10-15 | >75 | >10% | >50% |
| 127 | 10-15 | >75 | >10% | >60% |
| 128 | 10-15 | >75 | >10% | >70% |
| 129 | 10-15 | >75 | >10% | >80% |
| 130 | 10-15 | >75 | >10% | >90% |
| 131 | 10-15 | >75 | >15% | >50% |
| 132 | 10-15 | >75 | >15% | >60% |
| 133 | 10-15 | >75 | >15% | >70% |
| 134 | 10-15 | >75 | >15% | >80% |
| 135 | 10-15 | >75 | >15% | >90% |
| 136 | 10-15 | >75 | >5% | >50% |
| 137 | 10-15 | >75 | >5% | >60% |
| 138 | 10-15 | >75 | >5% | >70% |
| 139 | 10-15 | >75 | >5% | >80% |
| 140 | 10-15 | >75 | >5% | >90% |
| 141 | 10-15 | >75 | >10% | >50% |
| 142 | 10-15 | >75 | >10% | >60% |
| 143 | 10-15 | >75 | >10% | >70% |
| 144 | 10-15 | >75 | >10% | >80% |
| 145 | 10-15 | >75 | >10% | >90% |
| 146 | 10-15 | >75 | >15% | >50% |
| 147 | 10-15 | >75 | >15% | >60% |
| 148 | 10-15 | >75 | >15% | >70% |
| 149 | 10-15 | >75 | >15% | >80% |
| 150 | 10-15 | >75 | >15% | >90% |
| 151 | 10-15 | >100 | >5% | >50% |
| 152 | 10-15 | >100 | >5% | >60% |
| 153 | 10-15 | >100 | >5% | >70% |
| 154 | 10-15 | >100 | >5% | >80% |
| 155 | 10-15 | >100 | >5% | >90% |
| 156 | 10-15 | >100 | >10% | >50% |
| 157 | 10-15 | >100 | >10% | >60% |
| 158 | 10-15 | >100 | >10% | >70% |
| 159 | 10-15 | >100 | >10% | >80% |
| 160 | 10-15 | >100 | >10% | >90% |
| 161 | 10-15 | >100 | >15% | >50% |
| 162 | 10-15 | >100 | >15% | >60% |
| 163 | 10-15 | >100 | >15% | >70% |
| 164 | 10-15 | >100 | >15% | >80% |
| 165 | 10-15 | >100 | >15% | >90% |
| 166 | 10-15 | >100 | >5% | >50% |
| 167 | 10-15 | >100 | >5% | >60% |
| 168 | 10-15 | >100 | >5% | >70% |
| 169 | 10-15 | >100 | >5% | >80% |
| 170 | 10-15 | >100 | >5% | >90% |
| 171 | 10-15 | >100 | >10% | >50% |
| 172 | 10-15 | >100 | >10% | >60% |
| 173 | 10-15 | >100 | >10% | >70% |
| 174 | 10-15 | >100 | >10% | >80% |
| 175 | 10-15 | >100 | >10% | >90% |
| 176 | 10-15 | >100 | >15% | >50% |
| 177 | 10-15 | >100 | >15% | >60% |
| 178 | 10-15 | >100 | >15% | >70% |
| 179 | 10-15 | >100 | >15% | >80% |
| 180 | 10-15 | >100 | >15% | >90% |

### Glycosidic Bond Type Distribution

In certain variations, the oligosaccharide composition produced according to the methods described herein has a distribution of glycosidic bond linkages. The distribution of glycosidic bond types may be determined by any suitable methods known in the art, including, for example, proton NMR or two dimensional J-resolved nuclear magnetic resonance spectroscopy (2D-JRES NMR). In some variations, the distribution of glycosidic bond types described herein is determined by 2D-JRES NMR.

As described above, the oligosaccharide composition may comprise hexose sugar monomers (such as glucose) or pentose sugar monomers (such as xylose), or combinations thereof. It should be understood by one of skill in the art that certain types of glycosidic linkages may not be applicable to oligosaccharides comprising pentose sugar monomers.

In some variations, the oligosaccharide composition has a bond distribution with:
(i) α-(1,2) glycosidic linkages;
(ii) α-(1,3) glycosidic linkages;
(iii) α-(1,4) glycosidic linkages;
(iv) α-(1,6) glycosidic linkages;
(v) β-(1,2) glycosidic linkages;
(vi) β-(1,3) glycosidic linkages;
(vii) β-(1,4) glycosidic linkages; or
(viii) β-(1,6) glycosidic linkages,
or any combination of (i) to (viii) above.

For example, in some variations, the oligosaccharide composition has a bond distribution with a combination of (ii) and (vi) glycosidic linkages. In other variations, the oligosaccharide composition has a bond distribution with a combination of (i), (viii), and (iv) glycosidic linkages. In another variation, the oligosaccharide composition has a bond distribution with a combination of (i), (ii), (v), (vi), (vii), and (viii) glycosidic linkages.

In certain variations, the oligosaccharide composition has a bond distribution with any combination of (i), (ii), (iii), (v), (vi), and (vii) glycosidic linkages, and comprises oligosaccharides with pentose sugar monomers. In other variations, the oligosaccharide composition has a bond distribution with any combination of (i), (ii), (iii), (iv), (v), (vi), (vii) and (viii) glycosidic linkages, and comprises oligosaccharides with hexose sugar monomers. In still other variations, the oligosaccharide composition has a bond distribution with any combination of (i), (ii), (iii), (iv), (v), (vi), (vii) and (viii) glycosidic linkages, and comprises oligosaccharides with hexose sugar monomers, and oligosaccharides with pentose sugar monomers. In still other variations, the oligosaccharide composition has a bond distribution with any combination of (i), (ii), (iii), (iv), (v), (vi), (vii) and (viii) glycosidic linkages, and comprises oligosaccharides with hexose sugar monomers and pentose sugar monomers. In yet another variation, the oligosaccharide composition has a bond distribution with any combination of (i), (ii), (iii), (iv), (v), (vi), (vii) and (viii) glycosidic linkages, and comprises oligosaccharides with hexose sugar monomers, oligosaccharides with pentose sugar monomers, and oligosaccharides with hexose and pentose sugar monomers.

In some variations, the oligosaccharide composition has a glycosidic bond type distribution of less than 20 mol % α-(1,2) glycosidic linkages, less than 10 mol % α-(1,2) glycosidic linkages, less than 5 mol % α-(1,2) glycosidic linkages, between 0 to 25 mol % α-(1,2) glycosidic linkages, between 1 to 25 mol % α-(1,2) glycosidic linkages, between 0 to 20 mol % α-(1,2) glycosidic linkages, between 1 to 15 mol % α-(1,2) glycosidic linkages, between 0 to 10 mol % α-(1,2) glycosidic linkages, or between 1 to 10 mol % α-(1,2) glycosidic linkages.

In some variations, the oligosaccharide composition has a glycosidic bond type distribution of less than 50 mol % β-(1,2) glycosidic linkages, less than 40 mol % β-(1,2) glycosidic linkages, less than 35 mol % β-(1,2) glycosidic linkages, less than 30 mol % β-(1,2) glycosidic linkages, less than 25 mol % β-(1,2) glycosidic linkages, less than 10 mol % β-(1,2) glycosidic linkages, at least 1 mol % β-(1,2) glycosidic linkages, at least 5 mol % β-(1,2) glycosidic linkages, at least 10 mol % β-(1,2) glycosidic linkages, at least 15 mol % β-(1,2) glycosidic linkages, at least 20 mol % β-(1,2) glycosidic linkages, between 0 to 30 mol % β-(1,2) glycosidic linkages, between 1 to 30 mol % β-(1,2) glycosidic linkages, between 0 to 25 mol % (3-(1,2) glycosidic linkages, between 1 to 25 mol % β-(1,2) glycosidic linkages, between 10 to 30 mol % β-(1,2) glycosidic linkages, between 15 to 25 mol % β-(1,2) glycosidic linkages, between 0 to 10 mol % β-(1,2) glycosidic linkages, between 1 to 10 mol % β-(1,2) glycosidic linkages, between 10 to 50 mol % β-(1,2) glycosidic linkages, between 10 to 40 mol % β-(1,2) glycosidic linkages, between 20 to 35 mol % β-(1,2) glycosidic linkages, between 20 to 35 mol % β-(1,2) glycosidic linkages, between 20 to 50 mol % β-(1,2) glycosidic linkages, between 30 to 40 mol % β-(1,2) glycosidic linkages, between 10 to 30 mol % β-(1,2) glycosidic linkages, or between 10 to 20 mol % β-(1,2) glycosidic linkages.

In some variations, the oligosaccharide composition has a glycosidic bond type distribution of less than 40 mol % α-(1,3) glycosidic linkages, less than 30 mol % α-(1,3) glycosidic linkages, less than 25 mol % α-(1,3) glycosidic linkages, less than 20 mol % α-(1,3) glycosidic linkages, less than 15 mol % α-(1,3) glycosidic linkages, at least 1 mol % α-(1,3) glycosidic linkages, at least 5 mol % α-(1,3) glycosidic linkages, at least 10 mol % α-(1,3) glycosidic linkages, at least 15 mol % α-(1,3) glycosidic linkages, at least 20 mol % α-(1,3) glycosidic linkages, at least 25 mol % α-(1,3) glycosidic linkages, between 0 to 30 mol % α-(1,3) glycosidic linkages, between 1 to 30 mol % α-(1,3) glycosidic linkages, between 5 to 30 mol % α-(1,3) glycosidic linkages, between 10 to 25 mol % α-(1,3) glycosidic linkages, between 1 to 20 mol % α-(1,3) glycosidic linkages, or between 5 to 15 mol % α-(1,3) glycosidic linkages.

In some variations, the oligosaccharide composition has a glycosidic bond type distribution of less than 25 mol % β-(1,3) glycosidic linkages, less than 20 mol % β-(1,3) glycosidic linkages, less than 15 mol % β-(1,3) glycosidic linkages, less than 10 mol % β-(1,3) glycosidic linkages, at least 1 mol % β-(1,3) glycosidic linkages, at least 2 mol % β-(1,3) glycosidic linkages, at least 5 mol % β-(1,3) glycosidic linkages, at least 10 mol % β-(1,3) glycosidic linkages, at least 15 mol % β-(1,3) glycosidic linkages, between 1 to 20 mol % β-(1,3) glycosidic linkages, between 5 to 15 mol % β-(1,3) glycosidic linkages, between 1 to 15 mol % β-(1,3) glycosidic linkages, or between 2 to 10 mol % β-(1,3) glycosidic linkages.

In some variations, the oligosaccharide composition has a glycosidic bond type distribution of less than 20 mol % α-(1,4) glycosidic linkages, less than 15 mol % α-(1,4) glycosidic linkages, less than 10 mol % α-(1,4) glycosidic linkages, less than 9 mol % α-(1,4) glycosidic linkages, between 1 to 20 mol % α-(1,4) glycosidic linkages, between 1 to 15 mol % α-(1,4) glycosidic linkages, between 2 to 15 mol % α-(1,4) glycosidic linkages, between 5 to 15 mol % α-(1,4) glycosidic linkages, between 1 to 15 mol % α-(1,4) glycosidic linkages, or between 1 to 10 mol % α-(1,4) glycosidic linkages.

In some variations, the oligosaccharide composition has a glycosidic bond type distribution of less than 55 mol % β-(1,4) glycosidic linkages, less than 50 mol % β-(1,4) glycosidic linkages, less than 45 mol % β-(1,4) glycosidic linkages, less than 40 mol % β-(1,4) glycosidic linkages, less than 35 mol % β-(1,4) glycosidic linkages, less than 25 mol % β-(1,4) glycosidic linkages, less than 15 mol % β-(1,4) glycosidic linkages, less than 10 mol % β-(1,4) glycosidic linkages, at least 1 mol % β-(1,4) glycosidic linkages, at least 5 mol % β-(1,4) glycosidic linkages, at least 10 mol % β-(1,4) glycosidic linkages, at least 20 mol % β-(1,4) glycosidic linkages, at least 30 mol % β-(1,4) glycosidic linkages, between 0 to 55 mol % β-(1,4) glycosidic linkages, between 5 to 55 mol % β-(1,4) glycosidic linkages, between 10 to 50 mol % β-(1,4) glycosidic linkages, between 0 to 40 mol % β-(1,4) glycosidic linkages, between 1 to 40 mol % β-(1,4) glycosidic linkages, between 0 to 35 mol % β-(1,4) glycosidic linkages, between 1 to 35 mol % β-(1,4) glycosidic linkages, between 1 to 30 mol % 3-(1,4) glycosidic linkages, between 5 to 25 mol % β-(1,4) glycosidic linkages, between 10 to 25 mol % β-(1,4) glycosidic linkages, between 15 to 25 mol % β-(1,4) glycosidic linkages, between 0 to 15 mol % 3-(1,4) glycosidic linkages, between 1 to 15 mol % β-(1,4) glycosidic linkages, between 0 to 10 mol % (3-(1,4) glycosidic linkages, or between 1 to 10 mol % β-(1,4) glycosidic linkages.

In some variations, the oligosaccharide composition has a glycosidic bond type distribution of less than 30 mol % α-(1,6) glycosidic linkages, less than 25 mol % α-(1,6) glycosidic linkages, less than 20 mol % α-(1,6) glycosidic linkages, less than 19 mol % α-(1,6) glycosidic linkages, less than 15 mol % α-(1,6) glycosidic linkages, less than 10 mol % α-(1,6) glycosidic linkages, between 0 to 30 mol % α-(1,6) glycosidic linkages, between 1 to 30 mol % α-(1,6) glycosidic linkages, between 5 to 25 mol % α-(1,6) glycosidic linkages, between 0 to 25 mol % α-(1,6) glycosidic linkages, between 1 to 25 mol % α-(1,6) glycosidic linkages, between 0 to 20 mol % α-(1,6) glycosidic linkages, between 0 to 15 mol % α-(1,6) glycosidic linkages, between 1 to 15 mol % α-(1,6) glycosidic linkages, between 0 to 10 mol % α-(1,6) glycosidic linkages, or between 1 to 10 mol % α-(1,6) glycosidic linkages. In some embodiments, the oligosaccharide composition comprises oligosaccharides with hexose sugar monomers.

In some variations, the oligosaccharide composition has a glycosidic bond type distribution of less than 55 mol % β-(1,6) glycosidic linkages, less than 50 mol % β-(1,6) glycosidic linkages, less than 35 mol % β-(1,6) glycosidic linkages, less than 30 mol % β-(1,6) glycosidic linkages, at least 1 mol % β-(1,6) glycosidic linkages, at least 5 mol % β-(1,6) glycosidic linkages, at least 10 mol % β-(1,6) glycosidic linkages, at least 15 mol % β-(1,6) glycosidic linkages, at least 20 mol % β-(1,6) glycosidic linkages, at least 25 mol % β-(1,6) glycosidic linkages, at least 20 mol % β-(1,6) glycosidic linkages, at least 25 mol % $3-(1,6) glycosidic linkages, at least 30 mol % β-(1,6) glycosidic linkages, between 10 to 55 mol % β-(1,6) glycosidic linkages, between 5 to 55 mol % β-(1,6) glycosidic linkages, between 15 to 55 mol % β-(1,6) glycosidic linkages, between 20 to 55 mol % β-(1,6) glycosidic linkages, between 20 to 50 mol % β-(1,6) glycosidic linkages, between 25 to 55 mol % β-(1,6) glycosidic linkages, between 25 to 50 mol % β-(1,6) glycosidic linkages, between 5 to 40 mol % β-(1,6) glycosidic linkages, between 5 to 30 mol % β-(1,6) glycosidic linkages, between 10 to 35 mol % 3-(1,6) glycosidic linkages, between 5 to 20 mol % β-(1,6) glycosidic linkages, between 5 to 15 mol % (3-(1,6) glycosidic linkages, between 8 to 15 mol % β-(1,6) glycosidic linkages, or between 15 to 30 mol % β-(1,6) glycosidic linkages. In some embodiments, the oligosaccharide composition comprises oligosaccharides with hexose sugar monomers.

In some variations, the oligosaccharide composition has a glycosidic bond type distribution of at least 1 mol % α-(1,3) glycosidic linkages. In some variations, the oligosaccharide composition has a glycosidic bond type distribution of at least 10 mol % α-(1,3) glycosidic linkages.

In some variations, the oligosaccharide composition has a glycosidic bond type distribution of at least 1 mol % β-(1,3) glycosidic linkages. In some variations, the oligosaccharide composition has a glycosidic bond type distribution of at least 10 mol % β-(1,3) glycosidic linkages.

In some variations, the oligosaccharide composition has a glycosidic bond type distribution of at least 15 mol % β-(1,6) glycosidic linkages. In some variations, the oligosaccharide composition has a glycosidic bond type distribution of at least 10 mol % β-(1,6) glycosidic linkages.

In some variations, the oligosaccharide composition has a glycosidic bond type distribution of at least 15 mol % β-(1,2) glycosidic linkages. In some variations, the oligosaccharide composition has a glycosidic bond type distribution of at least 10 mol % β-(1,2) glycosidic linkages.

It should be understood that the glycosidic linkage distributions described herein for the various types of linkages (e.g., α-(1,2), α-(1,3), α-(1,4), α-(1,6), β-(1,2), β-(1,3), β-(1,4), or β-(1,6) glycosidic linkages) may be combined as if each and every combination were individually listed, as applicable.

In some variations, the distribution of glycosidic bond types described above for any of the oligosaccharide compositions herein is determined by two dimensional J-resolved nuclear magnetic resonance (2D-JRES NMR) spectroscopy.

In certain variations, the oligosaccharide composition comprises only hexose sugar monomers, and has any glycosidic bond type distribution as described herein. In some variations, the oligosaccharide composition comprises only pentose sugar monomers, and has any glycosidic bond type distribution as described herein, as applicable. In yet other variations, the oligosaccharide composition comprises both pentose and hexose sugar monomers, and has any glycosidic bond type distribution as described herein, as applicable.

It should be further understood that variations for the type of oligosaccharides present in the composition, as well as the degree of polymerization, glass transition temperature, and hygroscopicity of the oligosaccharide composition, may be combined as if each and every combination were listed separately. For example, in some variations, the oligosaccharide composition is made up of a plurality of oligosaccharides, wherein the composition has a glycosidic bond distribution of:
at least 1 mol % α-(1,3) glycosidic linkages;
at least 1 mol % β-(1,3) glycosidic linkages;
at least 15 mol % β-(1,6) glycosidic linkages;
less than 20 mol % α-(1,4) glycosidic linkages; and
less than 30 mol % α-(1,6) glycosidic linkages, and
wherein at least 10 dry wt % of the oligosaccharide composition has a degree of polymerization of at least 3. In some variations, at least 50 dry wt %, or between 65 and 80 dry wt % of the oligosaccharide composition has a degree of polymerization of at least 3.

For example, in some variations, the oligosaccharide composition has a glycosidic bond type distribution of less than 20 mol % α-(1,4) glycosidic linkages, and less than 30 mol % α-(1,6) glycosidic linkages. In some variations, at least 10 dry wt % of the oligosaccharide composition has a degree of polymerization of at least 3. In some variations, at least 50 dry wt %, or between 65 and 80 dry wt % of the oligosaccharide composition has a degree of polymerization of at least 3.

In another variation, the oligosaccharide composition comprises a glycosidic bond type distribution of between 0 to 15 mol % α-(1,2) glycosidic linkages; between 0 to 30 mol % (3-(1,2) glycosidic linkages; between 1 to 30 mol % α-(1,3) glycosidic linkages; between 1 to 20 mol % β-(1,3) glycosidic linkages; between 0 to 55 mol % β-(1,4) glycosidic linkages; and between 15 to 55 mol % β-(1,6) glycosidic linkages. In some variations, at least 10 dry wt % of the oligosaccharide composition has a degree of polymerization of at least 3. In some variations, at least 50 dry wt %, or between 65 and 80 dry wt % of the oligosaccharide composition has a degree of polymerization of at least 3.

In yet another variation, the oligosaccharide composition has a glycosidic bond type distribution of between 0 to 15 mol % α-(1,2) glycosidic linkages; between 10 to 30 mol % β-(1,2) glycosidic linkages; between 5 to 30 mol % α-(1,3) glycosidic linkages; between 1 to 20 mol % β-(1,3) glycosidic linkages; between 0 to 15 mol % β-(1,4) glycosidic linkages; between 20 to 55 mol % β-(1,6) glycosidic linkages; less than 20 mol % α-(1,4) glycosidic linkages; and less than 15 mol % α-(1,6) glycosidic linkages. In some variations, at least 10 dry wt % of the oligosaccharide composition has a degree of polymerization of at least 3. In some variations, at least 50 dry wt %, or between 65 and 80 dry wt % of the oligosaccharide composition has a degree of polymerization of at least 3.

In still other variations, the oligosaccharide composition has a glycosidic bond type distribution of between 0 to 10 mol % α-(1,2) glycosidic linkages, between 15 to 25 mol % β-(1,2) glycosidic linkages, between 10 to 25 mol % α-(1,3) glycosidic linkages, between 5 to 15 mol % β-(1,3) glycosidic linkages, between 5 to 15 mol % α-(1,4) glycosidic linkages, between 0 to 10 mol % β-(1,4) glycosidic linkages, between 0 to 10 mol % α-(1,6) glycosidic linkages, and between 25 to 50 mol % β-(1,6) glycosidic linkages. In some variations, at least 10 dry wt % of the oligosaccharide composition has a degree of polymerization of at least 3. In some variations, at least 50 dry wt %, or between 65 and 80 dry wt % of the oligosaccharide composition has a degree of polymerization of at least 3.

In certain variations, the oligosaccharide composition has a glycosidic bond type distribution of between 0 to 15 mol % α-(1,2) glycosidic linkages; between 0 to 15 mol % β-(1,2) glycosidic linkages; between 1 to 20 mol % α-(1,3) glycosidic linkages; between 1 to 15 mol % β-(1,3) glycosidic linkages; between 5 to 55 mol % β-(1,4) glycosidic linkages; between 15 to 55 mol % β-(1,6) glycosidic linkages; less than 20 mol % α-(1,4) glycosidic linkages; and less than 30 mol % α-(1,6) glycosidic linkages. In some variations, at least 10 dry wt % of the oligosaccharide composition has a degree of polymerization of at least 3. In some variations, at least 50 dry wt %, or between 65 and 80 dry wt % of the oligosaccharide composition has a degree of polymerization of at least 3.

In yet other variations, the oligosaccharide composition has a glycosidic bond type distribution of between 0 to 10 mol % α-(1,2) glycosidic linkages, between 0 to 10 mol % β-(1,2) glycosidic linkages, between 5 to 15 mol % α-(1,3) glycosidic linkages, between 2 to 10 mol % β-(1,3) glycosidic linkages, between 2 to 15 mol % α-(1,4) glycosidic linkages, between 10 to 50 mol % β-(1,4) glycosidic linkages, between 5 to 25 mol % α-(1,6) glycosidic linkages, and between 20 to 50 mol % β-(1,6) glycosidic linkages. In some variations, at least 10 dry wt % of the oligosaccharide composition has a degree of polymerization of at least 3. In some variations, at least 50 dry wt %, or between 65 and 80 dry wt % of the oligosaccharide composition has a degree of polymerization of at least 3.

In other variations, the oligosaccharide composition has a glycosidic bond type distribution of between 0 to 15 mol % α-(1,2) glycosidic linkages, between 0 to 30 mol % β-(1,2) glycosidic linkages, between 5 to 30 mol % α-(1,3) glycosidic linkages, between 1 to 20 mol % β-(1,3) glycosidic linkages, between 1 to 20 mol % α-(1,4) glycosidic linkages, between 0 to 40 mol % β-(1,4) glycosidic linkages, between 0 to 25 mol % α-(1,6) glycosidic linkages, and between 10 to 35 mol % β-(1,6) glycosidic linkages. In some variations, at least 10 dry wt % of the oligosaccharide composition has a degree of polymerization of at least 3. In some variations, at least 50 dry wt %, or between 65 and 80 dry wt % of the oligosaccharide composition has a degree of polymerization of at least 3.

In still other variations, the oligosaccharide composition has a glycosidic bond type distribution of between 0 to 10 mol % α-(1,2) glycosidic linkages, between 0 to 25 mol % β-(1,2) glycosidic linkages, between 10 to 25 mol % α-(1,3) glycosidic linkages, between 5 to 15 mol % β-(1,3) glycosidic linkages, between 5 to 15 mol % α-(1,4) glycosidic linkages, between 0 to 35 mol % β-(1,4) glycosidic linkages, between 0 to 20 mol % α-(1,6) glycosidic linkages, and between 15 to 30 mol % β-(1,6) glycosidic linkages. In some variations, at least 10 dry wt % of the oligosaccharide composition has a degree of polymerization of at least 3. In some variations, at least 50 dry wt %, or between 65 and 80 dry wt % of the oligosaccharide composition has a degree of polymerization of at least 3.

In still other variations, the oligosaccharide composition has a glycosidic bond type distribution of at least 1 mol % α-(1,3) glycosidic linkages, and at least 1 mol % β-(1,3) glycosidic linkages, wherein at least 10 dry wt % of the oligosaccharide composition has a degree of polymerization of at least 3. In some variations, the oligosaccharide composition further has a glycosidic bond type distribution of at least 15 mol % β-(1,6) glycosidic linkages. In yet other variations, at least 50 dry wt %, or between 65 and 80 dry wt % of the oligosaccharide composition has a degree of polymerization of at least 3.

In some variations, the oligosaccharide composition has a glycosidic bond type distribution of at least 10 mol % α-(1,3) glycosidic linkages; and at least 10 mol % β-(1,3) glycosidic linkages. In some variations, the oligosaccharide composition has a glycosidic bond type distribution of less than 9 mol % α-(1,4) glycosidic linkages; and less than 19 mol % α-(1,6) glycosidic linkages. In some variations, the oligosaccharide composition further has a glycosidic bond type distribution of at least 15 mol % β-(1,2) glycosidic linkages.

In other variations, the oligosaccharide composition has a glycosidic bond type distribution of less than 9 mol % α-(1,4) glycosidic linkages, and less than 19 mol % α-(1,6) glycosidic linkages.

In still other variations, the oligosaccharide composition has a glycosidic bond type distribution of between 0 to 20 mol % α-(1,2) glycosidic linkages; between 10 to 45 mol % β-(1,2) glycosidic linkages; between 1 to 30 mol % α-(1,3) glycosidic linkages; between 1 to 20 mol % β-(1,3) glycosidic linkages; between 0 to 55 mol % β-(1,4) glycosidic linkages; and between 10 to 55 mol % β-(1,6) glycosidic linkages.

In some variations, the oligosaccharide composition has a glycosidic bond type distribution of between 10 to 20 mol % α-(1,2) glycosidic linkages, between 23 to 31 mol % β-(1,2) glycosidic linkages, between 7 to 9 mol % α-(1,3) glycosidic linkages, between 4 to 6 mol % β-(1,3) glycosidic linkages, between 0 to 2 mol % α-(1,4) glycosidic linkages, between 18 to 22 mol % β-(1,4) glycosidic linkages, between 9 to 13 mol % α-(1,6) glycosidic linkages, and between 14 to 16 mol % β-(1,6) glycosidic linkages

In yet other variations, the oligosaccharide composition has a glycosidic bond type distribution of between 10 to 12 mol % α-(1,2) glycosidic linkages, between 31 to 39 mol % β-(1,2) glycosidic linkages, between 5 to 7 mol % α-(1,3) glycosidic linkages, between 2 to 4 mol % β-(1,3) glycosidic linkages, between 0 to 2 mol % α-(1,4) glycosidic linkages, between 19 to 23 mol % β-(1,4) glycosidic linkages, between 13 to 17 mol % α-(1,6) glycosidic linkages, and between 7 to 9 mol % β-(1,6) glycosidic linkages.

In some embodiments, which may be combined with any of the foregoing embodiments, at least 10 dry wt % of the oligosaccharide composition has a degree of polymerization of at least 3. In some variations, at least 50 dry wt %, or between 65 and 80 dry wt % of the oligosaccharide composition has a degree of polymerization of at least 3.

### Metabolizable Energy Content

As used herein, the “metabolizable energy content” measures the total amount of energy obtained through the digestion and metabolism of a food or food ingredient. In certain variations, the metabolizable energy content can be determined using the nitrogen-corrected true metabolizable energy content assay described, for example, in Parsons, C. M., L. M. Potter, and B. A. Bliss. 1982. True metabolizable energy corrected to nitrogen equilibrium. Poultry Sci. 61: 2241-2246.

In some variations, the oligosaccharide composition has a metabolizable energy content, on a dry matter basis, of less than 4 kcal/g, less than 3.9 kcal/g, less than 3.8 kcal/g, less than 3.7 kcal/g, less than 3.6 kcal/g, less than 3.5 kcal/g, less than 3.4 kcal/g, less than 3.3 kcal/g, less than 3.2 kcal/g, less than 3.1 kcal/g, less than 3 kcal/g, less than 2.9 kcal/g, less than 2.8 kcal/g, less than 2.7 kcal/g, less than 2.6 kcal/g, less than 2.5 kcal/g, less than 2.4 kcal/g, less than 2.3 kcal/g, less than 2.2 kcal/g, less than 2.1 kcal/g, less than 2 kcal/g, less than 1.9 kcal/g, less than 1.8 kcal/g, less than 1.7 kcal/g, less than 1.6 kcal/g, or less than 1.5 kcal/g.

In certain variations, the oligosaccharide composition has a metabolizable energy content, on a dry matter basis, of greater than 1 kcal/g and less than 2.5 kcal/g; or greater than 1 kcal/g and less than 2 kcal/g. In one variation, the oligosaccharide composition has a metabolizable energy content, on a dry matter basis, of between 1 kcal/g and 2.7 kcal/g, or between 1.1 kcal/g and 2.5 kcal/g, or between 1.1 and 2 kcal/g.

It should be understood that the oligosaccharide compositions described herein may be characterized based on the type of oligosaccharides present, degree of polymerization, digestibility, glass transition temperature, hygroscopicity, fiber content, glycosidic bond type distribution, and metabolizable energy content described herein, as if each and every combination were listed separately.

For example, in one variation, the oligosaccharide composition has:
(a) a glycosidic bond type distribution of:
   at least 10 mol % α-(1,3) glycosidic linkages; and
   at least 10 mol % β-(1,3) glycosidic linkages; and
(b) at least 10 dry wt % of the oligosaccharide composition has a degree of polymerization of at least 3; and
(c) a metabolizable energy content, on a dry matter basis, of less than 2.7 kcal/g.

For example, in another variation, the oligosaccharide composition has:
(a) a glycosidic bond type distribution of:
   at least 10 mol % α-(1,3) glycosidic linkages; and
   at least 10 mol % β-(1,3) glycosidic linkages; and
   less than 9 mol % α-(1,4) glycosidic linkages; and
   less than 19 mol % α-(1,6) glycosidic linkages; and
(b) at least 10 dry wt % of the oligosaccharide composition has a degree of polymerization of at least 3; and
(c) a metabolizable energy content, on a dry matter basis, of less than 2.7 kcal/g.

For example, in another variation, provided is a food ingredient that includes an oligosaccharide composition, wherein the oligosaccharide composition has:
(a) a glycosidic bond type distribution of:
   less than 9 mol % α-(1,4) glycosidic linkages; and
   less than 19 mol % α-(1,6) glycosidic linkages; and
(b) at least 10 dry wt % of the oligosaccharide composition has a degree of polymerization of at least 3; and
(c) a metabolizable energy content, on a dry matter basis, of less than 2.7 kcal/g.

In some variations, the oligosaccharide composition has a glycosidic bond type distribution of at least 15 mol % 3-(1,2) glycosidic linkages.

In one variation of the foregoing, the oligosaccharide composition further has:
(d) a digestibility of less than 0.20 g/g; or
(e) the glass transition temperature of at least 0° C., measured at less 10 wt % water; or
(f) the hygroscopicity of at least 5% moisture content, measure at 0.6 water activity; or
(g) the dietary fiber content of at least 50% on a dry mass basis; or
any combination of (d)-(g).

### Food Products

The oligosaccharide compositions produced according to the methods described herein may be suitable as an ingredient for foods, for example as a replacement or supplement for conventional carbohydrates. The oligosaccharide compositions may be added to foods to increase the dietary fiber content. In certain embodiments, increasing the dietary fiber content of a food product by including the one or more oligosaccharides has one or more beneficial health effects, including, for example, lowering the glycemic index of a food product, reducing cholesterol, attenuating blood dextrose, and/or maintaining gastrointestinal health.

The oligosaccharide compositions may also be added to foods to reduce the caloric content. For example, the oligosaccharide compositions may be used to fully or partially replacing nutritive sweeteners such as sucrose, fructose, or high-fructose corn syrup, reducing calorie content. The oligosaccharide compositions may also be used as a bulking agent, replacing fat, flour, or other ingredients in food, which may reduce calorie content. The oligosaccharide compositions may also be added to foods to improve food texture (e.g., softer, crunchier), to extend shelf life (e.g., depress water activity, reduce clumping), or to improve the processing characteristics (e.g., reduce clumping). For example, the oligosaccharide compositions may be used to reduce the sugar content and enhance the dietary fiber content of breakfast cereals, granola and other type of bars, yogurt, ice cream, breads, cookies, candy, cake mixes, and nutritional shakes and supplements.

### Methods of Producing Food Ingredients and Food Products

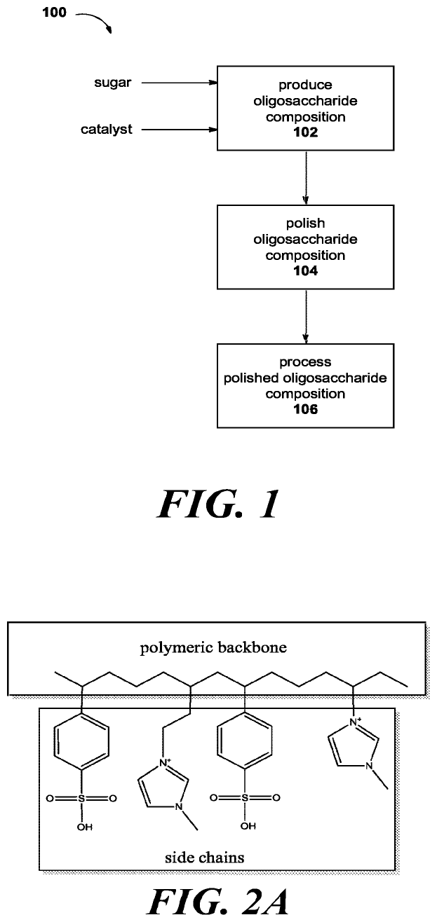

With reference to FIG. 1, process 100 depicts an exemplary process to produce an oligosaccharide composition from sugars, and such oligosaccharide composition produced can subsequently be polished and further processed to form a food ingredient, such as an oligosaccharide syrup or powder. In step 102, one or more sugars are combined with a catalyst in a reactor. The sugars may include, for example, monosaccharides, disaccharides, and/or trisaccharides. The catalyst has both acidic and ionic groups. In some variations, the catalyst is a polymeric catalyst that includes acidic monomers and ionic monomers. In other variations, the catalyst is a solid-supported catalyst that includes acidic moieties and ionic moieties.

In step 104, the oligosaccharide composition in step 102 is polished to remove fine solids, reduce color, and reduce conductivity, and/or modify the molecular weight distribution. Any suitable methods known in the art to polish the oligosaccharide composition may be used, including, for example, the use of filtration units, carbon or other absorbents, chromatographic separators, or ion exchange columns. For example, in one variation, the oligosaccharide composition is treated with powdered activated carbon to reduce color, microfiltered to remove fine solids, and passed over a strong-acid cationic exchange resin and a weak-base anionic exchange resin to remove salts. In another variation, the oligosaccharide composition is microfiltered to remove fine solids and passed over a weak-base anionic exchange resin. In yet another variation, the oligosaccharide composition is passed through a simulated moving bed chromatographic separator to remove low molecular mass species.

In step 106, the polished oligosaccharide composition undergoes further processing to produce either an oligosaccharide syrup or powder. For example, in one variation, the polished oligosaccharide is concentrated to form a syrup. Any suitable methods known in the art to concentrate a solution may be used, such as the use of a vacuum evaporator. In another variation, the polished oligosaccharide composition is spray dried to form a powder. Any suitable methods known in the art to spray dry a solution to form a powder may be used.

In other variations, process 100 may be modified to have additional steps. For example, the oligosaccharide composition produced in step 102 may be diluted (e.g., in a dilution tank) and then undergo a carbon treatment to decolorize the oligosaccharide composition prior to polishing in step 104. In other variations, the oligosaccharide composition produced in step 102 may undergo further processing in a simulated moving bed (SMB) separation step to reduce digestible carbohydrate content.

In other variations, process 100 may be modified to have fewer steps. For example, in one variation, step 106 to produce the oligosaccharide syrup or powder may be omitted, and the polished oligosaccharide composition of step 104 may be used directly as an ingredient to produce a food product.

Each of the steps in exemplary process 100, the reactants and processing conditions in each step, as well as the compositions produced in each step are described in further detail below.

### Feed Sugar

The feed sugar used to produce the oligosaccharide compositions may include one or more sugars. In some embodiments, the one or more sugars are selected from monosaccharides, disaccharides, trisaccharides, and short-chain oligosaccharides, or any mixtures thereof. In some embodiments, the one or more sugars are monosaccharides, such as one or more C5 or C6 monosaccharides. Exemplary monosaccharides include glucose, galactose, mannose, fructose, xylose, xylulose, and arabinose. In some embodiments, the one or more sugars are C5 monosaccharides. In other embodiments, the one or more sugars are C6 monosaccharides. In some embodiments, the one or more sugars are selected from glucose, galactose, mannose, lactose, or their corresponding sugar alcohols. In other embodiments, the one or more sugars are selected from fructose, xylose, arabinose, or their corresponding sugar alcohols. In some embodiments, the one or more sugars are disaccharides. Exemplary disaccharides include lactose, sucrose and cellobiose. In some embodiments, the one or more sugars are trisaccharides, such as maltotriose or raffinose. In some embodiments, the one or more sugars comprise a mixture of short-chain oligosaccharides, such as malto-dextrins. In certain embodiments, the one or more sugars are corn syrup obtained from the partial hydrolysis of corn starch. In a particular embodiment, the one or more sugars is corn syrup with a dextrose equivalent (DE) below 50 (e.g., 10 DE corn syrup, 18 DE corn syrup, 25 DE corn syrup, or 30 DE corn syrup).

In some embodiments, the method used to produce the oligosaccharide compositions involves combining two or more sugars with the catalyst to produce one or more oligosaccharides. In some embodiments, the two or more sugars are selected from glucose, galactose, mannose and lactose (e.g., glucose and galactose).

In other embodiments, the method used to produce the oligosaccharide compositions involves combining a mixture of sugars (e.g., monosaccharides, disaccharides, trisaccharides, etc., and/or other short oligosaccharides) with the catalyst to produce one or more oligosaccharides. In one embodiment, the method includes combining corn glucose syrup with the catalyst to produce one or more oligosaccharides.

In other embodiments, the method used to produce the oligosaccharide compositions involves combining a polysaccharide with the catalyst to produce one or more oligosaccharides. In some embodiments, the polysaccharide is selected from starch, guar gum, xanthan gum and acacia gum.

In other embodiments, the method used to produce the oligosaccharide compositions involves combining a mixture of sugars and sugar alcohols with the catalyst to produce one or more oligosaccharides. In particular embodiments, the method includes combining one or more sugars and one or more alcohols selected from the group consisting of glucitol, sorbitol, xylitol and arabinatol, with the catalyst to produce one or more oligosaccharides.

In certain variations, the feed sugar includes glucose, mannose, galactose, xylose, malto-dextrin, arabinose, or galactose, or any combinations thereof. The choice of feed sugars will impact the resulting oligosaccharide composition produced. For example, in one variation where the feed sugar is all glucose, the resulting oligosaccharide composition is a gluco-oligosaccharide. In another variation where the feed sugar is all mannose, the resulting oligosaccharide composition is a manno-oligosaccharide. In another variation wherein the feed sugar includes glucose and galactose, the resulting oligosaccharide composition is a gluco-galacto-oligosaccharide. In yet another variation where the feed sugar is all xylose, the resulting oligosaccharide composition is a xylo-oligosaccharide. In another variation where the feed sugar includes malto-dextrin, the resulting oligosaccharide composition is a gluco-oligosaccharide. In yet another variation where the feed sugar includes xylose, glucose and galactose, the resulting oligosaccharide composition is a gluco-galacto-xylo-oligosaccharide. In one variation where the feed sugar includes arabinose and xylose, the resulting oligosaccharide composition is an arabino-xylo-oligosaccharide. In another variation where the feed sugar includes glucose and xylose, the resulting oligosaccharide composition is a gluco-xylo-oligosaccharide. In yet another variation where the feed sugar includes glucose, galactose and xylose, the resulting oligosaccharide composition is a xylo-gluco-galacto-oligosaccharide.

In some variations to produce the oligosaccharide compositions herein, the sugars may be provided as a feed solution, in which the sugars are combined with water and fed into the reactor. In other variations, the sugars may be fed into the reactor as a solid and combined with water in the reactor.

The feed sugars used to produce the oligosaccharide compositions herein may be obtained from any commercially known sources, or produced according to any methods known in the art.

### Catalysts

The catalysts used in the methods described herein include polymeric catalysts and solid-supported catalysts.

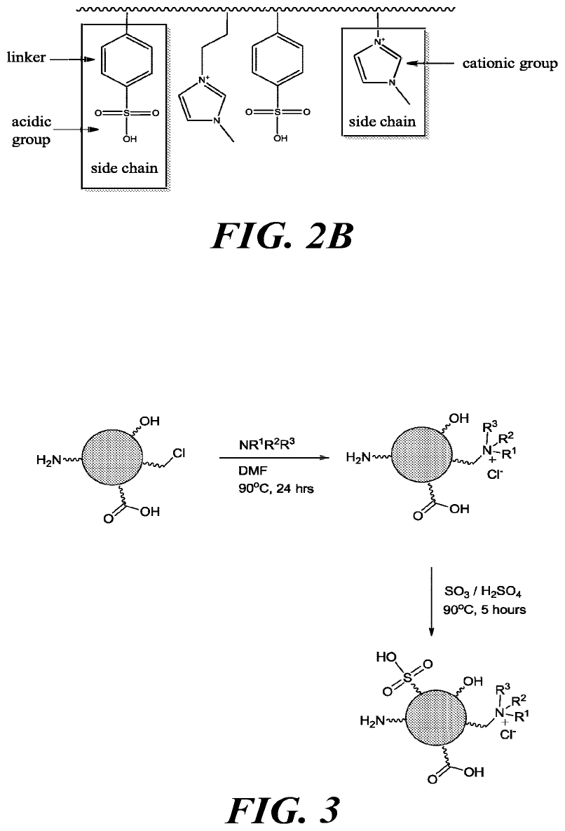

In some embodiments, the catalyst is a polymer made up of acidic monomers and ionic monomers (which are also referred to herein as “ionomers”) connected to form a polymeric backbone. Each acidic monomer includes at least one Bronsted-Lowry acid, and each ionic monomer includes at least one nitrogen-containing cationic group, at least one phosphorous-containing cationic group, or any combination thereof. In certain embodiments of the polymeric catalyst, at least some of the acidic and ionic monomers may independently include a linker connecting the Bronsted-Lowry acid or the cationic group (as applicable) to a portion of the polymeric backbone. For the acidic monomers, the Bronsted-Lowry acid and the linker together form a side chain. Similarly, for the ionic monomers, the cationic group and the linker together form a side chain. With reference to the portion of the polymeric catalyst depicted in FIGS. 2A and 2B, the side chains are pendant from the polymeric backbone.

In another aspect, the catalyst is solid-supported, having acidic moieties and ionic moieties each attached to a solid support. Each acidic moiety independently includes at least one Bronsted-Lowry acid, and each ionic moiety includes at least one nitrogen-containing cationic group, at least one phosphorous-containing cationic group, or any combination thereof. In certain embodiments of the solid-supported catalyst, at least some of the acidic and ionic moieties may independently include a linker connecting the Bronsted-Lowry acid or the cationic group (as applicable) to the solid support. With reference to FIG. 3, the catalyst produced is a solid-supported catalyst with acidic and ionic moieties.

### Acidic Monomers and Moieties

The polymeric catalysts include a plurality of acidic monomers, where as the solid-supported catalysts include a plurality of acidic moieties attached to a solid support.

In some embodiments, a plurality of acidic monomers (e.g., of a polymeric catalyst) or a plurality of acidic moieties (e.g., of a solid-supported catalyst) has at least one Bronsted-Lowry acid. In certain embodiments, a plurality of acidic monomers (e.g., of a polymeric catalyst) or a plurality of acidic moieties (e.g., of a solid-supported catalyst) has one Bronsted-Lowry acid or two Bronsted-Lowry acids. In certain embodiments, a plurality of the acidic monomers (e.g., of a polymeric catalyst) or a plurality of the acidic moieties (e.g., of a solid-supported catalyst) has one Bronsted-Lowry acid, while others have two Bronsted-Lowry acids.

In some embodiments, each Bronsted-Lowry acid is independently selected from sulfonic acid, phosphonic acid, acetic acid, isophthalic acid, and boronic acid. In certain embodiments, each Bronsted-Lowry acid is independently sulfonic acid or phosphonic acid. In one embodiment, each Bronsted-Lowry acid is sulfonic acid. It should be understood that the Bronsted-Lowry acids in an acidic monomer (e.g., of a polymeric catalyst) or an acidic moiety (e.g., of a solid-supported catalyst) may be the same at each occurrence or different at one or more occurrences.

In some embodiments, one or more of the acidic monomers of a polymeric catalyst are directly connected to the polymeric backbone, or one or more of the acidic moieties of a solid-supported catalyst are directly connected to the solid support. In other embodiments, one or more of the acidic monomers (e.g., of a polymeric catalyst) or one or more acidic moieties (e.g., of a solid-supported catalyst) each independently further includes a linker connecting the Bronsted-Lowry acid to the polymeric backbone or the solid support (as the case may be). In certain embodiments, some of the Bronsted-Lowry acids are directly connected to the polymeric backbone or the solid support (as the case may be), while other the Bronsted-Lowry acids are connected to the polymeric backbone or the solid support (as the case may be) by a linker.

In those embodiments where the Bronsted-Lowry acid is connected to the polymeric backbone or the solid support (as the case may be) by a linker, each linker is independently selected from unsubstituted or substituted alkyl linker, unsubstituted or substituted cycloalkyl linker, unsubstituted or substituted alkenyl linker, unsubstituted or substituted aryl linker, and unsubstituted or substituted heteroaryl linker. In certain embodiments, the linker is unsubstituted or substituted aryl linker, or unsubstituted or substituted heteroaryl linker. In certain embodiments, the linker is unsubstituted or substituted aryl linker. In one embodiment, the linker is a phenyl linker. In another embodiment, the linker is a hydroxyl-substituted phenyl linker.

In other embodiments, each linker in an acidic monomer (e.g., of a polymeric catalyst) or an acidic moiety (e.g., of a solid-supported catalyst) is independently selected from:
unsubstituted alkyl linker;
alkyl linker substituted 1 to 5 substituents independently selected from oxo, hydroxy, halo, amino;
unsubstituted cycloalkyl linker;
cycloalkyl linker substituted 1 to 5 substituents independently selected from oxo, hydroxy, halo, amino;
unsubstituted alkenyl linker;
alkenyl linker substituted 1 to 5 substituents independently selected from oxo, hydroxy, halo, amino;
unsubstituted aryl linker;
aryl linker substituted 1 to 5 substituents independently selected from oxo, hydroxy, halo, amino;
unsubstituted heteroaryl linker; or
heteroaryl linker substituted 1 to 5 substituents independently selected from oxo, hydroxy, halo, amino.

Further, it should be understood that some or all of the acidic monomers (e.g., of a polymeric catalyst) or one or more acidic moieties (e.g., of a solid-supported catalyst) connected to the polymeric backbone by a linker may have the same linker, or independently have different linkers.

In some embodiments, each acidic monomer (e.g., of a polymeric catalyst) and each acidic moiety (e.g., of a solid-supported catalyst) may independently have the structure of Formulas IA-VIA:

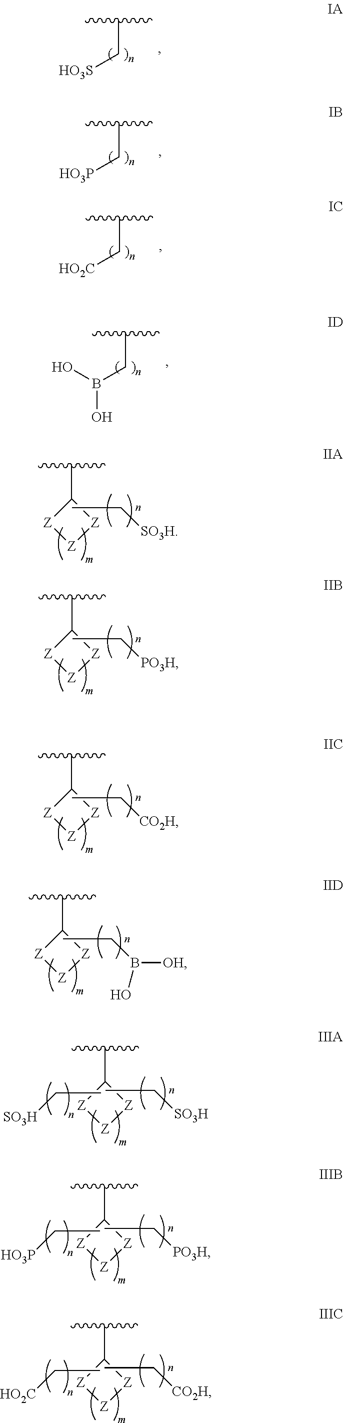

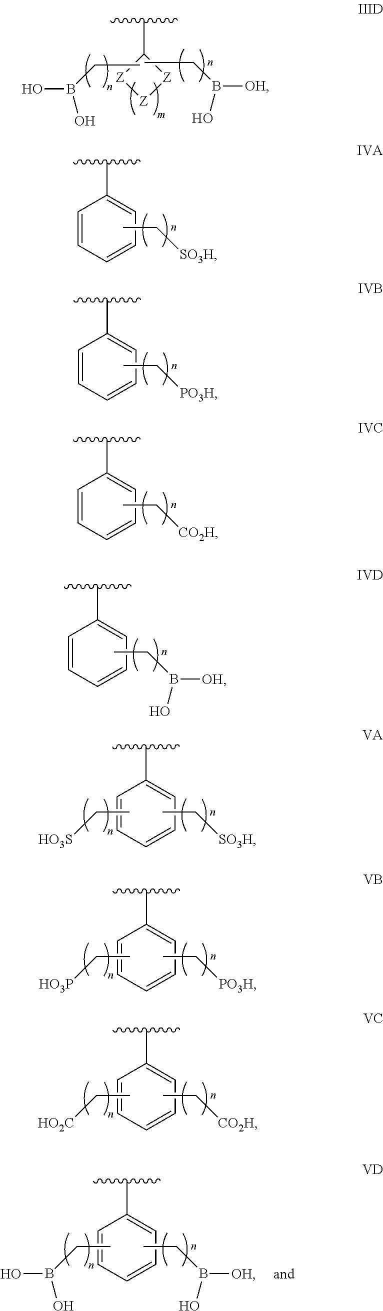

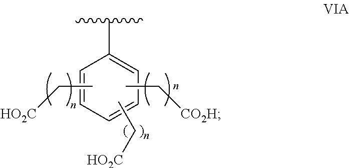

wherein:
each Z is independently C(R²)(R³), N(R⁴), S, S(R⁵)(R⁶), S(O)(R⁵)(R⁶), SO₂, or O, wherein any two adjacent Z can (to the extent chemically feasible) be joined by a double bond, or taken together to form cycloalkyl, heterocycloalkyl, aryl or heteroaryl;
each m is independently selected from 0, 1, 2, and 3;
each n is independently selected from 0, 1, 2, and 3;
each R², R³, and R⁴ is independently hydrogen, alkyl, heteroalkyl, cycloalkyl, heterocyclyl, aryl, or heteroaryl; and
each R⁵ and R⁶ is independently alkyl, heteroalkyl, cycloalkyl, heterocyclyl, aryl, or heteroaryl.

In some embodiments, each acidic monomer (e.g., of a polymeric catalyst) and each acidic moiety (e.g., of a solid-supported catalyst) may independently have the structure of Formulas IA, IB, IVA, or IVB. In other embodiments, each acidic monomer (e.g., of a polymeric catalyst) and each acidic moiety (e.g., of a solid-supported catalyst) may independently have the structure of Formulas IIA, IIB, IIC, IVA, IVB, or IVC. In other embodiments, each acidic monomer (e.g., of a polymeric catalyst) and each acidic moiety (e.g., of a solid-supported catalyst) may independently have the structure of Formulas IIIA, IIIB, or IIIC. In some embodiments, each acidic monomer (e.g., of a polymeric catalyst) and each acidic moiety (e.g., of a solid-supported catalyst) may independently have the structure of Formulas VA, VB, or VC. In some embodiments, each acidic monomer (e.g., of a polymeric catalyst) and each acidic moiety (e.g., of a solid-supported catalyst) may independently have the structure of Formula IA. In other embodiments, each acidic monomer (e.g., of a polymeric catalyst) and each acidic moiety (e.g., of a solid-supported catalyst) may independently have the structure of Formula IB.

In some embodiments, Z can be chosen from C(R₂)(R₃), N(R₄), SO₂, and O. In some embodiments, any two adjacent Z can be taken together to form a group selected from a heterocycloalkyl, aryl, and heteroaryl. In other embodiments, any two adjacent Z can be joined by a double bond. Any combination of these embodiments is also contemplated (as chemically feasible).

In some embodiments, m is 2 or 3. In other embodiments, n is 1, 2, or 3. In some embodiments, R¹ can be hydrogen, alkyl or heteroalkyl. In some embodiments, R¹ can be hydrogen, methyl, or ethyl. In some embodiments, each R², R³ and R⁴ can independently be hydrogen, alkyl, heterocyclyl, aryl, or heteroaryl. In other embodiments, each R², R³ and R⁴ can independently be heteroalkyl, cycloalkyl, heterocyclyl, or heteroaryl. In some embodiments, each R⁵ and R⁶ can independently be alkyl, heterocyclyl, aryl, or heteroaryl. In another embodiment, any two adjacent Z can be taken together to form cycloalkyl, heterocycloalkyl, aryl or heteroaryl.

In some embodiments, the polymeric catalysts and solid-supported catalysts described herein contain monomers or moieties, respectively, that have at least one Bronsted-Lowry acid and at least one cationic group. The Bronsted-Lowry acid and the cationic group can be on different monomers/moieties or on the same monomer/moiety.

In certain embodiments, the acidic monomers of the polymeric catalyst may have a side chain with a Bronsted-Lowry acid that is connected to the polymeric backbone by a linker. In certain embodiments, the acidic moieties of the solid-supported catalyst may have a Bronsted-Lowry acid that is attached to the solid support by a linker. Side chains (e.g., of a polymeric catalyst) or acidic moieties (e.g., of a solid-supported catalyst) with one or more Bronsted-Lowry acids connected by a linker can include, for example,

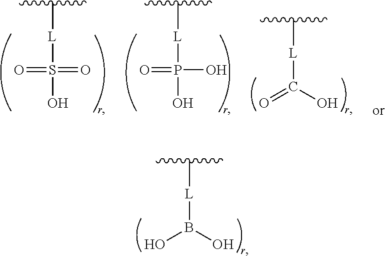

wherein:
L is an unsubstituted alkyl linker, alkyl linker substituted with oxo, unsubstituted cycloalkyl, unsubstituted aryl, unsubstituted heterocycloalkyl, and unsubstituted heteroaryl; and
r is an integer.

In certain embodiments, L is an alkyl linker. In other embodiments L is methyl, ethyl, propyl, or butyl. In yet other embodiments, the linker is ethanoyl, propanoyl, or benzoyl. In certain embodiments, r is 1, 2, 3, 4, or 5 (as applicable or chemically feasible).

In some embodiments, at least some of the acidic side chains (e.g., of a polymeric catalyst) and at least some of the acidic moieties (e.g., of a solid-supported catalyst) may be:

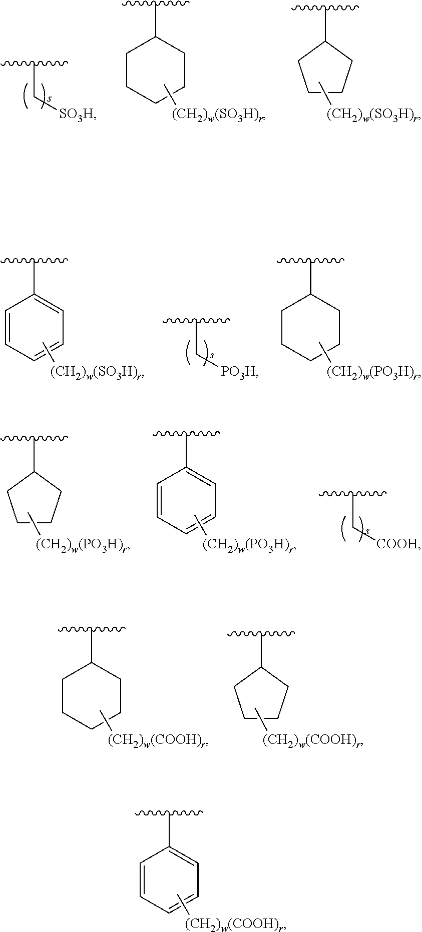

wherein:
s is 1 to 10;
each r is independently 1, 2, 3, 4, or 5 (as applicable or chemically feasible); and
w is 0 to 10.

In certain embodiments, s is 1 to 9, or 1 to 8, or 1 to 7, or 1 to 6, or 1 to 5, or 1 to 4, or 1 to 3, or 2, or 1. In certain embodiments, w is 0 to 9, or 0 to 8, or 0 to 7, or 0 to 6, or 0 to 5, or 0 to 4, or 0 to 3, or 0 to 2, 1 or 0).

In certain embodiments, at least some of the acidic side chains (e.g., of a polymeric catalyst) and at least some of the acidic moieties (e.g., of a solid-supported catalyst) may be:

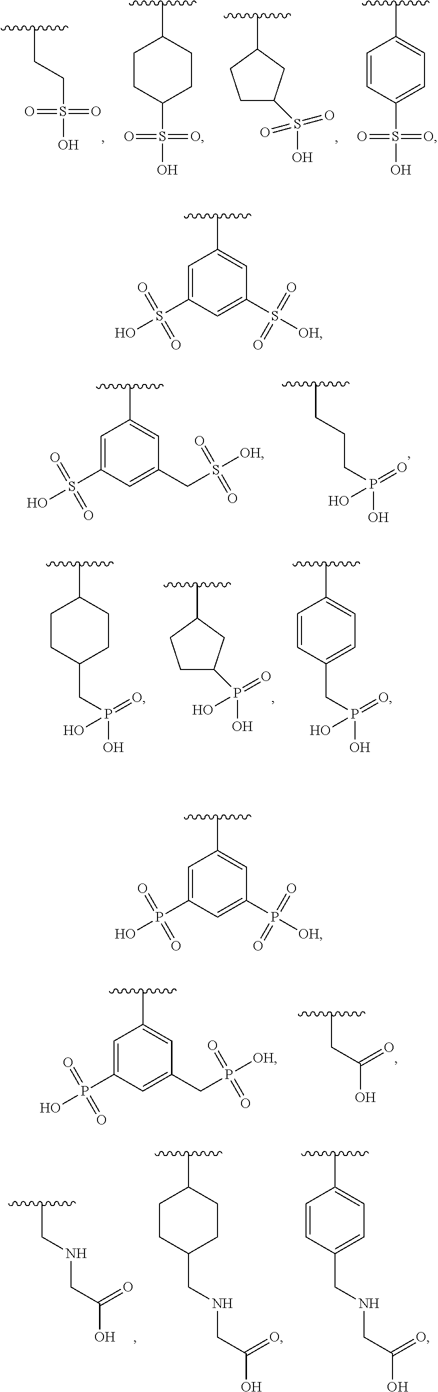

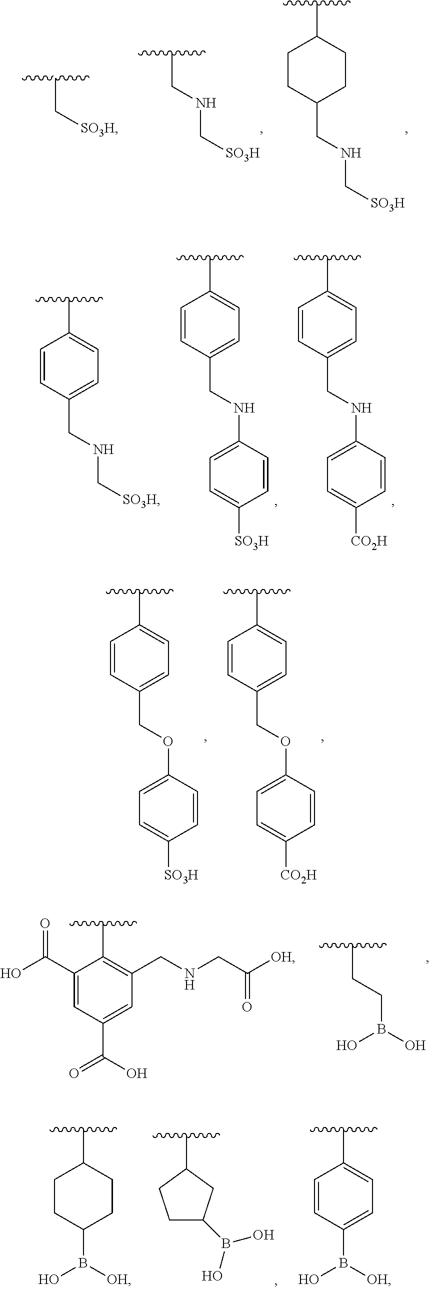

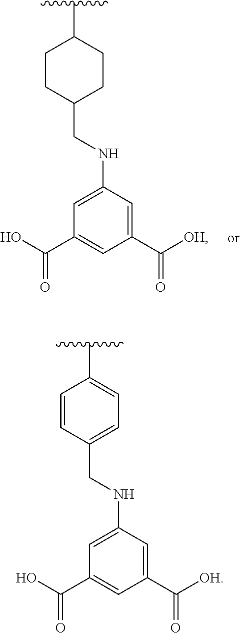 [SMILES: *C1CCC(CNc2cc(C(=O)O)cc(C(=O)O)c2)CC1 ; *c1ccc(CNc2cc(C(=O)O)cc(C(=O)O)c2)cc1]

In other embodiments, the acidic monomers (e.g., of a polymeric catalyst) can have a side chain with a Bronsted-Lowry acid that is directly connected to the polymeric backbone. In other embodiments, the acidic moieties (e.g., of a solid-supported catalyst) may be directly attached to a solid support. Side chains directly connect to the polymeric backbone (e.g., of a polymeric catalyst) or acidic moieties (e.g., of a solid-supported catalyst) directly attached to the solid support may can include, for example,

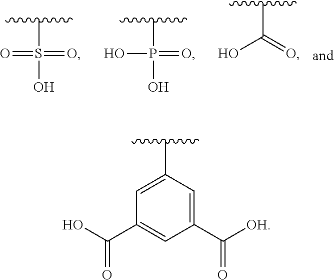

### Ionic Monomers and Moieties

The polymeric catalysts include a plurality of ionic monomers, where as the solid-supported catalysts includes a plurality of ionic moieties attached to a solid support.

In some embodiments, a plurality of ionic monomers (e.g., of a polymeric catalyst) or a plurality of ionic moieties (e.g., of a solid-supported catalyst) has at least one nitrogen-containing cationic group, at least one phosphorous-containing cationic group, or any combination thereof. In certain embodiments, a plurality of ionic monomers (e.g., of a polymeric catalyst) or a plurality of ionic moieties (e.g., of a solid-supported catalyst) has one nitrogen-containing cationic group or one phosphorous-containing cationic group. In some embodiments, a plurality of ionic monomers (e.g., of a polymeric catalyst) or a plurality of ionic moieties (e.g., of a solid-supported catalyst) has two nitrogen-containing cationic groups, two phosphorous-containing cationic group, or one nitrogen-containing cationic group and one phosphorous-containing cationic group. In other embodiments, a plurality of ionic monomers (e.g., of a polymeric catalyst) or a plurality of ionic moieties (e.g., of a solid-supported catalyst) has one nitrogen-containing cationic group or phosphorous-containing cationic group, while others have two nitrogen-containing cationic groups or phosphorous-containing cationic groups.

In some embodiments, a plurality of ionic monomers (e.g., of a polymeric catalyst) or a plurality of ionic moieties (e.g., of a solid-supported catalyst) can have one cationic group, or two or more cationic groups, as is chemically feasible. When the ionic monomers (e.g., of a polymeric catalyst) or ionic moieties (e.g., of a solid-supported catalyst) have two or more cationic groups, the cationic groups can be the same or different.

In some embodiments, each ionic monomer (e.g., of a polymeric catalyst) or each ionic moiety (e.g., of a solid-supported catalyst) is a nitrogen-containing cationic group. In other embodiments, each ionic monomer (e.g., of a polymeric catalyst) or each ionic moiety (e.g., of a solid-supported catalyst) is a phosphorous-containing cationic group. In yet other embodiments, at least some of ionic monomers (e.g., of a polymeric catalyst) or at least some of the ionic moieties (e.g., of a solid-supported catalyst) are a nitrogen-containing cationic group, whereas the cationic groups in other ionic monomers (e.g., of a polymeric catalyst) or ionic moieties (e.g., of a solid-supported catalyst) are a phosphorous-containing cationic group. In an exemplary embodiment, each cationic group in the polymeric catalyst or solid-supported catalyst is imidazolium. In another exemplary embodiment, the cationic group in some monomers (e.g., of a polymeric catalyst) or moieties (e.g., of a solid-supported catalyst) is imidazolium, while the cationic group in other monomers (e.g., of a polymeric catalyst) or moieties (e.g., of a solid-supported catalyst) is pyridinium. In yet another exemplary embodiment, each cationic group in the polymeric catalyst or solid-supported catalyst is a substituted phosphonium. In yet another exemplary embodiment, the cationic group in some monomers (e.g., of a polymeric catalyst) or moieties (e.g., of a solid-supported catalyst) is triphenyl phosphonium, while the cationic group in other monomers (e.g., of a polymeric catalyst) or moieties (e.g., of a solid-supported catalyst) is imidazolium.

In some embodiments, the nitrogen-containing cationic group at each occurrence can be independently selected from pyrrolium, imidazolium, pyrazolium, oxazolium, thiazolium, pyridinium, pyrimidinium, pyrazinium, pyridazinium, thiazinium, morpholinium, piperidinium, piperizinium, and pyrollizinium. In other embodiments, the nitrogen-containing cationic group at each occurrence can be independently selected from imidazolium, pyridinium, pyrimidinium, morpholinium, piperidinium, and piperizinium. In some embodiments, the nitrogen-containing cationic group can be imidazolium.

In some embodiments, the phosphorous-containing cationic group at each occurrence can be independently selected from triphenyl phosphonium, trimethyl phosphonium, triethyl phosphonium, tripropyl phosphonium, tributyl phosphonium, trichloro phosphonium, and trifluoro phosphonium. In other embodiments, the phosphorous-containing cationic group at each occurrence can be independently selected from triphenyl phosphonium, trimethyl phosphonium, and triethyl phosphonium. In other embodiments, the phosphorous-containing cationic group can be triphenyl phosphonium.

In some embodiments, one or more of the ionic monomers of a polymeric catalyst are directly connected to the polymeric backbone, or one or more of the ionic moieties of a solid-supported catalyst are directly connected to the solid support. In other embodiments, one or more of the ionic monomers (e.g., of a polymeric catalyst) or one or more ionic moieties (e.g., of a solid-supported catalyst) each independently further includes a linker connecting the cationic group to the polymeric backbone or the solid support (as the case may be). In certain embodiments, some of the cationic groups are directly connected to the polymeric backbone or the solid support (as the case may be), while other the cationic groups are connected to the polymeric backbone or the solid support (as the case may be) by a linker.

In those embodiments where the cationic group is connected to the polymeric backbone or the solid support (as the case may be) by a linker, each linker is independently selected from unsubstituted or substituted alkyl linker, unsubstituted or substituted cycloalkyl linker, unsubstituted or substituted alkenyl linker, unsubstituted or substituted aryl linker, and unsubstituted or substituted heteroaryl linker. In certain embodiments, the linker is unsubstituted or substituted aryl linker, or unsubstituted or substituted heteroaryl linker. In certain embodiments, the linker is unsubstituted or substituted aryl linker. In one embodiment, the linker is a phenyl linker. In another embodiment, the linker is a hydroxyl-substituted phenyl linker.

In other embodiments, each linker in an ionic monomer (e.g., of a polymeric catalyst) or an ionic moiety (e.g., of a solid-supported catalyst) is independently selected from:
unsubstituted alkyl linker;
alkyl linker substituted 1 to 5 substituents independently selected from oxo, hydroxy, halo, amino;
unsubstituted cycloalkyl linker;
cycloalkyl linker substituted 1 to 5 substituents independently selected from oxo, hydroxy, halo, amino;
unsubstituted alkenyl linker;
alkenyl linker substituted 1 to 5 substituents independently selected from oxo, hydroxy, halo, amino;
unsubstituted aryl linker;
aryl linker substituted 1 to 5 substituents independently selected from oxo, hydroxy, halo, amino;
unsubstituted heteroaryl linker; or
heteroaryl linker substituted 1 to 5 substituents independently selected from oxo, hydroxy, halo, amino.

Further, it should be understood that some or all of the ionic monomers (e.g., of a polymeric catalyst) or one or more ionic moieties (e.g., of a solid-supported catalyst) connected to the polymeric backbone by a linker may have the same linker, or independently have different linkers.

In some embodiments, each ionic monomer (e.g., of a polymeric catalyst) or each ionic moiety (e.g., of a solid-supported catalyst) is independently has the structure of Formulas VIIA-XIB:

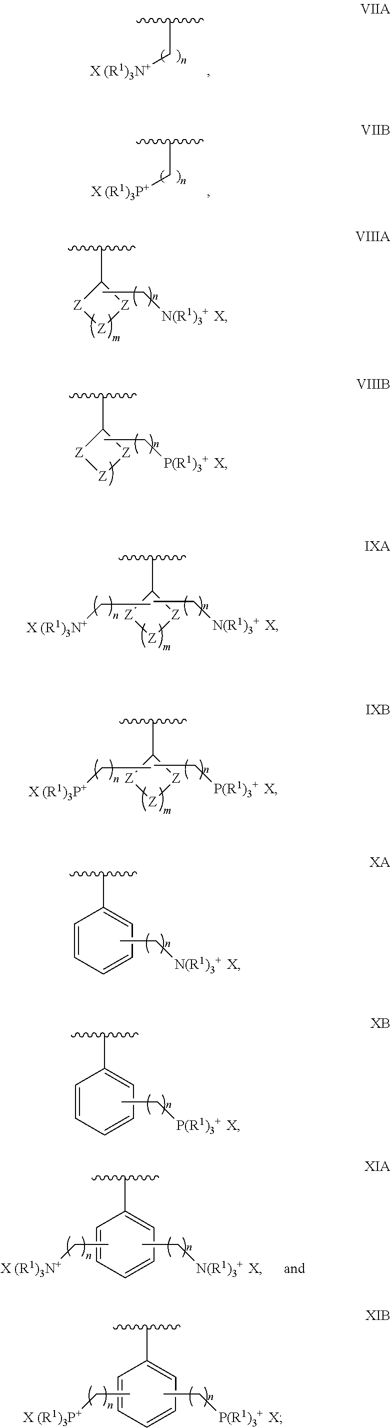

wherein:
each Z is independently C(R²)(R³), N(R⁴), S, S(R⁵)(R⁶), S(O)(R⁵)(R⁶), SO₂, or O, wherein any two adjacent Z can (to the extent chemically feasible) be joined by a double bond, or taken together to form cycloalkyl, heterocycloalkyl, aryl or heteroaryl;
each X is independently F⁻, Cl⁻, Br⁻, I⁻, NO₂⁻, NO₃⁻, SO₄²⁻, R⁷SO₄⁻, R⁷CO₂, PO₄²⁻, R⁷PO₃, or R⁷PO₂⁻, where SO₄²⁻ and PO₄²⁻ are each independently associated with at least two cationic groups at any X position on any ionic monomer, and
each m is independently 0, 1, 2, or 3;
each n is independently 0, 1, 2, or 3;
each R¹, R², R³ and R⁴ is independently hydrogen, alkyl, heteroalkyl, cycloalkyl, heterocyclyl, aryl, or heteroaryl;
each R⁵ and R⁶ is independently alkyl, heteroalkyl, cycloalkyl, heterocyclyl, aryl, or heteroaryl; and
each R⁷ is independently hydrogen, C₁₋₄alkyl, or C₁₋₄heteroalkyl.

In some embodiments, Z can be chosen from C(R²)(R³), N(R⁴), SO₂, and O. In some embodiments, any two adjacent Z can be taken together to form a group selected from a heterocycloalkyl, aryl and heteroaryl. In other embodiments, any two adjacent Z can be joined by a double bond. In some embodiments, each X can be Cl⁻, NO₃⁻, SO₄²⁻, R⁷SO₄⁻, or R⁷CO₂, where R⁷ can be hydrogen or C₁₋₄alkyl. In another embodiment, each X can be Cl⁻, Br⁻, I⁻, HSO₄⁻, HCO₂⁻, CH₃CO₂⁻, or NO₃⁻. In other embodiments, X is acetate. In other embodiments, X is bisulfate. In other embodiments, X is chloride. In other embodiments, X is nitrate.

In some embodiments, m is 2 or 3. In other embodiments, n is 1, 2, or 3. In some embodiments, each R², R³, and R⁴ can be independently hydrogen, alkyl, heterocyclyl, aryl, or heteroaryl. In other embodiments, each R², R³ and R⁴ can be independently heteroalkyl, cycloalkyl, heterocyclyl, or heteroaryl. In some embodiments, each R⁵ and R⁶ can be independently alkyl, heterocyclyl, aryl, or heteroaryl. In another embodiment, any two adjacent Z can be taken together to form cycloalkyl, heterocycloalkyl, aryl or heteroaryl.

In certain embodiments, the ionic monomers of the polymeric catalyst may have a side chain with a cationic group that is connected to the polymeric backbone by a linker. In certain embodiments, the ionic moieties of the solid-supported catalyst may have a cationic group that is attached to the solid support by a linker. Side chains (e.g., of a polymeric catalyst) or ionic moieties (e.g., of a solid-supported catalyst) with one or more cationic groups connected by a linker can include, for example,

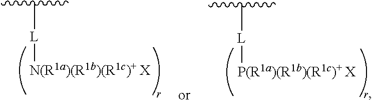

wherein:
L is an unsubstituted alkyl linker, alkyl linker substituted with oxo, unsubstituted cycloalkyl, unsubstituted aryl, unsubstituted heterocycloalkyl, and unsubstituted heteroaryl;
each R^{1a}, R^{1b} and R^{1c} are independently hydrogen or alkyl; or Ria and R are taken together with the nitrogen atom to which they are attached to form an unsubstituted heterocycloalkyl; or R^{1a} and R^{1b} are taken together with the nitrogen atom to which they are attached to form an unsubstituted heteroaryl or substituted heteroaryl, and R^{1c} is absent;
r is an integer; and
X is as described above for Formulas VIIA-XIB.

In other embodiments L is methyl, ethyl, propyl, butyl. In yet other embodiments, the linker is ethanoyl, propanoyl, or benzoyl. In certain embodiments, r is 1, 2, 3, 4, or 5 (as applicable or chemically feasible).

In other embodiments, each linker is independently selected from:
unsubstituted alkyl linker;
alkyl linker substituted 1 to 5 substituents independently selected from oxo, hydroxy, halo, amino;
unsubstituted cycloalkyl linker;
cycloalkyl linker substituted 1 to 5 substituents independently selected from oxo, hydroxy, halo, amino;
unsubstituted alkenyl linker;
alkenyl linker substituted 1 to 5 substituents independently selected from oxo, hydroxy, halo, amino;
unsubstituted aryl linker;
aryl linker substituted 1 to 5 substituents independently selected from oxo, hydroxy, halo, amino;
unsubstituted heteroaryl linker; or
heteroaryl linker substituted 1 to 5 substituents independently selected from oxo, hydroxy, halo, amino.

In certain embodiments, each linker is an unsubstituted alkyl linker or an alkyl linker with an oxo substituent. In one embodiment, each linker is —(CH₂)(CH₂)— or —(CH₂)(C═O). In certain embodiments, r is 1, 2, 3, 4, or 5 (as applicable or chemically feasible).

In some embodiments, at least some of the ionic side chains (e.g., of a polymeric catalyst) and at least some of the ionic moieties (e.g., of a solid-supported catalyst) may be:

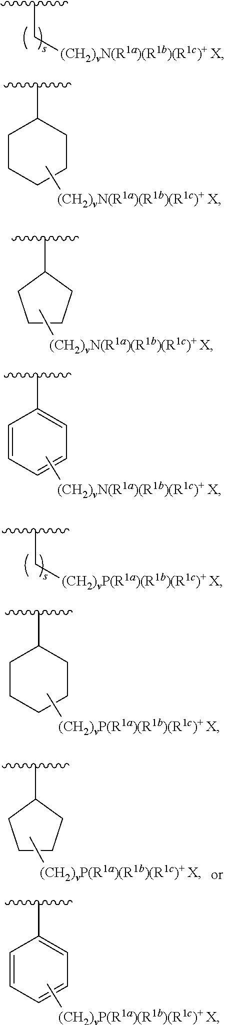

wherein:
each R^{1a}, R^{1b} and R^{1c} are independently hydrogen or alkyl; or R^{1a} and R^{1b} are taken together with the nitrogen atom to which they are attached to form an unsubstituted heterocycloalkyl; or R^{1a} and R^{1b} are taken together with the nitrogen atom to which they are attached to form an unsubstituted heteroaryl or substituted heteroaryl, and R^{1c} is absent;
s is an integer;
v is 0 to 10; and
X is as described above for Formulas VIIA-XIB.

In certain embodiments, s is 1 to 9, or 1 to 8, or 1 to 7, or 1 to 6, or 1 to 5, or 1 to 4, or 1 to 3, or 2, or 1. In certain embodiments, v is 0 to 9, or 0 to 8, or 0 to 7, or 0 to 6, or 0 to 5, or 0 to 4, or 0 to 3, or 0 to 2, 1 or 0).

In certain embodiments, at least some of the ionic side chains (e.g., of a polymeric catalyst) and at least some of the ionic moieties (e.g., of a solid-supported catalyst) may be:

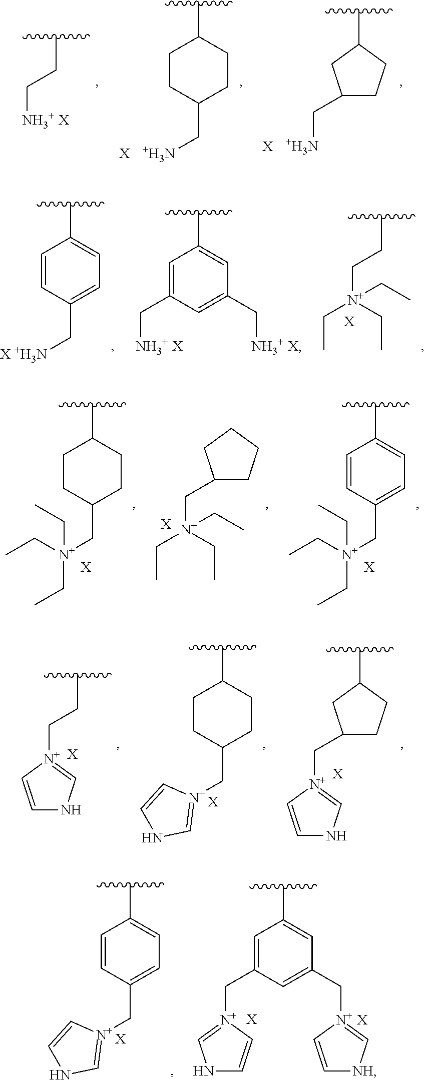

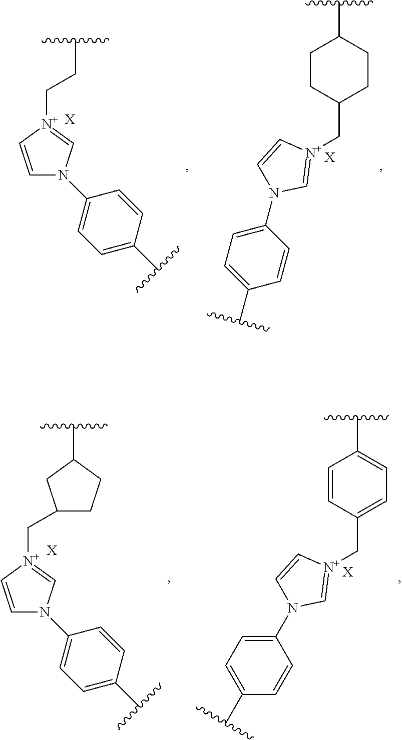

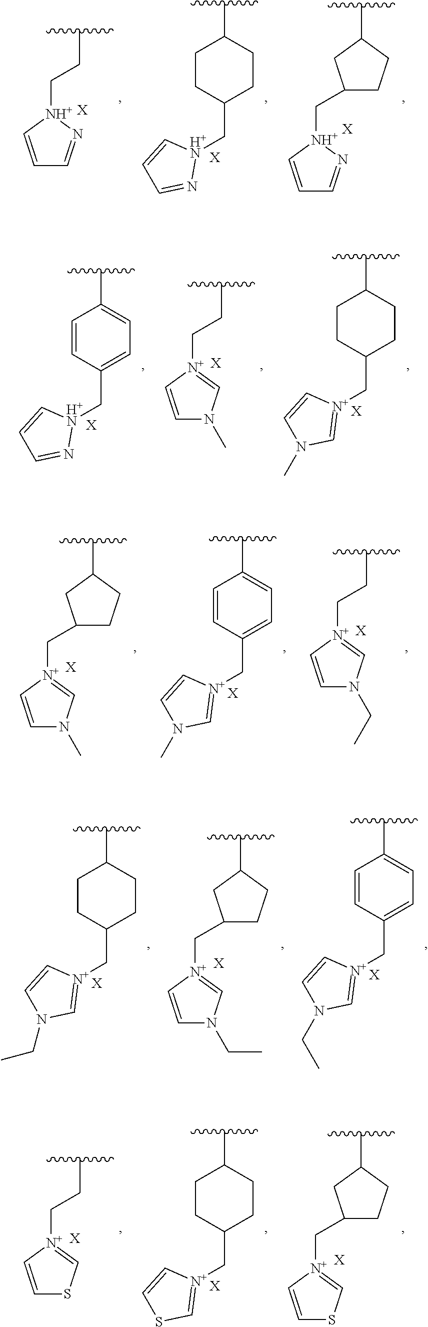

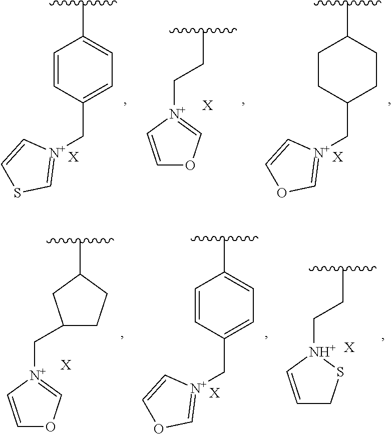

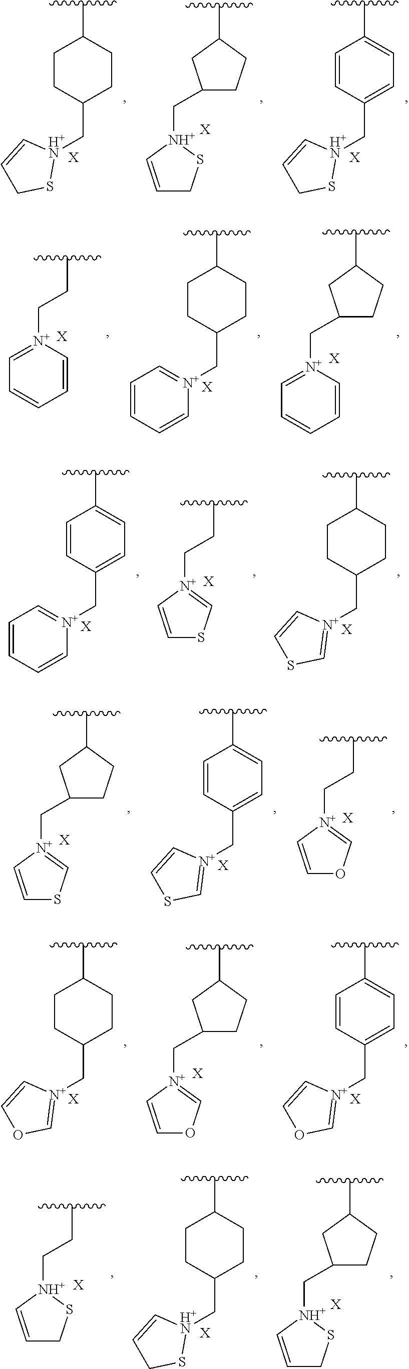

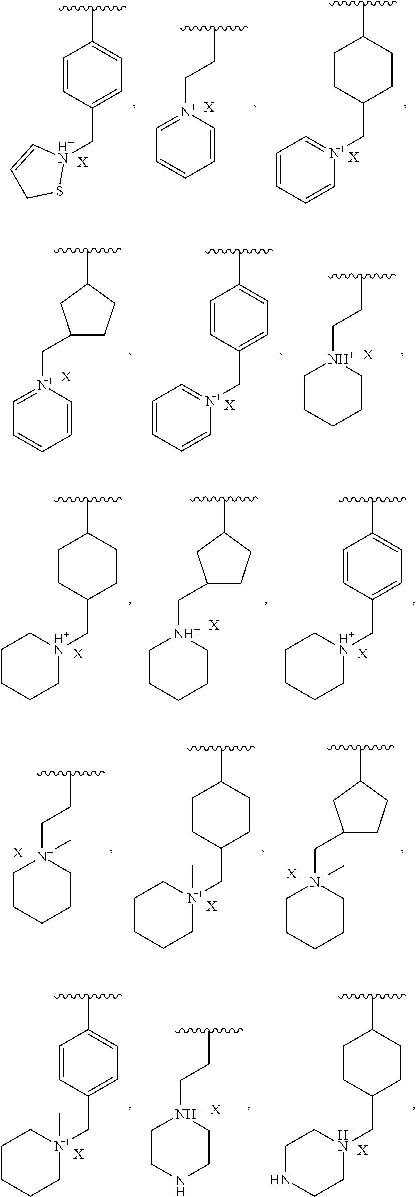

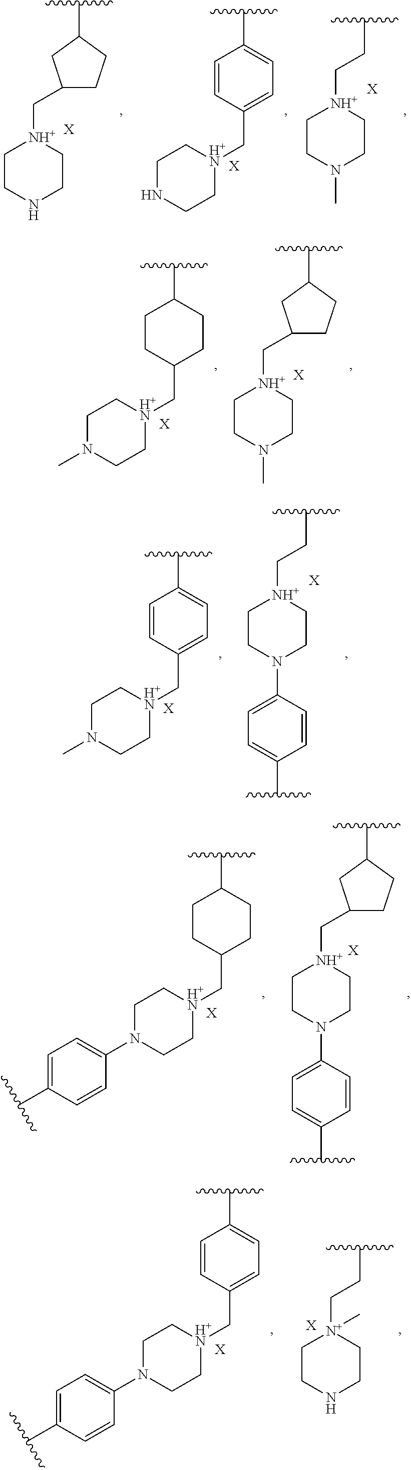

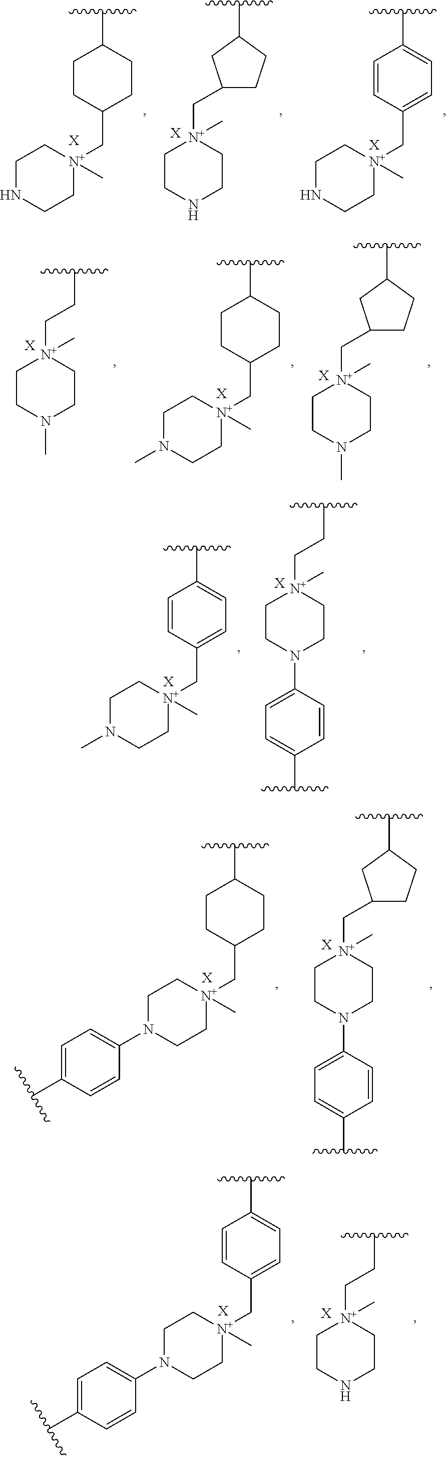

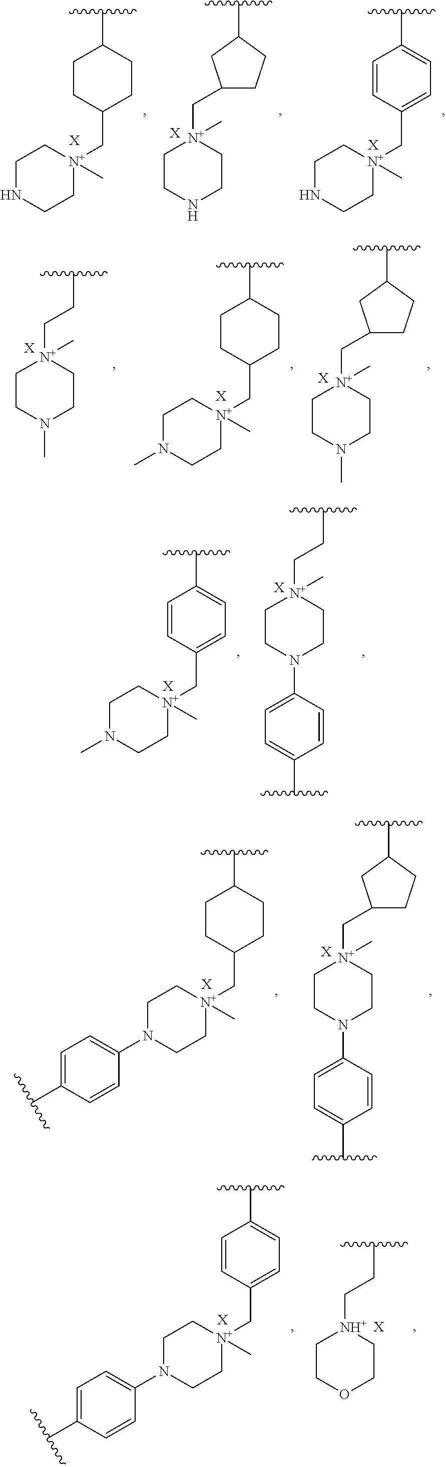

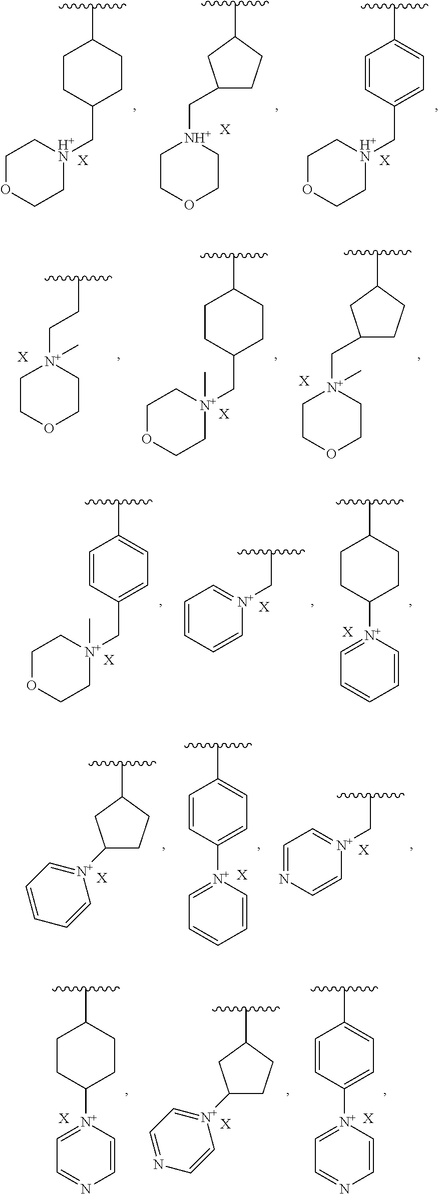

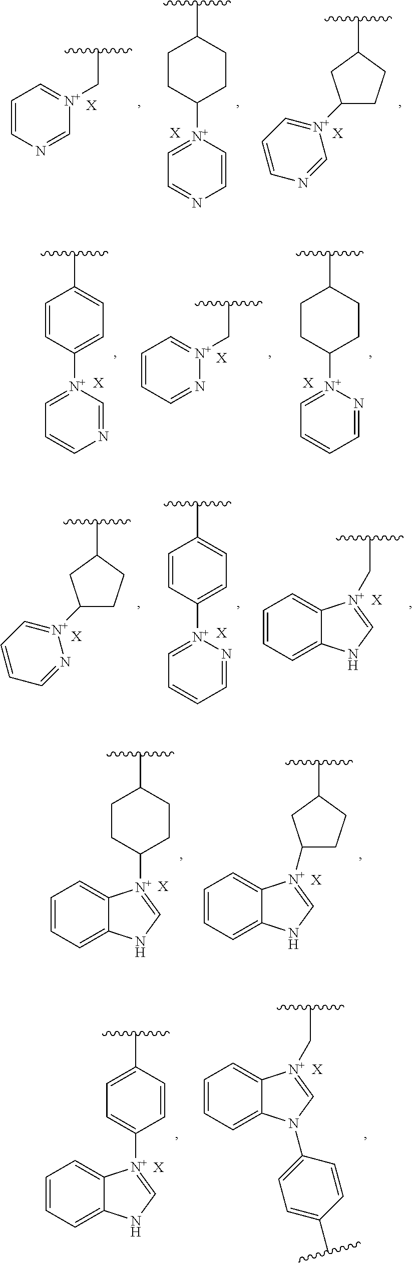

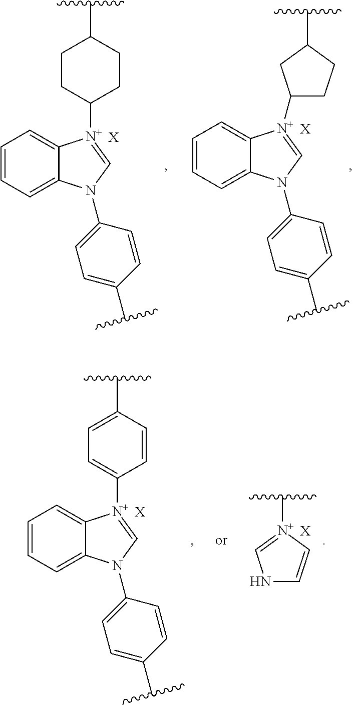

In other embodiments, the ionic monomers (e.g., of a polymeric catalyst) can have a side chain with a cationic group that is directly connected to the polymeric backbone. In other embodiments, the ionic moieties (e.g., of a solid-supported catalyst) can have a cationic group that is directly attached to the solid support. Side chains (e.g., of a polymeric catalyst) directly connect to the polymeric backbone or ionic moieties (e.g., of a solid-supported catalyst) directly attached to the solid support may can include, for example,

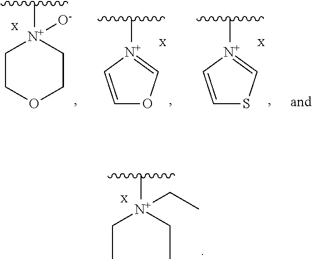

In some embodiments, the nitrogen-containing cationic group can be an N-oxide, where the negatively charged oxide (O−) is not readily dissociable from the nitrogen cation. Non-limiting examples of such groups include, for example,

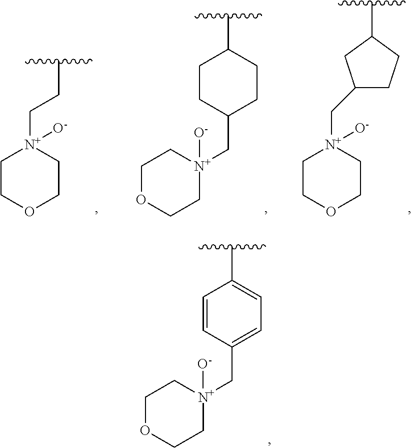 [SMILES: *CC[N+]1([O-])CCOCC1 ; *C1CCC(C[N+]2([O-])CCOCC2)CC1 ; *C1CCC(C[N+]2([O-])CCOCC2)C1 ; *c1ccc(C[N+]2([O-])CCOCC2)cc1]

In some embodiments, the phosphorous-containing side chain (e.g., of a polymeric catalyst) or moiety (e.g., of a solid-supported catalyst) is independently;

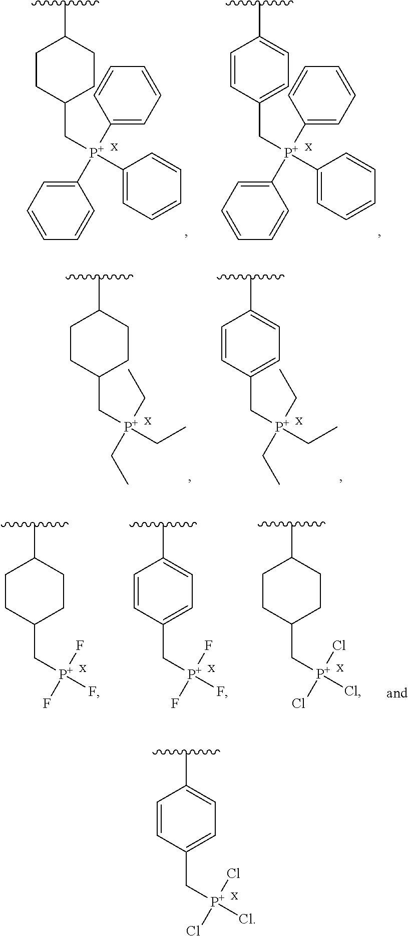

In other embodiments, the ionic monomers (e.g., of a polymeric catalyst) can have a side chain with a cationic group that is directly connected to the polymeric backbone. In other embodiments, the ionic moieties (e.g., of a solid-supported catalyst) can have a cationic group that is directly attached to the solid support. Side chains (e.g., of a polymeric catalyst) directly connect to the polymeric backbone or ionic moieties (e.g., of a solid-supported catalyst) directly attached to the solid support may can include, for example,

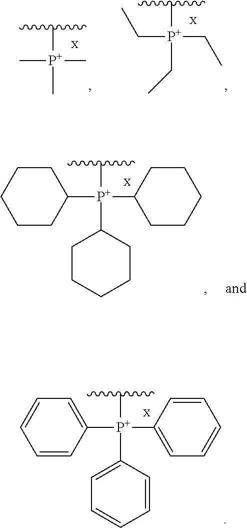

The ionic monomers (e.g., of a polymeric catalyst) or ionic moieties (e.g., of a solid-supported catalyst) can either all have the same cationic group, or can have different cationic groups. In some embodiments, each cationic group in the polymeric catalyst or solid-supported catalyst is a nitrogen-containing cationic group. In other embodiments, each cationic group in the polymeric catalyst or solid-supported catalyst is a phosphorous-containing cationic group. In yet other embodiments, the cationic group in some monomers or moieties of the polymeric catalyst or solid-supported catalyst, respectively, is a nitrogen-containing cationic group, whereas the cationic group in other monomers or moieties of the polymeric catalyst or solid-supported catalyst, respectively, is a phosphorous-containing cationic group. In an exemplary embodiment, each cationic group in the polymeric catalyst or solid-supported catalyst is imidazolium. In another exemplary embodiment, the cationic group in some monomers or moieties of the polymeric catalyst or solid-supported catalyst is imidazolium, while the cationic group in other monomers or moieties of the polymeric catalyst or solid-supported catalyst is pyridinium. In yet another exemplary embodiment, each cationic group in the polymeric catalyst or solid-supported catalyst is a substituted phosphonium. In yet another exemplary embodiment, the cationic group in some monomers or moieties of the polymeric catalyst or solid-supported catalyst is triphenyl phosphonium, while the cationic group in other monomers or moieties of the polymeric catalyst or solid-supported catalyst is imidazolium.

### Acidic-Ionic Monomers and Moieties

Some of the monomers in the polymeric catalyst contain both the Bronsted-Lowry acid and the cationic group in the same monomer. Such monomers are referred to as “acidic-ionic monomers”. Similarly, some of the moieties in the solid-supported catalyst contain both the Bronsted-Lowry acid and the cationic group in the same moieties. Such moieties are referred to as “acidic-ionic moieties”. For example, in exemplary embodiments, the acidic-ionic monomer (e.g., of a polymeric catalyst) or an acidic-ionic moiety (e.g., of a solid-supported catalyst) can contain imidazolium and acetic acid, or pyridinium and boronic acid.

In some embodiments, the monomers (e.g., of a polymeric catalyst) or moieties (e.g., of a solid-supported catalyst) include both Bronsted-Lowry acid(s) and cationic group(s), where either the Bronsted-Lowry acid is connected to the polymeric backbone (e.g., of a polymeric catalyst) or solid support (e.g., of a solid-supported catalyst) by a linker, and/or the cationic group is connected to the polymeric backbone (e.g., of a polymeric catalyst) or is attached to the solid support (e.g., of a solid-supported catalyst) by a linker.

It should be understood that any of the Bronsted-Lowry acids, cationic groups and linkers (if present) suitable for the acidic monomers/moieties and/or ionic monomers/moieties may be used in the acidic-ionic monomers/moieties.

In certain embodiments, the Bronsted-Lowry acid at each occurrence in the acidic-ionic monomer (e.g., of a polymeric catalyst) or the acidic-ionic moiety (e.g., of a solid-supported catalyst) is independently selected from sulfonic acid, phosphonic acid, acetic acid, isophthalic acid, and boronic acid. In certain embodiments, the Bronsted-Lowry acid at each occurrence in the acidic-ionic monomer (e.g., of a polymeric catalyst) or the acidic-ionic moiety (e.g., of a solid-supported catalyst) is independently sulfonic acid or phosphonic acid. In one embodiment, the Bronsted-Lowry acid at each occurrence in the acidic-ionic monomer (e.g., of a polymeric catalyst) or the acidic-ionic moiety (e.g., of a solid-supported catalyst) is sulfonic acid.

In some embodiments, the nitrogen-containing cationic group at each occurrence in the acidic-ionic monomer (e.g., of a polymeric catalyst) or the acidic-ionic moiety (e.g., of a solid-supported catalyst) is independently selected from pyrrolium, imidazolium, pyrazolium, oxazolium, thiazolium, pyridinium, pyrimidinium, pyrazinium, pyridazinium, thiazinium, morpholinium, piperidinium, piperizinium, and pyrollizinium. In one embodiment, the nitrogen-containing cationic group is imidazolium.

In some embodiments, the phosphorous-containing cationic group at each occurrence in the acidic-ionic monomer (e.g., of a polymeric catalyst) or the acidic-ionic moiety (e.g., of a solid-supported catalyst) is independently selected from triphenyl phosphonium, trimethyl phosphonium, triethyl phosphonium, tripropyl phosphonium, tributyl phosphonium, trichloro phosphonium, and trifluoro phosphonium. In one embodiment, the phosphorous-containing cationic group is triphenyl phosphonium.

In some embodiments, the polymeric catalyst or solid-supported catalyst can include at least one acidic-ionic monomer or moiety, respectively, connected to the polymeric backbone or solid support, wherein at least one acidic-ionic monomer or moiety includes at least one Bronsted-Lowry acid and at least one cationic group, and wherein at least one of the acidic-ionic monomers or moieties includes a linker connecting the acidic-ionic monomer to the polymeric backbone or solid support. The cationic group can be a nitrogen-containing cationic group or a phosphorous-containing cationic group as described herein. The linker can also be as described herein for either the acidic or ionic moieties. For example, the linker can be selected from unsubstituted or substituted alkyl linker, unsubstituted or substituted cycloalkyl linker, unsubstituted or substituted alkenyl linker, unsubstituted or substituted aryl linker, and unsubstituted or substituted heteroaryl linker.

In other embodiments, the monomers (e.g., of a polymeric catalyst) or moieties (e.g., of a solid-supported catalyst) can have a side chain containing both a Bronsted-Lowry acid and a cationic group, where the Bronsted-Lowry acid is directly connected to the polymeric backbone or solid support, the cationic group is directly connected to the polymeric backbone or solid support, or both the Bronsted-Lowry acid and the cationic group are directly connected to the polymeric backbone or solid support.

In certain embodiments, the linker is unsubstituted or substituted aryl linker, or unsubstituted or substituted heteroaryl linker. In certain embodiments, the linker is unsubstituted or substituted aryl linker. In one embodiment, the linker is a phenyl linker. In another embodiment, the linker is a hydroxyl-substituted phenyl linker.

Monomers of a polymeric catalyst that have side chains containing both a Bronsted-Lowry acid and a cationic group can also be called “acidic ionomers”. Acidic-ionic side chains (e.g., of a polymeric catalyst) or acidic-ionic moieties (e.g., of a solid-supported catalyst) that are connected by a linker can include, for example,

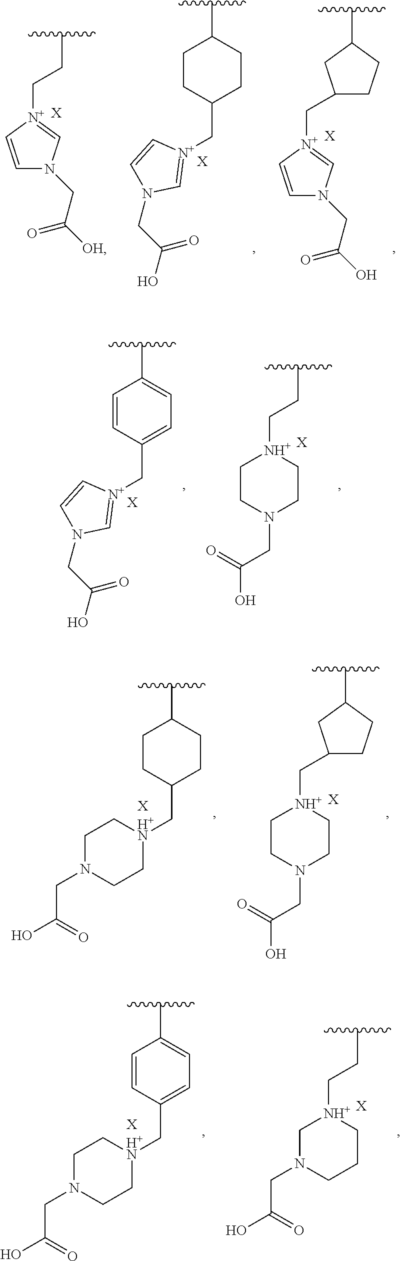

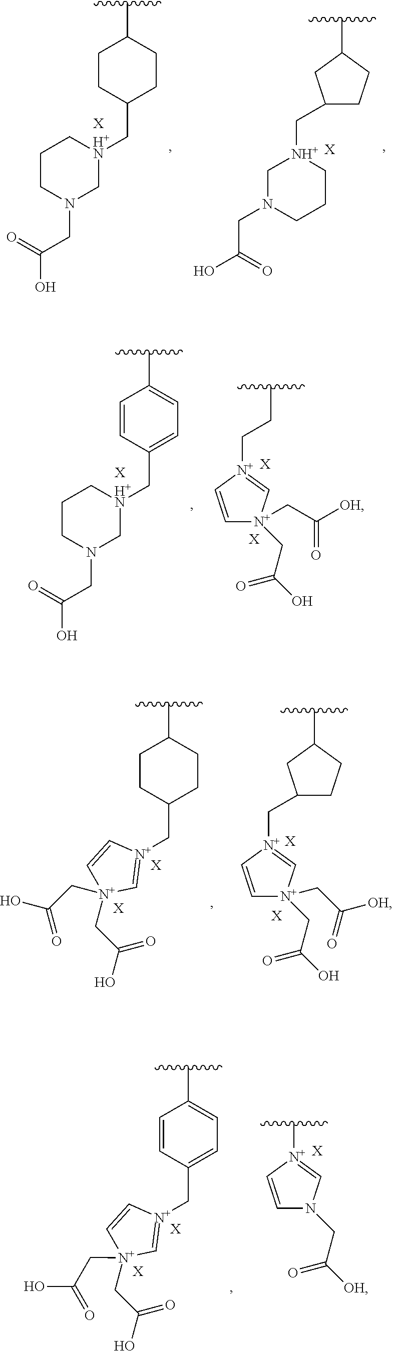

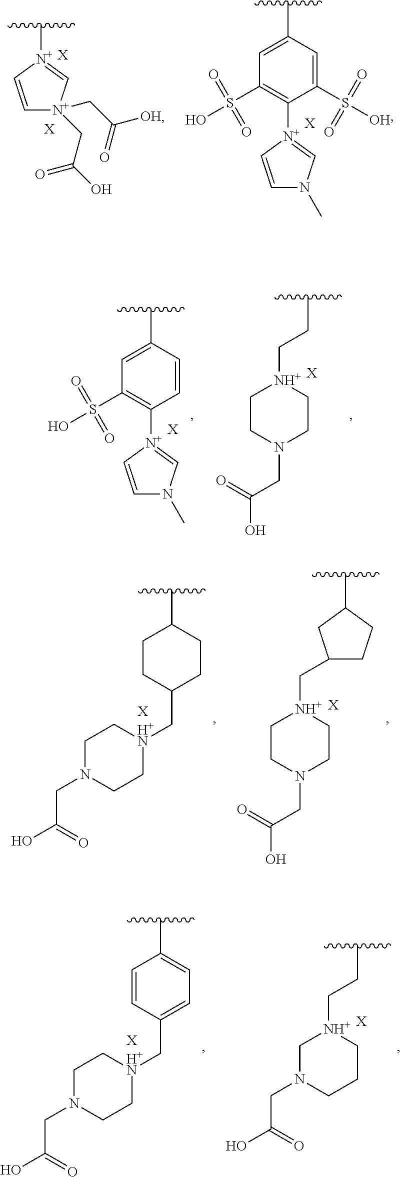

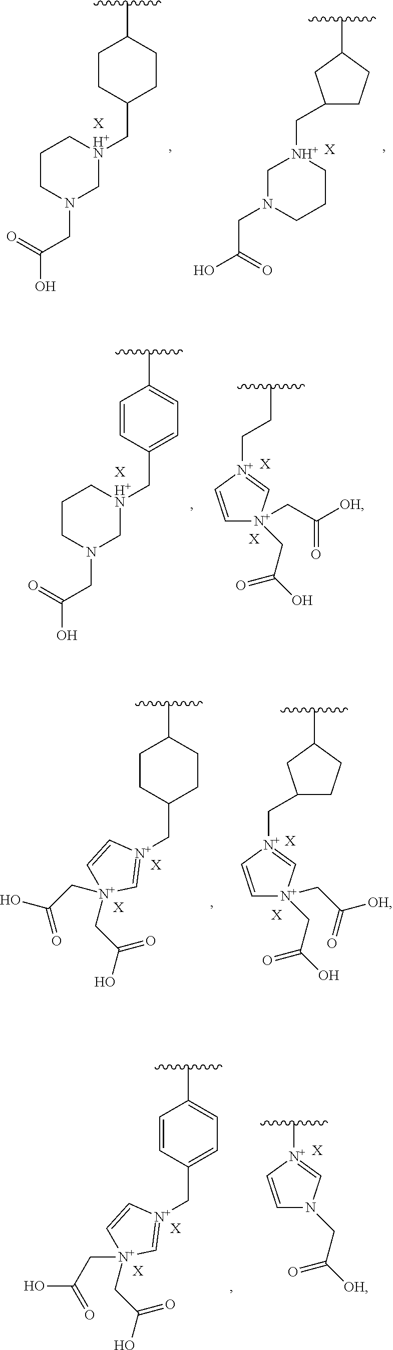

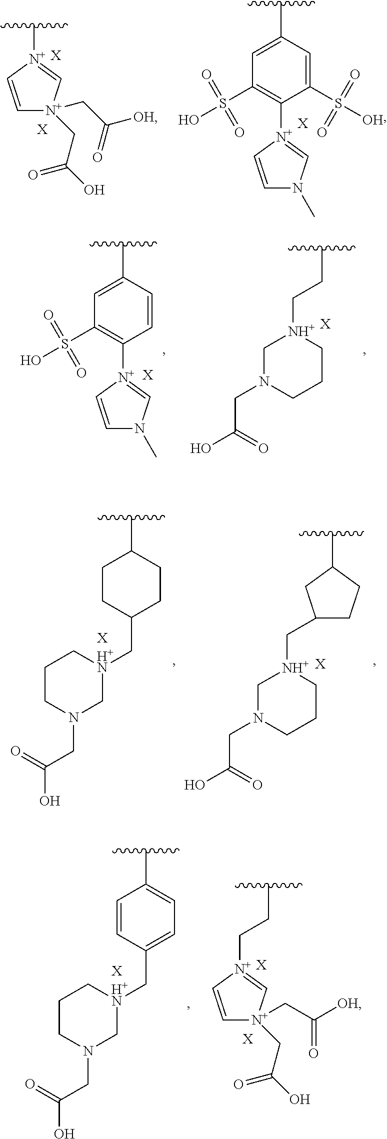

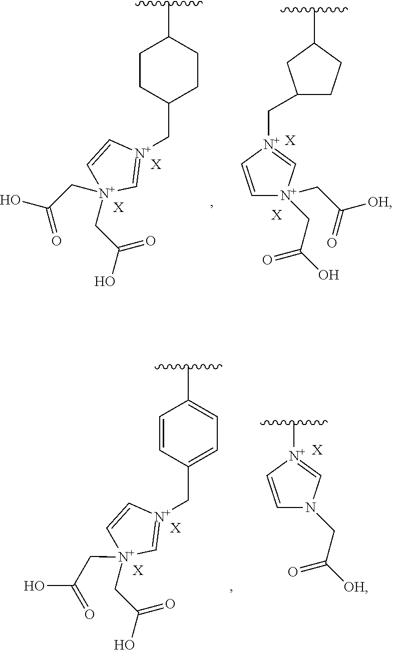

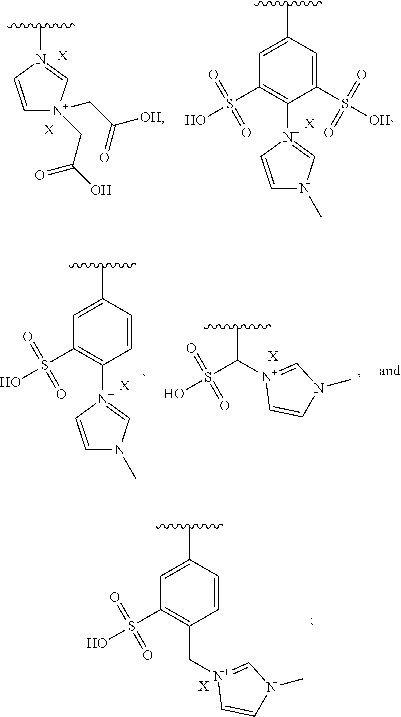

wherein:
each X is independently selected from F⁻, Cl⁻, Br⁻, I⁻, NO₂⁻, NO₃⁻, SO₄²⁻, R⁷SO₄⁻, R⁷CO₂⁻, PO₄²⁻, R⁷PO₃⁻, and R⁷PO₂⁻, where SO₄²⁻ and PO₄²⁻ are each independently associated with at least two Bronsted-Lowry acids at any X position on any side chain, and each R⁷ is independently selected from hydrogen, C₁₋₄alkyl, and C₁₋₄heteroalkyl.

In some embodiments, R¹ can be selected from hydrogen, alkyl, and heteroalkyl. In some embodiments, R¹ can be selected from hydrogen, methyl, or ethyl. In some embodiments, each X can be selected from Cl⁻, NO₃⁻, SO₄²⁻, R⁷SO₄⁻, and R⁷CO₂⁻, where R⁷ can be selected from hydrogen and C₁₋₄alkyl. In another embodiment, each X can be selected from Cl⁻, Br⁻, I⁻, HSO₄⁻, HCO₂⁻, CH₃CO₂⁻, and NO₃⁻. In other embodiments, X is acetate. In other embodiments, X is bisulfate. In other embodiments, X is chloride. In other embodiments, X is nitrate.

In some embodiments, the acidic-ionic side chain (e.g., of a polymeric catalyst) or the acidic-ionic moiety (e.g., of a solid-supported catalyst) is independently:

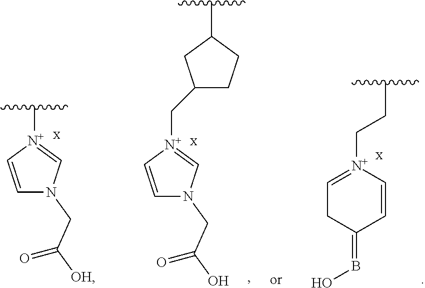

In some embodiments, the acidic-ionic side chain (e.g., of a polymeric catalyst) or the acidic-ionic moiety (e.g., of a solid-supported catalyst) is independently:

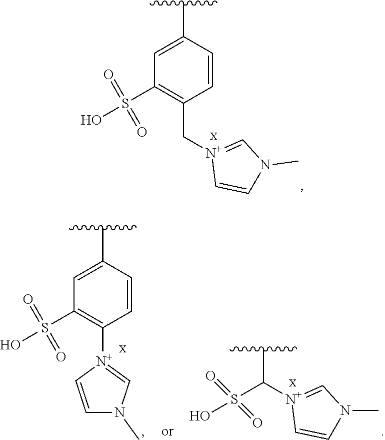

In other embodiments, the monomers (e.g., of a polymeric catalyst) or moieties (e.g., of a solid-supported catalyst) can have both a Bronsted-Lowry acid and a cationic group, where the Bronsted-Lowry acid is directly connected to the polymeric backbone or solid support, the cationic group is directly connected to the polymeric backbone or solid support, or both the Bronsted-Lowry acid and the cationic group are directly connected to the polymeric backbone or solid support. Such side chains in acidic-ionic monomers (e.g., of a polymeric catalyst) or moieties (e.g., of a solid-supported catalyst) can include, for example,

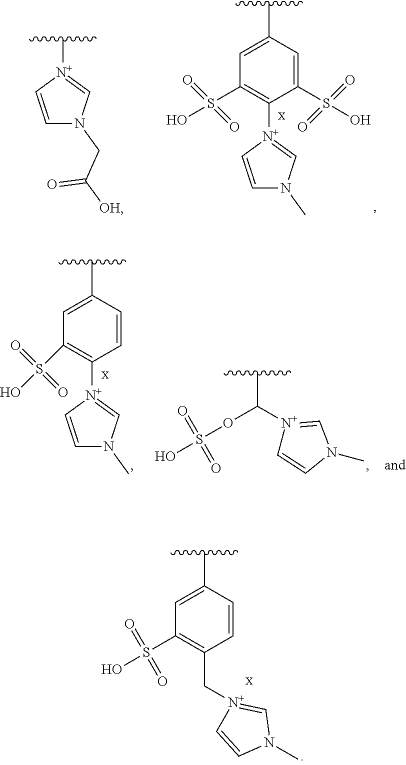

### Hydrophobic Monomers and Moieties

In some embodiments, the polymeric catalyst further includes hydrophobic monomers connected to form the polymeric backbone. Similarly, in some embodiments, the solid-supported catalyst further includes hydrophobic moieties attached to the solid support. In either instances, each hydrophobic monomer or moiety has at least one hydrophobic group. In certain embodiments of the polymeric catalyst or solid-supported catalyst, each hydrophobic monomer or moiety, respectively, has one hydrophobic group. In certain embodiments of the polymeric catalyst or solid-supported catalyst, each hydrophobic monomer or moiety has two hydrophobic groups. In other embodiments of the polymeric catalyst or solid-supported catalyst, some of the hydrophobic monomers or moieties have one hydrophobic group, while others have two hydrophobic groups.

In some embodiments of the polymeric catalyst or solid-supported catalyst, each hydrophobic group is independently selected from an unsubstituted or substituted alkyl, an unsubstituted or substituted cycloalkyl, an unsubstituted or substituted aryl, and an unsubstituted or substituted heteroaryl. In certain embodiments of the polymeric catalyst or solid-supported catalyst, each hydrophobic group is an unsubstituted or substituted aryl, or an unsubstituted or substituted heteroaryl. In one embodiment, each hydrophobic group is phenyl. Further, it should be understood that the hydrophobic monomers may either all have the same hydrophobic group, or may have different hydrophobic groups.

In some embodiments of the polymeric catalyst, the hydrophobic group is directly connected to form the polymeric backbone. In some embodiments of the solid-supported catalyst, the hydrophobic group is directly attached to the solid support.

### Other Characteristics of the Catalysts

In some embodiments, the acidic and ionic monomers make up a substantial portion of the polymeric catalyst. In some embodiments, the acidic and ionic moieties make up a substantial portion solid-supported catalyst. In certain embodiments, the acidic and ionic monomers or moieties make up at least about 30%, at least about 40%, at least about 50%, at least about 60%, at least about 70%, at least about 80%, at least about 90%, at least about 95%, or at least about 99% of the monomers or moieties of the catalyst, based on the ratio of the number of acidic and ionic monomers/moieties to the total number of monomers/moieties present in the catalyst.

In some embodiments, the polymeric catalyst or solid-supported catalyst has a total amount of Bronsted-Lowry acid of between about 0.1 and about 20 mmol, between about 0.1 and about 15 mmol, between about 0.01 and about 12 mmol, between about 0.05 and about 10 mmol, between about 1 and about 8 mmol, between about 2 and about 7 mmol, between about 3 and about 6 mmol, between about 1 and about 5, or between about 3 and about 5 mmol per gram of the polymeric catalyst or solid-supported catalyst.

In some embodiments of the polymeric catalyst or solid-supported catalyst, each ionic monomer further includes a counterion for each nitrogen-containing cationic group or phosphorous-containing cationic group. In certain embodiments of the polymeric catalyst or solid-supported catalyst, each counterion is independently selected from halide, nitrate, sulfate, formate, acetate, or organosulfonate. In some embodiments of the polymeric catalyst or solid-supported catalyst, the counterion is fluoride, chloride, bromide, or iodide. In one embodiment of the polymeric catalyst or solid-supported catalyst, the counterion is chloride. In another embodiment of the polymeric catalyst or solid-supported catalyst, the counterion is sulfate. In yet another embodiment of the polymeric catalyst or solid-supported catalyst, the counterion is acetate.

In some embodiments, the polymeric catalyst or solid-supported catalyst has a total amount of nitrogen-containing cationic groups and counterions or a total amount of phosphorous-containing cationic groups and counterions of between about 0.01 and about 10 mmol, between about 0.05 and about 10 mmol, between about 1 and about 8 mmol, between about 2 and about 6 mmol, or between about 3 and about 5 mmol per gram of the polymeric catalyst or solid-supported catalyst.

In some embodiments, the acidic and ionic monomers make up a substantial portion of the polymeric catalyst or solid-supported catalyst. In certain embodiments, the acidic and ionic monomers or moieties make up at least about 30%, at least about 40%, at least about 50%, at least about 60%, at least about 70%, at least about 80%, at least about 90%, at least about 95%, or at least about 99% of the monomers of the polymeric catalyst or solid-supported catalyst, based on the ratio of the number of acidic and ionic monomers or moieties to the total number of monomers or moieties present in the polymeric catalyst or solid-supported catalyst.

The ratio of the total number of acidic monomers or moieties to the total number of ionic monomers or moieties can be varied to tune the strength of the catalyst. In some embodiments, the total number of acidic monomers or moieties exceeds the total number of ionic monomers or moieties in the polymer or solid support. In other embodiments, the total number of acidic monomers or moieties is at least about 2, at least about 3, at least about 4, at least about 5, at least about 6, at least about 7, at least about 8, at least about 9 or at least about 10 times the total number of ionic monomers or moieties in the polymeric catalyst or solid-supported catalyst. In certain embodiments, the ratio of the total number of acidic monomers or moieties to the total number of ionic monomers or moieties is about 1:1, about 2:1, about 3:1, about 4:1, about 5:1, about 6:1, about 7:1, about 8:1, about 9:1 or about 10:1.

In some embodiments, the total number of ionic monomers or moieties exceeds the total number of acidic monomers or moieties in the catalyst. In other embodiments, the total number of ionic monomers or moieties is at least about 2, at least about 3, at least about 4, at least about 5, at least about 6, at least about 7, at least about 8, at least about 9 or at least about 10 times the total number of acidic monomers or moieties in the polymeric catalyst or solid-supported catalyst. In certain embodiments, the ratio of the total number of ionic monomers or moieties to the total number of acidic monomers or moieties is about 1:1, about 2:1, about 3:1, about 4:1, about 5:1, about 6:1, about 7:1, about 8:1, about 9:1 or about 10:1.

### Arrangement of Monomers in Polymeric Catalysts

In some embodiments of the polymeric catalysts, the acidic monomers, the ionic monomers, the acidic-ionic monomers and the hydrophobic monomers, where present, can be arranged in alternating sequence or in a random order as blocks of monomers. In some embodiments, each block has not more than twenty, fifteen, ten, six, or three monomers.

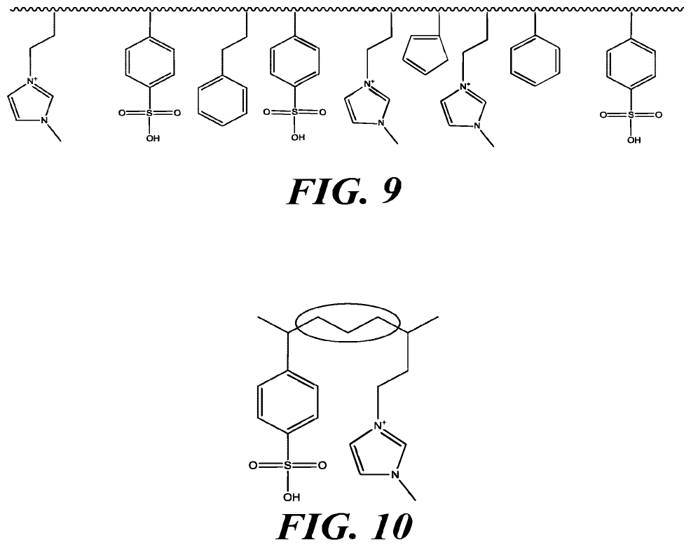

In some embodiments of the polymeric catalysts, the monomers of the polymeric catalyst are randomly arranged in an alternating sequence. With reference to the portion of the polymeric catalyst depicted in FIG. 9, the monomers are randomly arranged in an alternating sequence.

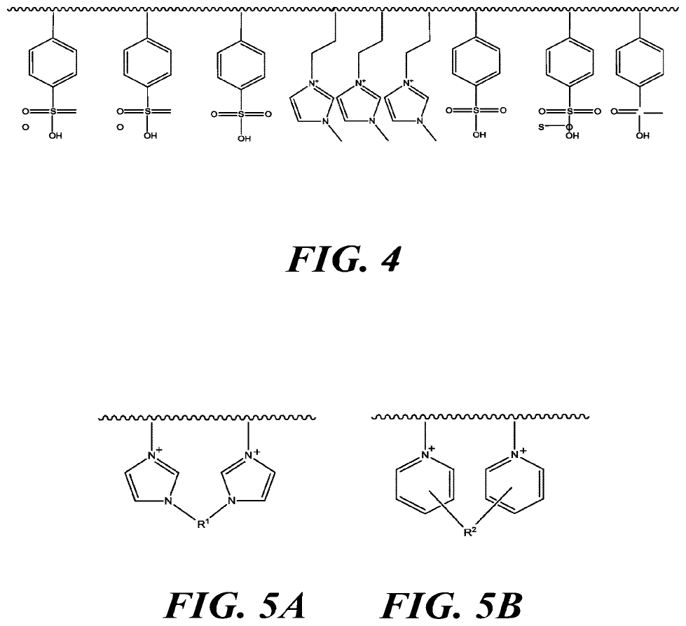

In other embodiments of the polymeric catalysts, the monomers of the polymeric catalyst are randomly arranged as blocks of monomers. With reference to the portion of the polymeric catalyst depicted in FIG. 4, the monomers are arranged in blocks of monomers. In certain embodiments where the acidic monomers and the ionic monomers are arranged in blocks of monomers, each block has no more than 20, 19, 18, 17, 16, 15, 14, 13, 12, 11, 10, 9, 8, 7, 6, 5, 4, or 3 monomers.

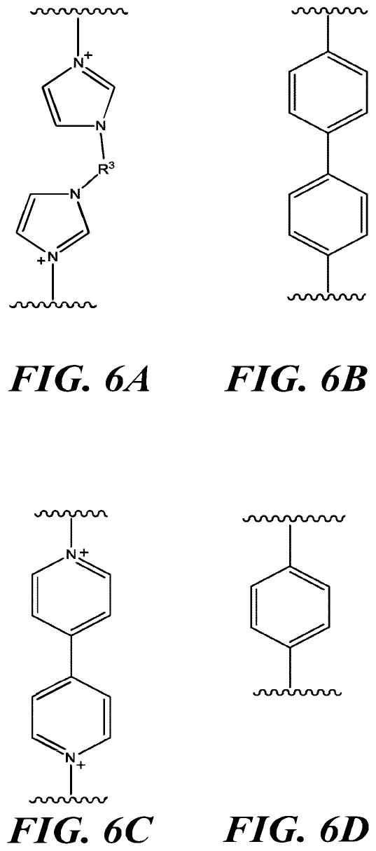

The polymeric catalysts described herein can also be cross-linked. Such cross-linked polymeric catalysts can be prepared by introducing cross-linking groups. In some embodiments, cross-linking can occur within a given polymeric chain, with reference to the portion of the polymeric catalysts depicted in FIGS. 5A and 5B. In other embodiments, cross-linking can occur between two or more polymeric chains, with reference to the portion of the polymeric catalysts in FIGS. 6A, 6B, 6C and 6D.

With reference to FIGS. 5A, 5B and 6A, it should be understood that R¹, R² and R³, respectively, are exemplary cross linking groups. Suitable cross-linking groups that can be used to form a cross-linked polymeric catalyst with the polymers described herein include, for example, substituted or unsubstituted divinyl alkanes, substituted or unsubstituted divinyl cycloalkanes, substituted or unsubstituted divinyl aryls, substituted or unsubstituted heteroaryls, dihaloalkanes, dihaloalkenes, and dihaloalkynes, where the substituents are those as defined herein. For example, cross-linking groups can include divinylbenzene, diallylbenzene, dichlorobenzene, divinylmethane, dichloromethane, divinylethane, dichloroethane, divinylpropane, dichloropropane, divinylbutane, dichlorobutane, ethylene glycol, and resorcinol. In one embodiment, the crosslinking group is divinyl benzene.

In some embodiments of the polymeric catalysts, the polymer is cross-linked. In certain embodiments, at least about 1%, at least about 2%, at least about 3%, at least about 4%, at least about 5%, at least about 6%, at least about 7%, at least about 8%, at least about 9%, at least about 10%, at least about 15%, at least about 20%, at least about 30%, at least about 40%, at least about 50%, at least about 60%, at least about 70%, at least about 80%, at least about 90% or at least about 99% of the polymer is cross-linked.

In some embodiments of the polymeric catalysts, the polymers described herein are not substantially cross-linked, such as less than about 0.9% cross-linked, less than about 0.5% cross-linked, less than about 0.1% cross-linked, less than about 0.01% cross-linked, or less than 0.001% cross-linked.

### Polymeric Backbones

In some embodiments, the polymeric backbone is formed from one or more substituted or unsubstituted monomers. Polymerization processes using a wide variety of monomers are well known in the art (see, e.g., International Union of Pure and Applied Chemistry, et al., IUPAC Gold Book, Polymerization. (2000)). One such process involves monomer(s) with unsaturated substitution, such as vinyl, propenyl, butenyl, or other such substitutent(s). These types of monomers can undergo radical initiation and chain polymerization.

In some embodiments, the polymeric backbone is formed from one or more substituted or unsubstituted monomers selected from ethylene, propylene, hydroxyethylene, acetaldehyde, styrene, divinyl benzene, isocyanates, vinyl chloride, vinyl phenols, tetrafluoroethylene, butylene, terephthalic acid, caprolactam, acrylonitrile, butadiene, ammonias, diammonias, pyrrole, imidazole, pyrazole, oxazole, thiazole, pyridine, pyrimidine, pyrazine, pyradizimine, thiazine, morpholine, piperidine, piperizines, pyrollizine, triphenylphosphonate, trimethylphosphonate, triethylphosphonate, tripropylphosphonate, tributylphosphonate, trichlorophosphonate, trifluorophosphonate, and diazole.

The polymeric backbone of the polymeric catalysts described herein can include, for example, polyalkylenes, polyalkenyl alcohols, polycarbonates, polyarylenes, polyaryletherketones, and polyamide-imides. In certain embodiments, the polymeric backbone can be selected from polyethylene, polypropylene, polyvinyl alcohol, polystyrene, polyurethane, polyvinyl chloride, polyphenol-aldehyde, polytetrafluoroethylene, polybutylene terephthalate, polycaprolactam, and poly(acrylonitrile butadiene styrene). In certain embodiments of the polymeric catalyst, the polymeric backbone is polyethyelene or polypropylene. In one embodiment of the polymeric catalyst, the polymeric backbone is polyethylene. In another embodiment of the polymeric catalyst, the polymeric backbone is polyvinyl alcohol. In yet another embodiment of the polymeric catalyst, the polymeric backbone is polystyrene.

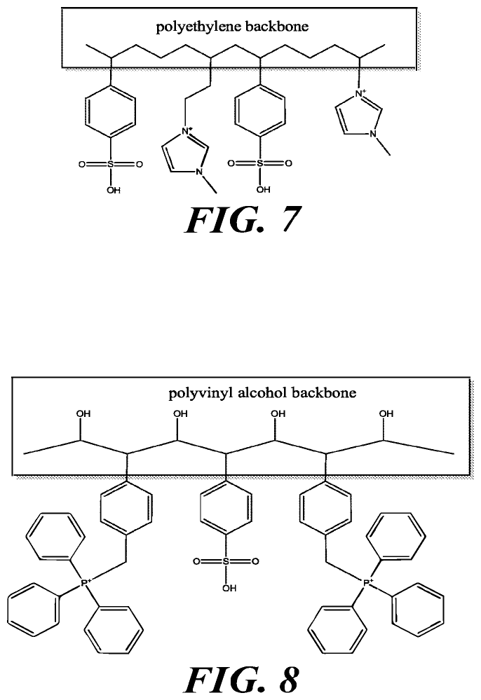

With reference to FIG. 7, in one embodiment, the polymeric backbone is polyethylene. With reference to FIG. 8, in another embodiment, the polymeric backbone is polyvinyl alcohol.

The polymeric backbone described herein can also include an ionic group integrated as part of the polymeric backbone. Such polymeric backbones can also be called “ionomeric backbones”. In certain embodiments, the polymeric backbone can be selected from: polyalkyleneammonium, polyalkylenediammonium, polyalkylenepyrrolium, polyalkylencimidazolium, polyalkylenepyrazolium, polyalkylencoxazolium, polyalkylenethiazolium, polyalkylenepyridinium, polyalkylenepyrimidinium, polyalkylenepyrazinium, polyalkylenepyridazinium, polyalkylenethiazinium, polyalkylenemorpholinium, polyalkylenepiperidinium, polyalkylenepiperizinium, polyalkylenepyrollizinium, polyalkylenetriphenylphosphonium, polyalkylenetrimethylphosphonium, polyalkylenetriethylphosphonium, polyalkylenetripropylphosphonium, polyalkylenetributylphosphonium, polyalkylenetrichlorophosphonium, polyalkylenetrifluorophosphonium, and polyalkylenediazolium, polyarylalkyleneammonium, polyarylalkylenediammonium, polyarylalkylenepyrrolium, polyarylalkyleneimidazolium, polyarylalkylenepyrazolium, polyarylalkyleneoxazolium, polyarylalkylenethiazolium, polyarylalkylenepyridinium, polyarylalkylenepyrimidinium, polyarylalkylenepyrazinium, polyarylalkylenepyridazinium, polyarylalkylenethiazinium, polyarylalkylenemorpholinium, polyarylalkylenepiperidinium, polyarylalkylenepiperizinium, polyarylalkylenepyrollizinium, polyarylalkylenetriphenylphosphonium, polyarylalkylenetrimethylphosphonium, polyarylalkylenetriethylphosphonium, polyarylalkylenetripropylphosphonium, polyarylalkylenetributylphosphonium, polyarylalkylenetrichlorophosphonium, polyarylalkylenetrifluorophosphonium, and polyarylalkylenediazolium.

Cationic polymeric backbones can be associated with one or more anions, including for example F⁻, Cl⁻, Br⁻, I⁻, NO₂⁻, NO₃⁻, SO₄²⁻, R⁷SO₄⁻, R⁷CO₂⁻, PO₄²⁻, R⁷PO₃⁻, and R⁷PO₂⁻, where R⁷ is selected from hydrogen, C₁₋₄alkyl, and C₁₋₄heteroalkyl. In one embodiment, each anion can be selected from Cl⁻, Br⁻, I⁻, HSO₄⁻, HCO₂⁻, CH₃CO₂⁻, and NO₃⁻. In other embodiments, each anion is acetate. In other embodiments, each anion is bisulfate. In other embodiments, each anion is chloride. In other embodiments, X is nitrate.

In other embodiments of the polymeric catalysts, the polymeric backbone is alkyleneimidazolium, which refers to an alkylene moiety, in which one or more of the methylene units of the alkylene moiety has been replaced with imidazolium. In one embodiment, the polymeric backbone is selected from polyethyleneimidazolium, polyprolyeneimidazolium, and polybutyleneimidazolium. It should further be understood that, in other embodiments of the polymeric backbone, when a nitrogen-containing cationic group or a phosphorous-containing cationic group follows the term “alkylene”, one or more of the methylene units of the alkylene moiety is substituted with that nitrogen-containing cationic group or phosphorous-containing cationic group.

In other embodiments, monomers having heteroatoms can be combined with one or more difunctionalized compounds, such as dihaloalkanes, di(alkylsulfonyloxy)alkanes, and di(arylsulfonyloxy)alkanes to form polymers. The monomers have at least two heteroatoms to link with the difunctionalized alkane to create the polymeric chain. These difunctionalized compounds can be further substituted as described herein. In some embodiments, the difunctionalized compound(s) can be selected from 1,2-dichloroethane, 1,2-dichloropropane, 1,3-dichloropropane, 1,2-dichlorobutane, 1,3-dichlorobutane, 1,4-dichlorobutane, 1,2-dichloropentane, 1,3-dichloropentane, 1,4-dichloropentane, 1,5-dichloropentane, 1,2-dibromoethane, 1,2-dibromopropane, 1,3-dibromopropane, 1,2-dibromobutane, 1,3-dibromobutane, 1,4-dibromobutane, 1,2-dibromopentane, 1,3-dibromopentane, 1,4-dibromopentane, 1,5-dibromopentane, 1,2-diiodoethane, 1,2-diiodopropane, 1,3-diiodopropane, 1,2-diiodobutane, 1,3-diiodobutane, 1,4-diiodobutane, 1,2-diiodopentane, 1,3-diiodopentane, 1,4-diiodopentane, 1,5-diiodopentane, 1,2-dimethanesulfoxyethane, 1,2-dimethanesulfoxypropane, 1,3-dimethanesulfoxypropane, 1,2-dimethanesulfoxybutane, 1,3-dimethanesulfoxybutane, 1,4-dimethanesulfoxybutane, 1,2-dimethanesulfoxypentane, 1,3-dimethanesulfoxypentane, 1,4-dimethanesulfoxypentane, 1,5-dimethanesulfoxypentane, 1,2-diethanesulfoxyethane, 1,2-diethanesulfoxypropane, 1,3-diethanesulfoxypropane, 1,2-diethanesulfoxybutane, 1,3-diethanesulfoxybutane, 1,4-diethanesulfoxybutane, 1,2-diethanesulfoxypentane, 1,3-diethanesulfoxypentane, 1,4-diethanesulfoxypentane, 1,5-diethanesulfoxypentane, 1,2-dibenzenesulfoxyethane, 1,2-dibenzenesulfoxypropane, 1,3-dibenzenesulfoxypropane, 1,2-dibenzenesulfoxybutane, 1,3-dibenzenesulfoxybutane, 1,4-dibenzenesulfoxybutane, 1,2-dibenzenesulfoxypentane, 1,3-dibenzenesulfoxypentane, 1,4-dibenzenesulfoxypentane, 1,5-dibenzenesulfoxypentane, 1,2-di-p-toluenesulfoxyethane, 1,2-di-p-toluenesulfoxypropane, 1,3-di-p-toluenesulfoxypropane, 1,2-di-p-toluenesulfoxybutane, 1,3-di-p-toluenesulfoxybutane, 1,4-di-p-toluenesulfoxybutane, 1,2-di-p-toluenesulfoxypentane, 1,3-di-p-toluene sulfoxypentane, 1,4-di-p-toluene sulfoxypentane, and 1,5-di-p-toluene sulfoxypentane.

Further, the number of atoms between side chains in the polymeric backbone can vary. In some embodiments, there are between zero and twenty atoms, zero and ten atoms, zero and six atoms, or zero and three atoms between side chains attached to the polymeric backbone.

In some embodiments, the polymer can be a homopolymer having at least two monomer units, and where all the units contained within the polymer are derived from the same monomer in the same manner. In other embodiments, the polymer can be a heteropolymer having at least two monomer units, and where at least one monomeric unit contained within the polymer that differs from the other monomeric units in the polymer. The different monomer units in the polymer can be in a random order, in an alternating sequence of any length of a given monomer, or in blocks of monomers.

Other exemplary polymers include, for example, polyalkylene backbones that are substituted with one or more groups selected from hydroxyl, carboxylic acid, unsubstituted and substituted phenyl, halides, unsubstituted and substituted amines, unsubstituted and substituted ammonias, unsubstituted and substituted pyrroles, unsubstituted and substituted imidazoles, unsubstituted and substituted pyrazoles, unsubstituted and substituted oxazoles, unsubstituted and substituted thiazoles, unsubstituted and substituted pyridines, unsubstituted and substituted pyrimidines, unsubstituted and substituted pyrazines, unsubstituted and substituted pyradizines, unsubstituted and substituted thiazines, unsubstituted and substituted morpholines, unsubstituted and substituted piperidines, unsubstituted and substituted piperizines, unsubstituted and substituted pyrollizines, unsubstituted and substituted triphenylphosphonates, unsubstituted and substituted trimethylphosphonates, unsubstituted and substituted triethylphosphonates, unsubstituted and substituted tripropylphosphonates, unsubstituted and substituted tributylphosphonates, unsubstituted and substituted trichlorophosphonates, unsubstituted and substituted trifluorophosphonates, and unsubstituted and substituted diazoles.

For the polymers as described herein, multiple naming conventions are well recognized in the art. For instance, a polyethylene backbone with a direct bond to an unsubstituted phenyl group (—CH₂—CH(phenyl)-CH₂—CH(phenyl)-) is also known as polystyrene. Should that phenyl group be substituted with an ethenyl group, the polymer can be named a polydivinylbenzene (—CH₂—CH(4-vinylphenyl)-CH₂—CH(4-vinylphenyl)-). Further examples of heteropolymers may include those that are functionalized after polymerization.

One suitable example would be polystyrene-co-divinylbenzene: (—CH₂—CH(phenyl)-CH₂—CH(4-ethylenephenyl)-CH₂—CH(phenyl)-CH₂—CH(4-ethylenephenyl)-). Here, the ethenyl functionality could be at the 2, 3, or 4 position on the phenyl ring.

With reference to FIG. 12, in yet another embodiment, the polymeric backbone is a polyalkyleneimidazolium.

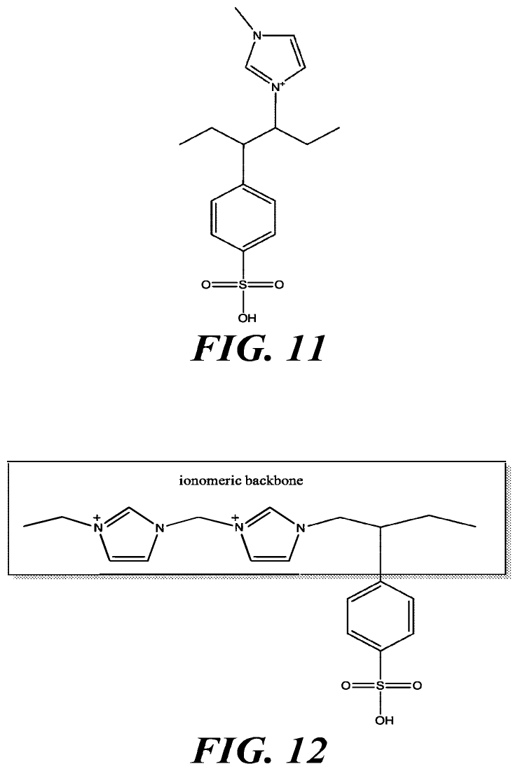

Further, the number of atoms between side chains in the polymeric backbone can vary. In some embodiments, there are between zero and twenty atoms, zero and ten atoms, or zero and six atoms, or zero and three atoms between side chains attached to the polymeric backbone. With reference to FIG. 10, in one embodiment, there are three carbon atoms between the side chain with the Bronsted-Lowry acid and the side chain with the cationic group. In another example, with reference to FIG. 11, there are zero atoms between the side chain with the acidic moiety and the side chain with the ionic moiety.

### Solid Particles for Polymeric Catalysts

The polymeric catalysts described herein can form solid particles. One of skill in the art would recognize the various known techniques and methods to make solid particles from the polymers described herein. For example, a solid particle can be formed through the procedures of emulsion or dispersion polymerization, which are known to one of skill in the art. In other embodiments, the solid particles can be formed by grinding or breaking the polymer into particles, which are also techniques and methods that are known to one of skill in the art. Methods known in the art to prepare solid particles include coating the polymers described herein on the surface of a solid core. Suitable materials for the solid core can include an inert material (e.g., aluminum oxide, corn cob, crushed glass, chipped plastic, pumice, silicon carbide, or walnut shell) or a magnetic material. Polymeric coated core particles can be made by dispersion polymerization to grow a cross-linked polymer shell around the core material, or by spray coating or melting.

Other methods known in the art to prepare solid particles include coating the polymers described herein on the surface of a solid core. The solid core can be a non-catalytic support. Suitable materials for the solid core can include an inert material (e.g., aluminum oxide, corn cob, crushed glass, chipped plastic, pumice, silicon carbide, or walnut shell) or a magnetic material. In one embodiment of the polymeric catalyst, the solid core is made up of iron. Polymeric coated core particles can be made by techniques and methods that are known to one of skill in the art, for example, by dispersion polymerization to grow a cross-linked polymer shell around the core material, or by spray coating or melting.

The solid supported polymer catalyst particle can have a solid core where the polymer is coated on the surface of the solid core. In some embodiments, at least about 5%, at least about 10%, at least about 20%, at least about 30%, at least about 40%, or at least about 50% of the catalytic activity of the solid particle can be present on or near the exterior surface of the solid particle. In some embodiments, the solid core can have an inert material or a magnetic material. In one embodiment, the solid core is made up of iron.

The solid particles coated with the polymer described herein have one or more catalytic properties. In some embodiments, at least about 50%, at least about 60%, at least about 70%, at least about 80% or at least about 90% of the catalytic activity of the solid particle is present on or near the exterior surface of the solid particle.

In some embodiments, the solid particle is substantially free of pores, for example, having no more than about 50%, no more than about 40%, no more than about 30%, no more than about 20%, no more than about 15%, no more than about 10%, no more than about 5%, or no more than about 1% of pores. Porosity can be measured by methods well known in the art, such as determining the Brunauer-Emmett-Teller (BET) surface area using the absorption of nitrogen gas on the internal and external surfaces of a material (Brunauer, S. et al., J. Am. Chem. Soc. 1938, 60:309). Other methods include measuring solvent retention by exposing the material to a suitable solvent (such as water), then removing it thermally to measure the volume of interior pores. Other solvents suitable for porosity measurement of the polymeric catalysts include, for example, polar solvents such as DMF, DMSO, acetone, and alcohols.

In other embodiments, the solid particles include a microporous gel resin. In yet other embodiments, the solid particles include a macroporous gel resin.

### Support of the Solid-Supported Catalysts

In certain embodiments of the solid-supported catalyst, the support may be selected from biochar, carbon, amorphous carbon, activated carbon, silica, silica gel, alumina, magnesia, titania, zirconia, clays (e.g., kaolinite), magnesium silicate, silicon carbide, zeolites (e.g., mordenite), ceramics, and any combinations thereof. In one embodiment, the support is carbon. The support for carbon support can be biochar, amorphous carbon, or activated carbon. In one embodiment, the support is activated carbon.

The carbon support can have a surface area from 0.01 to 50 m²/g of dry material. The carbon support can have a density from 0.5 to 2.5 kg/L. The support can be characterized using any suitable instrumental analysis methods or techniques known in the art, including for example scanning electron microscopy (SEM), powder X-ray diffraction (XRD), Raman spectroscopy, and Fourier Transform infrared spectroscopy (FTIR). The carbon support can be prepared from carbonaceous materials, including for example, shrimp shell, chitin, coconut shell, wood pulp, paper pulp, cotton, cellulose, hard wood, soft wood, wheat straw, sugarcane bagasse, cassava stem, corn stover, oil palm residue, bitumen, asphaltum, tar, coal, pitch, and any combinations thereof. One of skill in the art would recognize suitable methods to prepare the carbon supports used herein. See e.g., M. Inagaki, L. R. Radovic, Carbon, vol. 40, p. 2263 (2002), or A. G. Pandolfo and A. F. Hollenkamp, “Review: Carbon Properties and their role in supercapacitors,” Journal of Power Sources, vol. 157, pp. 11-27 (2006).

In other embodiments, the support is silica, silica gel, alumina, or silica-alumina. One of skill in the art would recognize suitable methods to prepare these silica- or alumina-based solid supports used herein. See e.g., Catalyst supports and supported catalysts, by A. B. Stiles, Butterworth Publishers, Stoneham MA, 1987.

In yet other embodiments, the support is a combination of a carbon support, with one or more other supports selected from silica, silica gel, alumina, magnesia, titania, zirconia, clays (e.g., kaolinite), magnesium silicate, silicon carbide, zeolites (e.g., mordenite), and ceramics.

### Definitions

“Bronsted-Lowry acid” refers to a molecule, or substituent thereof, in neutral or ionic form that is capable of donating a proton (hydrogen cation, He).

“Homopolymer” refers to a polymer having at least two monomer units, and where all the units contained within the polymer are derived from the same monomer. One suitable example is polyethylene, where ethylene monomers are linked to form a uniform repeating chain (—CH₂—CH₂—CH₂—). Another suitable example is polyvinyl chloride, having a structure (—CH₂—CHCl—CH₂—CHCl—) where the —CH₂—CHCl— repeating unit is derived from the H₂C═CHCl monomer.

1 “Heteropolymer” refers to a polymer having at least two monomer units, and where at least one monomeric unit differs from the other monomeric units in the polymer. Heteropolymer also refers to polymers having difunctionalized or trifunctionalized monomer units that can be incorporated in the polymer in different ways. The different monomer units in the polymer can be in a random order, in an alternating sequence of any length of a given monomer, or in blocks of monomers. One suitable example is polyethyleneimidazolium, where if in an alternating sequence, would be the polymer depicted in FIG. 12. Another suitable example is polystyrene-co-divinylbenzene, where if in an alternating sequence, could be (—CH₂—CH(phenyl)-CH₂—CH(4-ethylenephenyl)-CH₂—CH(phenyl)-CH₂—CH(4-ethylenephenyl)-). Here, the ethenyl functionality could be at the 2, 3, or 4 position on the phenyl ring.

As used herein, [symbol] denotes the attachment point of a moiety to the parent structure.

When a range of values is listed, it is intended to encompass each value and sub-range within the range. For example, “C₁₋₆ alkyl” (which may also be referred to as 1-6C alkyl, C₁-C₆ alkyl, or C₁₋₆ alkyl) is intended to encompass, C₁, C₂, C₃, C₄, C₅, C₆, C_{1 6}, C_{1 5}, C_{1 4}, C_{1 3}, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

“Alkyl” includes saturated straight-chained or branched monovalent hydrocarbon radicals, which contain only C and H when unsubstituted. In some embodiments, alkyl as used herein may have 1 to 10 carbon atoms (e.g., C₁₋₁₀ alkyl), 1 to 6 carbon atoms (e.g., C₁₋₆ alkyl), or 1 to 3 carbon atoms (e.g., C₁₋₃ alkyl). Representative straight-chained alkyls include, for example, methyl, ethyl, n-propyl, n-butyl, n-pentyl, and n-hexyl. Representative branched alkyls include, for example, isopropyl, sec-butyl, isobutyl, tert-butyl, isopentyl, 2-methylbutyl, 3-methylbutyl, 2-methylpentyl, 3-methylpentyl, 4-methylpentyl, 2-methylhexyl, 3-methylhexyl, 4-methylhexyl, 5-methylhexyl, and 2,3-dimethylbutyl. When an alkyl residue having a specific number of carbons is named, all geometric isomers having that number of carbons are intended to be encompassed and described; thus, for example, “butyl” is meant to include n-butyl, sec-butyl, iso-butyl, and tert-butyl; “propyl” includes n-propyl, and iso-propyl.

“Alkoxy” refers to the group —O-alkyl, which is attached to the parent structure through an oxygen atom. Examples of alkoxy may include methoxy, ethoxy, propoxy, and isopropoxy. In some embodiments, alkoxy as used herein has 1 to 6 carbon atoms (e.g., O—(C₁₋₆ alkyl)), or 1 to 4 carbon atoms (e.g., O—(C₁₋₄ alkyl)).

“Alkenyl” refers to straight-chained or branched monovalent hydrocarbon radicals, which contain only C and H when unsubstituted and at least one double bond. In some embodiments, alkenyl has 2 to 10 carbon atoms (e.g., C₂₋₁₀ alkenyl), or 2 to 5 carbon atoms (e.g., C₂₋₅ alkenyl). When an alkenyl residue having a specific number of carbons is named, all geometric isomers having that number of carbons are intended to be encompassed and described; thus, for example, “butenyl” is meant to include n-butenyl, sec-butenyl, and iso-butenyl. Examples of alkenyl may include —CH═CH₂, —CH₂—CH═CH₂ and —CH₂—CH═CH—CH═CH₂. The one or more carbon-carbon double bonds can be internal (such as in 2-butenyl) or terminal (such as in 1-butenyl). Examples of C₂₋₄ alkenyl groups include ethenyl (C2), 1-propenyl (C3), 2-propenyl (C3), 1-butenyl (C4), 2-butenyl (C4), and butadienyl (C4). Examples of C₂₋₆ alkenyl groups include the aforementioned C₂₋₄ alkenyl groups as well as pentenyl (C5), pentadienyl (C5), and hexenyl (C6). Additional examples of alkenyl include heptenyl (C7), octenyl (C8), and octatrienyl (C8).

“Alkynyl” refers to straight-chained or branched monovalent hydrocarbon radicals, which contain only C and H when unsubstituted and at least one triple bond. In some embodiments, alkynyl has 2 to 10 carbon atoms (e.g., C₂₋₁₀ alkynyl), or 2 to 5 carbon atoms (e.g., C₂₋₅ alkynyl). When an alkynyl residue having a specific number of carbons is named, all geometric isomers having that number of carbons are intended to be encompassed and described; thus, for example, “pentynyl” is meant to include n-pentynyl, sec-pentynyl, iso-pentynyl, and tert-pentynyl. Examples of alkynyl may include —C≡CH or —C≡C—CH₃.

In some embodiments, alkyl, alkoxy, alkenyl, and alkynyl at each occurrence may independently be unsubstituted or substituted by one or more of substituents. In certain embodiments, substituted alkyl, substituted alkoxy, substituted alkenyl, and substituted alkynyl at each occurrence may independently have 1 to 5 substituents, 1 to 3 substituents, 1 to 2 substituents, or 1 substituent. Examples of alkyl, alkoxy, alkenyl, and alkynyl substituents may include alkoxy, cycloalkyl, aryl, aryloxy, amino, amido, carbamate, carbonyl, oxo (═O), heteroalkyl (e.g., ether), heteroaryl, heterocycloalkyl, cyano, halo, haloalkoxy, haloalkyl, and thio. In certain embodiments, the one or more substituents of substituted alkyl, alkoxy, alkenyl, and alkynyl is independently selected from cycloalkyl, aryl, heteroalkyl (e.g., ether), heteroaryl, heterocycloalkyl, cyano, halo, haloalkoxy, haloalkyl, oxo, —ORₐ, —N(Rₐ)₂, —C(O)N(Rₐ)₂, —N(Rₐ)C(O)Rₐ, —C(O)Rₐ, —N(Rₐ)S(O)ₜRₐ (where t is 1 or 2), —SRₐ, and —S(O)ₜN(Rₐ)₂ (where t is 1 or 2). In certain embodiments, each Rₐ is independently hydrogen, alkyl, alkenyl, alkynyl, haloalkyl, heteroalkyl, cycloalkyl, aryl, heterocycloalkyl, heteroaryl (e.g., bonded through a ring carbon), —C(O)R′ and —S(O)ₜR′ (where t is 1 or 2), where each R′ is independently hydrogen, alkyl, alkenyl, alkynyl, haloalkyl, heteroalkyl, cycloalkyl, aryl, heterocycloalkyl, or heteroaryl. In one embodiment, Rₐ is independently hydrogen, alkyl, haloalkyl, cycloalkyl, aryl, aralkyl (e.g., alkyl substituted with aryl, bonded to parent structure through the alkyl group), heterocycloalkyl, or heteroaryl.

“Heteroalkyl”, “heteroalkenyl” and “heteroalkynyl” includes alkyl, alkenyl and alkynyl groups, respectively, wherein one or more skeletal chain atoms are selected from an atom other than carbon, e.g., oxygen, nitrogen, sulfur, phosphorus, or any combinations thereof. For example, heteroalkyl may be an ether where at least one of the carbon atoms in the alkyl group is replaced with an oxygen atom. A numerical range can be given, e.g., C₁₋₄ heteroalkyl which refers to the chain length in total, which in this example is 4 atoms long. For example, a —CH₂OCH₂CH₃ group is referred to as a “C₄” heteroalkyl, which includes the heteroatom center in the atom chain length description. Connection to the rest of the parent structure can be through, in one embodiment, a heteroatom, or, in another embodiment, a carbon atom in the heteroalkyl chain. Heteroalkyl groups may include, for example, ethers such as methoxyethanyl (—CH₂CH₂OCH₃), ethoxymethanyl (—CH₂OCH₂CH₃), (methoxymethoxy)ethanyl (—CH₂CH₂OCH₂OCH₃), (methoxymethoxy)methanyl (—CH₂OCH₂OCH₃) and (methoxyethoxy)methanyl (—CH₂OCH₂ CH₂OCH₃); amines such as —CH₂CH₂NHCH₃, —CH₂CH₂N(CH₃)₂, —CH₂NHCH₂CH₃, and —CH₂N(CH₂CH₃)(CH₃). In some embodiments, heteroalkyl, heteroalkenyl, or heteroalkynyl may be unsubstituted or substituted by one or more of substituents. In certain embodiments, a substituted heteroalkyl, heteroalkenyl, or heteroalkynyl may have 1 to 5 substituents, 1 to 3 substituents, 1 to 2 substituents, or 1 substituent. Examples for heteroalkyl, heteroalkenyl, or heteroalkynyl substituents may include the substituents described above for alkyl.

“Carbocyclyl” may include cycloalkyl, cycloalkenyl or cycloalkynyl. “Cycloalkyl” refers to a monocyclic or polycyclic alkyl group. “Cycloalkenyl” refers to a monocyclic or polycyclic alkenyl group (e.g., containing at least one double bond). “Cycloalkynyl” refers to a monocyclic or polycyclic alkynyl group (e.g., containing at least one triple bond). The cycloalkyl, cycloalkenyl, or cycloalkynyl can consist of one ring, such as cyclohexyl, or multiple rings, such as adamantyl. A cycloalkyl, cycloalkenyl, or cycloalkynyl with more than one ring can be fused, spiro or bridged, or combinations thereof. In some embodiments, cycloalkyl, cycloalkenyl, and cycloalkynyl has 3 to 10 ring atoms (i.e., C₃-C₁₀ cycloalkyl, C₃-C₁₀ cycloalkenyl, and C₃-C₁₀ cycloalkynyl), 3 to 8 ring atoms (e.g., C₃-C₈ cycloalkyl, C₃-C₈ cycloalkenyl, and C₃-C₈ cycloalkynyl), or 3 to 5 ring atoms (i.e., C₃-C₅ cycloalkyl, C₃-C₈ cycloalkenyl, and C₃-C₅ cycloalkynyl). In certain embodiments, cycloalkyl, cycloalkenyl, or cycloalkynyl includes bridged and spiro-fused cyclic structures containing no heteroatoms. In other embodiments, cycloalkyl, cycloalkenyl, or cycloalkynyl includes monocyclic or fused-ring polycyclic (i.e., rings which share adjacent pairs of ring atoms) groups. C₃₋₆ carbocyclyl groups may include, for example, cyclopropyl (C₃), cyclobutyl (C₄), cyclopentyl (C₅), cyclopentenyl (C₅), cyclohexyl (C), cyclohexenyl (C), and cyclohexadienyl (C₆). C₃-s carbocyclyl groups may include, for example, the aforementioned C₃₋₆ carbocyclyl groups as well as cycloheptyl (C₇), cycloheptadienyl (C₇), cycloheptatrienyl (C₇), cyclooctyl (C₈), bicyclo[2.2.1]heptanyl, and bicyclo[2.2.2]octanyl. C₃₋₁₀ carbocyclyl groups may include, for example, the aforementioned C₃ 8 carbocyclyl groups as well as octahydro-1H-indenyl, decahydronaphthalenyl, and spiro[4.5]decanyl.

1 “Heterocyclyl” refers to carbocyclyl as described above, with one or more ring heteroatoms independently selected from nitrogen, oxygen phosphorous, and sulfur. Heterocyclyl may include, for example, heterocycloalkyl, heterocycloalkenyl, and heterocycloalknyl. In some embodiments, heterocyclyl is a 3- to 18-membered non-aromatic monocyclic or polycyclic moiety that has at least one heteroatom selected from nitrogen, oxygen, phosphorous and sulfur. In certain embodiments, the heterocyclyl can be a monocyclic or polycyclic (e.g., bicyclic, tricyclic or tetracyclic), wherein polycyclic ring systems can be a fused, bridged or spiro ring system. Heterocyclyl polycyclic ring systems can include one or more heteroatoms in one or both rings.

An N-containing heterocyclyl moiety refers to an non-aromatic group in which at least one of the skeletal atoms of the ring is a nitrogen atom. The heteroatom(s) in the heterocyclyl group is optionally oxidized. One or more nitrogen atoms, if present, are optionally quaternized. In certain embodiments, heterocyclyl may also include ring systems substituted with one or more oxide (—O—) substituents, such as piperidinyl N-oxides. The heterocyclyl is attached to the parent molecular structure through any atom of the ring(s).

In some embodiments, heterocyclyl also includes ring systems with one or more fused carbocyclyl, aryl or heteroaryl groups, wherein the point of attachment is either on the carbocyclyl or heterocyclyl ring. In some embodiments, heterocyclyl is a 5-10 membered non-aromatic ring system having ring carbon atoms and 1-4 ring heteroatoms, wherein each heteroatom is independently selected from nitrogen, oxygen and sulfur (e.g., 5-10 membered heterocyclyl). In some embodiments, a heterocyclyl group is a 5-8 membered non-aromatic ring system having ring carbon atoms and 1-4 ring heteroatoms, wherein each heteroatom is independently selected from nitrogen, oxygen and sulfur (e.g., 5-8 membered heterocyclyl). In some embodiments, a heterocyclyl group is a 5-6 membered non-aromatic ring system having ring carbon atoms and 1-4 ring heteroatoms, wherein each heteroatom is independently selected from nitrogen, oxygen and sulfur (e.g., 5-6 membered heterocyclyl). In some embodiments, the 5-6 membered heterocyclyl has 1-3 ring heteroatoms selected from nitrogen, oxygen and sulfur. In some embodiments, the 5-6 membered heterocyclyl has 1-2 ring heteroatoms selected from nitrogen, oxygen and sulfur. In some embodiments, the 5-6 membered heterocyclyl has 1 ring heteroatom selected from nitrogen, oxygen and sulfur.

“Aryl” refers to an aromatic group having a single ring (e.g., phenyl), multiple rings (e.g., biphenyl), or multiple fused rings (e.g., naphthyl, fluorenyl, and anthryl). In some embodiments, aryl as used herein has 6 to 10 ring atoms (e.g., C₆-C₁₀ aromatic or C₆-C₁₀ aryl) which has at least one ring having a conjugated pi electron system. For example, bivalent radicals formed from substituted benzene derivatives and having the free valences at ring atoms are named as substituted phenylene radicals. In certain embodiments, aryl may have more than one ring where at least one ring is non-aromatic can be connected to the parent structure at either an aromatic ring position or at a non-aromatic ring position. In certain embodiments, aryl includes monocyclic or fused-ring polycyclic (i.e., rings which share adjacent pairs of ring atoms) groups.

“Heteroaryl” refers to an aromatic group having a single ring, multiple rings, or multiple fused rings, with one or more ring heteroatoms independently selected from nitrogen, oxygen, phosphorous, and sulfur. In some embodiments, heteroaryl is an aromatic, monocyclic or bicyclic ring containing one or more heteroatoms independently selected from nitrogen, oxygen and sulfur with the remaining ring atoms being carbon. In certain embodiments, heteroaryl is a 5- to 18-membered monocyclic or polycyclic (e.g., bicyclic or tricyclic) aromatic ring system (e.g., having 6, 10 or 14 pi electrons shared in a cyclic array) having ring carbon atoms and 1 to 6 ring heteroatoms provided in the aromatic ring system, wherein each heteroatom is independently selected from nitrogen, oxygen, phosphorous and sulfur (e.g., 5-18 membered heteroaryl). In certain embodiments, heteroaryl may have a single ring (e.g., pyridyl, pyridinyl, imidazolyl) or multiple condensed rings (e.g., indolizinyl, benzothienyl) which condensed rings may or may not be aromatic. In other embodiments, heteroaryl may have more than one ring where at least one ring is non-aromatic can be connected to the parent structure at either an aromatic ring position or at a non-aromatic ring position. In one embodiment, heteroaryl may have more than one ring where at least one ring is non-aromatic is connected to the parent structure at an aromatic ring position. Heteroaryl polycyclic ring systems can include one or more heteroatoms in one or both rings.

For example, in one embodiment, an N-containing “heteroaryl” refers to an aromatic group in which at least one of the skeletal atoms of the ring is a nitrogen atom. One or more heteroatom(s) in the heteroaryl group can be optionally oxidized. One or more nitrogen atoms, if present, are optionally quaternized. In other embodiments, heteroaryl may include ring systems substituted with one or more oxide (—O—) substituents, such as pyridinyl N-oxides. The heteroaryl may be attached to the parent molecular structure through any atom of the ring(s).

In other embodiments, heteroaryl may include ring systems with one or more fused aryl groups, wherein the point of attachment is either on the aryl or on the heteroaryl ring. In yet other embodiments, heteroaryl may include ring systems with one or more carbocyclyl or heterocyclyl groups wherein the point of attachment is on the heteroaryl ring. For polycyclic heteroaryl groups wherein one ring does not contain a heteroatom (e.g., indolyl, quinolinyl, and carbazolyl) the point of attachment can be on either ring, i.e., either the ring bearing a heteroatom (e.g., 2-indolyl) or the ring that does not contain a heteroatom (e.g., 5-indolyl). In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, phosphorous, and sulfur (e.g., 5-10 membered heteroaryl). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, phosphorous, and sulfur (e.g., 5-8 membered heteroaryl). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, phosphorous, and sulfur (e.g., 5-6 membered heteroaryl). In some embodiments, the 5-6 membered heteroaryl has 1-3 ring heteroatoms selected from nitrogen, oxygen, phosphorous, and sulfur. In some embodiments, the 5-6 membered heteroaryl has 1-2 ring heteroatoms selected from nitrogen, oxygen, phosphorous, and sulfur. In some embodiments, the 5-6 membered heteroaryl has 1 ring heteroatom selected from nitrogen, oxygen, phosphorous, and sulfur.

In some embodiments, carbocyclyl (including, for example, cycloalkyl, cycloalkenyl or cycloalkynyl), aryl, heteroaryl, and heterocyclyl at each occurrence may independently be unsubstituted or substituted by one or more of substituents. In certain embodiments, a substituted carbocyclyl (including, for example, substituted cycloalkyl, substituted cycloalkenyl or substituted cycloalkynyl), substituted aryl, substituted heteroaryl, substituted heterocyclyl at each occurrence may be independently may independently have 1 to 5 substituents, 1 to 3 substituents, 1 to 2 substituents, or 1 substituent. Examples of carbocyclyl (including, for example, cycloalkyl, cycloalkenyl or cycloalkynyl), aryl, heteroaryl, heterocyclyl substituents may include alkyl alkenyl, alkoxy, cycloalkyl, aryl, heteroalkyl (e.g., ether), heteroaryl, heterocycloalkyl, cyano, halo, haloalkoxy, haloalkyl, oxo (═O), —ORₐ, —N(Rₐ)₂, —C(O)N(Rₐ)₂, —N(Rₐ)C(O)Rₐ, —C(O)Rₐ, —N(Rₐ)S(O)ₜRₐ (where t is 1 or 2), —SRₐ, and —S(O)ₜN(Rₐ)₂ (where t is 1 or 2), wherein Rₐ is as described herein.

It should be understood that, as used herein, any moiety referred to as a “linker” refers to the moiety has having bivalency. Thus, for example, “alkyl linker” refers to the same residues as alkyl, but having bivalency. Examples of alkyl linkers include —CH₂—, —CH₂CH₂—, —CH₂CH₂CH₂—, and —CH₂CH₂CH₂CH₂—. “Alkenyl linker” refers to the same residues as alkenyl, but having bivalency. Examples of alkenyl linkers include —CH═CH—, —CH₂—CH═CH— and —CH₂—CH═CH—CH₂—. “Alkynyl linker” refers to the same residues as alkynyl, but having bivalency. Examples alkynyl linkers include —C≡C— or —C≡C—CH₂—. Similarly, “carbocyclyl linker”, “aryl linker”, “heteroaryl linker”, and “heterocyclyl linker” refer to the same residues as carbocyclyl, aryl, heteroaryl, and heterocyclyl, respectively, but having bivalency.

“Amino” or “amine” refers to —N(Rₐ)(R_{b}), where each Rₐ and R_{b} is independently selected from hydrogen, alkyl, alkenyl, alkynyl, haloalkyl, heteroalkyl (e.g., bonded through a chain carbon), cycloalkyl, aryl, heterocycloalkyl (e.g., bonded through a ring carbon), heteroaryl (e.g., bonded through a ring carbon), —C(O)R′ and —S(O)ₜR′ (where t is 1 or 2), where each R′ is independently hydrogen, alkyl, alkenyl, alkynyl, haloalkyl, heteroalkyl, cycloalkyl, aryl, heterocycloalkyl, or heteroaryl. It should be understood that, in one embodiment, amino includes amido (e.g., —NRₐC(O)R_{b}). It should be further understood that in certain embodiments, the alkyl, alkenyl, alkynyl, haloalkyl, heteroalkyl, cycloalkyl, aryl, heterocycloalkyl, or heteroaryl moiety of Rₐ and R_{b} may be further substituted as described herein. Rₐ and R_{b} may be the same or different. For example, in one embodiment, amino is —NH₂ (where Rₐ and R_{b} are each hydrogen). In other embodiments where Rₐ and R_{b} are other than hydrogen, Rₐ and R_{b} can be combined with the nitrogen atom to which they are attached to form a 3-, 4-, 5-, 6-, or 7-membered ring. Such examples may include 1-pyrrolidinyl and 4-morpholinyl.

“Ammonium” refers to —N(Rₐ)(R_{b})(R_{c})⁺, where each Rₐ, R_{b} and R_{c} is independently selected from hydrogen, alkyl, alkenyl, alkynyl, haloalkyl, heteroalkyl (e.g., bonded through a chain carbon), cycloalkyl, aryl, heterocycloalkyl (e.g., bonded through a ring carbon), heteroaryl (e.g., bonded through a ring carbon), —C(O)R′ and —S(O)ₜR′ (where t is 1 or 2), where each R′ is independently hydrogen, alkyl, alkenyl, alkynyl, haloalkyl, heteroalkyl, cycloalkyl, aryl, heterocycloalkyl, or heteroaryl; or any two of Rₐ, R_{b} and R_{c} may be taken together with the atom to which they are attached to form a cycloalkyl, heterocycloalkyl; or any three of Rₐ, R_{b} and R_{c} may be taken together with the atom to which they are attached to form aryl or heteroaryl. It should be further understood that in certain embodiments, the alkyl, alkenyl, alkynyl, haloalkyl, heteroalkyl, cycloalkyl, aryl, heterocycloalkyl, or heteroaryl moiety of any one or more of Rₐ, R_{b} and R_{c} may be further substituted as described herein. Rₐ, R_{b} and R_{c} may be the same or different.

In certain embodiments, “amino” also refers to N-oxides of the groups —N*(H)(Rₐ)O—, and —N*(Rₐ)(R_{b})O—, where Rₐ and R_{b} are as described herein, where the N-oxide is bonded to the parent structure through the N atom. N-oxides can be prepared by treatment of the corresponding amino group with, for example, hydrogen peroxide or m-chloroperoxybenzoic acid. The person skilled in the art is familiar with reaction conditions for carrying out the N-oxidation.

“Amide” or “amido” refers to a chemical moiety with formula —C(O) N(Rₐ)(R_{b}) or —NR^{a}C(O)R_{b}, where Rₐ and R_{b} at each occurrence are as described herein. In some embodiments, amido is a C₁₋₄ amido, which includes the amide carbonyl in the total number of carbons in the group. When a —C(O) N(Rₐ)(R_{b}) has Rₐ and R_{b} other than hydrogen, they can be combined with the nitrogen atom to form a 3-, 4-, 5-, 6-, or 7-membered ring.

“Carbonyl” refers to —C(O)Rₐ, where Rₐ is hydrogen, alkyl, alkenyl, alkynyl, haloalkyl, heteroalkyl, cycloalkyl, aryl, heterocycloalkyl, heteroaryl, —N(R′)₂, —S(O)ₜR′, where each R′ is independently hydrogen, alkyl, alkenyl, alkynyl, haloalkyl, heteroalkyl, cycloalkyl, aryl, heterocycloalkyl, or heteroaryl, and t is 1 or 2. In certain embodiments where each R′ are other than hydrogen, the two R′ moieties can be combined with the nitrogen atom to which they are attached to form a 3-, 4-, 5-, 6-, or 7-membered ring. It should be understood that, in one embodiment, carbonyl includes amido (e.g., —C(O) N(Rₐ)(R_{b})).

“Carbamate” refers to any of the following groups: —O—C(═O)—N(Rₐ)(R_{b}) and —N(Rₐ)—C(═O)—OR_{b}, wherein Rₐ and R_{b} at each occurrence are as described herein.

“Cyano” refers to a —CN group.

“Halo”, “halide”, or, alternatively, “halogen” means fluoro, chloro, bromo or iodo. The terms “haloalkyl,” “haloalkenyl,” “haloalkynyl” and “haloalkoxy” include alkyl, alkenyl, alkynyl and alkoxy moieties as described above, wherein one or more hydrogen atoms are replaced by halo. For example, where a residue is substituted with more than one halo groups, it may be referred to by using a prefix corresponding to the number of halo groups attached. For example, dihaloaryl, dihaloalkyl, and trihaloaryl refer to aryl and alkyl substituted with two (“di”) or three (“tri”) halo groups, which may be, but are not necessarily, the same halogen; thus, for example, 3,5-difluorophenyl, 3-chloro-5-fluorophenyl, 4-chloro-3-fluorophenyl, and 3,5-difluoro-4-chlorophenyl is within the scope of dihaloaryl. Other examples of a haloalkyl group include difluoromethyl (—CHF₂), trifluoromethyl (—CF₃), 2,2,2-trifluoroethyl, and 1-fluoromethyl-2-fluoroethyl. Each of the alkyl, alkenyl, alkynyl and alkoxy groups of haloalkyl, haloalkenyl, haloalkynyl and haloalkoxy, respectively, can be optionally substituted as defined herein. “Perhaloalkyl” refers to an alkyl or alkylene group in which all of the hydrogen atoms have been replaced with a halogen (e.g., fluoro, chloro, bromo, or iodo). In some embodiments, all of the hydrogen atoms are each replaced with fluoro. In some embodiments, all of the hydrogen atoms are each replaced with chloro. Examples of perhaloalkyl groups include —CF₃, —CF₂CF₃, —CF₂CF₂CF₃, —CCl₃, —CFCl₂, and —CF₂C₁.

“Thio” refers to —SRₐ, wherein Rₐ is as described herein. “Thiol” refers to the group —RₐSH, wherein Rₐ is as described herein.

“Sulfinyl” refers to —S(O)Rₐ. In some embodiments, sulfinyl is —S(O)N(Rₐ)(R_{b}). “Sulfonyl” refers to the —S(O₂)Rₐ. In some embodiments, sulfonyl is —S(O₂) N(Rₐ)(R_{b}) or —S(O₂)OH. For each of these moieties, it should be understood that Rₐ and R_{b} are as described herein.

“Moiety” refers to a specific segment or functional group of a molecule. Chemical moieties are often recognized chemical entities embedded in or appended to a molecule.

As used herein, the term “unsubstituted” means that for carbon atoms, only hydrogen atoms are present besides those valencies linking the atom to the parent molecular group. One example is propyl (—CH₂—CH₂—CH₃). For nitrogen atoms, valencies not linking the atom to the parent molecular group are either hydrogen or an electron pair. For sulfur atoms, valencies not linking the atom to the parent molecular group are either hydrogen, oxygen or electron pair(s).

As used herein, the term “substituted” or “substitution” means that at least one hydrogen present on a group (e.g., a carbon or nitrogen atom) is replaced with a permissible substituent, e.g., a substituent which upon substitution for the hydrogen results in a stable compound, e.g., a compound which does not spontaneously undergo transformation such as by rearrangement, cyclization, elimination, or other reaction. Unless otherwise indicated, a “substituted” group can have a substituent at one or more substitutable positions of the group, and when more than one position in any given structure is substituted, the substituent is either the same or different at each position. Substituents include one or more group(s) individually and independently selected from alkyl alkenyl, alkoxy, cycloalkyl, aryl, heteroalkyl (e.g., ether), heteroaryl, heterocycloalkyl, cyano, halo, haloalkoxy, haloalkyl, oxo (═O), —ORₐ, —N(Rₐ)₂, —C(O)N(Rₐ)₂, —N(Rₐ)C(O)Rₐ, —C(O)Rₐ, —N(Rₐ)S(O)ₜRₐ (where t is 1 or 2), —SRₐ, and —S(O)ₜN(Rₐ)₂ (where t is 1 or 2), wherein Rₐ is as described herein.

Where substituent groups are specified by their conventional chemical formulae, written from left to right, they equally encompass the chemically identical substituents that would result from writing the structure from right to left, e.g., —CH₂O— is equivalent to —OCH₂—.

Unless defined otherwise, all technical and scientific terms used herein have the same meaning as is commonly understood by one of skill in the art to which this specification pertains.

As used in the specification and claims, the singular form “a”, “an” and “the” includes plural references unless the context clearly dictates otherwise.

Reference to “about” a value or parameter herein includes (and describes) embodiments that are directed to that value or parameter per se. For example, description referring to “about x” includes description of “x” per se. In other instances, the term “about” when used in association with other measurements, or used to modify a value, a unit, a constant, or a range of values, refers to variations of between ±0.1% and ±15% of the stated number. For example, in one variation, “about 1” refers to a range between 0.85 and 1.15.

Reference to “between” two values or parameters herein includes (and describes) embodiments that include those two values or parameters per se. For example, description referring to “between x and y” includes description of “x” and “y” per se.

### Representative Examples of Catalysts

It should be understood that the polymeric catalysts and the solid-supported catalysts can include any of the Bronsted-Lowry acids, cationic groups, counterions, linkers, hydrophobic groups, cross-linking groups, and polymeric backbones or solid supports (as the case may be) described herein, as if each and every combination were listed separately. For example, in one embodiment, the catalyst can include benzenesulfonic acid (i.e., a sulfonic acid with a phenyl linker) connected to a polystyrene backbone or attached to the solid support, and an imidazolium chloride connected directly to the polystyrene backbone or attached directly to the solid support. In another embodiment, the polymeric catalyst can include boronyl-benzyl-pyridinium chloride (i.e., a boronic acid and pyridinium chloride in the same monomer unit with a phenyl linker) connected to a polystyrene backbone or attached to the solid support. In yet another embodiment, the catalyst can include benzenesulfonic acid and imidazolium sulfate each individually connected to a polyvinyl alcohol backbone or individually attached to the solid support.

In some embodiments, the polymeric catalyst is selected from:
poly [styrene-co-4-vinylbenzenesulfonic acid-co-3-methyl-1-(4-vinylbenzyl)-3H-imidazol-1-ium chloride-co-divinylbenzene];
poly [styrene-co-4-vinylbenzenesulfonic acid-co-3-methyl-1-(4-vinylbenzyl)-3H-imidazol-1-ium bisulfate-co-divinylbenzene];
poly [styrene-co-4-vinylbenzenesulfonic acid-co-3-methyl-1-(4-vinylbenzyl)-3H-imidazol-1-ium acetate-co-divinylbenzene];
poly [styrene-co-4-vinylbenzenesulfonic acid-co-3-methyl-1-(4-vinylbenzyl)-3H-imidazol-1-ium nitrate-co-divinylbenzene];
poly [styrene-co-4-vinylbenzenesulfonic acid-co-3-ethyl-1l-(4-vinylbenzyl)-3H-imidazol-1-ium chloride-co-divinylbenzene];
poly [styrene-co-4-vinylbenzenesulfonic acid-co-3-ethyl-1-(4-vinylbenzyl)-3H-imidazol-1-ium bisulfate-co-divinylbenzene];
poly [styrene-co-4-vinylbenzenesulfonic acid-co-3-ethyl-1-(4-vinylbenzyl)-3H-imidazol-1-ium acetate-co-divinylbenzene];
poly [styrene-co-4-vinylbenzenesulfonic acid-co-3-ethyl-1-(4-vinylbenzyl)-³H-imidazol-1-ium nitrate-co-divinylbenzene];
poly [styrene-co-4-vinylbenzenesulfonic acid-co-1-(4-vinylbenzyl)-3H-imidazol-1-ium chloride-co-divinylbenzene];
poly [styrene-co-4-vinylbenzenesulfonic acid-co-1-(4-vinylbenzyl)-3H-imidazol-1-ium iodide-co-divinylbenzene];
poly [styrene-co-4-vinylbenzenesulfonic acid-co-1-(4-vinylbenzyl)-3H-imidazol-1-ium bromide-co-divinylbenzene];
poly [styrene-co-4-vinylbenzenesulfonic acid-co-1-(4-vinylbenzyl)-3H-imidazol-1-ium bisulfate-co-divinylbenzene];
poly [styrene-co-4-vinylbenzenesulfonic acid-co-1-(4-vinylbenzyl)-3H-imidazol-1-ium acetate-co-divinylbenzene];
poly [styrene-co-4-vinylbenzenesulfonic acid-co-3-methyl-1-(4-vinylbenzyl)-3H-benzoimidazol-1-ium chloride-co-divinylbenzene];
poly [styrene-co-4-vinylbenzenesulfonic acid-co-3-methyl-1-(4-vinylbenzyl)-3H-benzoimidazol-1-ium bisulfate-co-divinylbenzene];
poly [styrene-co-4-vinylbenzenesulfonic acid-co-3-methyl-1-(4-vinylbenzyl)-3H-benzoimidazol-1-ium acetate-co-divinylbenzene];
poly [styrene-co-4-vinylbenzenesulfonic acid-co-3-methyl-1-(4-vinylbenzyl)-3H-benzoimidazol-1-ium formate-co-divinylbenzene];
poly [styrene-co-4-vinylbenzenesulfonic acid-co-1-(4-vinylbenzyl)-pyridinium-chloride-co-divinylbenzene];
poly [styrene-co-4-vinylbenzenesulfonic acid-co-1-(4-vinylbenzyl)-pyridinium-bisulfate-co-divinylbenzene];
poly [styrene-co-4-vinylbenzenesulfonic acid-co-1-(4-vinylbenzyl)-pyridinium-acetate-co-divinylbenzene];
poly [styrene-co-4-vinylbenzenesulfonic acid-co-1-(4-vinylbenzyl)-pyridinium-nitrate-co-divinylbenzene];
poly[styrene-co-4-vinylbenzenesulfonic acid-co-1-(4-vinylbenzyl)-pyridinium-chloride-co-3-methyl-1-(4-vinylbenzyl)-3H-imidazol-1-ium bisulfate-co-divinylbenzene];
poly[styrene-co-4-vinylbenzenesulfonic acid-co-1-(4-vinylbenzyl)-pyridinium-bromide-co-3-methyl-1-(4-vinylbenzyl)-3H-imidazol-1-ium bisulfate-co-divinylbenzene];
poly[styrene-co-4-vinylbenzenesulfonic acid-co-1-(4-vinylbenzyl)-pyridinium-iodide-co-3-methyl-1-(4-vinylbenzyl)-3H-imidazol-1-ium bisulfate-co-divinylbenzene];
poly[styrene-co-4-vinylbenzenesulfonic acid-co-1-(4-vinylbenzyl)-pyridinium-bisulfate-co-3-methyl-1-(4-vinylbenzyl)-3H-imidazol-1-ium bisulfate-co-divinylbenzene];
poly[styrene-co-4-vinylbenzenesulfonic acid-co-1-(4-vinylbenzyl)-pyridinium-acetate-co-3-methyl-1-(4-vinylbenzyl)-3H-imidazol-1-ium bisulfate-co-divinylbenzene];
poly[styrene-co-4-vinylbenzenesulfonic acid-co-4-methyl-4-(4-vinylbenzyl)-morpholin-4-ium chloride-co-divinylbenzene];
poly[styrene-co-4-vinylbenzenesulfonic acid-co-4-methyl-4-(4-vinylbenzyl)-morpholin-4-ium bisulfate-co-divinylbenzene];
poly[styrene-co-4-vinylbenzenesulfonic acid-co-4-methyl-4-(4-vinylbenzyl)-morpholin-4-ium acetate-co-divinylbenzene];
poly[styrene-co-4-vinylbenzenesulfonic acid-co-4-methyl-4-(4-vinylbenzyl)-morpholin-4-ium formate-co-divinylbenzene];
poly[styrene-co-4-vinylhenzenesulfonic acid-co-triphenyl-(4-vinylbenzyl)-phosphonium chloride-co-divinylbenzene];
poly[styrene-co-4-vinylbenzenesulfonic acid-co-triphenyl-(4-vinylbenzyl)-phosphonium bisulfate-co-divinylbenzene];
poly[styrene-co-4-vinylbenzenesulfonic acid-co-triphenyl-(4-vinylbenzyl)-phosphonium acetate-co-divinylbenzene];
poly[styrene-co-4-vinylbenzenesulfonic acid-co-1-methyl-1-(4-vinylbenzyl)-piperdin-1-ium chloride-co-divinylbenzene];
poly[styrene-co-4-vinylbenzenesulfonic acid-co-1-methyl-1-(4-vinylbenzyl)-piperdin-1-ium bisulfate-co-divinylbenzene];
poly[styrene-co-4-vinylbenzenesulfonic acid-co-1-methyl-1-(4-vinylbenzyl)-piperdin-1-ium acetate-co-divinylbenzene];
poly[styrene-co-4-vinylbenzenesulfonic acid-co-4-(4-vinylbenzyl)-morpholine-4-oxide-co-divinyl benzene];
poly[styrene-co-4-vinylbenzenesulfonic acid-co-triethyl-(4-vinylbenzyl)-ammonium chloride-co-divinylbenzene];
poly[styrene-co-4-vinylbenzenesulfonic acid-co-triethyl-(4-vinylbenzyl)-ammonium bisulfate-co-divinylbenzene];
poly[styrene-co-4-vinylbenzenesulfonic acid-co-triethyl-(4-vinylbenzyl)-ammonium acetate-co-divinylbenzene];
poly[styrene-co-3-methyl-1-(4-vinylbenzyl)-3H-imidazol-1-ium chloride-co-4-boronyl-1-(4-vinylbenzyl)-pyridinium chloride-co-divinylbenzene];
poly[styrene-co-3-methyl-1-(4-vinylbenzyl)-3H-imidazol-1-ium chloride-co-1-(4-vinylphenyl)methylphosphonic acid-co-divinylbenzene];
poly[styrene-co-3-methyl-1-(4-vinylbenzyl)-3H-imidazol-1-ium bisulfate-co-1-(4-vinylphenyl)methylphosphonic acid-co-divinylbenzene];
poly[styrene-co-3-methyl-1-(4-vinylbenzyl)-3H-imidazol-1-ium acetate-co-1-(4-vinylphenyl)methylphosphonic acid-co-divinylbenzene];
poly[styrene-co-3-methyl-1-(4-vinylbenzyl)-3H-imidazol-1-ium nitrate-co-1-(4-vinylphenyl)methylphosphonic acid-co-divinylbenzene];
poly[styrene-co-4-vinylbenzenesulfonic acid-co-vinylbenzylchloride-co-1-methyl-2-vinyl-pyridinium chloride-co-divinylbenzene];
poly[styrene-co-4-vinylbenzenesulfonic acid-co-vinylbenzylchloride-co-1-methyl-2-vinyl-pyridinium bisulfate-co-divinylbenzene];
poly[styrene-co-4-vinylbenzenesulfonic acid-co-vinylbenzylchloride-co-1-methyl-2-vinyl-pyridinium acetate-co-divinylbenzene];
poly[styrene-co-4-vinylbenzenesulfonic acid-co-4-(4-vinylbenzyl)-morpholine-4-oxide-co-divinyl benzene];
poly [styrene-co-4-vinylphenylphosphonic acid-co-3-methyl-1-(4-vinylbenzyl)-3H-imidazol-1-ium chloride-co-divinylbenzene];
poly [styrene-co-4-vinylphenylphosphonic acid-co-3-methyl-1-(4-vinylbenzyl)-3H-imidazol-1-ium bisulfate-co-divinylbenzene];
poly [styrene-co-4-vinylphenylphosphonic acid-co-3-methyl-1-(4-vinylbenzyl)-3H-imidazol-1-ium acetate-co-divinylbenzene];
poly[styrene-co-3-carboxymethyl-1-(4-vinylbenzyl)-3H-imidazol-1-ium chloride-co-divinylbenzene];
poly[styrene-co-3-carboxymethyl-1-(4-vinylbenzyl)-3H-imidazol-1-ium bisulfate-co-divinylbenzene];
poly[styrene-co-3-carboxymethyl-1-(4-vinylbenzyl)-3H-imidazol-1-ium acetate-co-divinylbenzene];
poly[styrene-co-5-(4-vinylbenzylamino)-isophthalic acid-co-3-methyl-1-(4-vinylbenzyl)-3H-imidazol-1-ium chloride-co-divinylbenzene];
poly[styrene-co-5-(4-vinylbenzylamino)-isophthalic acid-co-3-methyl-1-(4-vinylbenzyl)-3H-imidazol-1-ium bisulfate-co-divinylbenzene];
poly[styrene-co-5-(4-vinylbenzylamino)-isophthalic acid-co-3-methyl-1-(4-vinylbenzyl)-3H-imidazol-1-ium acetate-co-divinylbenzene];
poly[styrene-co-(4-vinylbenzylamino)-acetic acid-co-3-methyl-1-(4-vinylbenzyl)-3H-imidazol-1-ium chloride-co-divinylbenzene];
poly[styrene-co-(4-vinylbenzylamino)-acetic acid-co-3-methyl-1-(4-vinylbenzyl)-3H-imidazol-1-ium bisulfate-co-divinylbenzene];
poly[styrene-co-(4-vinylbenzylamino)-acetic acid-co-3-methyl-1-(4-vinylbenzyl)-3H-imidazol-1-ium acetate-co-divinylbenzene];
poly(styrene-co-4-vinylbenzenesulfonic acid-co-vinylbenzylmethylimidazolium chloride-co-vinylbenzylmethylmorpholinium chloride-co-vinylbenzyltriphenyl phosphonium chloride-co-divinylbenzene);
poly(styrene-co-4-vinylbenzenephosphonic acid-co-vinylbenzylmethylimidazolium chloride-co-vinylbenzylmethylmorpholinium chloride-co-vinylbenzyltriphenyl phosphonium chloride-co-divinylbenzene);
poly(styrene-co-4-vinylbenzenesulfonic acid-co-vinylbenzylmethylimidazolium bisulfate-co-vinylbenzylmethylmorpholinium bisulfate-co-vinylbenzyltriphenyl phosphonium bisulfate-co-divinylbenzene);
poly(styrene-co-4-vinylbenzenephosphonic acid-co-vinylbenzylmethylimidazolium bisulfate-co-vinylbenzylmethylmorpholinium bisulfate-co-vinylbenzyltriphenyl phosphonium bisulfate-co-divinylbenzene);
poly(styrene-co-4-vinylbenzenesulfonic acid-co-vinylbenzylmethylimidazolium acetate-co-vinylbenzylmethylmorpholinium acetate-co-vinylbenzyltriphenyl phosphonium acetate-co-divinylbenzene);
poly(styrene-co-4-vinylbenzenephosphonic acid-co-vinylbenzylmethylimidazolium acetate-co-vinylbenzylmethylmorpholinium acetate-co-vinylbenzyltriphenyl phosphonium acetate-co-divinylbenzene);
poly(styrene-co-4-vinylbenzenesulfonic acid-co-vinylbenzylmethylmorpholinium chloride-co-vinylbenzyltriphenylphosphonium chloride-co-divinylbenzene);
poly(styrene-co-4-vinylbenzenephosphonic acid-co-vinylbenzylmethylmorpholinium chloride-co-vinylbenzyltriphenylphosphonium chloride-co-divinylbenzene);
poly(styrene-co-4-vinylbenzenesulfonic acid-co-vinylbenzylmethylmorpholinium bisulfate-co-vinylbenzyltriphenylphosphonium bisulfate-co-divinylbenzene);
poly(styrene-co-4-vinylbenzenephosphonic acid-co-vinylbenzylmethylmorpholinium bisulfate-co-vinylbenzyltriphenylphosphonium bisulfate-co-divinylbenzene);
poly(styrene-co-4-vinylbenzenesulfonic acid-co-vinylbenzylmethylmorpholinium acetate-co-vinylbenzyltriphenylphosphonium bisulfate-co-divinylbenzene);
poly(styrene-co-4-vinylbenzenephosphonic acid-co-vinylbenzylmethylmorpholinium acetate-co-vinylbenzyltriphenylphosphonium bisulfate-co-divinylbenzene) poly(styrene-co-4-vinylbenzenesulfonic acid-co-vinylmethylimidazolium chloride-co-divinylbenzene);
poly(styrene-co-4-vinylbenzenesulfonic acid-co-vinylmethylimidazolium bisulfate-co-divinylbenzene);
poly(styrene-co-4-vinylbenzenesulfonic acid-co-vinylmethylimidazolium acetate-co-divinylbenzene);
poly(styrene-co-4-vinylbenzenesulfonic acid-co-vinylmethylimidazolium nitrate-co-divinylbenzene);
poly(styrene-co-4-vinylbenzenephosphonic acid-co-vinylmethylimidazolium chloride-co-divinylbenzene);
poly(styrene-co-4-vinylbenzenephosphonic acid-co-vinylmethylimidazolium bisulfate-co-divinylbenzene);
poly(styrene-co-4-vinylbenzenephosphonic acid-co-vinylmethylimidazolium acetate-co-divinylbenzene);
poly(styrene-co-4-vinylhenzenesulfonic acid-co-vinylbenzyltriphenylphosphonium chloride-co-divinylbenzene);
poly(styrene-co-4-vinylbenzenesulfonic acid-co-vinylbenzyltriphenylphosphonium bisulfate-co-divinylbenzene);
poly(styrene-co-4-vinylbenzenesulfonic acid-co-vinylbenzyltriphenylphosphonium acetate-co-divinylbenzene);
poly(styrene-co-4-vinylbenzenephosphonic acid-co-vinylbenzyltriphenylphosphonium chloride-co-divinylbenzene);
poly(styrene-co-4-vinylbenzenephosphonic acid-co-vinylbenzyltriphenylphosphonium bisulfate-co-divinylbenzene);
poly(styrene-co-4-vinylbenzenephosphonic acid-co-vinylbenzyltriphenylphosphonium acetate-co-divinylbenzene);
poly(styrene-co-4-vinylbenzenesulfonic acid-co-vinylbenzylmethylimidazolium chloride-co-divinylbenzene);
poly(styrene-co-4-vinylbenzenesulfonic acid-co-vinylbenzylmethylimidazolium bisulfate-co-divinylbenzene);
poly(styrene-co-4-vinylbenzenesulfonic acid-co-vinylbenzylmethylimidazolium acetate-co-divinylbenzene);
poly(styrene-co-4-vinylbenzenephosphonic acid-co-vinylbenzylmethylimidazolium chloride-co-divinylbenzene);
poly(styrene-co-4-vinylbenzenephosphonic acid-co-vinylbenzylmethylimidazolium bisulfate-co-divinylbenzene);
poly(styrene-co-4-vinylbenzenephosphonic acid-co-vinylbenzylmethylimidazolium acetate-co-divinylbenzene);
poly(styrene-co-4-vinylbenzenesulfonic acid-co-vinylbenzyltriphenylphosphonium chloride-co-divinylbenzene);
poly(styrene-co-4-vinylbenzenesulfonic acid-co-vinylbenzyltriphenylphosphonium bisulfate-co-divinylbenzene);
poly(styrene-co-4-vinylbenzenesulfonic acid-co-vinylbenzyltriphenylphosphonium acetate-co-divinylbenzene);
poly(styrene-co-4-vinylbenzenephosphonic acid-co-vinylbenzyltriphenylphosphonium chloride-co-divinylbenzene);
poly(styrene-co-4-vinylbenzenephosphonic acid-co-vinylbenzyltriphenylphosphonium bisulfate-co-divinylbenzene);
poly(styrene-co-4-vinylbenzenephosphonic acid-co-vinylbenzyltriphenylphosphonium acetate-co-divinylbenzene);
poly(butyl-vinylimidazolium chloride-co-butylimidazolium bisulfate-co-4-vinylbenzenesulfonic acid);
poly(butyl-vinylimidazolium bisulfate-co-butylimidazolium bisulfate-co-4-vinylbenzenesulfonic acid);
poly(benzyl alcohol-co-4-vinylbenzylalcohol sulfonic acid-co-vinylbenzyltriphenylphosphonium chloride-co-divinylbenzyl alcohol); and
poly(benzyl alcohol-co-4-vinylbenzylalcohol sulfonic acid-co-vinylbenzyltriphenylphosphonium bisulfate-co-divinylbenzyl alcohol).

In some embodiments, the solid-supported catalyst is selected from:
amorphous carbon-supported pyrrolium chloride sulfonic acid;
amorphous carbon-supported imidazolium chloride sulfonic acid;
amorphous carbon-supported pyrazolium chloride sulfonic acid;
amorphous carbon-supported oxazolium chloride sulfonic acid;
amorphous carbon-supported thiazolium chloride sulfonic acid;
amorphous carbon-supported pyridinium chloride sulfonic acid;
amorphous carbon-supported pyrimidinium chloride sulfonic acid;
amorphous carbon-supported pyrazinium chloride sulfonic acid;
amorphous carbon-supported pyridazinium chloride sulfonic acid;
amorphous carbon-supported thiazinium chloride sulfonic acid;
amorphous carbon-supported morpholinium chloride sulfonic acid;
amorphous carbon-supported piperidinium chloride sulfonic acid;
amorphous carbon-supported piperizinium chloride sulfonic acid;
amorphous carbon-supported pyrollizinium chloride sulfonic acid;
amorphous carbon-supported triphenyl phosphonium chloride sulfonic acid;
amorphous carbon-supported trimethyl phosphonium chloride sulfonic acid;
amorphous carbon-supported triethyl phosphonium chloride sulfonic acid;
amorphous carbon-supported tripropyl phosphonium chloride sulfonic acid;
amorphous carbon-supported tributyl phosphonium chloride sulfonic acid;
amorphous carbon-supported trifluoro phosphonium chloride sulfonic acid;
amorphous carbon-supported pyrrolium bromide sulfonic acid;
amorphous carbon-supported imidazolium bromide sulfonic acid;
amorphous carbon-supported pyrazolium bromide sulfonic acid;
amorphous carbon-supported oxazolium bromide sulfonic acid;
amorphous carbon-supported thiazolium bromide sulfonic acid;
amorphous carbon-supported pyridinium bromide sulfonic acid;
amorphous carbon-supported pyrimidinium bromide sulfonic acid;
amorphous carbon-supported pyrazinium bromide sulfonic acid;
amorphous carbon-supported pyridazinium bromide sulfonic acid;
amorphous carbon-supported thiazinium bromide sulfonic acid;
amorphous carbon-supported morpholinium bromide sulfonic acid;
amorphous carbon-supported piperidinium bromide sulfonic acid;
amorphous carbon-supported piperizinium bromide sulfonic acid;
amorphous carbon-supported pyrollizinium bromide sulfonic acid;
amorphous carbon-supported triphenyl phosphonium bromide sulfonic acid;
amorphous carbon-supported trimethyl phosphonium bromide sulfonic acid;
amorphous carbon-supported triethyl phosphonium bromide sulfonic acid;
amorphous carbon-supported tripropyl phosphonium bromide sulfonic acid;
amorphous carbon-supported tributyl phosphonium bromide sulfonic acid;
amorphous carbon-supported trifluoro phosphonium bromide sulfonic acid;
amorphous carbon-supported pyrrolium bisulfate sulfonic acid;
amorphous carbon-supported imidazolium bisulfate sulfonic acid;
amorphous carbon-supported pyrazolium bisulfate sulfonic acid;
amorphous carbon-supported oxazolium bisulfate sulfonic acid;
amorphous carbon-supported thiazolium bisulfate sulfonic acid;
amorphous carbon-supported pyridinium bisulfate sulfonic acid;
amorphous carbon-supported pyrimidinium bisulfate sulfonic acid;
amorphous carbon-supported pyrazinium bisulfate sulfonic acid;
amorphous carbon-supported pyridazinium bisulfate sulfonic acid;
amorphous carbon-supported thiazinium bisulfate sulfonic acid;
amorphous carbon-supported morpholinium bisulfate sulfonic acid;
amorphous carbon-supported piperidinium bisulfate sulfonic acid;
amorphous carbon-supported piperizinium bisulfate sulfonic acid;
amorphous carbon-supported pyrollizinium bisulfate sulfonic acid;
amorphous carbon-supported triphenyl phosphonium bisulfate sulfonic acid;
amorphous carbon-supported trimethyl phosphonium bisulfate sulfonic acid;
amorphous carbon-supported triethyl phosphonium bisulfate sulfonic acid;
amorphous carbon-supported tripropyl phosphonium bisulfate sulfonic acid;
amorphous carbon-supported tributyl phosphonium bisulfate sulfonic acid;
amorphous carbon-supported trifluoro phosphonium bisulfate sulfonic acid;
amorphous carbon-supported pyrrolium formate sulfonic acid;
amorphous carbon-supported imidazolium formate sulfonic acid;
amorphous carbon-supported pyrazolium formate sulfonic acid;
amorphous carbon-supported oxazolium formate sulfonic acid;
amorphous carbon-supported thiazolium formate sulfonic acid;
amorphous carbon-supported pyridinium formate sulfonic acid;
amorphous carbon-supported pyrimidinium formate sulfonic acid;
amorphous carbon-supported pyrazinium formate sulfonic acid;
amorphous carbon-supported pyridazinium formate sulfonic acid;
amorphous carbon-supported thiazinium formate sulfonic acid;
amorphous carbon supported morpholinium formate sulfonic acid;
amorphous carbon-supported piperidinium formate sulfonic acid;
amorphous carbon-supported piperizinium formate sulfonic acid;
amorphous carbon-supported pyrollizinium formate sulfonic acid;
amorphous carbon-supported triphenyl phosphonium formate sulfonic acid;
amorphous carbon-supported trimethyl phosphonium formate sulfonic acid;
amorphous carbon-supported triethyl phosphonium formate sulfonic acid;
amorphous carbon-supported tripropyl phosphonium formate sulfonic acid;
amorphous carbon-supported tributyl phosphonium formate sulfonic acid;
amorphous carbon-supported trifluoro phosphonium formate sulfonic acid;
amorphous carbon-supported pyrrolium acetate sulfonic acid;
amorphous carbon-supported imidazolium acetate sulfonic acid;
amorphous carbon-supported pyrazolium acetate sulfonic acid;
amorphous carbon-supported oxazolium acetate sulfonic acid;
amorphous carbon-supported thiazolium acetate sulfonic acid;
amorphous carbon-supported pyridinium acetate sulfonic acid;
amorphous carbon-supported pyrimidinium acetate sulfonic acid;
amorphous carbon-supported pyrazinium acetate sulfonic acid;
amorphous carbon-supported pyridazinium acetate sulfonic acid;
amorphous carbon-supported thiazinium acetate sulfonic acid;
amorphous carbon-supported morpholinium acetate sulfonic acid;
amorphous carbon-supported piperidinium acetate sulfonic acid;
amorphous carbon-supported piperizinium acetate sulfonic acid;
amorphous carbon-supported pyrollizinium acetate sulfonic acid;
amorphous carbon-supported triphenyl phosphonium acetate sulfonic acid;
amorphous carbon-supported trimethyl phosphonium acetate sulfonic acid;
amorphous carbon-supported triethyl phosphonium acetate sulfonic acid;
amorphous carbon-supported tripropyl phosphonium acetate sulfonic acid;
amorphous carbon-supported tributyl phosphonium acetate sulfonic acid;
amorphous carbon-supported trifluoro phosphonium acetate sulfonic acid;
amorphous carbon-supported pyrrolium chloride phosphonic acid;
amorphous carbon-supported imidazolium chloride phosphonic acid;
amorphous carbon-supported pyrazolium chloride phosphonic acid;
amorphous carbon-supported oxazolium chloride phosphonic acid;
amorphous carbon-supported thiazolium chloride phosphonic acid;
amorphous carbon-supported pyridinium chloride phosphonic acid;
amorphous carbon-supported pyrimidinium chloride phosphonic acid;
amorphous carbon-supported pyrazinium chloride phosphonic acid;
amorphous carbon-supported pyridazinium chloride phosphonic acid;
amorphous carbon-supported thiazinium chloride phosphonic acid;
amorphous carbon-supported morpholinium chloride phosphonic acid;
amorphous carbon-supported piperidinium chloride phosphonic acid;
amorphous carbon-supported piperizinium chloride phosphonic acid;
amorphous carbon-supported pyrollizinium chloride phosphonic acid;
amorphous carbon-supported triphenyl phosphonium chloride phosphonic acid;
amorphous carbon-supported trimethyl phosphonium chloride phosphonic acid;
amorphous carbon-supported triethyl phosphonium chloride phosphonic acid;
amorphous carbon-supported tripropyl phosphonium chloride phosphonic acid;
amorphous carbon-supported tributyl phosphonium chloride phosphonic acid;
amorphous carbon-supported trifluoro phosphonium chloride phosphonic acid;
amorphous carbon-supported pyrrolium bromide phosphonic acid;
amorphous carbon-supported imidazolium bromide phosphonic acid;
amorphous carbon-supported pyrazolium bromide phosphonic acid;
amorphous carbon-supported oxazolium bromide phosphonic acid;
amorphous carbon-supported thiazolium bromide phosphonic acid;
amorphous carbon-supported pyridinium bromide phosphonic acid;
amorphous carbon-supported pyrimidinium bromide phosphonic acid;
amorphous carbon-supported pyrazinium bromide phosphonic acid;
amorphous carbon-supported pyridazinium bromide phosphonic acid;
amorphous carbon-supported thiazinium bromide phosphonic acid;
amorphous carbon-supported morpholinium bromide phosphonic acid;
amorphous carbon-supported piperidinium bromide phosphonic acid;
amorphous carbon-supported piperizinium bromide phosphonic acid;
amorphous carbon-supported pyrollizinium bromide phosphonic acid;
amorphous carbon-supported triphenyl phosphonium bromide phosphonic acid;
amorphous carbon-supported trimethyl phosphonium bromide phosphonic acid;
amorphous carbon-supported triethyl phosphonium bromide phosphonic acid;
amorphous carbon-supported tripropyl phosphonium bromide phosphonic acid;
amorphous carbon-supported tributyl phosphonium bromide phosphonic acid;
amorphous carbon-supported trifluoro phosphonium bromide phosphonic acid;
amorphous carbon-supported pyrrolium bisulfate phosphonic acid;
amorphous carbon-supported imidazolium bisulfate phosphonic acid;
amorphous carbon-supported pyrazolium bisulfate phosphonic acid;
amorphous carbon-supported oxazolium bisulfate phosphonic acid;
amorphous carbon-supported thiazolium bisulfate phosphonic acid;
amorphous carbon-supported pyridinium bisulfate phosphonic acid;
amorphous carbon-supported pyrimidinium bisulfate phosphonic acid;
amorphous carbon-supported pyrazinium bisulfate phosphonic acid;
amorphous carbon-supported pyridazinium bisulfate phosphonic acid;
amorphous carbon-supported thiazinium bisulfate phosphonic acid;
amorphous carbon-supported morpholinium bisulfate phosphonic acid;
amorphous carbon-supported piperidinium bisulfate phosphonic acid;
amorphous carbon-supported piperizinium bisulfate phosphonic acid;
amorphous carbon-supported pyrollizinium bisulfate phosphonic acid;
amorphous carbon-supported triphenyl phosphonium bisulfate phosphonic acid;
amorphous carbon-supported trimethyl phosphonium bisulfate phosphonic acid;
amorphous carbon-supported triethyl phosphonium bisulfate phosphonic acid;
amorphous carbon-supported tripropyl phosphonium bisulfate phosphonic acid;
amorphous carbon-supported tributyl phosphonium bisulfate phosphonic acid;
amorphous carbon-supported trifluoro phosphonium bisulfate phosphonic acid;
amorphous carbon-supported pyrrolium formate phosphonic acid;
amorphous carbon-supported imidazolium formate phosphonic acid;
amorphous carbon-supported pyrazolium formate phosphonic acid;
amorphous carbon-supported oxazolium formate phosphonic acid;
amorphous carbon-supported thiazolium formate phosphonic acid;
amorphous carbon-supported pyridinium formate phosphonic acid;
amorphous carbon-supported pyrimidinium formate phosphonic acid;
amorphous carbon-supported pyrazinium formate phosphonic acid;
amorphous carbon-supported pyridazinium formate phosphonic acid;
amorphous carbon-supported thiazinium formate phosphonic acid;
amorphous carbon-supported morpholinium formate phosphonic acid;
amorphous carbon-supported piperidinium formate phosphonic acid;
amorphous carbon-supported piperizinium formate phosphonic acid;
amorphous carbon-supported pyrollizinium formate phosphonic acid;
amorphous carbon-supported triphenyl phosphonium formate phosphonic acid;
amorphous carbon-supported trimethyl phosphonium formate phosphonic acid;
amorphous carbon-supported triethyl phosphonium formate phosphonic acid;
amorphous carbon-supported tripropyl phosphonium formate phosphonic acid;
amorphous carbon-supported tributyl phosphonium formate phosphonic acid;
amorphous carbon-supported trifluoro phosphonium formate phosphonic acid;
amorphous carbon-supported pyrrolium acetate phosphonic acid;
amorphous carbon-supported imidazolium acetate phosphonic acid;
amorphous carbon-supported pyrazolium acetate phosphonic acid;
amorphous carbon-supported oxazolium acetate phosphonic acid;
amorphous carbon-supported thiazolium acetate phosphonic acid;
amorphous carbon-supported pyridinium acetate phosphonic acid;
amorphous carbon-supported pyrimidinium acetate phosphonic acid;
amorphous carbon-supported pyrazinium acetate phosphonic acid;
amorphous carbon-supported pyridazinium acetate phosphonic acid;
amorphous carbon-supported thiazinium acetate phosphonic acid;
amorphous carbon-supported morpholinium acetate phosphonic acid;
amorphous carbon-supported piperidinium acetate phosphonic acid;
amorphous carbon-supported piperizinium acetate phosphonic acid;
amorphous carbon-supported pyrollizinium acetate phosphonic acid;
amorphous carbon-supported triphenyl phosphonium acetate phosphonic acid;
amorphous carbon-supported trimethyl phosphonium acetate phosphonic acid;
amorphous carbon-supported triethyl phosphonium acetate phosphonic acid;
amorphous carbon-supported tripropyl phosphonium acetate phosphonic acid;
amorphous carbon-supported tributyl phosphonium acetate phosphonic acid;
amorphous carbon-supported trifluoro phosphonium acetate phosphonic acid;
amorphous carbon-supported ethanoyl-triphosphonium sulfonic acid;
amorphous carbon-supported ethanoyl-methylmorpholinium sulfonic acid; and
amorphous carbon-supported ethanoyl-imidazolium sulfonic acid.

In other embodiments, the solid-supported catalyst is selected from:
activated carbon-supported pyrrolium chloride sulfonic acid;
activated carbon-supported imidazolium chloride sulfonic acid;
activated carbon-supported pyrazolium chloride sulfonic acid;
activated carbon-supported oxazolium chloride sulfonic acid;
activated carbon-supported thiazolium chloride sulfonic acid;
activated carbon-supported pyridinium chloride sulfonic acid;
activated carbon-supported pyrimidinium chloride sulfonic acid;
activated carbon-supported pyrazinium chloride sulfonic acid;
activated carbon-supported pyridazinium chloride sulfonic acid;
activated carbon-supported thiazinium chloride sulfonic acid;
activated carbon-supported morpholinium chloride sulfonic acid;
activated carbon-supported piperidinium chloride sulfonic acid;
activated carbon-supported piperizinium chloride sulfonic acid;
activated carbon-supported pyrollizinium chloride sulfonic acid;
activated carbon-supported triphenyl phosphonium chloride sulfonic acid;
activated carbon-supported trimethyl phosphonium chloride sulfonic acid;
activated carbon-supported triethyl phosphonium chloride sulfonic acid;
activated carbon-supported tripropyl phosphonium chloride sulfonic acid;
activated carbon-supported tributyl phosphonium chloride sulfonic acid;
activated carbon-supported trifluoro phosphonium chloride sulfonic acid;
activated carbon-supported pyrrolium bromide sulfonic acid;
activated carbon-supported imidazolium bromide sulfonic acid;
activated carbon-supported pyrazolium bromide sulfonic acid;
activated carbon-supported oxazolium bromide sulfonic acid;
activated carbon-supported thiazolium bromide sulfonic acid;
activated carbon-supported pyridinium bromide sulfonic acid;
activated carbon-supported pyrimidinium bromide sulfonic acid;
activated carbon-supported pyrazinium bromide sulfonic acid;
activated carbon-supported pyridazinium bromide sulfonic acid;
activated carbon-supported thiazinium bromide sulfonic acid;
activated carbon-supported morpholinium bromide sulfonic acid;
activated carbon-supported piperidinium bromide sulfonic acid;
activated carbon-supported piperizinium bromide sulfonic acid;
activated carbon-supported pyrollizinium bromide sulfonic acid;
activated carbon-supported triphenyl phosphonium bromide sulfonic acid;
activated carbon-supported trimethyl phosphonium bromide sulfonic acid;
activated carbon-supported triethyl phosphonium bromide sulfonic acid;
activated carbon-supported tripropyl phosphonium bromide sulfonic acid;
activated carbon-supported tributyl phosphonium bromide sulfonic acid;
activated carbon-supported trifluoro phosphonium bromide sulfonic acid;
activated carbon-supported pyrrolium bisulfate sulfonic acid;
activated carbon-supported imidazolium bisulfate sulfonic acid;
activated carbon-supported pyrazolium bisulfate sulfonic acid;
activated carbon-supported oxazolium bisulfate sulfonic acid;
activated carbon-supported thiazolium bisulfate sulfonic acid;
activated carbon-supported pyridinium bisulfate sulfonic acid;
activated carbon-supported pyrimidinium bisulfate sulfonic acid;
activated carbon-supported pyrazinium bisulfate sulfonic acid;
activated carbon-supported pyridazinium bisulfate sulfonic acid;
activated carbon-supported thiazinium bisulfate sulfonic acid;
activated carbon-supported morpholinium bisulfate sulfonic acid;
activated carbon-supported piperidinium bisulfate sulfonic acid;
activated carbon-supported piperizinium bisulfate sulfonic acid;
activated carbon-supported pyrollizinium bisulfate sulfonic acid;
activated carbon-supported triphenyl phosphonium bisulfate sulfonic acid;
activated carbon-supported trimethyl phosphonium bisulfate sulfonic acid;
activated carbon-supported triethyl phosphonium bisulfate sulfonic acid;
activated carbon-supported tripropyl phosphonium bisulfate sulfonic acid;
activated carbon-supported tributyl phosphonium bisulfate sulfonic acid;
activated carbon-supported trifluoro phosphonium bisulfate sulfonic acid;
activated carbon-supported pyrrolium formate sulfonic acid;
activated carbon-supported imidazolium formate sulfonic acid;
activated carbon-supported pyrazolium formate sulfonic acid;
activated carbon-supported oxazolium formate sulfonic acid;
activated carbon-supported thiazolium formate sulfonic acid;
activated carbon-supported pyridinium formate sulfonic acid;
activated carbon-supported pyrimidinium formate sulfonic acid;
activated carbon-supported pyrazinium formate sulfonic acid;
activated carbon-supported pyridazinium formate sulfonic acid;
activated carbon-supported thiazinium formate sulfonic acid;
activated carbon supported morpholinium formate sulfonic acid;
activated carbon-supported piperidinium formate sulfonic acid;
activated carbon-supported piperizinium formate sulfonic acid;
activated carbon-supported pyrollizinium formate sulfonic acid;
activated carbon-supported triphenyl phosphonium formate sulfonic acid;
activated carbon-supported trimethyl phosphonium formate sulfonic acid;
activated carbon-supported triethyl phosphonium formate sulfonic acid;
activated carbon-supported tripropyl phosphonium formate sulfonic acid;
activated carbon-supported tributyl phosphonium formate sulfonic acid;
activated carbon-supported trifluoro phosphonium formate sulfonic acid;
activated carbon-supported pyrrolium acetate sulfonic acid;
activated carbon-supported imidazolium acetate sulfonic acid;
activated carbon-supported pyrazolium acetate sulfonic acid;
activated carbon-supported oxazolium acetate sulfonic acid;
activated carbon-supported thiazolium acetate sulfonic acid;
activated carbon-supported pyridinium acetate sulfonic acid;
activated carbon-supported pyrimidinium acetate sulfonic acid;
activated carbon-supported pyrazinium acetate sulfonic acid;
activated carbon-supported pyridazinium acetate sulfonic acid;
activated carbon-supported thiazinium acetate sulfonic acid;
activated carbon-supported morpholinium acetate sulfonic acid;
activated carbon-supported piperidinium acetate sulfonic acid;
activated carbon-supported piperizinium acetate sulfonic acid;
activated carbon-supported pyrollizinium acetate sulfonic acid;
activated carbon-supported triphenyl phosphonium acetate sulfonic acid;
activated carbon-supported trimethyl phosphonium acetate sulfonic acid;
activated carbon-supported triethyl phosphonium acetate sulfonic acid;
activated carbon-supported tripropyl phosphonium acetate sulfonic acid;
activated carbon-supported tributyl phosphonium acetate sulfonic acid;
activated carbon-supported trifluoro phosphonium acetate sulfonic acid;
activated carbon-supported pyrrolium chloride phosphonic acid;
activated carbon-supported imidazolium chloride phosphonic acid;
activated carbon-supported pyrazolium chloride phosphonic acid;
activated carbon-supported oxazolium chloride phosphonic acid;
activated carbon-supported thiazolium chloride phosphonic acid;
activated carbon-supported pyridinium chloride phosphonic acid;
activated carbon-supported pyrimidinium chloride phosphonic acid;
activated carbon-supported pyrazinium chloride phosphonic acid;
activated carbon-supported pyridazinium chloride phosphonic acid;
activated carbon-supported thiazinium chloride phosphonic acid;
activated carbon-supported morpholinium chloride phosphonic acid;
activated carbon-supported piperidinium chloride phosphonic acid;
activated carbon-supported piperizinium chloride phosphonic acid;
activated carbon-supported pyrollizinium chloride phosphonic acid;
activated carbon-supported triphenyl phosphonium chloride phosphonic acid;
activated carbon-supported trimethyl phosphonium chloride phosphonic acid;
activated carbon-supported triethyl phosphonium chloride phosphonic acid;
activated carbon-supported tripropyl phosphonium chloride phosphonic acid;
activated carbon-supported tributyl phosphonium chloride phosphonic acid;
activated carbon-supported trifluoro phosphonium chloride phosphonic acid;
activated carbon-supported pyrrolium bromide phosphonic acid;
activated carbon-supported imidazolium bromide phosphonic acid;
activated carbon-supported pyrazolium bromide phosphonic acid;
activated carbon-supported oxazolium bromide phosphonic acid;
activated carbon-supported thiazolium bromide phosphonic acid;
activated carbon-supported pyridinium bromide phosphonic acid;
activated carbon-supported pyrimidinium bromide phosphonic acid;
activated carbon-supported pyrazinium bromide phosphonic acid;
activated carbon-supported pyridazinium bromide phosphonic acid;
activated carbon-supported thiazinium bromide phosphonic acid;
activated carbon-supported morpholinium bromide phosphonic acid;
activated carbon-supported piperidinium bromide phosphonic acid;
activated carbon-supported piperizinium bromide phosphonic acid;
activated carbon-supported pyrollizinium bromide phosphonic acid;
activated carbon-supported triphenyl phosphonium bromide phosphonic acid;
activated carbon-supported trimethyl phosphonium bromide phosphonic acid;
activated carbon-supported triethyl phosphonium bromide phosphonic acid;
activated carbon-supported tripropyl phosphonium bromide phosphonic acid;
activated carbon-supported tributyl phosphonium bromide phosphonic acid;
activated carbon-supported trifluoro phosphonium bromide phosphonic acid;
activated carbon-supported pyrrolium bisulfate phosphonic acid;
activated carbon-supported imidazolium bisulfate phosphonic acid;
activated carbon-supported pyrazolium bisulfate phosphonic acid;
activated carbon-supported oxazolium bisulfate phosphonic acid;
activated carbon-supported thiazolium bisulfate phosphonic acid;
activated carbon-supported pyridinium bisulfate phosphonic acid;
activated carbon-supported pyrimidinium bisulfate phosphonic acid;
activated carbon-supported pyrazinium bisulfate phosphonic acid;
activated carbon-supported pyridazinium bisulfate phosphonic acid;
activated carbon-supported thiazinium bisulfate phosphonic acid;
activated carbon-supported morpholinium bisulfate phosphonic acid;
activated carbon-supported piperidinium bisulfate phosphonic acid;
activated carbon-supported piperizinium bisulfate phosphonic acid;
activated carbon-supported pyrollizinium bisulfate phosphonic acid;
activated carbon-supported triphenyl phosphonium bisulfate phosphonic acid;
activated carbon-supported trimethyl phosphonium bisulfate phosphonic acid;
activated carbon-supported triethyl phosphonium bisulfate phosphonic acid;
activated carbon-supported tripropyl phosphonium bisulfate phosphonic acid;
activated carbon-supported tributyl phosphonium bisulfate phosphonic acid;
activated carbon-supported trifluoro phosphonium bisulfate phosphonic acid;
activated carbon-supported pyrrolium formate phosphonic acid;
activated carbon-supported imidazolium formate phosphonic acid;
activated carbon-supported pyrazolium formate phosphonic acid;
activated carbon-supported oxazolium formate phosphonic acid;
activated carbon-supported thiazolium formate phosphonic acid;
activated carbon-supported pyridinium formate phosphonic acid;
activated carbon-supported pyrimidinium formate phosphonic acid;
activated carbon-supported pyrazinium formate phosphonic acid;
activated carbon-supported pyridazinium formate phosphonic acid;
activated carbon-supported thiazinium formate phosphonic acid;
activated carbon-supported morpholinium formate phosphonic acid;
activated carbon-supported piperidinium formate phosphonic acid;
activated carbon-supported piperizinium formate phosphonic acid;
activated carbon-supported pyrollizinium formate phosphonic acid;
activated carbon-supported triphenyl phosphonium formate phosphonic acid;
activated carbon-supported trimethyl phosphonium formate phosphonic acid;
activated carbon-supported triethyl phosphonium formate phosphonic acid;
activated carbon-supported tripropyl phosphonium formate phosphonic acid;
activated carbon-supported tributyl phosphonium formate phosphonic acid;
activated carbon-supported trifluoro phosphonium formate phosphonic acid;
activated carbon-supported pyrrolium acetate phosphonic acid;
activated carbon-supported imidazolium acetate phosphonic acid;
activated carbon-supported pyrazolium acetate phosphonic acid;
activated carbon-supported oxazolium acetate phosphonic acid;
activated carbon-supported thiazolium acetate phosphonic acid;
activated carbon-supported pyridinium acetate phosphonic acid;
activated carbon-supported pyrimidinium acetate phosphonic acid;
activated carbon-supported pyrazinium acetate phosphonic acid;
activated carbon-supported pyridazinium acetate phosphonic acid;
activated carbon-supported thiazinium acetate phosphonic acid;
activated carbon-supported morpholinium acetate phosphonic acid;
activated carbon-supported piperidinium acetate phosphonic acid;
activated carbon-supported piperizinium acetate phosphonic acid;
activated carbon-supported pyrollizinium acetate phosphonic acid;
activated carbon-supported triphenyl phosphonium acetate phosphonic acid;
activated carbon-supported trimethyl phosphonium acetate phosphonic acid;
activated carbon-supported triethyl phosphonium acetate phosphonic acid;
activated carbon-supported tripropyl phosphonium acetate phosphonic acid;
activated carbon-supported tributyl phosphonium acetate phosphonic acid;
activated carbon-supported trifluoro phosphonium acetate phosphonic acid;
activated carbon-supported ethanoyl-triphosphonium sulfonic acid;
activated carbon-supported ethanoyul-methylmorpholinium sulfonic acid; and
activated carbon-supported ethanoyl-imidazolium sulfonic acid.

Methods to prepare the polymeric and solid-supported catalysts described herein can be found in WO 2014/031956, which is hereby incorporated herein specifically with respect to paragraphs [0345]-[0380] and [0382]-[0472].

### Reaction Conditions for Catalytic Oligosaccharide Formation

In some embodiments, the feed sugar and catalyst (e.g., polymeric catalyst or solid-supported catalyst) are allowed to react for at least 1 hour, at least 2 hours, at least 3 hours, at least 4 hours, at least 6 hours, at least 8 hours, at least 16 hours, at least 24 hours, at least 36 hours, or at least 48 hours; or between 1-24 hours, between 2-12 hours, between 3-6 hours, between 1-96 hours, between 12-72 hours, or between 12-48 hours.

In some embodiments, the degree of polymerization of the one or more oligosaccharides produced according to the methods described herein can be regulated by the reaction time. For example, in some embodiments, the degree of polymerization of the one or more oligosaccharides is increased by increasing the reaction time, while in other embodiments, the degree of polymerization of the one or more oligosaccharides is decreased by decreasing the reaction time.

### Reaction Temperature

In some embodiments, the reaction temperature is maintained in the range of about 25° C. to about 150° C. In certain embodiments, the temperature is from about 30° C. to about 125° C., about 60° C. to about 120° C., about 80° C. to about 115° C., about 90° C. to about 110° C., about 95° C. to about 105° C., or about 100° C. to 110° C.

### Amount of Feed Sugar

The amount of the feed sugar used in the methods described herein relative to the amount solvent used may affect the rate of reaction and yield. The amount of the feed sugar used may be characterized by the dry solids content. In certain embodiments, dry solids content refers to the total solids of a slurry as a percentage on a dry weight basis. In some embodiments, the dry solids content of the feed sugar is between about 5 wt % to about 95 wt %, between about 10 wt % to about 80 wt %, between about 15 to about 75 wt %, or between about 15 to about 50 wt %.

### Amount of Catalyst

The amount of the catalyst used in the methods described herein may depend on several factors including, for example, the selection of the type of feed sugar, the concentration of the feed sugar, and the reaction conditions (e.g., temperature, time, and pH). In some embodiments, the weight ratio of the catalyst to the feed sugar is about 0.01 g/g to about 50 g/g, about 0.01 g/g to about 5 g/g, about 0.05 g/g to about 1.0 g/g, about 0.05 g/g to about 0.5 g/g, about 0.05 g/g to about 0.2 g/g, or about 0.1 g/g to about 0.2 g/g.

### Solvent

In certain embodiments, the methods of using the catalyst are carried out in an aqueous environment. One suitable aqueous solvent is water, which may be obtained from various sources. Generally, water sources with lower concentrations of ionic species (e.g., salts of sodium, phosphorous, ammonium, or magnesium) are preferable, as such ionic species may reduce effectiveness of the catalyst. In some embodiments where the aqueous solvent is water, the water has a resistivity of at least 0.1 megaohm-centimeters, of at least 1 megaohm-centimeters, of at least 2 megaohm-centimeters, of at least 5 megaohm-centimeters, or of at least 10 megaohm-centimeters.

### Water Content

Moreover, as the dehydration reaction of the methods progresses, water is produced with each coupling of the one or more sugars. In certain embodiments, the methods described herein may further include monitoring the amount of water present in the reaction mixture and/or the ratio of water to sugar or catalyst over a period of time. In some embodiments, the method further includes removing at least a portion of water produced in the reaction mixture (e.g., by removing at least about any of 10%, 20%, 30%, 40%, 50%, 60%, 70%, 80%, 90%, 95%, 97%, 99%, or 100%, such as by vacuum distillation). It should be understood, however, that the amount of water to sugar may be adjusted based on the reaction conditions and specific catalyst used.

Any method known in the art may be used to remove water in the reaction mixture, including, for example, by vacuum filtration, vacuum distillation, heating, and/or evaporation. In some embodiments, the method comprises including water in the reaction mixture.

In some aspects, provided herein are methods of producing an oligosaccharide composition, by: combining a feed sugar and a catalyst having acidic and ionic moieties to form a reaction mixture, wherein water is produced in the reaction mixture; and removing at least a portion of the water produced in the reaction mixture. In certain variations, at least a portion of water is removed to maintain a water content in the reaction mixture of less than 99%, less than 90%, less than 80%, less than 70%, less than 60%, less than 50%, less than 40%, less than 30%, less than 20%, less than 10%, less than 5%, or less than 1% by weight.

In some embodiments, the degree of polymerization of the one or more oligosaccharides produced according to the methods described herein can be regulated by adjusting or controlling the concentration of water present in the reaction mixture. For example, in some embodiments, the degree of polymerization of the one or more oligosaccharides is increased by decreasing the water concentration, while in other embodiments, the degree of polymerization of the one or more oligosaccharides is decreased by increasing the water concentration. In some embodiments, the water content of the reaction is adjusted during the reaction to regulate the degree of polymerization of the one or more oligosaccharides produced.

### Batch Versus Continuous Processing

Generally, the catalyst and the feed sugar are introduced into an interior chamber of a reactor, either concurrently or sequentially. The reaction can be performed in a batch process or a continuous process. For example, in one embodiment, method is performed in a batch process, where the contents of the reactor are continuously mixed or blended, and all or a substantial amount of the products of the reaction are removed. In one variation, the method is performed in a batch process, where the contents of the reactor are initially intermingled or mixed but no further physical mixing is performed. In another variation, the method is performed in a batch process, wherein once further mixing of the contents, or periodic mixing of the contents of the reactor, is performed (e.g., at one or more times per hour), all or a substantial amount of the products of the reaction are removed after a certain period of time.

In some embodiments, the method is repeated in a sequential batch process, wherein at least a portion of the catalyst is separated from at least a portion of the oligosaccharide composition produced (e.g., as described in more detail infra) and is recycled by further contacting additional feed sugar.

For example, in one aspect, provided is a method for producing an oligosaccharide composition, by:
a) combining feed sugar with a catalyst to form a reaction mixture;
   wherein the catalyst comprises acidic monomers and ionic monomers connected to form a polymeric backbone, or
   wherein the catalyst comprises a solid support, acidic moieties attached to the solid support, and ionic moieties attached to the solid support; and
b) producing an oligosaccharide composition from at least a portion of the reaction mixture;
c) separating the oligosaccharide composition from the catalyst;
d) combining additional feed sugar with the separated catalyst to form additional reaction mixture; and
e) producing additional oligosaccharide composition from at least a portion of the additional reaction mixture.

In some of embodiments wherein the method is performed in a batch process, the catalyst is recycled (e.g., steps (c)-(e) above are repeated) at least 1, at least 2, at least 3, at least 4, at least 5, at least 6, at least 7, at least 8, at least 9 or at least 10 times. In some of these embodiments, the catalyst retains at least 80% activity (e.g., at least 90%, 95%, 96%, 97%, 98%, or 99% activity) after being recycled 1, 2, 3, 4, 5, 6, 7, 8, 9 or 10 times, when compared to the catalytic activity under identical conditions prior to being recycled.

In other embodiments, the method is performed in a continuous process, where the contents flow through the reactor with an average continuous flow rate but with no explicit mixing. After introduction of the catalyst and the feed sugar into the reactor, the contents of the reactor are continuously or periodically mixed or blended, and after a period of time, less than all of the products of the reaction are removed. In one variation, method is performed in a continuous process, where the mixture containing the catalyst and one or more sugars is not actively mixed. Additionally, mixing of catalyst and feed sugar may occur as a result of the redistribution of catalysts settling by gravity, or the non-active mixing that occurs as the material flows through a continuous reactor. In some embodiments of the methods, the steps of combining the feed sugar with a catalyst and isolating the oligosaccharide composition produced are performed concurrently.

### Reactors

The reactors used for the methods described herein may be open or closed reactors suitable for use in containing the chemical reactions described herein. Suitable reactors may include, for example, a fed-batch stirred reactor, a batch stirred reactor, a continuous flow stirred reactor with ultrafiltration, a continuous plug-flow column reactor, an attrition reactor, or a reactor with intensive stirring induced by an electromagnetic field. See e.g., Fernanda de Castilhos Corazza, Flavio Faria de Moraes, Gisella Maria Zanin and Ivo Neitzel, Optimal control in fed-batch reactor for the cellobiose hydrolysis, Acta Scientiarum. Technology, 25: 33-38 (2003); Gusakov, A. V., and Sinitsyn, A. P., Kinetics of the enzymatic hydrolysis of cellulose: 1. A mathematical model for a batch reactor process, Enz. Microb. Technol., 7: 346-352 (1985); Ryu, S. K., and Lee, J. M., Bioconversion of waste cellulose by using an attrition bioreactor, Biotechnol. Bioeng. 25: 53-65(1983); Gusakov, A. V., Sinitsyn, A. P., Davydkin, I. Y., Davydkin, V. Y., Protas, O. V., Enhancement of enzymatic cellulose hydrolysis using a novel type of bioreactor with intensive stirring induced by electromagnetic field, Appl. Biochem. Biotechnol., 56: 141-153(1996). Other suitable reactor types may include, for example, fluidized bed, upflow blanket, immobilized, and extruder type reactors for hydrolysis and/or fermentation.

In certain embodiments where the method is performed as a continuous process, the reactor may include a continuous mixer, such as a screw mixer. The reactors may be generally fabricated from materials that are capable of withstanding the physical and chemical forces exerted during the processes described herein. In some embodiments, such materials used for the reactor are capable of tolerating high concentrations of strong liquid acids; however, in other embodiments, such materials may not be resistant to strong acids.

It should also be understood that additional feed sugar and/or catalyst may be added to the reactor, either at the same time or one after the other.

### Recyclability of Catalysts

The catalysts containing acidic and ionic groups used in the methods of producing oligosaccharide compositions as described herein may be recycled. Thus, in one aspect, provided herein are methods of producing oligosaccharide compositions using recyclable catalysts.

Any method known in the art may be used to separate the catalyst for reuse, including, for example, centrifugation, filtration (e.g., vacuum filtration), and gravity settling.

The methods described herein may be performed as batch or continuous processes. Recycling in a batch process may involve, for example, recovering the catalyst from the reaction mixture and reusing the recovered catalyst in one or more subsequent reaction cycles. Recycling in a continuous process may involve, for example, introducing additional feed sugar into the reactor, without additional of fresh catalyst.

In some of embodiments wherein at least a portion of the catalyst is recycled, the catalyst is recycled at least 1, at least 2, at least 3, at least 4, at least 5, at least 6, at least 7, at least 8, at least 9 or at least 10 times. In some of these embodiments, the catalyst retains at least 80%, at least 90%, at least 95%, at least 96%, at least 97%, at least 98%, or at least 99% activity after being recycled 1, 2, 3, 4, 5, 6, 7, 8, 9 or 10 times, when compared to the catalytic activity under identical conditions prior to being recycled.

As used herein, the “catalyst activity” refers to the effective first order kinetic rate constant for the molar conversion of reactants, k=−ln(1−X(t))/t. The molar conversion of the reactant A at time t is defined as X_{A}(t)=1−mol(A,t)/mol(A,0), where mol(A,t) refers to the number of moles of species A present in the reaction mixture at time t and mol(A,0) refers to the number of moles of species A present at the start of the reaction, t=0. In practice, the number of moles of the reactant A is often measured at several points in time, t₁, t₂, t₃, . . . , tₙ during a single reaction cycle and used to calculate the conversions X_{A}(t₁), X_{A}(t₂), . . . X_{A}(tₙ) at the corresponding times. The first order rate constant k is then calculated by fitting the data for X_{A}(t).

As used herein, a reaction “cycle” refers to one period of use within a sequence of uses of the catalyst. For example, in a batch process, a reaction cycle corresponds to the discrete steps of charging a reactor system with reactants and catalyst, heating the reaction under suitable conditions to convert the reactants, maintaining the reaction conditions for a specified residence time, separating the reaction products from the catalyst, and recovering the catalyst for re-use. In a continuous process, a cycle refers a single reactor space time during the operation of the continuous process. For example, in a 1,000 liter reactor with a continuous volumetric flow of 200 liters per hour, the continuous reactor space time is two hours, and the first two hour period of continuous operation is the first reaction cycle, the next two hour period of continuous operation is the second reaction cycle, etc.

As used herein, the “loss of activity” or “activity loss” of a catalyst is determined by the average fractional reduction in the catalyst activity between consecutive cycles. For example, if the catalyst activity in reaction cycle 1 is k(1) and the catalyst activity in reaction cycle 2 is k(2), then the loss in catalyst activity between cycle 1 and cycle 2 is calculated as [k(2)−k(1)]/k(1). Over N reaction cycles, the loss of activity is then determined as

$$\frac{1}{( N - 1 )} {\sum }_{i = 2}^{N} \frac{k ( i ) - k ( i - 1 )}{k ( i )} ,$$

measured in units of fractional loss per cycle.

In some variations, the rate constant for the conversion of additional feed sugar is less than 20% lower than the rate constant for the conversion of the reactant feed sugar in the first reaction. In certain variations, the rate constant for conversion of the additional feed sugar is less than 15%, less than 12%, less than 10%, less than 8%, less than 6%, less than 4%, less than 2%, or less than 1% lower than the rate constant for the conversion of the reactant feed sugar in the first reaction. In some variations, the loss of activity is less than 20% per cycle, less than 15% per cycle, less than 10% per cycle, less than 8% per cycle, less than 4% per cycle, less than 2% per cycle, less than 1% per cycle, less than 0.5% per cycle, or less than 0.2% per cycle.

As used herein “catalyst lifetime” refers to the average number of cycles that a catalyst particle can be re-used before it no longer effectively catalyzes the conversion of additional reactant feed sugar. The catalyst lifetime is calculated as the reciprocal of the loss of activity. For example, if the loss of activity is 1% per cycle, then the catalyst lifetime is 100 cycles. In some variations, the catalyst lifetime is at least 1 cycle, at least 2 cycles, at least 10 cycles, at least 50 cycles, at least 100 cycles, at least 200 cycles, at least 500 cycles.

In certain embodiments, a portion of the total mass of the catalyst in a reaction may be removed and replaced with fresh catalyst between reaction cycles. For example, in some variations, 0.1% of the mass of the catalyst may be replaced between reaction cycles, 1% of the mass of the catalyst may be replaced between reaction cycles, 2% of the mass of the catalyst may be replaced between reaction cycles, 5% of the mass of the catalyst may be replaced between reaction cycles, 10% of the mass of the catalyst may be replaced between reaction cycles, or 20% of the mass of the catalyst may be replaced between reaction cycles.

As used herein, the “catalyst make-up rate” refers to the fraction of the catalyst mass that is replaced with fresh catalyst between reaction cycles.

### Additional Processing Steps

With reference again to FIG. 1, process 100 may be modified to have additional processing steps. Additional processing steps may include, for example, polishing steps. Polishing steps may include, for example, separation, dilution, concentration, filtration, demineralization, chromatographic separation, or decolorization, or any combination thereof. For example, in one embodiment process 100 is modified to include a dilution step and a decolorization step. In another embodiment process 100 is modified to include a filtration step and a drying step.

### Decolorization

In some embodiments, the methods described herein further include a decolorization step. The one or more oligosaccharides produced may undergo a decolorization step using any method known in the art, including, for example, treatment with an absorbent, activated carbon, chromatography (e.g., using ion exchange resin), hydrogenation, and/or filtration (e.g., microfiltration).

In certain embodiments, the one or more oligosaccharides produced are contacted with a color-absorbing material at a particular temperature, at a particular concentration, and/or for a particular duration of time. In some embodiments, the mass of the color absorbing species contacted with the one or more oligosaccharides is less than 50% of the mass of the one or more oligosaccharides, less than 35% of the mass of the one or more oligosaccharides, less than 20% of the mass of the one or more oligosaccharides, less than 10% of the mass of the one or more oligosaccharides, less than 5% of the mass of the one or more oligosaccharides, less than 2% of the mass of the one or more oligosaccharides, or less than 1% of the mass of the one or more oligosaccharides.

In some embodiments, the one or more oligosaccharides are contacted with a color absorbing material. In certain embodiments, the one or more oligosaccharides are contacted with a color absorbing material for less than 10 hours, less than 5 hours, less than 1 hour, or less than 30 minutes. In a particular embodiment, the one or more oligosaccharides are contacted with a color absorbing material for 1 hour.

In certain embodiments, the one or more oligosaccharides are contacted with a color absorbing material at a temperature from 20 to 100 degrees Celsius, 30 to 80 degrees Celsius, 40 to 80 degrees Celsius, or 40 to 65 degrees Celsius. In a particular embodiment, the one or more oligosaccharides are contacted with a color absorbing material at a temperature of 50 degrees Celsius.

In certain embodiments, the color absorbing material is activated carbon. In one embodiment, the color absorbing material is powdered activated carbon. In other embodiments, the color absorbing material is an ion exchange resin. In one embodiment, the color absorbing material is a strong base cationic exchange resin in a chloride form. In another embodiment, the color absorbing material is cross-linked polystyrene. In yet another embodiment, the color absorbing material is cross-linked polyacrylate. In certain embodiments, the color absorbing material is Amberlite FPA91, Amberlite FPA98, Dowex 22, Dowex Marathon MSA, or Dowex Optipore SD-2.

### Dentineralization

In some embodiments, the one or more oligosaccharides produced are contacted with a material to remove salts, minerals, and/or other ionic species. In certain embodiments, the one or more oligosaccharides are flowed through an anionic/cationic exchange column pair. In one embodiment, the anionic exchange column contains a weak base exchange resin in a hydroxide form and the cationic exchange column contains a strong acid exchange resin in a protonated form.

### Separation and Concentration

In some embodiments, the methods described herein further include isolating the one or more oligosaccharides produced. In certain variations, isolating the one or more oligosaccharides comprises separating at least a portion of the one or more oligosaccharides from at least a portion of the catalyst, using any method known in the art, including, for example, centrifugation, filtration (e.g., vacuum filtration, membrane filtration), and gravity settling. In some embodiments, isolating the one or more oligosaccharides comprises separating at least a portion of the one or more oligosaccharides from at least a portion of any unreacted sugar, using any method known in the art, including, for example, filtration (e.g., membrane filtration), chromatography (e.g., chromatographic fractionation), differential solubility, and centrifugation (e.g., differential centrifugation).

In some embodiments, the methods described herein further include a concentration step. For example, in some embodiments, the isolated oligosaccharides undergo evaporation (e.g., vacuum evaporation) to produce a concentrated oligosaccharide composition. In other embodiments, the isolated oligosaccharides undergo a spray drying step to produce an oligosaccharide powder. In certain embodiments, the isolated oligosaccharides undergo both an evaporation step and a spray drying step.

### Bond Refactoring

The sugar used in the methods described herein typically have α-1,4 bonds, and when used as reactants in the methods described herein, at least a portion of the α-1,4 bonds are converted into β-1,4 bonds, α-1,3 bonds, β-1,3 bonds, α-1,6 bonds, and β-1,6 bonds.

Thus, in certain aspects, provided is a method of producing an oligosaccharide composition, by:
combining feed sugar with a catalyst to form a reaction mixture,
   wherein the feed sugar has α-1,4 bonds, and
   wherein the catalyst has acidic monomers and ionic monomers connected to form a polymeric backbone, or wherein the catalyst comprises a solid support, acidic moieties attached to the solid support, and ionic moieties attached to the solid support; and
converting at least a portion of the α-1,4 bonds in the feed sugar to one or more non-α-1,4 bonds selected from the group consisting of β-1,4 bonds, α-1,3 bonds, β-1,3 bonds, α-1,6 bonds, and β-1,6 bonds to produce an oligosaccharide composition from at least a portion of the reaction mixture.

It should generally be understood that α-1,4 bonds may also be referred to herein as α(1→4) bonds, and similarly, β-1,4 bonds, α-1,3 bonds, β-1,3 bonds, α-1,6 bonds, and β-1,6 bonds may be referred to as β(1→4), α(1→3), β(1→3), α(1→6), and β(1→6) bonds, respectively.

One of skill in the art would recognize that α-1,4 bonds are typically digestible by a human, whereas β-1,4 bonds, α-1,3 bonds, β-1,3 bonds, α-1,6 bonds, and β-1,6 are typically less digestible or indigestible by humans.

### Enumerated Embodiments

The following enumerated embodiments are representative of some aspects of the invention.
1. A method of producing a polished oligosaccharide composition, comprising:
   combining feed sugar with a catalyst to form a reaction mixture,
      wherein the catalyst comprises acidic monomers and ionic monomers connected to form a polymeric backbone, or
      wherein the catalyst comprises a solid support, acidic moieties attached to the solid support, and ionic moieties attached to the solid support; and
   producing an oligosaccharide composition from at least a portion of the reaction mixture; and
   polishing the oligosaccharide composition to produce a polished oligosaccharide composition.
2. A method of producing a food ingredient, comprising:
   combining feed sugar with a catalyst to form a reaction mixture,
      wherein the catalyst comprises acidic monomers and ionic monomers connected to form a polymeric backbone, or
      wherein the catalyst comprises a solid support, acidic moieties attached to the solid support, and ionic moieties attached to the solid support; and
   producing an oligosaccharide composition from at least a portion of the reaction mixture;
   polishing the oligosaccharide composition to produce a polished oligosaccharide composition; and
   forming a food ingredient from the polished oligosaccharide composition.
3. The method of embodiment 1 or 2, wherein the feed sugar comprises glucose, galactose, fructose, mannose, arabinose, or xylose, or any combinations thereof.
4. The method of embodiment 1 or 3, wherein the oligosaccharide composition comprises a gluco-oligosaccharide, a galacto-oligosaccharide, a fructo-oligosaccharide, a manno-oligosaccharide, an arabino-oligosaccharide, a xylo-oligosaccharide, a gluco-galacto-oligosaccharide, a gluco-fructo-oligosaccharide, a gluco-manno-oligosaccharide, a gluco-arabino-oligosaccharide, a gluco-xylo-oligosaccharide, a galacto-fructo-oligosaccharide, a galacto-manno-oligosaccharide, a galacto-arabino-oligosaccharide, a galacto-xylo-oligosaccharide, a fructo-manno-oligosaccharide, a fructo-arabino-oligosaccharide, a fructo-xylo-oligosaccharide, a manno-arabino-oligosaccharide, a manno-xylo-oligosaccharide, an arabino-xylo-oligosaccharide, or a xylo-gluco-galacto-oligosaccharide, or any combinations thereof.
5. The method of any one of embodiments 1 to 4, further comprising:
   separating at least a portion of the catalyst in the reaction mixture from the oligosaccharide composition produced.
6. The method of embodiment 5, further comprising:
   combining additional feed sugar with the separated catalyst to form an additional reaction mixture; and
   producing an additional oligosaccharide composition from at least a portion of the additional reaction mixture.
7. The method of any one of embodiments 1 to 6, wherein the oligosaccharide composition has a degree of polymerization of at least three.
8. The method of any one of embodiments 2 to 7, wherein the food ingredient is a syrup.
9. The method of any one of embodiments 2 to 7, wherein the forming of the food ingredient from the polished oligosaccharide composition comprises spray drying the polished oligosaccharide composition to form the food ingredient.
10. The method of embodiment 9, wherein the food ingredient is a powder.
11. The method of any one of embodiments 1 to 10, wherein the catalyst comprises acidic monomers and ionic monomers connected to form a polymeric backbone.
12. The method of embodiment 11, wherein each acidic monomer independently comprises at least one Bronsted-Lowry acid.
13. The method of embodiment 12, wherein the at least one Bronsted-Lowry acid at each occurrence in the catalyst is independently selected from the group consisting of sulfonic acid,
phosphonic acid, acetic acid, isophthalic acid, boronic acid, and perfluorinated acid.
14. The method of embodiment 13, wherein the at least one Bronsted-Lowry acid at each occurrence in the catalyst is independently selected from the group consisting of sulfonic acid and phosphonic acid.
15. The method of embodiment 13, wherein the at least one Bronsted-Lowry acid at each occurrence in the catalyst is sulfonic acid.
16. The method of embodiment 13, wherein the at least one Bronsted-Lowry acid at each occurrence in the catalyst is phosphonic acid.
17. The method of embodiment 13, wherein the at least one Bronsted-Lowry acid at each occurrence in the catalyst is acetic acid.
18. The method of embodiment 13, wherein the at least one Bronsted-Lowry acid at each occurrence in the catalyst is isophthalic acid.
19. The method of embodiment 13, wherein the at least one Bronsted-Lowry acid at each occurrence in the catalyst is boronic acid.
20. The method of embodiment 13, wherein the at least one Bronsted-Lowry acid at each occurrence in the catalyst is perfluorinated acid.
21. The method of any one of embodiments 12 to 20, wherein one or more of the acidic monomers are directly connected to the polymeric backbone.
22. The method of any one of embodiments 12 to 20, wherein one or more of the acidic monomers each further comprise a linker connecting the Bronsted-Lowry acid to the polymeric backbone.
23. The method of embodiment 22, wherein the linker at each occurrence is independently selected from the group consisting of unsubstituted or substituted alkylene, unsubstituted or substituted cycloalkylene, unsubstituted or substituted alkenylene, unsubstituted or substituted arylene, unsubstituted or substituted heteroarylene, unsubstituted or substituted alkylene ether, unsubstituted or substituted alkylene ester, and unsubstituted or substituted alkylene carbamate.
24. The method of embodiment 22, wherein the Bronsted-Lowry acid and the linker form a side chain, wherein each side chain is independently selected from the group consisting of:

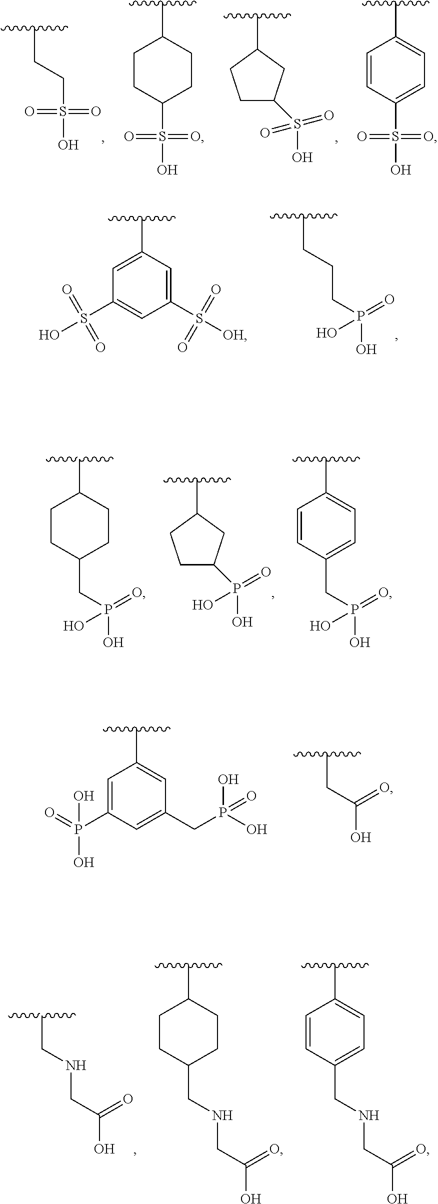

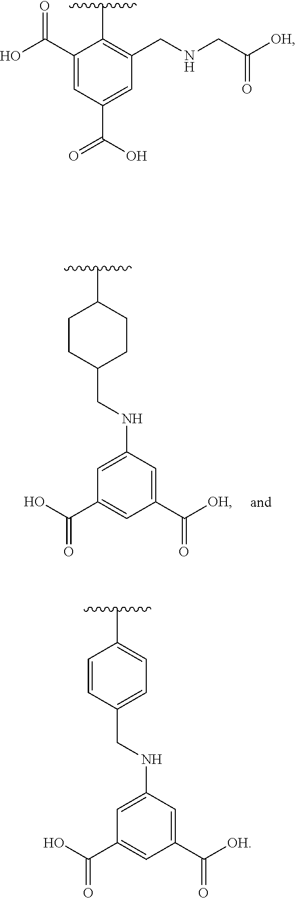 [SMILES: *c1c(CNCC(=O)O)cc(C(=O)O)cc1C(=O)O ; *C1CCC(CNc2cc(C(=O)O)cc(C(=O)O)c2)CC1 ; *c1ccc(CNc2cc(C(=O)O)cc(C(=O)O)c2)cc1]

25. The method of any one of embodiments 11 to 24, wherein each ionic monomer independently comprises at least one nitrogen-containing cationic group, at least one phosphorous-containing cationic group, or a combination thereof.
26. The method of embodiment 25, wherein the nitrogen-containing cationic group at each occurrence is independently selected from the group consisting of pyrrolium, imidazolium, pyrazolium, oxazolium, thiazolium, pyridinium, pyrimidinium, pyrazinium, pyridazinium, thiazinium, morpholinium, piperidinium, piperizinium, and pyrollizinium.
27. The method of embodiment 25, wherein the phosphorous-containing cationic group at each occurrence is independently selected from the group consisting of triphenyl phosphonium, trimethyl phosphonium, triethyl phosphonium, tripropyl phosphonium, tributyl phosphonium, trichloro phosphonium, and trifluoro phosphonium.
28. The method of any one of embodiments 11 to 27, wherein one or more of the ionic monomers are directly connected to the polymeric backbone.
29. The method of any one of embodiments 11 to 27, wherein one or more of the ionic monomers each further comprise a linker connecting the nitrogen-containing cationic group or the phosphorous-containing cationic group to the polymeric backbone.
30. The method of embodiment 29, wherein the linker at each occurrence is independently selected from the group consisting of unsubstituted or substituted alkylene, unsubstituted or substituted cycloalkylene, unsubstituted or substituted alkenylene, unsubstituted or substituted arylene, unsubstituted or substituted heteroarylene, unsubstituted or substituted alkylene ether, unsubstituted or substituted alkylene ester, and unsubstituted or substituted alkylene carbamate.
31. The method of embodiment 29, wherein the nitrogen-containing cationic group and the linker form a side chain, wherein each side chain is independently selected from the group consisting of:

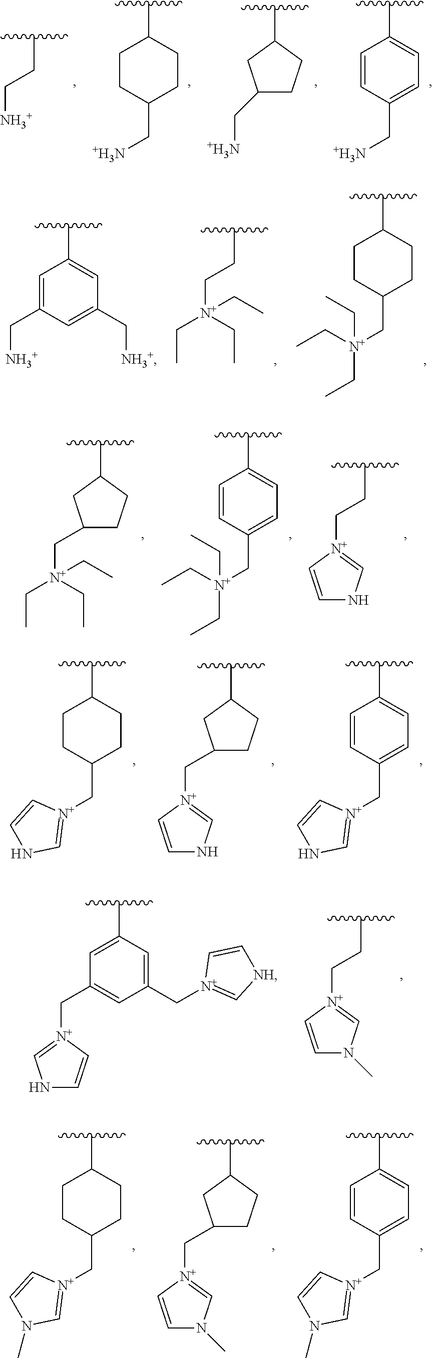

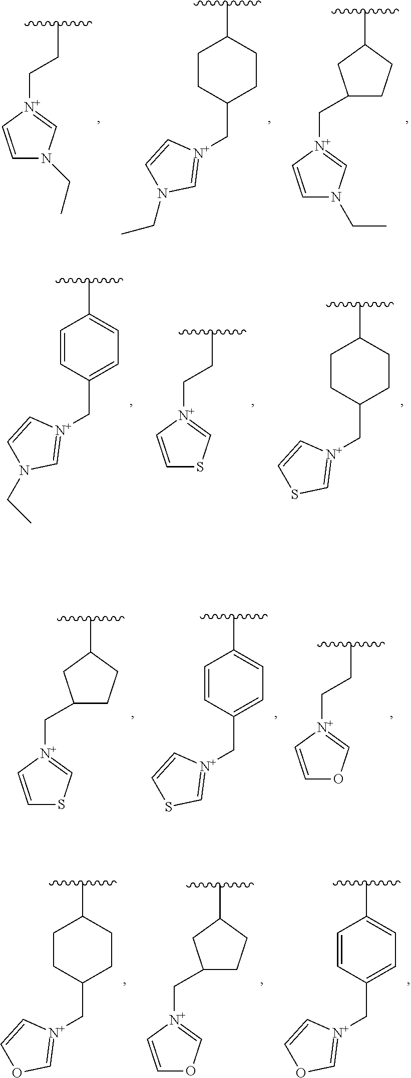

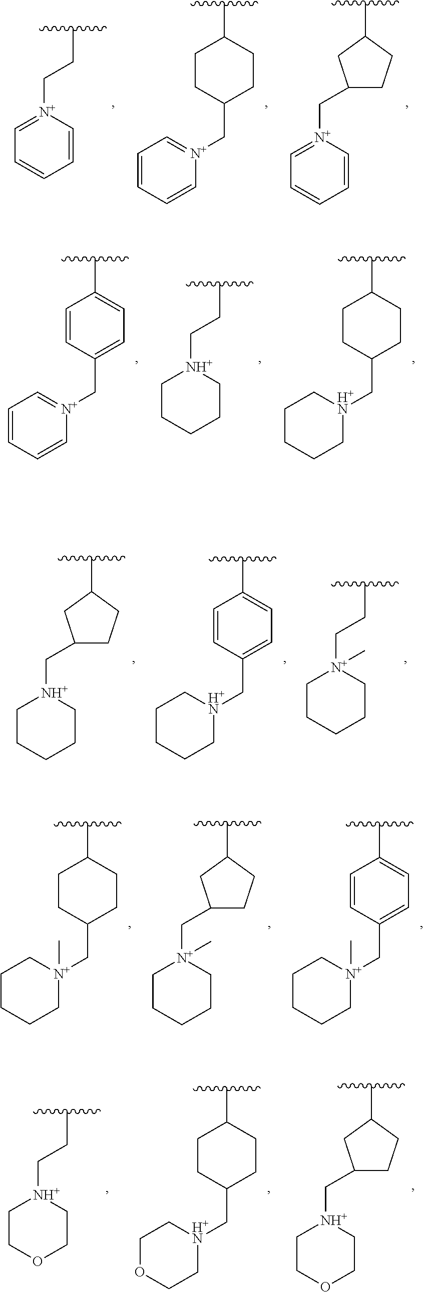

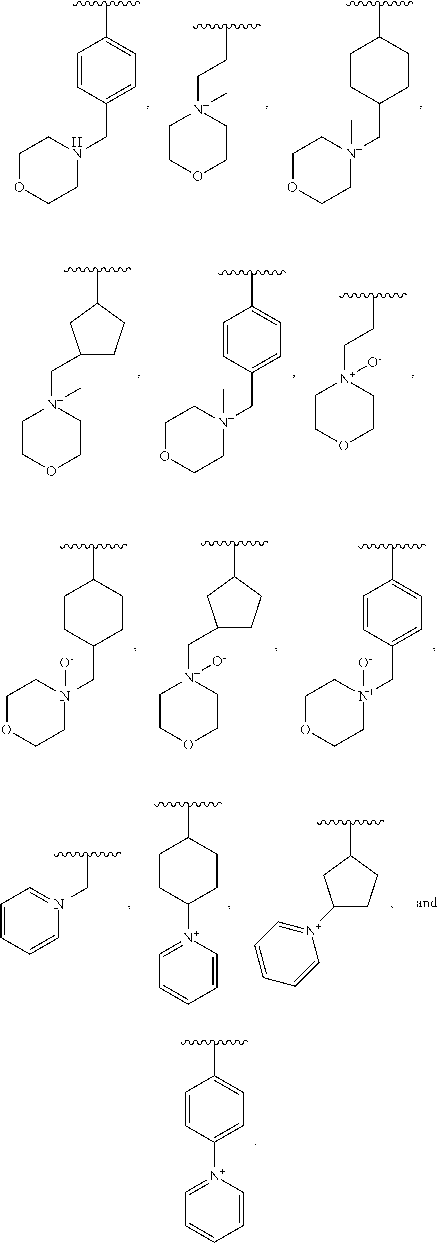

32. The method of embodiment 29, wherein the phosphorous-containing cationic group and the linker form a side chain, wherein each side chain is independently selected from the group consisting of:

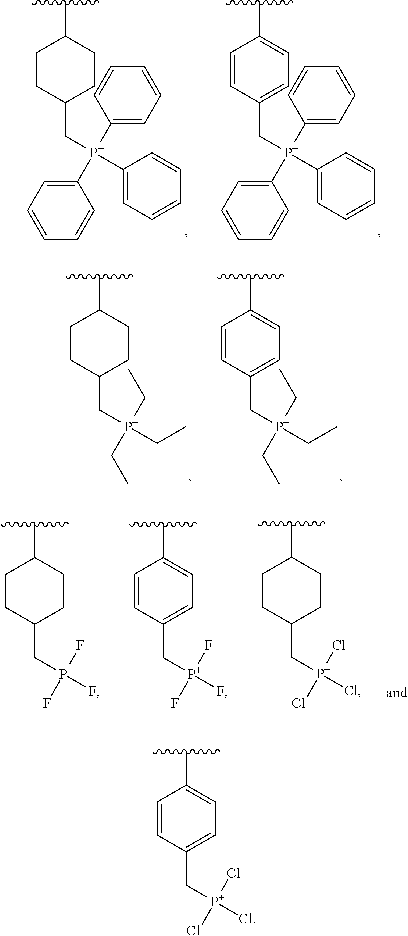

33. The method of any one of embodiments 11 to 32, wherein the polymeric backbone is selected from the group consisting of polyethylene, polypropylene, polyvinyl alcohol, polystyrene, polyurethane, polyvinyl chloride, polyphenol-aldehyde, polytetrafluoroethylene, polybutylene terephthalate, polycaprolactam, poly(acrylonitrile butadiene styrene), polyalkyleneammonium, polyalkylenediammonium, polyalkylenepyrrolium, polyalkyleneimidazolium, polyalkylenepyrazolium, polyalkyleneoxazolium, polyalkylenethiazolium, polyalkylenepyridinium, polyalkylenepyrimidinium, polyalkylenepyrazinium, polyalkylenepyridazinium, polyalkylenethiazinium, polyalkylenemorpholinium, polyalkylenepiperidinium, polyalkylenepiperizinium, polyalkylenepyrollizinium, polyalkylenetriphenylphosphonium, polyalkylenetrimethylphosphonium, polyalkylenetriethylphosphonium, polyalkylenetripropylphosphonium, polyalkylenetributylphosphonium, polyalkylenetrichlorophosphonium, polyalkylenetrifluorophosphonium, and polyalkylenediazolium.
34. The method of any one of embodiments 11 to 33, further comprising hydrophobic monomers connected to the polymeric backbone, wherein each hydrophobic monomer comprises a hydrophobic group.
35. The method of embodiment 34, wherein the hydrophobic group at each occurrence is independently selected from the group consisting of an unsubstituted or substituted alkyl, an unsubstituted or substituted cycloalkyl, an unsubstituted or substituted aryl, or an unsubstituted or substituted heteroaryl.
36. The method of embodiment 34 or 35, wherein the hydrophobic group is directly connected to the polymeric backbone.
37. The method of any one of embodiments 11 to 36, further comprising acidic-ionic monomers connected to the polymeric backbone, wherein each acidic-ionic monomer comprises a Bronsted-Lowry acid and a cationic group.
38. The method of embodiment 37, wherein the cationic group is a nitrogen-containing cationic group or a phosphorous-containing cationic group.
39. The method of embodiment 37 or 38, wherein one or more of the acidic-ionic monomers each further comprise a linker connecting the Bronsted-Lowry acid or the cationic group to the polymeric backbone.
40. The method of embodiment 39, wherein the linker at each occurrence is independently selected from the group consisting of unsubstituted or substituted alkylene, unsubstituted or substituted cycloalkylene, unsubstituted or substituted alkenylene, unsubstituted or substituted arylene, unsubstituted or substituted heteroarylene, unsubstituted or substituted alkylene ether, unsubstituted or substituted alkylene ester, and unsubstituted or substituted alkylene carbamate.
41. The method of embodiment 39, wherein the Bronsted-Lowry acid, the cationic group and the linker form a side chain, wherein each side chain is independently selected from the group consisting of:

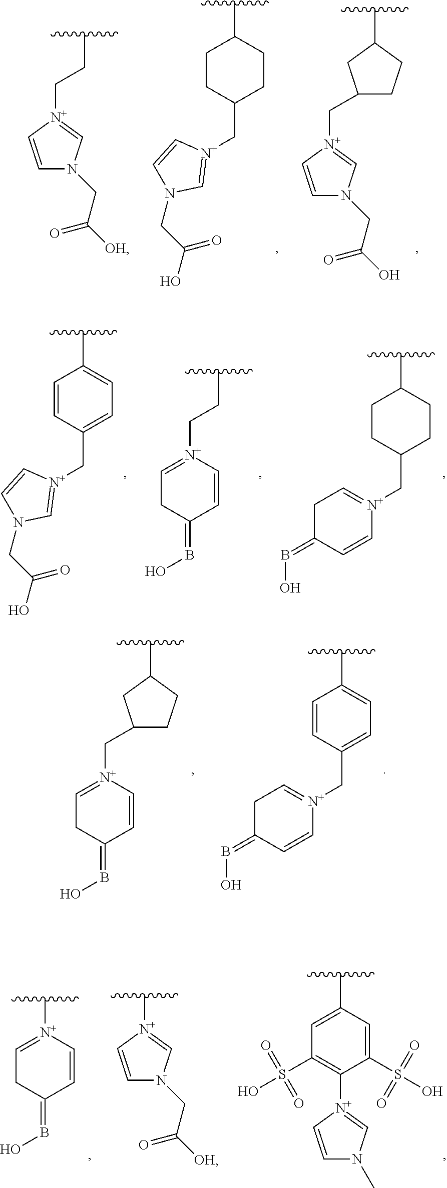

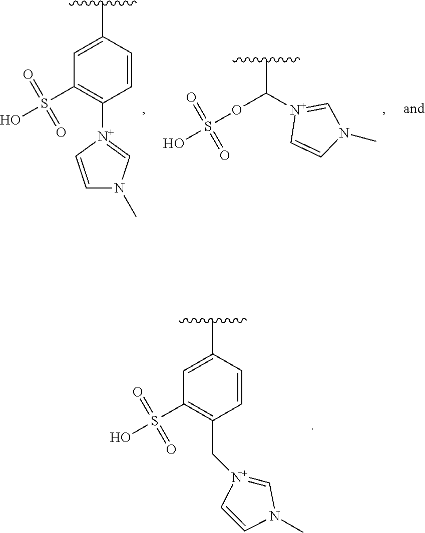 [SMILES: *c1ccc(-[n+]2ccn(C)c2)c(S(=O)(=O)O)c1 ; *C(OS(=O)(=O)O)[n+]1ccn(C)c1 ; *c1ccc(C[n+]2ccn(C)c2)c(S(=O)(=O)O)c1]

42. The method of any one of embodiments 1 to 10, wherein the catalyst comprises a solid support, acidic moieties attached to the solid support, and ionic moieties attached to the solid support.
43. The method of embodiment 42, wherein the solid support comprises a material, wherein the material is selected from the group consisting of carbon, silica, silica gel, alumina, magnesia,
titania, zirconia, clays, magnesium silicate, silicon carbide, zeolites, ceramics, and any combinations thereof.
44. The method of embodiment 43, wherein the material is selected from the group consisting of carbon, magnesia, titania, zirconia, clays, zeolites, ceramics, and any combinations thereof.
45. The method of any one of embodiments 42 to 44, wherein each acidic moiety independently has at least one Bronsted-Lowry acid.
46. The method of embodiment 45, wherein each Bronsted-Lowry acid is independently selected from the group consisting of sulfonic acid, phosphonic acid, acetic acid, isophthalic acid, boronic acid, and perfluorinated acid.
47. The method of embodiment 46, wherein each Bronsted-Lowry acid is independently sulfonic acid or phosphonic acid.
48. The method of embodiment 46, wherein each Bronsted-Lowry acid is sulfonic acid.
49. The method of embodiment 46, wherein each Bronsted-Lowry acid is phosphonic acid.
50. The method of embodiment 46, wherein each Bronsted-Lowry acid is acetic acid.
51. The method of embodiment 46, wherein each Bronsted-Lowry acid is isophthalic acid.
52. The method of embodiment 46, wherein each Bronsted-Lowry acid is boronic acid.
53. The method of embodiment 46, wherein each Bronsted-Lowry acid is perfluorinated acid.
54. The method of any one of embodiments 42 to 53, wherein one or more of the acidic moieties are directly attached to the solid support.
55. The method of any one of embodiments 42 to 53, wherein one or more of the acidic moieties are attached to the solid support by a linker.
56. The method of embodiment 55, wherein the linker at each occurrence is independently selected from the group consisting of unsubstituted or substituted alkylene, unsubstituted or substituted cycloalkylene, unsubstituted or substituted alkenylene, unsubstituted or substituted arylene, unsubstituted or substituted heteroarylene, unsubstituted or substituted alkylene ether, unsubstituted or substituted alkylene ester, and unsubstituted or substituted alkylene carbamate.
57. The method of embodiment 55, wherein each acidic moiety independently has at least one Bronsted-Lowry acid, wherein the Bronsted-Lowry acid and the linker form a side chain, wherein each side chain is independently selected from the group consisting of:

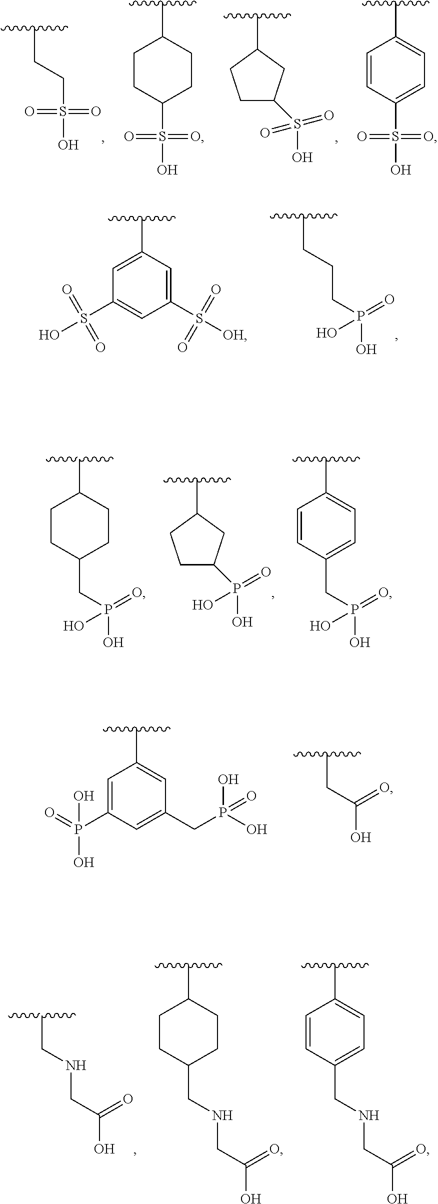

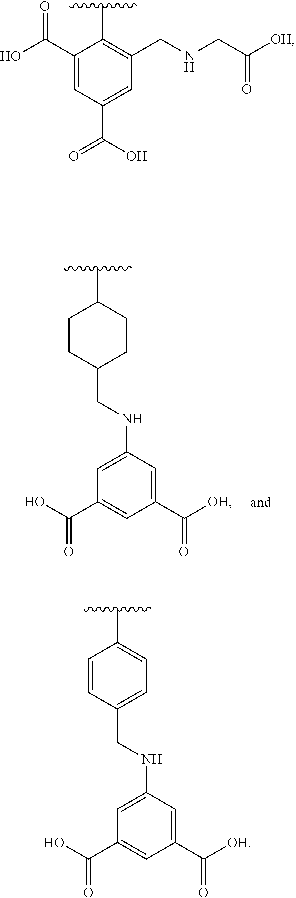 [SMILES: *c1c(CNCC(=O)O)cc(C(=O)O)cc1C(=O)O ; *C1CCC(CNc2cc(C(=O)O)cc(C(=O)O)c2)CC1 ; *c1ccc(CNc2cc(C(=O)O)cc(C(=O)O)c2)cc1]

58. The method of any one of embodiments 42 to 57, wherein each ionic moiety independently has at least one nitrogen-containing cationic group or at least one phosphorous-containing cationic group, or a combination thereof.
59. The method of any one of embodiments 42 to 57, wherein each ionic moiety is selected from the group consisting of pyrrolium, imidazolium, pyrazolium, oxazolium, thiazolium, pyridinium, pyrimidinium, pyrazinium, pyridazinium, thiazinium, morpholinium, piperidinium, piperizinium, pyrollizinium, phosphonium, trimethyl phosphonium, triethyl phosphonium, tripropyl phosphonium, tributyl phosphonium, trichloro phosphonium, triphenyl phosphonium and trifluoro phosphonium.
60. The method of embodiment 58, wherein each ionic moiety independently has at least one nitrogen-containing cationic group, and wherein each nitrogen-containing cationic group is independently selected from the group consisting of pyrrolium, imidazolium, pyrazolium, oxazolium, thiazolium, pyridinium, pyrimidinium, pyrazinium, pyridazinium, thiazinium, morpholinium, piperidinium, piperizinium, and pyrollizinium.
61. The method of embodiment 58, wherein each ionic moiety independently has at least one phosphorous-containing cationic group, and wherein each phosphorous-containing cationic group is independently selected from the group consisting of triphenyl phosphonium, trimethyl phosphonium, triethyl phosphonium, tripropyl phosphonium, tributyl phosphonium, trichloro phosphonium, and trifluoro phosphonium.
62. The method of any one of embodiments 42 to 61, wherein one or more of the ionic moieties are directed attached to the solid support.
63. The method of any one of embodiments 42 to 61, wherein one or more of the ionic moieties are attached to the solid support by a linker.
64. The method of embodiment 63, wherein each linker is independently selected from the group consisting of unsubstituted or substituted alkyl linker, unsubstituted or substituted cycloalkyl linker, unsubstituted or substituted alkenyl linker, unsubstituted or substituted aryl linker, unsubstituted or substituted heteroaryl linker, unsubstituted or substituted alkyl ether linker, unsubstituted or substituted alkyl ester linker, and unsubstituted or substituted alkyl carbamate linker.
65. The method of embodiment 63, wherein each ionic moiety independently has at least one nitrogen-containing cationic group, wherein the nitrogen-containing cationic group and the linker form a side chain, wherein each side chain is independently selected from the group consisting of:

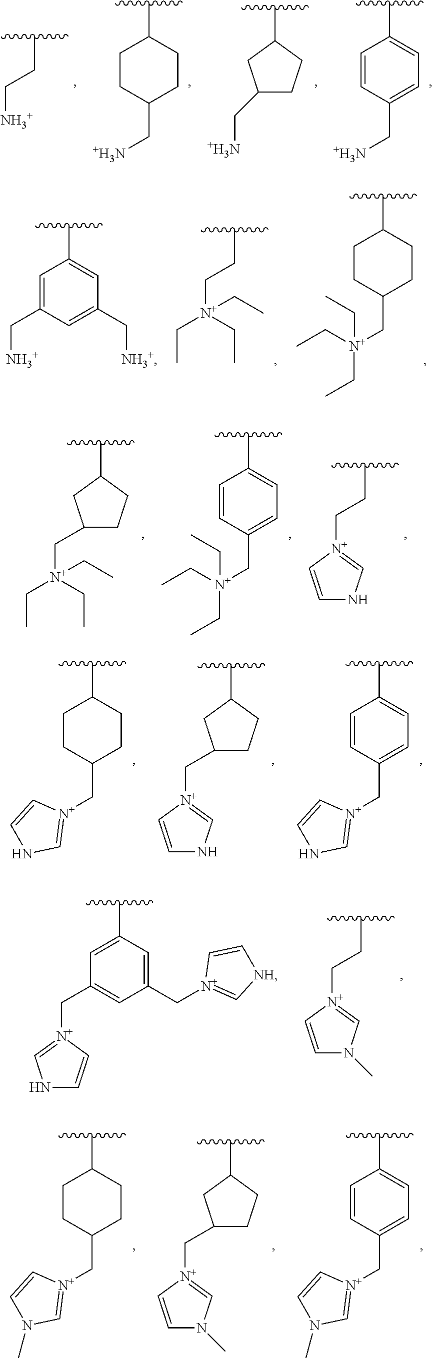

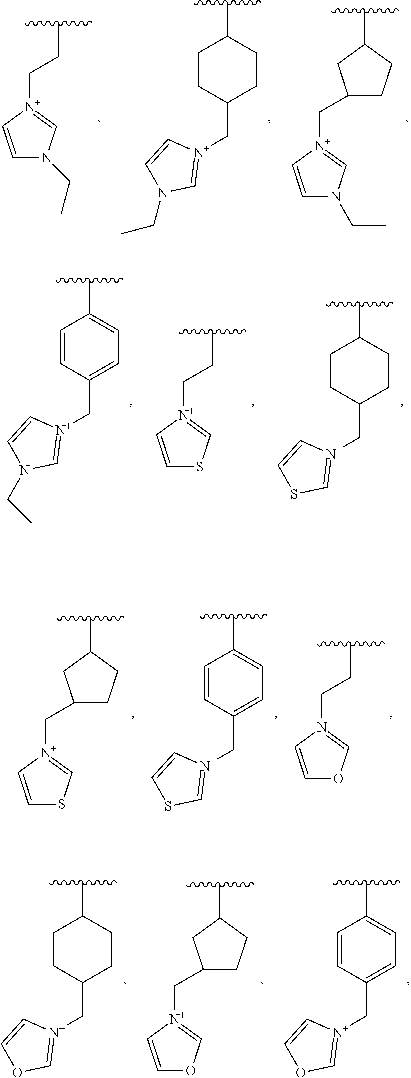

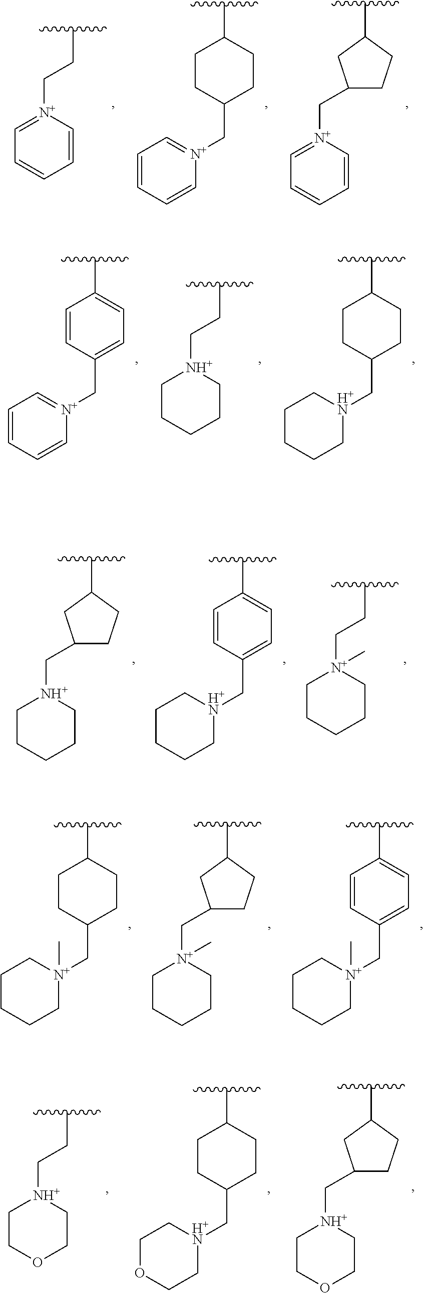

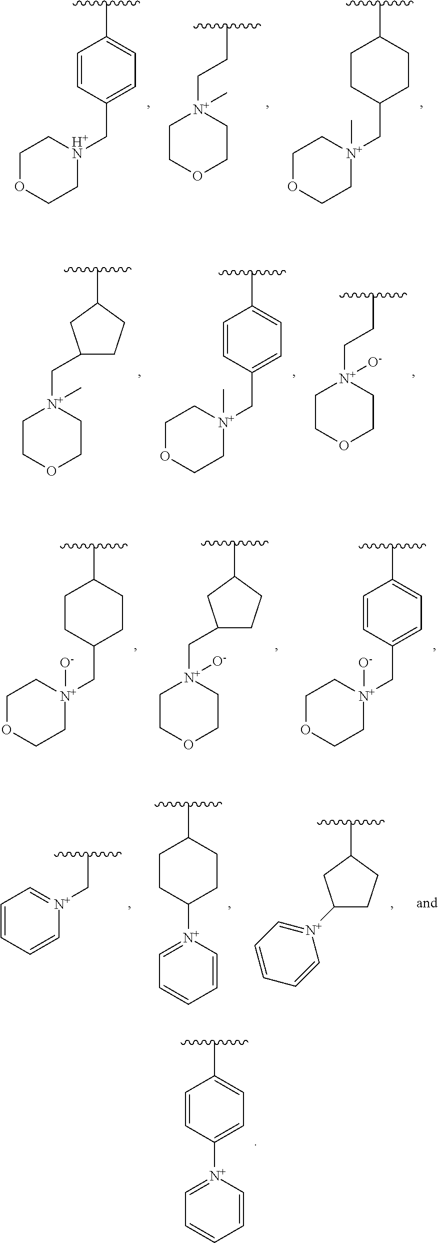

66. The method of embodiment 63, wherein each ionic moiety independently has at least one phosphorous-containing cationic group, wherein the phosphorous-containing cationic group and the linker form a side chain, wherein each side chain is independently selected from the group consisting of:

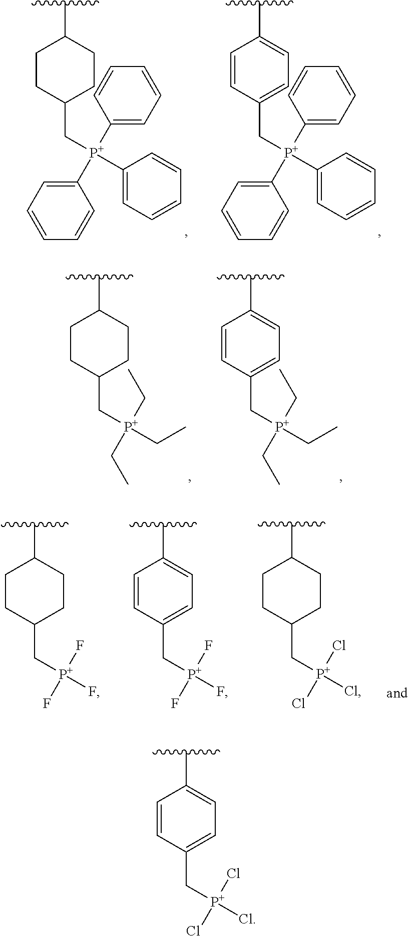

67. The method of any one of embodiments 42 to 66, further comprising hydrophobic moieties attached to the solid support.
68. The method of embodiment 67, wherein each hydrophobic moiety is selected from the group consisting of an unsubstituted or substituted alkyl, an unsubstituted or substituted cycloalkyl, an unsubstituted or substituted aryl, and an unsubstituted or substituted heteroaryl.
69. The method of any one of embodiments 42 to 68, further comprising acidic-ionic moieties attached to the solid support, wherein each acidic-ionic moiety comprises a Bronsted-Lowry acid and a cationic group.
70. The method of embodiment 69, wherein the cationic group is a nitrogen-containing cationic group or a phosphorous-containing cationic group.
71. The method of embodiment 69 or 70, wherein one or more of the acidic-ionic monomers each further comprise a linker connecting the Bronsted-Lowry acid or the cationic group to the polymeric backbone.
72. The method of embodiment 71, wherein the linker at each occurrence is independently selected from the group consisting of unsubstituted or substituted alkylene, unsubstituted or substituted cycloalkylene, unsubstituted or substituted alkenylene, unsubstituted or substituted arylene, unsubstituted or substituted heteroarylene, unsubstituted or substituted alkylene ether, unsubstituted or substituted alkylene ester, and unsubstituted or substituted alkylene carbamate.
73. The method of embodiment 71, wherein the Bronsted-Lowry acid, the cationic group and the linker form a side chain, wherein each side chain is independently selected from the group consisting of:

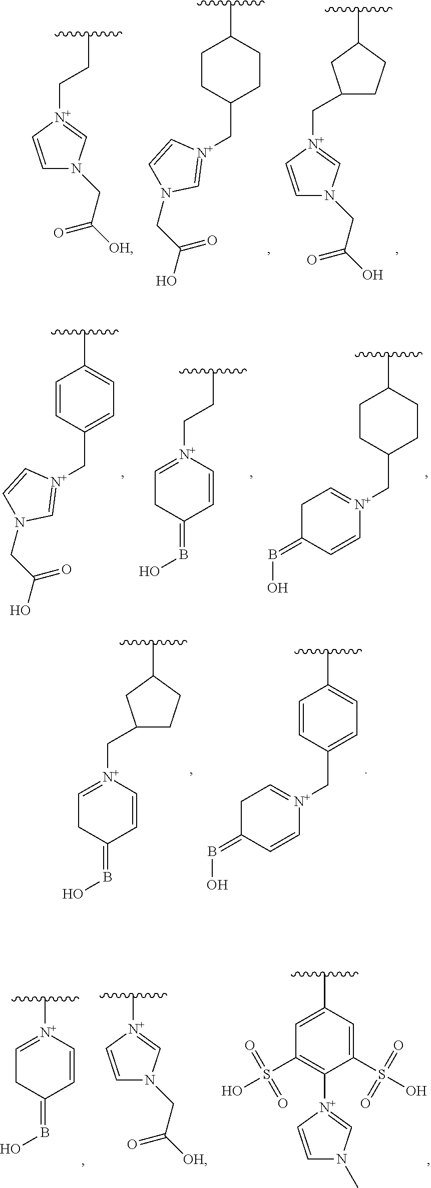

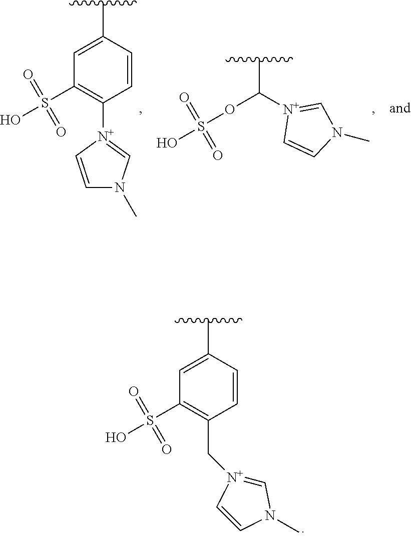 [SMILES: *c1ccc(-[n+]2ccn(C)c2)c(S(=O)(=O)O)c1 ; *C(OS(=O)(=O)O)[n+]1ccn(C)c1 ; *c1ccc(C[n+]2ccn(C)c2)c(S(=O)(=O)O)c1]

74. The method of any one of embodiments 42 to 73, wherein the material is carbon, and wherein the carbon is selected from the group consisting of biochar, amorphous carbon, and activated carbon.
75. The method of any one of embodiments 1 to 10, wherein the catalyst is selected from the group consisting of:
poly [styrene-co-4-vinylbenzenesulfonic acid-co-3-methyl-1-(4-vinylbenzyl)-3H-imidazol-1-ium chloride-co-divinylbenzene];
poly [styrene-co-4-vinylbenzenesulfonic acid-co-3-methyl-1-(4-vinylbenzyl)-3H-imidazol-1-ium bisulfate-co-divinylbenzene];
poly [styrene-co-4-vinylbenzenesulfonic acid-co-3-methyl-1-(4-vinylbenzyl)-3H-imidazol-1-ium acetate-co-divinylbenzene];
poly [styrene-co-4-vinylbenzenesulfonic acid-co-3-methyl-1-(4-vinylbenzyl)-3H-imidazol-1-ium nitrate-co-divinylbenzene];
poly [styrene-co-4-vinylbenzenesulfonic acid-co-3-ethyl-1-(4-vinylbenzyl)-3H-imidazol-1-ium chloride-co-divinylbenzene];
poly [styrene-co-4-vinylbenzenesulfonic acid-co-3-ethyl-1-(4-vinylbenzyl)-3H-imidazol-1-ium bisulfate-co-divinylbenzene];
poly [styrene-co-4-vinylbenzenesulfonic acid-co-3-ethyl-1-(4-vinylbenzyl)-3H-imidazol-1-ium acetate-co-divinylbenzene];
poly [styrene-co-4-vinylbenzenesulfonic acid-co-3-ethyl-1-(4-vinylbenzyl)-3H-imidazol-1-ium nitrate-co-divinylbenzene];
poly [styrene-co-4-vinylbenzenesulfonic acid-co-1-(4-vinylbenzyl)-3H-imidazol-1-ium chloride-co-divinylbenzene];
poly [styrene-co-4-vinylbenzenesulfonic acid-co-1-(4-vinylbenzyl)-3H-imidazol-1-ium iodide-co-divinylbenzene];
poly [styrene-co-4-vinylbenzenesulfonic acid-co-1-(4-vinylbenzyl)-3H-imidazol-1-ium bromide-co-divinylbenzene];
poly [styrene-co-4-vinylbenzenesulfonic acid-co-1-(4-vinylbenzyl)-3H-imidazol-1-ium bisulfate-co-divinylbenzene];
poly [styrene-co-4-vinylbenzenesulfonic acid-co-1-(4-vinylbenzyl)-3H-imidazol-1-ium acetate-co-divinylbenzene];
poly [styrene-co-4-vinylbenzenesulfonic acid-co-3-methyl-1-(4-vinylbenzyl)-3H-benzoimidazol-1-ium chloride-co-divinylbenzene];
poly [styrene-co-4-vinylbenzenesulfonic acid-co-3-methyl-1-(4-vinylbenzyl)-3H-benzoimidazol-1-ium bisulfate-co-divinylbenzene];
poly [styrene-co-4-vinylbenzenesulfonic acid-co-3-methyl-1-(4-vinylbenzyl)-3H-benzoimidazol-1-ium acetate-co-divinylbenzene];
poly [styrene-co-4-vinylbenzenesulfonic acid-co-3-methyl-1-(4-vinylbenzyl)-3H-benzoimidazol-1-ium formate-co-divinylbenzene];
poly [styrene-co-4-vinylbenzenesulfonic acid-co-1-(4-vinylbenzyl)-pyridinium-chloride-co-divinylbenzene];
poly [styrene-co-4-vinylbenzenesulfonic acid-co-1-(4-vinylbenzyl)-pyridinium-bisulfate-co-divinylbenzene];
poly [styrene-co-4-vinylbenzenesulfonic acid-co-1-(4-vinylbenzyl)-pyridinium-acetate-co-divinylbenzene];
poly [styrene-co-4-vinylbenzenesulfonic acid-co-1-(4-vinylbenzyl)-pyridinium-nitrate-co-divinylbenzene];
poly[styrene-co-4-vinylbenzenesulfonic acid-co-1-(4-vinylbenzyl)-pyridinium-chloride-co-3-methyl-1-(4-vinylbenzyl)-3H-imidazol-1-ium bisulfate-co-divinylbenzene];
poly[styrene-co-4-vinylbenzenesulfonic acid-co-1-(4-vinylbenzyl)-pyridinium-bromide-co-3-methyl-1-(4-vinylbenzyl)-3H-imidazol-1-ium bisulfate-co-divinylbenzene];
poly[styrene-co-4-vinylbenzenesulfonic acid-co-1-(4-vinylbenzyl)-pyridinium-iodide-co-3-methyl-1-(4-vinylbenzyl)-3H-imidazol-1-ium bisulfate-co-divinylbenzene];
poly[styrene-co-4-vinylbenzenesulfonic acid-co-1-(4-vinylbenzyl)-pyridinium-bisulfate-co-3-methyl-1-(4-vinylbenzyl)-3H-imidazol-1-ium bisulfate-co-divinylbenzene];
poly[styrene-co-4-vinylbenzenesulfonic acid-co-1-(4-vinylbenzyl)-pyridinium-acetate-co-3-methyl-1-(4-vinylbenzyl)-3H-imidazol-1-ium bisulfate-co-divinylbenzene];
poly[styrene-co-4-vinylbenzenesulfonic acid-co-4-methyl-4-(4-vinylbenzyl)-morpholin-4-ium chloride-co-divinylbenzene];
poly[styrene-co-4-vinylbenzenesulfonic acid-co-4-methyl-4-(4-vinylbenzyl)-morpholin-4-ium bisulfate-co-divinylbenzene];
poly[styrene-co-4-vinylbenzenesulfonic acid-co-4-methyl-4-(4-vinylbenzyl)-morpholin-4-ium acetate-co-divinylbenzene];
poly[styrene-co-4-vinylbenzenesulfonic acid-co-4-methyl-4-(4-vinylbenzyl)-morpholin-4-ium formate-co-divinylbenzene];
poly[styrene-co-4-vinylbenzenesulfonic acid-co-triphenyl-(4-vinylbenzyl)-phosphonium chloride-co-divinylbenzene];
poly[styrene-co-4-vinylbenzenesulfonic acid-co-triphenyl-(4-vinylbenzyl)-phosphonium bisulfate-co-divinylbenzene];
poly[styrene-co-4-vinylbenzenesulfonic acid-co-triphenyl-(4-vinylbenzyl)-phosphonium acetate-co-divinylbenzene];
poly[styrene-co-4-vinylbenzenesulfonic acid-co-1-methyl-1-(4-vinylbenzyl)-piperdin-1-ium chloride-co-divinylbenzene];
poly[styrene-co-4-vinylbenzenesulfonic acid-co-1-methyl-1-(4-vinylbenzyl)-piperdin-1-ium bisulfate-co-divinylbenzene];
poly[styrene-co-4-vinylbenzenesulfonic acid-co-1-methyl-1-(4-vinylbenzyl)-piperdin-1-ium acetate-co-divinylbenzene];
poly[styrene-co-4-vinylbenzenesulfonic acid-co-4-(4-vinylbenzyl)-morpholine-4-oxide-co-divinyl benzene];
poly[styrene-co-4-vinylbenzenesulfonic acid-co-triethyl-(4-vinylbenzyl)-ammonium chloride-co-divinylbenzene];
poly[styrene-co-4-vinylbenzenesulfonic acid-co-triethyl-(4-vinylbenzyl)-ammonium bisulfate-co-divinylbenzene];
poly[styrene-co-4-vinylbenzenesulfonic acid-co-triethyl-(4-vinylbenzyl)-ammonium acetate-co-divinylbenzene];
poly[styrene-co-3-methyl-1-(4-vinylbenzyl)-3H-imidazol-1-ium chloride-co-4-boronyl-1-(4-vinylbenzyl)-pyridinium chloride-co-divinylbenzene];
poly[styrene-co-3-methyl-1-(4-vinylbenzyl)-3H-imidazol-1-ium chloride-co-1-(4-vinylphenyl)methylphosphonic acid-co-divinylbenzene];
poly[styrene-co-3-methyl-1-(4-vinylbenzyl)-3H-imidazol-1-ium bisulfate-co-1-(4-vinylphenyl)methylphosphonic acid-co-divinylbenzene];
poly[styrene-co-3-methyl-1-(4-vinylbenzyl)-3H-imidazol-1-ium acetate-co-1-(4-vinylphenyl)methylphosphonic acid-co-divinylbenzene];
poly[styrene-co-3-methyl-1-(4-vinylbenzyl)-3H-imidazol-1-ium nitrate-co-1-(4-vinylphenyl)methylphosphonic acid-co-divinylbenzene];
poly[styrene-co-4-vinylbenzenesulfonic acid-co-vinylbenzylchloride-co-1-methyl-2-vinyl-pyridinium chloride-co-divinylbenzene];
poly[styrene-co-4-vinylbenzenesulfonic acid-co-vinylbenzylchloride-co-1-methyl-2-vinyl-pyridinium bisulfate-co-divinylbenzene];
poly[styrene-co-4-vinylbenzenesulfonic acid-co-vinylbenzylchloride-co-1-methyl-2-vinyl-pyridinium acetate-co-divinylbenzene];
poly[styrene-co-4-vinylbenzenesulfonic acid-co-4-(4-vinylbenzyl)-morpholine-4-oxide-co-divinyl benzene];
poly [styrene-co-4-vinylphenylphosphonic acid-co-3-methyl-1-(4-vinylbenzyl)-3H-imidazol-1-ium chloride-co-divinylbenzene];
poly [styrene-co-4-vinylphenylphosphonic acid-co-3-methyl-1-(4-vinylbenzyl)-3H-imidazol-1-ium bisulfate-co-divinylbenzene];
poly [styrene-co-4-vinylphenylphosphonic acid-co-3-methyl-1-(4-vinylbenzyl)-3H-imidazol-1-ium acetate-co-divinylbenzene];
poly[styrene-co-3-carboxymethyl-1-(4-vinylbenzyl)-3H-imidazol-1-ium chloride-co-divinylbenzene];
poly[styrene-co-3-carboxymethyl-1-(4-vinylbenzyl)-3H-imidazol-1-ium bisulfate-co-divinylbenzene];
poly[styrene-co-3-carboxymethyl-1-(4-vinylbenzyl)-3H-imidazol-1-ium acetate-co-divinylbenzene];
poly[styrene-co-5-(4-vinylbenzylamino)-isophthalic acid-co-3-methyl-1-(4-vinylbenzyl)-3H-imidazol-1-ium chloride-co-divinylbenzene];
poly[styrene-co-5-(4-vinylbenzylamino)-isophthalic acid-co-3-methyl-1-(4-vinylbenzyl)-3H-imidazol-1-ium bisulfate-co-divinylbenzene];
poly[styrene-co-5-(4-vinylbenzylamino)-isophthalic acid-co-3-methyl-1-(4-vinylbenzyl)-3H-imidazol-1-ium acetate-co-divinylbenzene];
poly[styrene-co-(4-vinylbenzylamino)-acetic acid-co-3-methyl-1-(4-vinylbenzyl)-3H-imidazol-1-ium chloride-co-divinylbenzene];
poly[styrene-co-(4-vinylbenzylamino)-acetic acid-co-3-methyl-1-(4-vinylbenzyl)-3H-imidazol-1-ium bisulfate-co-divinylbenzene];
poly[styrene-co-(4-vinylbenzylamino)-acetic acid-co-3-methyl-1-(4-vinylbenzyl)-3H-imidazol-1-ium acetate-co-divinylbenzene];
poly(styrene-co-4-vinylhenzenesulfonic acid-co-vinylbenzylmethylimidazolium chloride-co-vinylbenzylmethylmorpholinium chloride-co-vinylbenzyltriphenyl phosphonium chloride-co-divinylbenzene);
poly(styrene-co-4-vinylbenzenephosphonic acid-co-vinylbenzylmethylimidazolium chloride-co-vinylbenzylmethylmorpholinium chloride-co-vinylbenzyltriphenyl phosphonium chloride-co-divinylbenzene);
poly(styrene-co-4-vinylbenzenesulfonic acid-co-vinylbenzylmethylimidazolium bisulfate-co-vinylbenzylmethylmorpholinium bisulfate-co-vinylbenzyltriphenyl phosphonium bisulfate-co-divinylbenzene);
poly(styrene-co-4-vinylbenzenephosphonic acid-co-vinylbenzylmethylimidazolium bisulfate-co-vinylbenzylmethylmorpholinium bisulfate-co-vinylbenzyltriphenyl phosphonium bisulfate-co-divinylbenzene);
poly(styrene-co-4-vinylbenzenesulfonic acid-co-vinylbenzylmethylimidazolium acetate-co-vinylbenzylmethylmorpholinium acetate-co-vinylbenzyltriphenyl phosphonium acetate-co-divinylbenzene);
poly(styrene-co-4-vinylbenzenephosphonic acid-co-vinylbenzylmethylimidazolium acetate-co-vinylbenzylmethylmorpholinium acetate-co-vinylbenzyltriphenyl phosphonium acetate-co-divinylbenzene);
poly(styrene-co-4-vinylbenzenesulfonic acid-co-vinylbenzylmethylmorpholinium chloride-co-vinylbenzyltriphenylphosphonium chloride-co-divinylbenzene);
poly(styrene-co-4-vinylbenzenephosphonic acid-co-vinylbenzylmethylmorpholinium chloride-co-vinylbenzyltriphenylphosphonium chloride-co-divinylbenzene);
poly(styrene-co-4-vinylbenzenesulfonic acid-co-vinylbenzylmethylmorpholinium bisulfate-co-vinylbenzyltriphenylphosphonium bisulfate-co-divinylbenzene);
poly(styrene-co-4-vinylbenzenephosphonic acid-co-vinylbenzylmethylmorpholinium bisulfate-co-vinylbenzyltriphenylphosphonium bisulfate-co-divinylbenzene);
poly(styrene-co-4-vinylbenzenesulfonic acid-co-vinylbenzylmethylmorpholinium acetate-co-vinylbenzyltriphenylphosphonium bisulfate-co-divinylbenzene);
poly(styrene-co-4-vinylbenzenephosphonic acid-co-vinylbenzylmethylmorpholinium acetate-co-vinylbenzyltriphenylphosphonium bisulfate-co-divinylbenzene) poly(styrene-co-4-vinylbenzenesulfonic acid-co-vinylmethylimidazolium chloride-co-divinylbenzene);
poly(styrene-co-4-vinylbenzenesulfonic acid-co-vinylmethylimidazolium bisulfate-co-divinylbenzene);
poly(styrene-co-4-vinylhenzenesulfonic acid-co-vinylmethylimidazolium acetate-co-divinylbenzene);
poly(styrene-co-4-vinylbenzenesulfonic acid-co-vinylmethylimidazolium nitrate-co-divinylbenzene);
poly(styrene-co-4-vinylbenzenephosphonic acid-co-vinylmethylimidazolium chloride-co-divinylbenzene);
poly(styrene-co-4-vinylbenzenephosphonic acid-co-vinylmethylimidazolium bisulfate-co-divinylbenzene);
poly(styrene-co-4-vinylbenzenephosphonic acid-co-vinylmethylimidazolium acetate-co-divinylbenzene);
poly(styrene-co-4-vinylbenzenesulfonic acid-co-vinylbenzyltriphenylphosphonium chloride-co-divinylbenzene);
poly(styrene-co-4-vinylbenzenesulfonic acid-co-vinylbenzyltriphenylphosphonium bisulfate-co-divinylbenzene);
poly(styrene-co-4-vinylbenzenesulfonic acid-co-vinylbenzyltriphenylphosphonium acetate-co-divinylbenzene);
poly(styrene-co-4-vinylbenzenephosphonic acid-co-vinylbenzyltriphenylphosphonium chloride-co-divinylbenzene);
poly(styrene-co-4-vinylbenzenephosphonic acid-co-vinylbenzyltriphenylphosphonium bisulfate-co-divinylbenzene);
poly(styrene-co-4-vinylbenzenephosphonic acid-co-vinylbenzyltriphenylphosphonium acetate-co-divinylbenzene);
poly(styrene-co-4-vinylbenzenesulfonic acid-co-vinylbenzylmethylimidazolium chloride-co-divinylbenzene);
poly(styrene-co-4-vinylbenzenesulfonic acid-co-vinylbenzylmethylimidazolium bisulfate-co-divinylbenzene);
poly(styrene-co-4-vinylbenzenesulfonic acid-co-vinylbenzylmethylimidazolium acetate-co-divinylbenzene);
poly(styrene-co-4-vinylbenzenephosphonic acid-co-vinylbenzylmethylimidazolium chloride-co-divinylbenzene);
poly(styrene-co-4-vinylbenzenephosphonic acid-co-vinylbenzylmethylimidazolium bisulfate-co-divinylbenzene);
poly(styrene-co-4-vinylbenzenephosphonic acid-co-vinylbenzylmethylimidazolium acetate-co-divinylbenzene);
poly(styrene-co-4-vinylbenzenesulfonic acid-co-vinylbenzyltriphenylphosphonium chloride-co-divinylbenzene);
poly(styrene-co-4-vinylbenzenesulfonic acid-co-vinylbenzyltriphenylphosphonium bisulfate-co-divinylbenzene);
poly(styrene-co-4-vinylbenzenesulfonic acid-co-vinylbenzyltriphenylphosphonium acetate-co-divinylbenzene);
poly(styrene-co-4-vinylbenzenephosphonic acid-co-vinylbenzyltriphenylphosphonium chloride-co-divinylbenzene);
poly(styrene-co-4-vinylbenzenephosphonic acid-co-vinylbenzyltriphenylphosphonium bisulfate-co-divinylbenzene);
poly(styrene-co-4-vinylbenzenephosphonic acid-co-vinylbenzyltriphenylphosphonium acetate-co-divinylbenzene);
poly(butyl-vinylimidazolium chloride-co-butylimidazolium bisulfate-co-4-vinylbenzenesulfonic acid);
poly(butyl-vinylimidazolium bisulfate-co-butylimidazolium bisulfate-co-4-vinylbenzenesulfonic acid);
poly(benzyl alcohol-co-4-vinylbenzylalcohol sulfonic acid-co-vinylbenzyltriphenylphosphonium chloride-co-divinylbenzyl alcohol); and
poly(benzyl alcohol-co-4-vinylbenzylalcohol sulfonic acid-co-vinylbenzyltriphenylphosphonium bisulfate-co-divinylbenzyl alcohol).
   76. The method of any one of embodiments 1 to 10, wherein the catalyst is selected from the group consisting of:
carbon-supported pyrrolium chloride sulfonic acid;
carbon-supported imidazolium chloride sulfonic acid;
carbon-supported pyrazolium chloride sulfonic acid;
carbon-supported oxazolium chloride sulfonic acid;
carbon-supported thiazolium chloride sulfonic acid;
carbon-supported pyridinium chloride sulfonic acid;
carbon-supported pyrimidinium chloride sulfonic acid;
carbon-supported pyrazinium chloride sulfonic acid;
carbon-supported pyridazinium chloride sulfonic acid;
carbon-supported thiazinium chloride sulfonic acid;
carbon-supported morpholinium chloride sulfonic acid;
carbon-supported piperidinium chloride sulfonic acid;
carbon-supported piperizinium chloride sulfonic acid;
carbon-supported pyrollizinium chloride sulfonic acid;
carbon-supported triphenyl phosphonium chloride sulfonic acid;
carbon-supported trimethyl phosphonium chloride sulfonic acid;
carbon-supported triethyl phosphonium chloride sulfonic acid;
carbon-supported tripropyl phosphonium chloride sulfonic acid;
carbon-supported tributyl phosphonium chloride sulfonic acid;
carbon-supported trifluoro phosphonium chloride sulfonic acid;
carbon-supported pyrrolium bromide sulfonic acid;
carbon-supported imidazolium bromide sulfonic acid;
carbon-supported pyrazolium bromide sulfonic acid;
carbon-supported oxazolium bromide sulfonic acid;
carbon-supported thiazolium bromide sulfonic acid;
carbon-supported pyridinium bromide sulfonic acid;
carbon-supported pyrimidinium bromide sulfonic acid;
carbon-supported pyrazinium bromide sulfonic acid;
carbon-supported pyridazinium bromide sulfonic acid;
carbon-supported thiazinium bromide sulfonic acid;
carbon-supported morpholinium bromide sulfonic acid;
carbon-supported piperidinium bromide sulfonic acid;
carbon-supported piperizinium bromide sulfonic acid;
carbon-supported pyrollizinium bromide sulfonic acid;
carbon-supported triphenyl phosphonium bromide sulfonic acid;
carbon-supported trimethyl phosphonium bromide sulfonic acid;
carbon-supported triethyl phosphonium bromide sulfonic acid;
carbon-supported tripropyl phosphonium bromide sulfonic acid;
carbon-supported tributyl phosphonium bromide sulfonic acid;
carbon-supported trifluoro phosphonium bromide sulfonic acid;
carbon-supported pyrrolium bisulfate sulfonic acid;
carbon-supported imidazolium bisulfate sulfonic acid;
carbon-supported pyrazolium bisulfate sulfonic acid;
carbon-supported oxazolium bisulfate sulfonic acid;
carbon-supported thiazolium bisulfate sulfonic acid;
carbon-supported pyridinium bisulfate sulfonic acid;
carbon-supported pyrimidinium bisulfate sulfonic acid;
carbon-supported pyrazinium bisulfate sulfonic acid;
carbon-supported pyridazinium bisulfate sulfonic acid;
carbon-supported thiazinium bisulfate sulfonic acid;
carbon-supported morpholinium bisulfate sulfonic acid;
carbon-supported piperidinium bisulfate sulfonic acid;
carbon-supported piperizinium bisulfate sulfonic acid;
carbon-supported pyrollizinium bisulfate sulfonic acid;
carbon-supported triphenyl phosphonium bisulfate sulfonic acid;
carbon-supported trimethyl phosphonium bisulfate sulfonic acid;
carbon-supported triethyl phosphonium bisulfate sulfonic acid;
carbon-supported tripropyl phosphonium bisulfate sulfonic acid;
carbon-supported tributyl phosphonium bisulfate sulfonic acid;
carbon-supported trifluoro phosphonium bisulfate sulfonic acid;
carbon-supported pyrrolium formate sulfonic acid;
carbon-supported imidazolium formate sulfonic acid;
carbon-supported pyrazolium formate sulfonic acid;
carbon-supported oxazolium formate sulfonic acid;
carbon-supported thiazolium formate sulfonic acid;
carbon-supported pyridinium formate sulfonic acid;
carbon-supported pyrimidinium formate sulfonic acid;
carbon-supported pyrazinium formate sulfonic acid;
carbon-supported pyridazinium formate sulfonic acid;
carbon-supported thiazinium formate sulfonic acid;
carbon supported morpholinium formate sulfonic acid;
carbon-supported piperidinium formate sulfonic acid;
carbon-supported piperizinium formate sulfonic acid;
carbon-supported pyrollizinium formate sulfonic acid;
carbon-supported triphenyl phosphonium formate sulfonic acid;
carbon-supported trimethyl phosphonium formate sulfonic acid;
carbon-supported triethyl phosphonium formate sulfonic acid;
carbon-supported tripropyl phosphonium formate sulfonic acid;
carbon-supported tributyl phosphonium formate sulfonic acid;
carbon-supported trifluoro phosphonium formate sulfonic acid;
carbon-supported pyrrolium acetate sulfonic acid;
carbon-supported imidazolium acetate sulfonic acid;
carbon-supported pyrazolium acetate sulfonic acid;
carbon-supported oxazolium acetate sulfonic acid;
carbon-supported thiazolium acetate sulfonic acid;
carbon-supported pyridinium acetate sulfonic acid;
carbon-supported pyrimidinium acetate sulfonic acid;
carbon-supported pyrazinium acetate sulfonic acid;
carbon-supported pyridazinium acetate sulfonic acid;
carbon-supported thiazinium acetate sulfonic acid;
carbon-supported morpholinium acetate sulfonic acid;
carbon-supported piperidinium acetate sulfonic acid;
carbon-supported piperizinium acetate sulfonic acid;
carbon-supported pyrollizinium acetate sulfonic acid;
carbon-supported triphenyl phosphonium acetate sulfonic acid;
carbon-supported trimethyl phosphonium acetate sulfonic acid;
carbon-supported triethyl phosphonium acetate sulfonic acid;
carbon-supported tripropyl phosphonium acetate sulfonic acid;
carbon-supported tributyl phosphonium acetate sulfonic acid;
carbon-supported trifluoro phosphonium acetate sulfonic acid;
carbon-supported pyrrolium chloride phosphonic acid;
carbon-supported imidazolium chloride phosphonic acid;
carbon-supported pyrazolium chloride phosphonic acid;
carbon-supported oxazolium chloride phosphonic acid;
carbon-supported thiazolium chloride phosphonic acid;
carbon-supported pyridinium chloride phosphonic acid;
carbon-supported pyrimidinium chloride phosphonic acid;
carbon-supported pyrazinium chloride phosphonic acid;
carbon-supported pyridazinium chloride phosphonic acid;
carbon-supported thiazinium chloride phosphonic acid;
carbon-supported morpholinium chloride phosphonic acid;
carbon-supported piperidinium chloride phosphonic acid;
carbon-supported piperizinium chloride phosphonic acid;
carbon-supported pyrollizinium chloride phosphonic acid;
carbon-supported triphenyl phosphonium chloride phosphonic acid;
carbon-supported trimethyl phosphonium chloride phosphonic acid;
carbon-supported triethyl phosphonium chloride phosphonic acid;
carbon-supported tripropyl phosphonium chloride phosphonic acid;
carbon-supported tributyl phosphonium chloride phosphonic acid;
carbon-supported trifluoro phosphonium chloride phosphonic acid;
carbon-supported pyrrolium bromide phosphonic acid;
carbon-supported imidazolium bromide phosphonic acid;
carbon-supported pyrazolium bromide phosphonic acid;
carbon-supported oxazolium bromide phosphonic acid;
carbon-supported thiazolium bromide phosphonic acid;
carbon-supported pyridinium bromide phosphonic acid;
carbon-supported pyrimidinium bromide phosphonic acid;
carbon-supported pyrazinium bromide phosphonic acid;
carbon-supported pyridazinium bromide phosphonic acid;
carbon-supported thiazinium bromide phosphonic acid;
carbon-supported morpholinium bromide phosphonic acid;
carbon-supported piperidinium bromide phosphonic acid;
carbon-supported piperizinium bromide phosphonic acid;
carbon-supported pyrollizinium bromide phosphonic acid;
carbon-supported triphenyl phosphonium bromide phosphonic acid;
carbon-supported trimethyl phosphonium bromide phosphonic acid;
carbon-supported triethyl phosphonium bromide phosphonic acid;
carbon-supported tripropyl phosphonium bromide phosphonic acid;
carbon-supported tributyl phosphonium bromide phosphonic acid;
carbon-supported trifluoro phosphonium bromide phosphonic acid;
carbon-supported pyrrolium bisulfate phosphonic acid;
carbon-supported imidazolium bisulfate phosphonic acid;
carbon-supported pyrazolium bisulfate phosphonic acid;
carbon-supported oxazolium bisulfate phosphonic acid;
carbon-supported thiazolium bisulfate phosphonic acid;
carbon-supported pyridinium bisulfate phosphonic acid;
carbon-supported pyrimidinium bisulfate phosphonic acid;
carbon-supported pyrazinium bisulfate phosphonic acid;
carbon-supported pyridazinium bisulfate phosphonic acid;
carbon-supported thiazinium bisulfate phosphonic acid;
carbon-supported morpholinium bisulfate phosphonic acid;
carbon-supported piperidinium bisulfate phosphonic acid;
carbon-supported piperizinium bisulfate phosphonic acid;
carbon-supported pyrollizinium bisulfate phosphonic acid;
carbon-supported triphenyl phosphonium bisulfate phosphonic acid;
carbon-supported trimethyl phosphonium bisulfate phosphonic acid;
carbon-supported triethyl phosphonium bisulfate phosphonic acid;
carbon-supported tripropyl phosphonium bisulfate phosphonic acid;
carbon-supported tributyl phosphonium bisulfate phosphonic acid;
carbon-supported trifluoro phosphonium bisulfate phosphonic acid;
carbon-supported pyrrolium formate phosphonic acid;
carbon-supported imidazolium formate phosphonic acid;
carbon-supported pyrazolium formate phosphonic acid;
carbon-supported oxazolium formate phosphonic acid;
carbon-supported thiazolium formate phosphonic acid;
carbon-supported pyridinium formate phosphonic acid;
carbon-supported pyrimidinium formate phosphonic acid;
carbon-supported pyrazinium formate phosphonic acid;
carbon-supported pyridazinium formate phosphonic acid;
carbon-supported thiazinium formate phosphonic acid;
carbon-supported morpholinium formate phosphonic acid;
carbon-supported piperidinium formate phosphonic acid;
carbon-supported piperizinium formate phosphonic acid;
carbon-supported pyrollizinium formate phosphonic acid;
carbon-supported triphenyl phosphonium formate phosphonic acid;
carbon-supported trimethyl phosphonium formate phosphonic acid;
carbon-supported triethyl phosphonium formate phosphonic acid;
carbon-supported tripropyl phosphonium formate phosphonic acid;
carbon-supported tributyl phosphonium formate phosphonic acid;
carbon-supported trifluoro phosphonium formate phosphonic acid;
carbon-supported pyrrolium acetate phosphonic acid;
carbon-supported imidazolium acetate phosphonic acid;
carbon-supported pyrazolium acetate phosphonic acid;
carbon-supported oxazolium acetate phosphonic acid;
carbon-supported thiazolium acetate phosphonic acid;
carbon-supported pyridinium acetate phosphonic acid;
carbon-supported pyrimidinium acetate phosphonic acid;
carbon-supported pyrazinium acetate phosphonic acid;
carbon-supported pyridazinium acetate phosphonic acid;
carbon-supported thiazinium acetate phosphonic acid;
carbon-supported morpholinium acetate phosphonic acid;
carbon-supported piperidinium acetate phosphonic acid;
carbon-supported piperizinium acetate phosphonic acid;
carbon-supported pyrollizinium acetate phosphonic acid;
carbon-supported triphenyl phosphonium acetate phosphonic acid;
carbon-supported trimethyl phosphonium acetate phosphonic acid;
carbon-supported triethyl phosphonium acetate phosphonic acid;
carbon-supported tripropyl phosphonium acetate phosphonic acid;
carbon-supported tributyl phosphonium acetate phosphonic acid;
carbon-supported trifluoro phosphonium acetate phosphonic acid;
carbon-supported ethanoyl-triphosphonium sulfonic acid;
carbon-supported ethanoyl-methylmorpholinium sulfonic acid; and
carbon-supported ethanoyl-imidazolium sulfonic acid.
   77. The method of any one of embodiments 1 to 76, wherein the catalyst has a catalyst activity loss of less than 1% per cycle.
   78. A method of manufacturing a food product, comprising: combining a food ingredient produced according to the method of any one of embodiments 2 to 77 with other ingredients to manufacture a food product.
   79. A polished oligosaccharide composition produced according to the method of any one of embodiments 1 and 3 to 78.
   80. A food ingredient produced according to the method of any one of embodiments 2 to 78.
   81. A food product produced according to the method of embodiment 80.
   82. An oligosaccharide composition for use as a food ingredient or for use in a food product, wherein the oligosaccharide composition is produced by:
      combining feed sugar with a catalyst to form a reaction mixture,
         wherein the catalyst comprises acidic monomers and ionic monomers connected to form a polymeric backbone, or
         wherein the catalyst comprises a solid support, acidic moieties attached to the solid support, and ionic moieties attached to the solid support; and
      producing the oligosaccharide composition from at least a portion of the reaction mixture.
   83. A food ingredient, comprising an oligosaccharide composition, wherein:
      (a) the oligosaccharide composition has a glycosidic bond type distribution of:
         at least 10 mol % α-(1,3) glycosidic linkages; and
         at least 10 mol % β-(1,3) glycosidic linkages; and
      (b) at least 10 dry wt % of the oligosaccharide composition has a degree of polymerization of at least 3; and
      (c) a metabolizable energy content, on a dry matter basis, of less than 4 kcal/g.
   84. The food ingredient of embodiment 83, wherein the oligosaccharide composition has a glycosidic bond type distribution of less than 9 mol % α-(1,4) glycosidic linkages, and less than 19 mol % α-(1,6) glycosidic linkages.
   85. A food ingredient, comprising an oligosaccharide composition, wherein:
      (a) the oligosaccharide composition has a glycosidic bond type distribution of:
         less than 9 mol % α-(1,4) glycosidic linkages; and
         less than 19 mol % α-(1,6) glycosidic linkages; and
      (b) at least 10 dry wt % of the oligosaccharide composition has a degree of polymerization of at least 3; and
      (c) a metabolizable energy content, on a dry matter basis, of less than 4 kcal/g.
   86. The food ingredient of any one of embodiments 83 to 85, wherein the oligosaccharide composition has a glycosidic bond type distribution of at least 15 mol % 1-(1,2) glycosidic linkages.
   87. The food ingredient of any one of embodiments 83 to 86, wherein the oligosaccharide composition comprises an oligosaccharide selected from the group consisting of a gluco-oligosaccharide, a galacto-oligosaccharide, a fructo-oligosaccharide, a manno-oligosaccharide, a gluco-galacto-oligosaccharide, a gluco-fructo-oligosaccharide, a gluco-manno-oligosaccharide, a gluco-arabino-oligosaccharide, a gluco-xylo-oligosaccharide, a galacto-fructo-oligosaccharide, a galacto-manno-oligosaccharide, a galacto-arabino-oligosaccharide, a galacto-xylo-oligosaccharide, a fructo-manno-oligosaccharide, a fructo-arabino-oligosaccharide, a fructo-xylo-oligosaccharide, a manno-arabino-oligosaccharide, and a manno-xylo-oligosaccharide, or any combinations thereof.
   88. The food ingredient of any one of embodiments 83 to 87, wherein the oligosaccharide composition comprises an oligosaccharide selected from the group consisting of an arabino-oligosaccharide, a xylo-oligosaccharide, and an arabino-xylo-oligosaccharide, or any combinations thereof.
   89. The food ingredient of any one of embodiments 83 to 86, wherein the oligosaccharide composition comprises a gluco-oligosaccharide, a galacto-oligosaccharide, a fructo-oligosaccharide, a manno-oligosaccharide, a gluco-galacto-oligosaccharide, a gluco-fructo-oligosaccharide, a gluco-manno-oligosaccharide, a gluco-arabino-oligosaccharide, a gluco-xylo-oligosaccharide, a galacto-fructo-oligosaccharide, a galacto-manno-oligosaccharide, a galacto-arabino-oligosaccharide, a galacto-xylo-oligosaccharide, a fructo-manno-oligosaccharide, a fructo-arabino-oligosaccharide, a fructo-xylo-oligosaccharide, a manno-arabino-oligosaccharide, a manno-xylo-oligosaccharide, or a xylo-gluco-galacto-oligosaccharide, or any combinations thereof.
   90. The food ingredient of any one of embodiments 83 to 89, wherein the oligosaccharide composition has a glycosidic bond type distribution of:
      between 0 to 20 mol % α-(1,2) glycosidic linkages;
      between 0 to 45 mol % β-(1,2) glycosidic linkages;
      between 1 to 30 mol % α-(1,3) glycosidic linkages;
      between 1 to 20 mol % β-(1,3) glycosidic linkages;
      between 0 to 55 mol % β-(1,4) glycosidic linkages; and
   between 10 to 55 mol % β-(1,6) glycosidic linkages
   91. The food ingredient of any one of embodiments 84 to 90, wherein at least 50 dry wt % of the oligosaccharide composition has a degree of polymerization of at least 3.
   92. The food ingredient of any one of embodiments 84 to 90, wherein between 65 and 80 dry wt % of the oligosaccharide composition has a degree of polymerization of at least 3.
   93. The food ingredient of any one of embodiments 84 to 90, wherein at least 50 dry wt % of the oligosaccharide composition comprises one or more gluco-oligosaccharides.
   94. The food ingredient of any one of embodiments 84 to 90, wherein at least 50 dry wt % of the oligosaccharide composition comprises one or more gluco-galacto-oligosaccharides.
   95. The food ingredient of any one of embodiments 84 to 94, wherein the oligosaccharide composition has a glycosidic bond type distribution of:
      between 0 to 20 mol % α-(1,2) glycosidic linkages;
      between 10 to 45 mol % β-(1,2) glycosidic linkages;
      between 1 to 30 mol % α-(1,3) glycosidic linkages;
      between 1 to 20 mol % β-(1,3) glycosidic linkages;
      between 0 to 55 mol % β-(1,4) glycosidic linkages;
      between 10 to 55 mol % β-(1,6) glycosidic linkages;
      less than 9 mol % α-(1,4) glycosidic linkages; and
      less than 19 mol % 0α-(1,6) glycosidic linkages.
   96. The food ingredient of any one of embodiments 84 to 94, wherein the oligosaccharide composition has a glycosidic bond type distribution of:
      between 0 to 15 mol % α-(1,2) glycosidic linkages;
      between 0 to 15 mol % β-(1,2) glycosidic linkages;
      between 1 to 20 mol % α-(1,3) glycosidic linkages;
      between 1 to 15 mol % β-(1,3) glycosidic linkages;
      between 5 to 55 mol % β-(1,4) glycosidic linkages;
      between 15 to 55 mol % β-(1,6) glycosidic linkages;
      less than 20 mol % α-(1,4) glycosidic linkages; and
      less than 30 mol % α-(1,6) glycosidic linkages.
   97. The food ingredient of any one of embodiments 84 to 96, wherein the oligosaccharide composition has a digestibility of less than 0.20 g/g.
   98. The food ingredient of any one of embodiments 84 to 97, wherein the oligosaccharide composition has a glass transition temperature of between −20 and 115° C. when measured at less than 10% moisture content.
   99. The food ingredient of any one of embodiments 84 to 98, wherein the oligosaccharide composition has a hygroscopicity of at least 5%, when measured at a water activity of 0.6
   100. The food ingredient of any one of embodiments 84 to 99, wherein the oligosaccharide composition has a fiber content of at least 80% on a dry mass basis.
   101. The food ingredient of any one of embodiments 84 to 100, wherein the oligosaccharide composition has a metabolizable energy content, on a dry matter basis, of less than 2 kcal/g, or less than 1.5 kcal/g; or between 1 kcal/g and 2.7 kcal/g, or between 1.1 kcal/g and 2.5 kcal/g, or between 1.1 and 2 kcal/g.
   102. The food ingredient of any one of embodiments 84 to 101, wherein the oligosaccharide composition is a functionalized oligosaccharide composition.
   103. The food ingredient of any one of embodiments 84 to 102, wherein the food ingredient is a syrup.
   104. The food ingredient of any one of embodiments 84 to 102, wherein the food ingredient is a powder.
   105. A food product comprising a food ingredient of any one of embodiments 80, 83 to 104.
   106. The food product of embodiment 105, wherein the food product is for human consumption
   107. The food product of embodiment 105 and 106, wherein the food product is a breakfast cereal, granola, yogurt, ice cream, bread, cookie, candy, cake mix, a nutritional shake, or a nutritional supplement.

### EXAMPLES

The following Examples are merely illustrative and are not meant to limit any aspects of the present disclosure in any way. Except where otherwise indicated, commercial reagents were purified prior to use following the guidelines of Perrin and Armarego (Perrin, D. D. & Armarego, W. L. F., Purification of Laboratory Chemicals, 3rd ed.; Pergamon Press, Oxford (1988)). Nitrogen gas for use in chemical reactions was of ultra-pure grade and was dried over phosphorous pentoxide or calcium chloride as required. Unless indicated otherwise, at bench-scale, all non-aqueous reagents were transferred under an inert atmosphere via syringe or Schlenk flask. Where necessary, chromatographic purification of reactants or products was performed using forced-flow chromatography on 60 mesh silica gel according to the method described in Still et al., J. Org. Chem., 43: 2923 (1978). Thin-layer chromatography (TLC) was performed using silica-coated glass plates. Visualization of the developed chromatographic plate was performed using either cerium molybdate (i.e., Hanessian) stain or KMnO₄ stain, with gentle heating as required. Fourier-Transform Infrared (FTIR) spectroscopic analysis of solid samples was performed on a Perkin-Elmer 1600 instrument using a horizontal attenuated total reflectance (ATR) configuration with a zinc selenide crystal.

The total dissolved solids content of soluble oligosaccharide compositions was determined by refractive index using a Hanna Instruments digital refractometer, Model HI 96801, with concentrations reported in units of Brix.

The moisture content of reagents was determined using a Mettler-Toledo MJ-33 moisture-analyzing balance with a sample size of 0.5-1.0 g and a heating cut-off temperature of 115° C. All moisture contents were determined as the average percent weight (% wt) loss on drying obtained from triplicate measurements.

The sugar, sugar alcohol, organic acid, furanic aldehyde and oligosaccharide content of reaction mixtures was determined by a combination of high performance liquid chromatography (HPLC) and spectrophotometric methods. HPLC determination of soluble sugars and sugar alcohols was performed on a Hewlett-Packard 1100 Series instrument equipped with a refractive index (RI) detector at 40° C. using a 30 cm×7.8 mm BioRad Aminex HPX-87P column at 80° C. with water at 0.6 mL/min as the mobile phase. The sugar column was protected by both a lead-exchanged sulfonated-polystyrene guard column and a tri-alkylammoniumhydroxide anionic-exchange guard column. All HPLC samples were microfiltered using a 0.2 μm syringe filter prior to injection. Sample concentrations were determined by reference to calibrations generated from a standard solution containing glucose, xylose, arabinose, galactose, sorbitol, and xylitol, in known concentrations.

The concentrations of sugar dehydration products, including anhydro-sugars, anhydro-sugar alcohols, organic acids, and furanic aldehydes, was determined by high performance liquid chromatography (HPLC) on a Hewlett-Packard 1100 Series instrument equipped with a refractive index (RI) detector at 30° C. using a 30 cm×7.8 mm BioRad Aminex HPX-87H column at 50° C. with 50 mM sulfuric acid at 0.65 mL/min as the mobile phase. The analytical column was protected by a sulfonated-polystyrene guard column and all HPLC samples were microfiltered using a 0.2 m syringe filter prior to injection. Sample concentrations were determined by reference to calibrations generated from a standard solution containing formic acid, acetic acid, levulinic acid, 5-hydroxymethylfurfural, and 2-furaldehyde or a standard solution containing sorbitol, 1,4-anhydrosorbitol, 1,5-anhydrosorbitol and isosorbide (1,4:3,6-Dianhydro-D-sorbitol).

The average degree of polymerization (DP) for oligosaccharides was determined as the number average of species containing one, two, three, four, five, six, seven, eight, nine, ten to fifteen, and greater than fifteen, anhydrosugar monomer units. The concentrations of oligosaccharides corresponding to these different DPs was determined by high performance liquid chromatography (HPLC) on a Hewlett-Packard 1100 Series instrument equipped with a refractive index (RI) detector at 40° C. using a 30 cm×7.8 mm BioRad Aminex HPX-87A column at 80° C. with water at 0.4 mL/min as the mobile phase. The analytical column was protected by a silver-coordinated, sulfonated-polystyrene guard column and all HPLC samples were microfiltered using a 0.2 m syringe filter prior to injection.

The conversion X(t) of monomeric (DP 1) sugars or sugar alcohols at time t was determined according to

$$X ( t ) = 1 - \frac{mol ( DP 1 , t )}{mol ( DP 1 , 0 )} ,$$

where mol(DP1,t) denotes the total moles of monomeric sugars or sugar alcohols present in the reaction at time t and mol(DP1,0) denotes the total moles of monomeric sugars or sugar alcohols initially charged to the reaction. Similarly, the yield to a given sugar dehydration species B was determined according to

$${Y}_{B} ( t ) = \frac{mol ( B , t )}{mol ( DP 1 , 0 )} ,$$

where mol(B,t) denotes the total moles of species B at reaction time t. Finally, the molar selectivity to a given product B was determined as the ratio of yield to conversion, namely S(t)=Y_{B}(t)/X(t).

The catalytic activity at a given reaction temperature and catalyst loading was determined as the effective first order rate constant for the conversion of reactants, k₁=−ln(1−X(t))t. The rate constant was calculated from reaction time-course data, typically by averaging the rate constant determined at multiple reaction times. The loss of catalyst activity upon re-use was determined as the fractional decrease in k₁ between consecutive cycles. The average loss of activity was determined as the arithmetic average of the catalyst activity loss computed for each consecutive reaction cycle.

The production of bi-products, such as polyfuranics, solid humins, and other poly-condensation products, was determined by inference from the reaction molar balance. Specifically, the molar yield to bi-products was determined as the arithmetic difference of the conversion and the sum of the yields to all quantifiable species.

The viscosity of solutions mixtures was determined using a Brookfield viscosometer mounted above a temperature-controlled oil bath used to set the temperature of the solution being measured from room temperature up to approximately 140 degrees Celsius.

The acid content of catalyst samples and aqueous solutions was determined using a Hana Instruments 902-C autotitrator with sodium hydroxide as the titrant, calibrated against a standard solution of potassium hydrogen phthalate (KHP). A known dry mass of solid catalyst was suspended in 40 mL of 10% sodium chloride solution at 60′C for 120 minutes prior to titration. The catalyst acidity was determined by dividing the total proton equivalents determined by titration by the dry mass of the dispensed catalyst and was reported in units of mmol H+/g dry catalyst.

The ionic content of catalyst samples was determined by titration against standardized silver nitrate solution. Solid catalyst for analysis was washed repeatedly on a fritted glass funnel with 100 mL volumes of 10% hydrochloric acid solution, followed by washing repeatedly with distilled water until the effluent eluted neutral. A sample of the acid-washed catalyst with known dry mass was then suspended in 40 mL of a 50% v/v solution of dimethylformamide (DMF) in water at 60° C. for 120 minutes prior to titration to a potassium chromate endpoint. The catalyst ionic content was determined by dividing the total chloride ion equivalents determined by titration by the dry mass of the dispensed catalyst and was reported in units of mmol ionic groups/g dry catalyst.

Concentration of liquid samples was performed using a Buchi r124 series rotary evaporator unit. For oligosaccharide solutions in water, a bath temperature of approximately 60 degrees Celsius was used. Vacuum pressure of 50-150 mTorr was provided by an oil-immersion pump, which was protected by an acetone-dry ice trap to prevent volatilized solvents from being drawn into the pump system.

The fiber content of oligosaccharides was determined by the following procedure. An aliquot of the sample was first analyzed for oligosaccharide and sugar content by HPLC, as described above. Sodium maleate buffer was prepared by dissolving 11.6 g of maleic acid into 1600 mL of deionized water, after which the pH was adjusted to exactly 6.0 with 4M sodium hydroxide solution. Then, 0.6 g of calcium chloride dehydrate and 0.4 g of sodium azide were dissolved in the mixture before adjusting the total volume to 2 liters. Trizma base solution was prepared by dissolving 90.8 g of Tris buffer salt (Sigma Catalog #T-1503) into 1 L of deionized water. Immediately prior to analysis, fresh digestion reagent was prepared by dissolving 0.1 g of purified porcine α-amylase (150,000 U/g) in 290 mL of the sodium maleate buffer. After stirring for 5 minutes, 0.3 mL of amyloglucosidase (3,300 U/mL in 50% v/v glycerol) was added to the solution followed by gentle mixing by inversion. The digestibility of samples was determined by dispensing 1.000 g of sample (dry solids basis) into a 250 mL plastic bottle (Nalgene, screw cap) and wetting or diluting the sample with 1 mL of 200 proof ethanol. 30 mL of the digestion reagent were then added and the bottle was capped and incubated at 37 degrees Celsius in an orbital shaker at 150 RPM for 16 hours. After the incubation period, the digestion was terminated by adding 3.0 mL of the Trizma base solution and heating the mixture to 95-100 degrees Celsius for 20 minutes in a boiling water bath with intermittent mixing. The sample was then cooled to 60 degrees Celsius, 0.1 mL of protease (50 mg/mL, 250 Tyrosine U/mL in 50% v/v glycerol) was added, and the mixture was incubated at 60 degrees Celsius for 30 minutes with orbital shaking at 150 RPM. 4.0 mL of acetic acid was then added to bring the final pH to 4.3. Aliquots of the digest were then analyzed by HPLC for their oligosaccharide and sugar content, as described above. The indigestibility was calculated by mass balance. Specifically, the mass of DP3+ oligos (DP at least three) following the digestion procedure was divided by the mass of DP3+ oligos present in the initial sample prior to the digestion procedure. The percent fiber was calculated by multiplying the % indigestible DP3+ oligosaccharides by the mass fraction of DP3+ oligosaccharides in the original sample.

The glass transition temperature Tg of oligosaccharide compositions was determined as follows. Samples were freeze dried for 3 days and the resulting powder was stored at −25 degrees Celsius prior to analysis. For analysis by differential scanning calorimetry (DSC), approximately 10 mg of sample was equilibrated at −50 degrees Celsius, heated at 10 degrees Celsius per minute to an annealing temperature below that of the onset of thermal decomposition (as verified by thermal gravimetric analysis), held isothermally for 3 minutes, cooled to −50 degrees Celsius at −25 degrees Celsius per minute, held isothermally for three minutes, and then heated to acquire the DSC scan. The onset, midpoint, and endpoint values for the glass transition were obtained from the second heating cycle. All measurements were performed in at least duplicate.

The hygroscopicity of samples was obtained by dispensing a known mass of dry oligosaccharide composition onto an aluminum weighing dish with known mass. Samples were placed in desiccators containing saturated salt solutions with known water activity and equilibrated to constant mass at 25 degrees Celsius. Specifically, moisture contents were obtained for the water activities listed in Table 2.

TABLE 2

| Saturated Salt Solution | Water Activity |
|---|---|
| LiCl | 0.1130 |
| MgCl₂ | 0.3278 |
| K₂CO₃ | 0.4316 |
| NaBr | 0.5757 |
| SrCl₂ | 0.7085 |
| NaCl | 0.7529 |
| KCl | 0.8434 |
| K₂SO₄ | 0.9730 |

The moisture content was determined by thermogravimetric analysis (TGA), using a program that heated the sample from 25 degrees Celsius to 180 degrees Celsius at 10 degrees Celsius per minute. Moisture sorption isotherms were constructed by plotting the moisture content versus water activity.

### Example 1

### Preparation of Catalyst

This Example demonstrates the preparation and characterization of poly-(styrene sulfonic acid-co-vinylbenzylimidazolium sulfate-co-divinylbenzene).

To a 30 L jacketed glass reactor, housed within a walk-in fume hood and equipped with a 2 inch bottom drain port and a multi-element mixer attached to an overhead air-driven stirrer, was charged 14 L of N,N-dimethylformamide (DMF, ACS Reagent Grade, Sigma-Aldrich, St. Louis, MO, USA) and 2.1 kg of 1H-imidazole (ACS Reagent Grade, Sigma-Aldrich, St. Louis, MO, USA) at room temperature. The DMF was stirred to dissolve the imidazole. To the reactor was then added 7.0 kg of cross-linked poly-(styrene-co-divinylbenzene-co-vinylbenzyl chloride) to form a stirred suspension. The reaction mixture was heated to 90 degrees Celsius by pumping heated bath fluid through the reactor jacket, and the reaction mixture was allowed to react for 24 hours, after which it was gradually cooled.

Then, the DMF and residual unreacted 1H-imidazole was drained from the resin, after which the retained resin was washed repeatedly with acetone to remove residual heavy solvent or unreacted reagents. The reaction yielded cross-linked poly-(styrene-co-divinylbenzene-co-1H-imidazolium chloride) as off-white spherical resin beads. The resin beads were removed from the reactor and heated at 70 degrees Celsius in air to dry.

The cleaned 30 L reactor system was charged with 2.5 L of 95% sulfuric acid (ACS Reagent Grade) and then approximately 13 L of oleum (20% free SO₃ content by weight, Puritan Products, Inc., Philadelphia, PA, USA). To the stirred acid solution was gradually added 5.1 kg of the cross-linked poly-(styrene-co-divinylbenzene-co-1H-imidazolium chloride). After the addition, the reactor was flushed with dry nitrogen gas, the stirred suspension was heated to 90 degrees Celsius by pumping heated bath fluid through the reactor jacket, and the suspension was maintained at 90 degrees Celsius for approximately four hours. After completion of the reaction, the mixture was allowed to cool to approximately 60 degrees Celsius and the residual sulfuric acid mixture was drained from the reactor. The resin was washed with 80 wt % sulfuric acid solution, followed by 60 wt % sulfuric acid solution. Then the resin was washed repeatedly with distilled water until the pH of the wash water was above 5.0, as determined by pH paper, to yield the solid catalyst. The acid functional density of catalyst was determined to be at least 2.0 mmol H+/g dry resin by ion-exchange acid-base titration.

### Example 2

### Preparation of Oligosaccharide Samples

This Example demonstrates the preparation oligosaccharides from different feed sugars using a catalyst with acidic and ionic moieties. The catalyst was used was poly-(styrene sulfonic acid-co-vinylbenzylimidazolium sulfate-co-divinylbenzene), prepared according to the procedure as described in Example 1 above. Various oligosaccharides were prepared at 100 g scale using the feed sugars and polishing steps listed in Table 3.

TABLE 3
Feed sugar and polishing steps used in preparation of oligosaccharides

| | Oligosaccharide | | |
|---|---|---|---|
| Reaction | Produced | Feed Sugars | Polishing Steps |
| 1 | gluco- | dextrose (95 DE | microfiltration, |
| | oligosaccharide | syrup) | decolorization, |
| | | | demineralziation |
| 2 | gluco- | dextrose (95 DE | microfiltration, |
| | oligosaccharide | syrup) | decolorization, |
| | | | demineralziation |
| 3 | gluco-galacto- | lactose | microfiltration, |
| | oligosaccharide | | decolorization, |
| | | | demineralziation |
| 4 | gluco-galacto- | lactose | microfiltration, |
| | oligosaccharide | | decolorization, |
| | | | demineralziation |
| 5 | gluco/sorbitol- | 90% dextrose, 10% | microfiltration, |
| | oligosaccharide | sorbitol | decolorization, |
| | | | demineralziation |
| 6 | gluco- | dextrose | microfiltration |
| | oligosaccharide | | |
| 7 | gluco- | dextrose | microfiltration, |
| | oligosaccharide | | decolorization |
| 8 | gluco- | dextrose | microfiltration, |
| | oligosaccharide | | demineralization |
| 9 | xylo- | xylose | microfiltration, |
| | oligosaccharide | | decolorization, |
| | | | demineralziation |
| 10 | gluco- | dextrose | microfiltration, |
| | oligosaccharide | | decolorization, |
| | | | demineralziation |
| 11 | arabino-galacto- | 50/50 | microfiltration, |
| | oligosaccharide | arabinose/galactose | decolorization, |
| | | | demineralziation |
| 12 | gluco- | 90% glucose, 10% | microfiltration, |
| | oligosaccharide | glycerol | decolorization, |
| | | | demineralziation |
| 13 | gluco- | dextrose (95 DE | microfiltration, |
| | oligosaccharide | syrup) | decolorization, |
| | | | demineralziation |
| 14 | gluco-galacto- | lactose | microfiltration, |
| | oligosaccharides | | decolorization, |
| | | | demineralziation |
| 15 | gluco-xylo- | 75% glucose/25% | microfiltration, |
| | oligoaccharide | xylose | decolorization, |
| | | | demineralziation |
| 16 | gluco-xylo- | 25% glucose/75% | microfiltration, |
| | oligoaccharide | xylose | decolorization, |
| | | | demineralziation |
| 17 | gluco-xylo- | 33% glucose/33% | microfiltration, |
| | galacto- | xylose/33% | decolorization, |
| | oligosaccharide | galactose | demineralziation |
| 18 | xylo-gluco- | 12.5% glucose/75% | microfiltration, |
| | galacto- | xylose/12.5% | decolorization, |
| | oligosaccharide | galactose | demineralziation |

For each preparation, the feed sugars were dispensed into a 400 mL glass cylindrical reactor and gradually heated to 105° C. by heating the walls of the reactor with a temperature-controlled oil bath. Mixing was provided by an overhead mechanical stirrer equipped with a stainless steel three-blade impeller, where the ratio of the diameter of the mixing element to the diameter of the reaction vessel was approximately 0.8. During the heating process, the minimum volume of water required to bring the sugars into a viscous syrup was dispensed. The feed sugar concentration in each case was approximately 75% g sugar/g syrup and the viscosity was approximately 400-600 cP. Once at temperature, catalyst was dispensed into the reactor at a total loading of 0.2 g of dry catalyst per dry gram of feed sugar. With mixing at a stir rate of approximately 100 RPM, the catalyst formed a viscous suspension, which was maintained for approximately three hours at 105° C. Over the course of the reaction, the solution thickened as oligosaccharides formed and water evaporated from the reaction vessel, with an increase in viscosity to approximately 1,000-2,000 cP. The final moisture content of the reaction mixture was determined to be approximately 5%. After three hours, 100 mL of de-ionized water was dispensed into the reactor to dilute the oligosaccharide composition to approximately 50 Brix. The mixture was cooled to room temperature and the resulting oligosaccharide syrup was separated from the catalyst by vacuum filtration through a coarse membrane (pore size 50-100 micron). During filtration, additional water was used to wash residual soluble species from the catalyst, resulting in further dilution of the oligosaccharide compositions to approximately 25 Brix.

The recovered syrup from each preparation went through polishing steps as listed in Table 2. Decolorization was performed by dispensing approximately 100 mL of syrup into a 300 mL cylindrical glass vessel and heating the syrup to 65° C. using an external temperature-controlled oil bath to heat the walls of the vessel. Mixing was provided by magnetic stirring at a stir rate of 250 RPM. Powdered activated carbon (EXP-798, Cabot Corp.) was dispensed into the mixture at a loading of 1%-2% g dry activated carbon per gram solids to form a dark stirred suspension. The suspension was maintained at 65° C. for one hour, after which it was vacuum microfiltered through a 0.2 micron polyether sulfone membrane, to produce a decolorized syrup with no detectable suspended solids. Demineralization to remove salts, organic acid side-products (e.g., levulinic acid), and any other soluble ionic species was performed by ion exchange. The composition was passed through a series of two columns, the first containing a food grade strong acid cationic exchange resin (Chemra GmbH, Hamburg, Germany), with a contact time of 60 minutes at room temperature. The eluted product was then passed through a column containing a weak-base anionic exchange resin (Chemra GmbH, Hamburg, Germany), with a contact time of 60 minutes at room temperature.

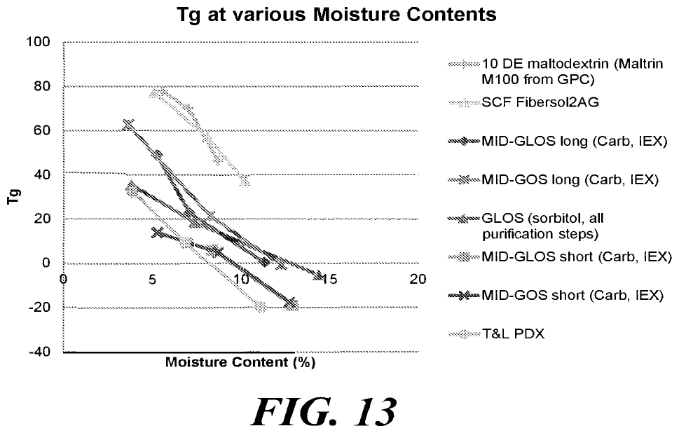

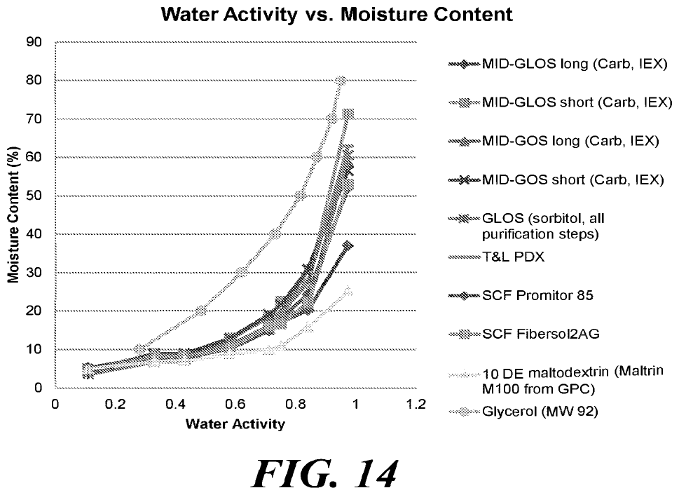

Samples of the resulting oligosaccharide composition were concentrated by vacuum rotary evaporation. The resulting products were analyzed by HPLC to determine their DP distribution, and glass transition temperature, hygroscopicity, and digestibility to determine fiber content analyses were performed as described above, as summarized in Table 4 below and FIGS. 13 and 14.

TABLE 4
Properties of produced oligosaccharides

| | | | | | | % MC @ | |
|---|---|---|---|---|---|---|---|
| | DP3+ | DP2 | DP1 | | | 0.58 Aw | Fiber |
| Reaction | (g/g) | (g/g) | (g/g) | DP | Tg | (g/g) | Content |
| 1 | 83.0% | 6.8% | 9.4% | 10 | 57.9 | 11.37 | 79% |
| 2 | 70.1% | 12.1% | 17.0% | 6 | 22.29 | 12.9 | 67% |
| 3 | 89.6% | 5.0% | 5.0% | 12 | 81.36 | 12.46 | 90% |
| 4 | 72.8% | 11.4% | 14.9% | 7 | 29.02 | 12.89 | 73% |
| 5 | 73.8% | 10.2% | 16.0% | 6 | 63.26 | 11.58 | 72% |
| 6 | 83.0% | 6.8% | 9.4% | 10 | 76.35 | 13.38 | 79% |
| 7 | 83.0% | 6.8% | 9.4% | 10 | 46.65 | 14.41 | 79% |
| 8 | 83.0% | 6.8% | 9.4% | 10 | 70.33 | 11.56 | 79% |
| 9 | 68.8% | 13.4% | 17.8% | 8 | 46.9 | n/d | 69% |
| 10 | 79.0% | 8.2% | 12.8% | 8 | 50 | n/d | 75% |
| 11 | 72.5% | 13.3% | 14.2% | 8 | 49.6 | n/d | 73% |
| 12 | 40.6% | 17.9% | 41.5% | 3 | 10.5 | n/d | 40% |
| 13 | 88.0% | 5.7% | 6.3% | 10 | 78.3 | n/d | 79% |
| 14 | 90.2% | 4.3% | 5.5% | 12 | 97.6 | n/d | 90% |
| 15 | 50.1% | 24.2% | 25.7% | 4 | 22.1 | n/d | 50% |
| 16 | 52.8% | 21.9% | 25.3% | 3 | 22.1 | n/d | 53% |
| 17 | 57.4% | 22.0% | 20.6% | 3 | 18.3 | n/d | 57% |
| 18 | 55.0% | 21.1% | 23.9% | 3 | 9.1 | n/d | 55% |

### Example 3

### Preparation of Yogurt Containing an Oligosaccharide Composition

This Example demonstrates the use of an oligosaccharide composition in the preparation of a yogurt food product. The oligosaccharide composition used was prepared according to the conditions of Reaction 2, as described in Example 2 above, using the catalyst prepared as described in Example 1. A high-fiber yogurt was produced by combining 10 g of the oligosaccharide composition with 2% milk, 5 g non-fat dry milk powder, and diluting the mixture to 200 mL. The mixture was inoculated with yogurt culture and was fermented for 24 hours produce the final yogurt product.

### Example 4

Preparation of a Breakfast Cereal Coated with an Oligosaccharide Composition

This Example demonstrates the use of an oligosaccharide composition in the coating of a breakfast cereal food product. The oligosaccharide composition used was prepared according to the conditions of Reaction 3, as described in Example 2 above, using the catalyst prepared as described in Example 1. Approximately 3 g of the oligosaccharide composition was suspended in 190 proof ethanol (Everclear, Luxco, USA). The resulting suspension was mixed with a 28 g serving of Cheerios breakfast cereal (General Mills Inc., USA) and mixed gently to achieve a uniform coating. The alcohol was evaporated at slightly elevated temperature to produce the coated cereal product, with approximately four times the dietary fiber content of the uncoated cereal.

### Example 5A

### Preparation of Chocolate Chip Cookies Containing an Oligosaccharide Composition

This Example demonstrates the use of an oligosaccharide composition in the preparation of a chocolate chip cookie food product.

The oligosaccharide composition used was prepared according to the conditions of Reaction 2, as described in Example 2 above, using the catalyst prepared as described in Example 1.

Chocolate chip cookies were prepared according to the Original Toll House Cookie Recipe (Nestle S.A., Switzerland) with the formulation described in Table 5, containing an oligosaccharide composition. The resulting cookie product contained approximately 2.89 g of soluble dietary fiber per serving. The fiber content was calculated from the fiber content of the ingredients plus the fiber content of the oligosaccharides.

TABLE 5
Composition of Chocolate Chip Cookie

| | | |
|---|---|---|
| Flour (all purpose) | 173 | g |
| Baking Soda | 2.5 | g |
| Salt | 3.0 | g |
| Butter | 30.36 | g |
| Shortening | 88.71 | g |
| Sugar | 23.00 | g |
| Brown Sugar | 50.00 | g |
| Vanilla | 1 | g |
| Egg | 60 | g |
| Chocolate Chips | 225 | g |
| Nuts | 35 | g |
| Fiber (Oligosaccharide Composition | 55.10 | g |
| Reaction #2 from Example 2) | | |

### Example 5B

### Preparation of Chocolate Brownies Containing and Oligosaccharide Composition

This Example demonstrates the use of an oligosaccharide composition in the preparation of a chocolate brownie food product. The oligosaccharide composition used was prepared according to the conditions of Reaction 2, as described in Example 2 above, using the catalyst prepared as described in Example 1. Chocolate brownies were prepared according to the formulation described in Table 6, containing an oligosaccharide composition. The resulting chocolate brownie product contained approximately 3 grams of soluble dietary fiber per serving. The fiber content was calculated from the fiber content of the ingredients plus the fiber content of the oligosaccharides.

TABLE 6
Composition of Chocolate Brownies

| | | |
|---|---|---|
| Brown Sugar | 51 | g |
| Shortening | 50 | g |
| Butter | 27 | g |
| Cocoa | 18 | g |
| Vanilla | 6 | g |
| Cake Flour | 70 | g |
| Canola Oil | 51 | g |
| Chocolate Chips | 45 | g |
| Walnuts | 35 | g |
| Raisins | 36 | g |
| Baking Powder | 8.25 | g |
| Fiber (Oligosaccharide Composition | 57 | g |
| Reaction #2 from Example 2) | | |

### Example 6

### Effect of Water Concentration on Oligosaccharide Yield and Degree of Polymerization

This Example demonstrates the effect that reaction water content has on overall oligosaccharide yield and the degree of polymerization in the preparation of oligosaccharides from different feed sugars using a catalyst with acidic and ionic moieties.

The catalyst was used was poly-(styrene sulfonic acid-co-vinylbenzylimidazolium sulfate-co-divinylbenzene), prepared according to the procedure as described in Example 1 above.

Each reaction was performed on a 100 g scale. To a 400 mL glass cylindrical reactor were added a known mass of water and a known mass of feed sugar, as described in Table 7. The resulting sugar/water mixture was mixed continuously and gradually brought to temperature by heating the walls of the reaction vessel using a temperature-controlled oil bath. Mixing was provided by an overhead mechanical stirrer equipped with a stainless steel three-blade impeller, where the ratio of the diameter of the mixing element to the diameter of the reaction vessel was approximately 0.8.

Once at temperature, catalyst was dispensed into the reactor at a total loading of 0.2 g of dry catalyst per dry gram of starting sugar, resulting in a stirred suspension. The stirred suspension was maintained at temperature for approximately three hours. At 0, 1, 2 and 3 hours, an 250 mg aliquot of the reaction mixture was diluted into 10 mL of deionized water and analyzed by HPLC to determine the concentrations of sugars and the concentration distribution of oligosaccharides with respect to their degree of polymerization (DP).

Over the course of the reaction, the water evaporation rate was controlled by adjusting the flow of air over the reaction mixture. This resulted in different final water contents for the various reactions. The moisture content at the end of each reaction was determined by drying a 0.5 g aliquot of the reaction mixture to constant mass under vacuum (P=10 mTorr) at 65° C.

The yields to DP2 and DP3+ oligosaccharides as a function of the final reaction water content for the various reactions are summarized in Table 7. The results indicate that controlling the water so the final reaction water content is below about 10% g/g, the yield of DP3+ oligosaccharides achieved is above about 57% mol/mol.

TABLE 7
Reaction conditions and yield of DP2 and DP3+ oligosaccharides

| | | | | Final | | |
|---|---|---|---|---|---|---|
| | | Feed | Starting | Water | DP2 | DP3+ |
| Reaction | Feed | Sugar | Water | Content | Yield | Yield |
| Number | Sugar | Mass (g) | Mass (g) | (g/g) | (mol/mol) | (mol/mol) |
| 1 | dextrose | 100 | 13 | 5% | 7% | 83% |
| 2 | dextrose | 100 | 13 | 8% | 10% | 71% |
| 7 | xylose | 100 | 13 | 8% | 13% | 71% |
| 3 | dextrose | 100 | 13 | 10% | 16% | 57% |
| 4 | dextrose | 100 | 13 | 12% | 9% | 41% |
| 5 | dextrose | 100 | 23 | 20% | 22% | 18% |
| 6 | dextrose | 100 | 58 | 50% | 23% | 6% |

### Example 7

### Refactoring of 18DE Corn Syrup to an Indigestible Gluco-Oligosaccharide

This Example demonstrates the refactoring of corn syrup. A feed sugar that is digestible to a human was reacted with the catalyst prepared according to the procedure as described in Example 1 above, at 100 g scale to convert it to an indigestible carbohydrate in a single step procedure. The catalyst used was poly-(styrene sulfonic acid-co-vinylbenzylimidazolium sulfate-co-divinylbenzene). Corn syrup (malto-dextrin), with an initial average degree of polymerization (DP) of 9 and an initial dextrose equivalent (DE) of 18, was analyzed for its digestibility by α-amylase/aminoglucosidase. It was found that 0.942 g/g (or 94.2%) of the DP3+ component and 0.675 g/g (or 67.5%) of the DP2 component of the corn syrup were digested to glucose, indicating that the chemical structure of the starting oligosaccharides consisted predominantly of α(1→4) glycosidic linkages.

100 g of the 18 DE corn syrup was combined with 25.8 g of de-ionized water and 20.2 dry g of the catalyst prepared according to the procedure as described in Example 1 above in a 400 mL glass cylindrical reactor. The resulting mixture was mixed continuously and gradually heated to 105° C. by heating the walls of the reaction vessel using a temperature-controlled oil bath. Mixing was provided by an overhead mechanical stirrer equipped with a stainless steel three-blade impeller, where the ratio of the diameter of the mixing element to the diameter of the reaction vessel was approximately 0.8. The stirred suspension was maintained at temperature for approximately four hours. At 0, 1, 2, 3, and 4 hours, a 250 mg aliquot of the reaction mixture was diluted into 10 mL of deionized water and analyzed by HPLC to determine the concentrations of sugars and the concentration distribution of oligosaccharides with respect to their degree of polymerization (DP).

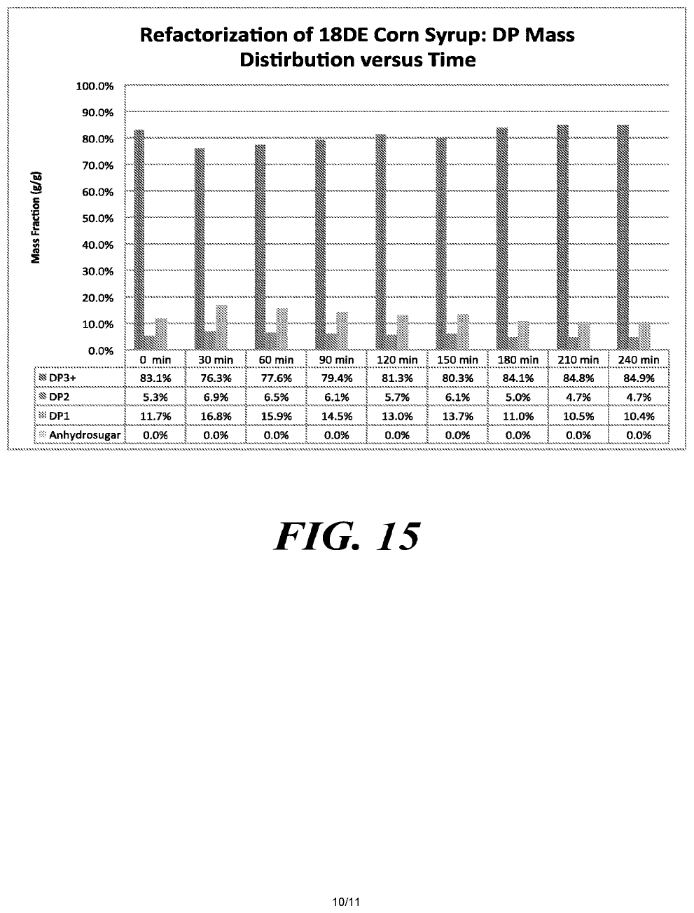

The distribution over DP over the course of the reaction is shown in FIG. 15. At no point during the reaction did the mass fraction of DP3+ species decrease below 76% g/g, indicating that minimal hydrolysis of the starting corn syrup took place. The mass fraction of glucose (DP1) was maintained between about 10% and 17% throughout the reaction.

Following the reaction, approximately 100 g of de-ionized water was added to dilute the mixture to about 50 Brix. The resulting gluco-oligosaccharide syrup was separated from the catalyst by vacuum filtration using a fritted glass funnel (pore size 50-100 micron). Additional water was used to wash the catalyst to remove additional soluble species, resulting in a final syrup concentration of approximately 25 Brix. The syrup was concentrated to 75 Brix by vacuum rotary evaporation.

The resulting gluco-oligosaccharide composition was analyzed for digestibility. It was found that only 0.108 g/g (or 10.8%) of the DP3+ component and 0.088 g/g (or 8.8%) of the DP2 component were digestible, indicating that the α(1→4) glycosidic linkages in the starting oligosaccharide had been effectively refactored into other, non human-digestible, linkage types. Analysis of the DP2 component by HPLC indicated the presence of β(1→4), α(1→3), β(1→3), α(1→6), and β(1→6) linkages in the product species.

### Example 8

Determination of Metabolizable energy content

In this Example, the metabolizable energy content of two oligosaccharide compositions prepared according to the methods described herein was determined.

### Materials and Methods

### Oligosaccharide Compositions

Sample #1 was a gluco-oligosasccharide composition produced from oligomerization of dextrose, prepared according to the method described in Example 2, Reaction #1 (see Table 3). Sample #2 was a gluco-oligosaccharide composition produced by refactoring 18DE maltodextrin (starch), prepared according to the method described in Example 7.

### Assays

Two precision-fed rooster assays utilizing conventional Single Comb White Leghorn roosters and cecectomized Single Comb White Leghorn roosters were conducted. After 24 hours of feed withdrawal, 5 conventional roosters and 5 cecectomized roosters were tube-fed an average of 34.4 grams (dry matter basis) of the test substrates (Samples #1 and #2) using the precision-fed rooster assay. Following crop intubation, excreta (urine and feces) were collected for 48 hours on plastic trays placed under each individual cage. Excreta samples then were lyophilized, weighed, and ground prior to analysis. The two samples and the excreta produced after the animals were dosed with these samples were analyzed for dry matter (DM) at 105° C., according to the procedure set forth in method AOAC 934.01 (c.f., Official Methods of Analysis, 17^{th} edition, Association of Official Analytical Chemists, International, 2006).

N or crude protein (CP) (determined using a TruMac® N, LECO Corporation, St. Joseph, MI, USA), and gross energy (GE) using a bomb calorimeter. The TMEₙ values, corrected for endogenous energy excretion using many fasted birds over many years, were calculated using the following equation:

$${TME}_{n} ( kcal / g ) = \frac{{EI}_{\underline{fed}} - ( {EE}_{\underline{fed}} + 8.22 * {N}_{\underline{fed}} ) + ( {EE}_{\underline{fasted}} + 8.22 * {N}_{\underline{fasted}} )}{FI}$$

where:
EI_{fed} is the gross energy intake of the test substrate consumed;
EE_{fed} is the energy in the excreta collected from fed birds;
8.22 is the correction factor for uric acid;
N_{fed} is the grams of nitrogen retained by the fed birds;
EE_{fasted} is the energy in the excreta collected from the fasted birds;
N_{fasted} is the grams of nitrogen retained by the fasted birds (1.1256 g); and
FI is the grams of dry test substrate consumed.

The method described above was used to determine the nitrogen-corrected true metabolizable energy content. The database with conventional and cecectomized birds indicate that values for endogenous energy excretion and endogenous energy coming from N excretion by fasted birds were 16.74 kcal/g and 9.25 kcal/g, respectively.

### Results

The TMEₙ of the two samples are summarized in Table 8 below. The TMEₙ of Sample #1 was 1.72 kcal/g when evaluated using conventional roosters and 1.39 kcal/g when evaluated using cecectomized roosters. The TMEn of Sample #2 was 1.17 kcal/g when evaluated using conventional roosters and 1.19 kcal/g when evaluated using cecectomized roosters.

TABLE 8
Metabolizable energy content, expressed on a dry matter basis (DMB),
of two oligosaccharides fed to conventional and cecectomized roosters.

| | | | Metabolizable energy content, | |
|---|---|---|---|---|
| | | | kcal/g, DMB | |
| | Dry | Gross energy, | Conventional | Cecectomized |
| Substrate | matter, % | kcal/g, DMB | roosters | roosters |
| Sample #1 | 65.49 | 4.53 | 1.72^{B} | 1.39 |
| Sample #2 | 60.24 | 4.32 | 1.17^{A} | 1.19 |
| Standard | — | — | 0.17 | 0.07 |
| Error in the | | | | |
| Mean (SEM) | | | | |
| P-value | — | — | 0.048 | 0.068 |

^{AB}Values in the same column not sharing a common superscript letter are statistically distinct with a significance of p < 0.05.

Dry matter content and gross energy content of Sample #1 were both slightly higher than was the case for Sample #2, a difference of ˜8.4% and 4.7%, respectively. The TMEₙ values for both samples were low, whether evaluated using the conventional rooster or the cecectomized rooster. Sample #1 was observed to have a significantly higher TMEₙ value (P=0.048) than did Sample #2 when evaluated using the conventional roosters (38.1% difference). In the cecectomized roosters, Sample #1 was observed to have a higher TMEₙ value than did Sample #2 (15.5% difference). A significant trend was observed at P=0.07.

When comparing TMEₙ values of Sample #1 using conventional and cecectomized roosters, dosing of the cecectomized roosters with Sample #1 resulted in a 21.2% lower TMEₙ value than was noted for the conventional roosters. This change may be attributed to the relative contribution of the cecal microbiota and their ability to ferment the non-digestible carbohydrate fraction of this oligosaccharide composition. In other words, it is believed that the presence of an active microbiota in the paired ceca of the bird results in 0.33 kcal/g additional energy available to the animal via fermentation processes. However, this was not the case for Sample #2 in that TMEn values using the conventional rooster and the cecectomized rooster were nearly identical (avg., 1.18 kcal/g).

The two oligosaccharides compositions tested in this Example were surprisingly observed to have a lower TMEₙ concentration as compared to other commercially available carbohydrate sources commonly used in the food industry. Such comparison is summarized in Table 9 below. The TMEₙ data for the HCl-treated corn syrup, phosphoric acid-treated corn syrup, and soluble corn fiber are found in the following reference: De Godoy et al., J Anim. Sci. 2014 June; 92(6):2 447-57. The data for Samples #1 and #2 are based on the data in Table 8 above for conventional roosters.

TABLE 9
Metabolizable energy content comparison with commercially
available carbohydrate sources

| Carbohydrate Source | Metabolizable energy content (kcal/g) |
|---|---|
| HCl-trcatcd corn syrup | 1.8 |
| Phosphoric acid-treated corn syrup | 2.3 |
| Soluble corn fiber | 1.5 |
| Sample #1 | 1.72 |
| Sample #2 | 1.17 |

The data in this Example suggest that the two oligosaccharides tested would be suitable for use as low energy substrates having application in food products where lower caloric ingredients are desired.

1-20. (canceled)

21. A method of manufacturing a food product, comprising: combining
(1) a first food ingredient, comprising an oligosaccharide composition, wherein:
(a) the oligosaccharide composition has a glycosidic bond type distribution of:
at least 10 mol % α-(1,3) glycosidic linkages; and
at least 10 mol % β-(1,3) glycosidic linkages; and
(b) at least 10 dry wt % of the oligosaccharide composition has a degree of polymerization of at least 3; and
(c) a metabolizable energy content, on a dry matter basis, of less than 4 kcal/g; and
(2) a second food ingredient.

22. The method of claim 21, wherein the oligosaccharide composition has a glycosidic bond type distribution of less than 9 mol % α-(1,4) glycosidic linkages, and less than 19 mol % α-(1,6) glycosidic linkages.

23. The method of claim 21, wherein the oligosaccharide composition has a glycosidic bond type distribution of at least 15 mol % β-(1,2) glycosidic linkages.

24. The method of claim 21, wherein the oligosaccharide composition has a metabolizable energy content, on a dry matter basis, of less than 2.7 kcal/g.

25. The method of claim 21, wherein the oligosaccharide composition comprises a gluco-oligosaccharide, a galacto-oligosaccharide, a fructo-oligosaccharide, a manno-oligosaccharide, an arabino-oligosaccharide, a xylo-oligosaccharide, a gluco-galacto-oligosaccharide, a gluco-fructo-oligosaccharide, a gluco-manno-oligosaccharide, a gluco-arabino-oligosaccharide, a gluco-xylo-oligosaccharide, a galacto-fructo-oligosaccharide, a galacto-manno-oligosaccharide, a galacto-arabino-oligosaccharide, a galacto-xylo-oligosaccharide, a fructo-manno-oligosaccharide, a fructo-arabino-oligosaccharide, a fructo-xylo-oligosaccharide, a manno-arabino-oligosaccharide, a manno-xylo-oligosaccharide, an arabino-xylo-oligosaccharide, or a xylo-gluco-galacto-oligosaccharide, or any combinations thereof.

26. The method of claim 21, wherein the oligosaccharide composition comprises an oligosaccharide selected from the group consisting of an arabino-oligosaccharide, a xylo-oligosaccharide, and an arabino-xylo-oligosaccharide, or any combinations thereof.

27. The method of claim 21, wherein the oligosaccharide composition has a glycosidic bond type distribution of:
between 0 to 20 mol % α-(1,2) glycosidic linkages;
between 0 to 45 mol % β-(1,2) glycosidic linkages;
between 1 to 30 mol % α-(1,3) glycosidic linkages;
between 1 to 20 mol % β-(1,3) glycosidic linkages;
between 0 to 55 mol % β-(1,4) glycosidic linkages; and
between 10 to 55 mol % β-(1,6) glycosidic linkages

28. The method of claim 21, wherein at least 50 dry wt % of the oligosaccharide composition has a degree of polymerization of at least 3.

29. The method of claim 21, wherein at least 50 dry wt % of the oligosaccharide composition comprises one or more gluco-oligosaccharides, or one or more gluco-galacto-oligosaccharides.

30. The method of claim 21, wherein the oligosaccharide composition has a glycosidic bond type distribution of:
between 0 to 20 mol % α-(1,2) glycosidic linkages;
between 10 to 45 mol % β-(1,2) glycosidic linkages;
between 1 to 30 mol % α-(1,3) glycosidic linkages;
between 1 to 20 mol % β-(1,3) glycosidic linkages;
between 0 to 55 mol % β-(1,4) glycosidic linkages;
between 10 to 55 mol % β-(1,6) glycosidic linkages;
less than 9 mol % α-(1,4) glycosidic linkages; and
less than 19 mol % α-(1,6) glycosidic linkages.

31. The method of claim 21, wherein the oligosaccharide composition has a glycosidic bond type distribution of:
between 0 to 15 mol % α-(1,2) glycosidic linkages;
between 0 to 15 mol % β-(1,2) glycosidic linkages;
between 1 to 20 mol % α-(1,3) glycosidic linkages;
between 1 to 15 mol % β-(1,3) glycosidic linkages;
between 5 to 55 mol % β-(1,4) glycosidic linkages;
between 15 to 55 mol % β-(1,6) glycosidic linkages;
less than 20 mol % α-(1,4) glycosidic linkages; and
less than 30 mol % α-(1,6) glycosidic linkages.

32. The method of claim 21, wherein the oligosaccharide composition is a functionalized oligosaccharide composition.

33. The method of claim 21, wherein the oligosaccharide composition is a syrup or a powder.
